SEQUENCE LISTING (506 sequences)

SEQ ID NO: 1 - protein, 4 aa, synthetic construct
  REGION 1..4: note = Synthetic
YGVS

SEQ ID NO: 2 - protein, 4 aa, synthetic construct
  REGION 1..4: note = Synthetic
YGVT

SEQ ID NO: 3 - protein, 4 aa, synthetic construct
  REGION 1..4: note = Synthetic
YGMS

SEQ ID NO: 4 - protein, 4 aa, synthetic construct
  REGION 1..4: note = Synthetic
YSIL

SEQ ID NO: 5 - protein, 4 aa, synthetic construct
  REGION 1..4: note = Synthetic
YAML

SEQ ID NO: 6 - protein, 4 aa, synthetic construct
  REGION 1..4: note = Synthetic
YAIL

SEQ ID NO: 7 - protein, 4 aa, synthetic construct
  REGION 1..4: note = Synthetic
YSML

SEQ ID NO: 8 - protein, 5 aa, synthetic construct
  REGION 1..5: note = Synthetic
TYWMW

SEQ ID NO: 9 - protein, 5 aa, synthetic construct
  REGION 1..5: note = Synthetic
TYWMC

SEQ ID NO: 10 - protein, 4 aa, synthetic construct
  REGION 1..4: note = Synthetic
YAMT

SEQ ID NO: 11 - protein, 4 aa, synthetic construct
  REGION 1..4: note = Synthetic
YAVT

SEQ ID NO: 12 - protein, 4 aa, synthetic construct
  REGION 1..4: note = Synthetic
NAMT

SEQ ID NO: 13 - protein, 17 aa, synthetic construct
  REGION 1..17: note = Synthetic
YIYPAYHNTYYATWVNG

SEQ ID NO: 14 - protein, 17 aa, synthetic construct
  REGION 1..17: note = Synthetic
YIYPAFHNTYYATWVNG

SEQ ID NO: 15 - protein, 17 aa, synthetic construct
  REGION 1..17: note = Synthetic
YIYPAFHNRYYANWVNG

SEQ ID NO: 16 - protein, 17 aa, synthetic construct
  REGION 1..17: note = Synthetic
YIYPTYHNTYYATWVNG

SEQ ID NO: 17 - protein, 17 aa, synthetic construct
  REGION 1..17: note = Synthetic
YIYPAFHNAYYANWVNG

SEQ ID NO: 18 - protein, 17 aa, synthetic construct
  REGION 1..17: note = Synthetic
YIYPAFHNTYYASWVNG

SEQ ID NO: 19 - protein, 17 aa, synthetic construct
  REGION 1..17: note = Synthetic
YIYPAFPNTYYATWVNG

SEQ ID NO: 20 - protein, 17 aa, synthetic construct
  REGION 1..17: note = Synthetic
YIYPAFHNTYYANWVNG

SEQ ID NO: 21 - protein, 17 aa, synthetic construct
  REGION 1..17: note = Synthetic
CIYISGGSTYYANWAKG

SEQ ID NO: 22 - protein, 17 aa, synthetic construct
  REGION 1..17: note = Synthetic
SIYISGGSTYYANWAKG

SEQ ID NO: 23 - protein, 17 aa, synthetic construct
  REGION 1..17: note = Synthetic
CIYISGGTTYFASWATG

SEQ ID NO: 24 - protein, 17 aa, synthetic construct
  REGION 1..17: note = Synthetic
SIYISGGTTYFASWATG

SEQ ID NO: 25 - protein, 17 aa, synthetic construct
  REGION 1..17: note = Synthetic
CIYISGATTYFANWATG

SEQ ID NO: 26 - protein, 17 aa, synthetic construct
  REGION 1..17: note = Synthetic
CIYISGGTTYYASWAKG

SEQ ID NO: 27 - protein, 17 aa, synthetic construct
  REGION 1..17: note = Synthetic
CIYISGGSTYYASWAKG

SEQ ID NO: 28 - protein, 17 aa, synthetic construct
  REGION 1..17: note = Synthetic
CIYISGGTTYYANWAKG

SEQ ID NO: 29 - protein, 17 aa, synthetic construct
  REGION 1..17: note = Synthetic
CIYFSGGSTYYANWAKG

SEQ ID NO: 30 - protein, 17 aa, synthetic construct
  REGION 1..17: note = Synthetic
CIYISGGTTYFANWAKG

SEQ ID NO: 31 - protein, 18 aa, synthetic construct
  REGION 1..18: note = Synthetic
CIYVGSGSSTYYASWAKG

SEQ ID NO: 32 - protein, 18 aa, synthetic construct
  REGION 1..18: note = Synthetic
PIYVGSGSSTYYASWAKG

SEQ ID NO: 33 - protein, 18 aa, synthetic construct
  REGION 1..18: note = Synthetic
SIYVGSGSSTYYASWAKG

SEQ ID NO: 34 - protein, 18 aa, synthetic construct
  REGION 1..18: note = Synthetic
CIYVGSGGSTYYASWAKG

SEQ ID NO: 35 - protein, 16 aa, synthetic construct
  REGION 1..16: note = Synthetic
FVALRGNIYYANWAKG

SEQ ID NO: 36 - protein, 16 aa, synthetic construct
  REGION 1..16: note = Synthetic
FIGIRGHIYYANWAKG

SEQ ID NO: 37 - protein, 16 aa, synthetic construct
  REGION 1..16: note = Synthetic
FIGIRGNIYYANWAKG

SEQ ID NO: 38 - protein, 16 aa, synthetic construct
  REGION 1..16: note = Synthetic
FVGIYGDFYYANWAKG

SEQ ID NO: 39 - protein, 11 aa, synthetic construct
  REGION 1..11: note = Synthetic
DTGSTDYNFNL

SEQ ID NO: 40 - protein, 11 aa, synthetic construct
  REGION 1..11: note = Synthetic
DTGTTDYNFNL

SEQ ID NO: 41 - protein, 11 aa, synthetic construct
  REGION 1..11: note = Synthetic
DAGTTDYNFNL

SEQ ID NO: 42 - protein, 11 aa, synthetic construct
  REGION 1..11: note = Synthetic
DAGSTDYKFNL

SEQ ID NO: 43 - protein, 11 aa, synthetic construct
  REGION 1..11: note = Synthetic
DAGGTDYNFNL

SEQ ID NO: 44 - protein, 11 aa, synthetic construct
  REGION 1..11: note = Synthetic
DAGNTDYNFNL

SEQ ID NO: 45 - protein, 11 aa, synthetic construct
  REGION 1..11: note = Synthetic
DAGSTDYNLNL

SEQ ID NO: 46 - protein, 11 aa, synthetic construct
  REGION 1..11: note = Synthetic
DAGGTDYNYNL

SEQ ID NO: 47 - protein, 11 aa, synthetic construct
  REGION 1..11: note = Synthetic
DAGGTDYAYNL

SEQ ID NO: 48 - protein, 11 aa, synthetic construct
  REGION 1..11: note = Synthetic
DAGSTDYYFNL

SEQ ID NO: 49 - protein, 11 aa, synthetic construct
  REGION 1..11: note = Synthetic
DAGSVDYNFNL

SEQ ID NO: 50 - protein, 11 aa, synthetic construct
  REGION 1..11: note = Synthetic
DAGSTDYNFNL

SEQ ID NO: 51 - protein, 11 aa, synthetic construct
  REGION 1..11: note = Synthetic
DDGTASYYLNL

SEQ ID NO: 52 - protein, 11 aa, synthetic construct
  REGION 1..11: note = Synthetic
DDGTTSYYLNL

SEQ ID NO: 53 - protein, 11 aa, synthetic construct
  REGION 1..11: note = Synthetic
NDGTTSYYLNL

SEQ ID NO: 54 - protein, 11 aa, synthetic construct
  REGION 1..11: note = Synthetic
DDGSTSYYLNL

SEQ ID NO: 55 - protein, 11 aa, synthetic construct
  REGION 1..11: note = Synthetic
NDGSTNYYLNL

SEQ ID NO: 56 - protein, 11 aa, synthetic construct
  REGION 1..11: note = Synthetic
DDGSVSYYLNL

SEQ ID NO: 57 - protein, 13 aa, synthetic construct
  REGION 1..13: note = Synthetic
GATNNVFMNYFNL

SEQ ID NO: 58 - protein, 13 aa, synthetic construct
  REGION 1..13: note = Synthetic
GATNNVFRNYFNL

SEQ ID NO: 59 - protein, 12 aa, synthetic construct
  REGION 1..12: note = Synthetic
GGLYTGYSYFDL

SEQ ID NO: 60 - protein, 12 aa, synthetic construct
  REGION 1..12: note = Synthetic
GGLWTGNSYFDL

SEQ ID NO: 61 - protein, 11 aa, synthetic construct
  REGION 1..11: note = Synthetic
QASEDIESYLA

SEQ ID NO: 62 - protein, 11 aa, synthetic construct
  REGION 1..11: note = Synthetic
QASQSIYSYLS

SEQ ID NO: 63 - protein, 11 aa, synthetic construct
  REGION 1..11: note = Synthetic
QASQSISSYLA

SEQ ID NO: 64 - protein, 11 aa, synthetic construct
  REGION 1..11: note = Synthetic
QASQSIESYLA

SEQ ID NO: 65 - protein, 11 aa, synthetic construct
  REGION 1..11: note = Synthetic
QASEDIENYLA

SEQ ID NO: 66 - protein, 11 aa, synthetic construct
  REGION 1..11: note = Synthetic
QASEDIESYSA

SEQ ID NO: 67 - protein, 11 aa, synthetic construct
  REGION 1..11: note = Synthetic
QASENIESYLA

SEQ ID NO: 68 - protein, 11 aa, synthetic construct
  REGION 1..11: note = Synthetic
QASKNIDSNLA

SEQ ID NO: 69 - protein, 11 aa, synthetic construct
  REGION 1..11: note = Synthetic
QASEDIERYLA

SEQ ID NO: 70 - protein, 11 aa, synthetic construct
  REGION 1..11: note = Synthetic
QASEDIYKLLA

SEQ ID NO: 71 - protein, 11 aa, synthetic construct
  REGION 1..11: note = Synthetic
QASEDIYSLLA

SEQ ID NO: 72 - protein, 11 aa, synthetic construct
  REGION 1..11: note = Synthetic
QASEDIYRLLA

SEQ ID NO: 73 - protein, 11 aa, synthetic construct
  REGION 1..11: note = Synthetic
QASQSIYNNFA

SEQ ID NO: 74 - protein, 11 aa, synthetic construct
  REGION 1..11: note = Synthetic
QASEDIYNLLA

SEQ ID NO: 75 - protein, 11 aa, synthetic construct
  REGION 1..11: note = Synthetic
QASEDISSNLG

SEQ ID NO: 76 - protein, 11 aa, synthetic construct
  REGION 1..11: note = Synthetic
QASEDISSNLA

SEQ ID NO: 77 - protein, 11 aa, synthetic construct
  REGION 1..11: note = Synthetic
QASESLSSYLA

SEQ ID NO: 78 - protein, 11 aa, synthetic construct
  REGION 1..11: note = Synthetic
QASESISRYLA

SEQ ID NO: 79 - protein, 11 aa, synthetic construct
  REGION 1..11: note = Synthetic
QASENINRYLA

SEQ ID NO: 80 - protein, 11 aa, synthetic construct
  REGION 1..11: note = Synthetic
QASESINRYLA

SEQ ID NO: 81 - protein, 7 aa, synthetic construct
  REGION 1..7: note = Synthetic
EASKLAS

SEQ ID NO: 82 - protein, 7 aa, synthetic construct
  REGION 1..7: note = Synthetic
EATKLTS

SEQ ID NO: 83 - protein, 7 aa, synthetic construct
  REGION 1..7: note = Synthetic
DASTLAS

SEQ ID NO: 84 - protein, 7 aa, synthetic construct
  REGION 1..7: note = Synthetic
KASTLAS

SEQ ID NO: 85 - protein, 7 aa, synthetic construct
  REGION 1..7: note = Synthetic
AASTLAS

SEQ ID NO: 86 - protein, 7 aa, synthetic construct
  REGION 1..7: note = Synthetic
EASKLPS

SEQ ID NO: 87 - protein, 7 aa, synthetic construct
  REGION 1..7: note = Synthetic
AASDLES

SEQ ID NO: 88 - protein, 7 aa, synthetic construct
  REGION 1..7: note = Synthetic
GASNLES

SEQ ID NO: 89 - protein, 7 aa, synthetic construct
  REGION 1..7: note = Synthetic
DASDLAS

SEQ ID NO: 90 - protein, 7 aa, synthetic construct
  REGION 1..7: note = Synthetic
DASTLTS

SEQ ID NO: 91 - protein, 7 aa, synthetic construct
  REGION 1..7: note = Synthetic
GASTLAS

SEQ ID NO: 92 - protein, 7 aa, synthetic construct
  REGION 1..7: note = Synthetic
RAATLAS

SEQ ID NO: 93 - protein, 7 aa, synthetic construct
  REGION 1..7: note = Synthetic
RASTLAS

SEQ ID NO: 94 - protein, 12 aa, synthetic construct
  REGION 1..12: note = Synthetic
QQALTVGNVDNP

SEQ ID NO: 95 - protein, 12 aa, synthetic construct
  REGION 1..12: note = Synthetic
QQALTVGDVDNP

SEQ ID NO: 96 - protein, 12 aa, synthetic construct
  REGION 1..12: note = Synthetic
QQDLTVGNVDNP

SEQ ID NO: 97 - protein, 12 aa, synthetic construct
  REGION 1..12: note = Synthetic
QQALTIGNVDNP

SEQ ID NO: 98 - protein, 12 aa, synthetic construct
  REGION 1..12: note = Synthetic
HQALTIGNVDNP

SEQ ID NO: 99 - protein, 12 aa, synthetic construct
  REGION 1..12: note = Synthetic
QQVLTIGNVDNP

SEQ ID NO: 100 - protein, 12 aa, synthetic construct
  REGION 1..12: note = Synthetic
QQGLTIGNVDNP

SEQ ID NO: 101 - protein, 12 aa, synthetic construct
  REGION 1..12: note = Synthetic
QQALTIGYVDNP

SEQ ID NO: 102 - protein, 12 aa, synthetic construct
  REGION 1..12: note = Synthetic
QQAYTIGNIDNS

SEQ ID NO: 103 - protein, 12 aa, synthetic construct
  REGION 1..12: note = Synthetic
QQAYTIGNIDNA

SEQ ID NO: 104 - protein, 12 aa, synthetic construct
  REGION 1..12: note = Synthetic
QQAYTIGNIDNT

SEQ ID NO: 105 - protein, 12 aa, synthetic construct
  REGION 1..12: note = Synthetic
QQAYTIGNIDNP

SEQ ID NO: 106 - protein, 12 aa, synthetic construct
  REGION 1..12: note = Synthetic
QQGYTIGNIDNS

SEQ ID NO: 107 - protein, 12 aa, synthetic construct
  REGION 1..12: note = Synthetic
QQGYTIGNIDNA

SEQ ID NO: 108 - protein, 12 aa, synthetic construct
  REGION 1..12: note = Synthetic
QQAYTVGNIDNA

SEQ ID NO: 109 - protein, 12 aa, synthetic construct
  REGION 1..12: note = Synthetic
QQGYTIGNIDNT

SEQ ID NO: 110 - protein, 12 aa, synthetic construct
  REGION 1..12: note = Synthetic
QTSYYIDDGVNG

SEQ ID NO: 111 - protein, 12 aa, synthetic construct
  REGION 1..12: note = Synthetic
QSSYYIDDGVNG

SEQ ID NO: 112 - protein, 12 aa, synthetic construct
  REGION 1..12: note = Synthetic
QQGYGYSTVGNA

SEQ ID NO: 113 - protein, 120 aa, synthetic construct
  REGION 1..120: note = Synthetic
QEQLKESGGGLVQPGGSLTLSCKASGFDFNNYGVSWVRQAPGKGLEWIGYIYPAYHNTYYATWVNGRFTISSHNAQNTLYLQLNTLTAADTATYFCARDTGSTDYNFNLWGPGTLVTVSS

SEQ ID NO: 114 - protein, 120 aa, synthetic construct
  REGION 1..120: note = Synthetic
QEQLKESGGGLGQPGGSLTLSCKASGFDFNNYGVSWVRQAPGKGLEWIGYIYPAYHNTYYATWVNGRFTISSHNAQNTLYLQLNTLTAADTATYFCARDTGTTDYNFNLWGPGTLVTVSS

SEQ ID NO: 115 - protein, 120 aa, synthetic construct
  REGION 1..120: note = Synthetic
QEQLKESGGGLVQPGGSLKLSCKASGFDFNKYGVSWVRQAPGKGLEWIAYIYPAFHNTYYATWVNGRFTISSHNAQNTLYLQLNTLTAADTATYFCARDAGTTDYNFNLWGPGTLVTVSS

SEQ ID NO: 116 - protein, 120 aa, synthetic construct
  REGION 1..120: note = Synthetic
QEQLKESGGGLVQPGGSLKLSCKASGFDFNAYGVSWVRQAPGKGLEWIAYIYPAFHNTYYATWVNGRFTISSHNAQNTLYLQLNTLTAADTATYFCARDAGTTDYNFNLWGPGTLVTVSS

SEQ ID NO: 117 - protein, 120 aa, synthetic construct
  REGION 1..120: note = Synthetic
QEQLKESGGGLVQPGGSLKLSCKASGFDFNNYGVSWVRQAPGKGLEWIAYIYPAFHNRYYANWVNGRFTISSDNAQNTVYLQLNSLTAADTATYFCARDAGSTDYKFNLWGPGTLVTVSS

SEQ ID NO: 118 - protein, 120 aa, synthetic construct
  REGION 1..120: note = Synthetic
QEQLKESGGGLVQPGGSLTLSCKASGFDFNNYGVSWVRQAPGKGLEWIAYIYPTYHNTYYATWVNGRFTISSHNAQNTLYLQLNTLTAADTATYFCARDAGGTDYNFNLWGPGTLVTVSS

SEQ ID NO: 119 - protein, 120 aa, synthetic construct
  REGION 1..120: note = Synthetic
QEQLKESGGGLVQPGGSLTLSCKASGFDFNNYGVSWVRQAPGKGLEWIGYIYPAYHNTYYATWVNGRFTISSHNAQNTLYLQLNTLTAADTATYFCARDAGNTDYNFNLWGPGTLVTVSS

SEQ ID NO: 120 - protein, 120 aa, synthetic construct
  REGION 1..120: note = Synthetic
QEQLKESGGGLVQPGGSLTLSCKASGFDFNYYGVSWVRQAPGKGLEWIGYIYPAYHNTYYATWVNGRFTISSHNAQNTLYLQLNTLTAADTATYFCARDAGSTDYNLNLWGPGTLVTVSS

SEQ ID NO: 121 - protein, 120 aa, synthetic construct
  REGION 1..120: note = Synthetic
QEQLKESGGGLVQPGGSLKLSCKASGFDFNEYGVSWVRQAPGKGLEWIAYIYPAFHNTYYATWVNGRFTISSHNAQNTLYLQLNTLTAADTATYFCARDAGTTDYNFNLWGPGTLVTVSS

SEQ ID NO: 122 - protein, 120 aa, synthetic construct
  REGION 1..120: note = Synthetic
QEQLVESGGGLVQPGGSLTLSCKGSGFDFNEYGVSWVRQAPGKGLEWIAYIYPAFHNTYYATWVNGRFTISSHNAQNTLYLQLHTLTAADTATYFCARDAGTTDYNFNLWGPGTLVTVSS

SEQ ID NO: 123 - protein, 120 aa, synthetic construct
  REGION 1..120: note = Synthetic
QEQLKESGGGLVQPGGSLKLSCKASGFDFNSYGVSWVRQAPGKGLEWIAYIYPAFHNAYYANWVNGRFTISSDNAQNTVDLQLNSLTAADTATYFCARDAGGTDYNYNLWGPGTLVTVSS

SEQ ID NO: 124 - protein, 120 aa, synthetic construct
  REGION 1..120: note = Synthetic
QEQLKESGGGLVQPGGSLKLSCKASGFDFNNYGVSWVRQAPGKGLEWIAYIYPAFHNTYYASWVNGRFTISSHNAQNTLYLQLNSLTAADTATYFCARDAGGTDYAYNLWGPGTLVTVSS

SEQ ID NO: 125 - protein, 120 aa, synthetic construct
  REGION 1..120: note = Synthetic
QEQLKESGGGLVRLGDPLQLSCKASGFDFNNYGVTWVRQAPGKGLEWIAYIYPAFPNTYYATWVNGRFTISPHNAQNTLYLQLNSLTAADTATYFCARDAGSTDYYFNLWGPGTLATVSS

SEQ ID NO: 126 - protein, 120 aa, synthetic construct
  REGION 1..120: note = Synthetic
QEQLKESGGGLVQPGGSLKLSCKASGFDFNNYGVSWVRQAPGKGLEWIAYIYPAFHNTYYATWVNGRFTISSHNAQNTLYLQLNSLTAADTATYFCARDAGSVDYNFNLWGPGTLVTVSS

SEQ ID NO: 127 - protein, 120 aa, synthetic construct
  REGION 1..120: note = Synthetic
QEQLKESGGGLVQPGGSLKLSCKASGFDFNNYGMSWVRQAPGKGLEWIAYIYPAFHNTYYANWVNGRFTISSDNAQNTVDLQLNSLTAADTATYFCARDAGSTDYNFNLWGPGTLVTVSS

SEQ ID NO: 128 - protein, 120 aa, synthetic construct; identical to SEQ ID NO: 127
  REGION 1..120: note = Synthetic

SEQ ID NO: 129 - protein, 118 aa, synthetic construct
  REGION 1..118: note = Synthetic
QSLEESGGDLVQPGASLTLTCKASGFDLNNYSILWVRQAPGKGLEWIGCIYISGGSTYYANWAKGRFAISKTSSTTVTLQMTSLTAADTATYFCARDDGTASYYLNLWGPGTLVTVSS

SEQ ID NO: 130 - protein, 118 aa, synthetic construct
  REGION 1..118: note = Synthetic
QSLEESGGDLVQPGASLTLTCKASGFDLNNYSILWVRQAPGKGLEWIGSIYISGGSTYYANWAKGRFAISKTSSTTVTLQMTSLTAADTATYFCARDDGTASYYLNLWGPGTLVTVSS

SEQ ID NO: 131 - protein, 118 aa, synthetic construct
  REGION 1..118: note = Synthetic
QSLEESGGDLVQPGASLTLTCKASGFDLNNYAMLWVRQAPGKGLEWIGCIYISGGTTYFASWATGRFAISKTSSTTVTLQMTSLTAADTATYFCARDDGTTSYYLNLWGPGTLVTVSS

SEQ ID NO: 132 - protein, 118 aa, synthetic construct
  REGION 1..118: note = Synthetic
QSLEESGGDLVQPGASLTLTCKASGFDLNNYAMLWVRQAPGKGLEWIGSIYISGGTTYFASWATGRFAISKTSSTTVTLQMTSLTAADTATYFCARDDGTTSYYLNLWGPGTLVTVSS

SEQ ID NO: 133 - protein, 118 aa, synthetic construct
  REGION 1..118: note = Synthetic
QSLEESGGDLVQPGASLTLTCKASGFDLNNYAMLWVRQAPGKGLEWIGCIYISGGTTYFASWATGRFAISKTSSTTLTLQMTTLPPPDTATYFCAKNDGTTSYYLNLWGPGTLVTVSS

SEQ ID NO: 134 - protein, 118 aa, synthetic construct
  REGION 1..118: note = Synthetic
QSLEESGGDLVQPGASLTLTCKASGFDLNNYAILWVRQAPGKGLEWIGCIYISGATTYFANWATGRFAISKTSSTTVTLQMTSLTAADTATYFCARDDGTTSYYLNLWGPGTLVTVSS

SEQ ID NO: 135 - protein, 118 aa, synthetic construct; identical to SEQ ID NO: 131
  REGION 1..118: note = Synthetic

SEQ ID NO: 136 - protein, 118 aa, synthetic construct; identical to SEQ ID NO: 131
  REGION 1..118: note = Synthetic

SEQ ID NO: 137 - protein, 118 aa, synthetic construct
  REGION 1..118: note = Synthetic
QSLEESGGDLVQPGASLTLTCKASGFDLNNYAMLWVRQAPGKGLEWIGCIYISGGTTYFASWATGRFAISKTSSTTVTLQMTSLTAADTATYFCARDDGSTSYYLNLWGPGTLVTVSS

SEQ ID NO: 138 - protein, 118 aa, synthetic construct
  REGION 1..118: note = Synthetic
QSLEESGGDLVQPGASLTLTCKASGFDLNNYAMLWVRQAPGKGLEWIGCIYISGGTTYFASWATGRFAISRTSSTTVTLQMTSLTAADTATYFCARDDGSTSYYLNLWGPGTLVTVSS

SEQ ID NO: 139 - protein, 118 aa, synthetic construct
  REGION 1..118: note = Synthetic
QSLEESGGNLAHPGASLRLTCTASGFYLNNYAMLWVRQAPGKGLEWIGCLYISGGTTYFASWATGRFAISKTSSTTGTLQMTSLTAADTATYFCARNDGSTNYYLNLWGPGTLVTISS

SEQ ID NO: 140 - protein, 118 aa, synthetic construct
  REGION 1..118: note = Synthetic
QSLEESGGDLVQPGASLTLTCRASGFDLNNYSMLWVRQAPGKGLEWIGCIYISGGTTYFASWATGRFAISKTSSTTVTLQMTSLTAADTATYFCARDDGSTSYYLNLWGPGTLVTVSS

SEQ ID NO: 141 - protein, 118 aa, synthetic construct
  REGION 1..118: note = Synthetic
QSLEESGGDLVQPGASLTLTCRASGFDLNNYSMLWVRQAPGKGLEWIGCIYISGGTTYFASWATGRFAISKTSSTTVTLQMTILTAADTATYFCARDDGSTSYYLNLWGPGTLVTVSS

SEQ ID NO: 142 - protein, 118 aa, synthetic construct; identical to SEQ ID NO: 141
  REGION 1..118: note = Synthetic

SEQ ID NO: 143 - protein, 118 aa, synthetic construct
  REGION 1..118: note = Synthetic
QSLEESGGDLVQPGASLTLTCKASGFDLNNYSILWVRQAPGKGLEWIGCIYISGGTTYFASWATGRFAISKTSSTTVTLQMTSLTAADTATYFCARDDGTTSYYLNLWGPGTLVTVSS

SEQ ID NO: 144 - protein, 118 aa, synthetic construct
  REGION 1..118: note = Synthetic
QSLEESGGDLVQPGASLTLTCKAAGFDLNNYSMLWVRQAPGKGLEWIGCIYISGGTTYYASWAKGRFTISKTSSTTVTLQMTSLTAADTATYFCARDDGSTSYYLNLWGPGTLVTVSS

SEQ ID NO: 145 - protein, 118 aa, synthetic construct
  REGION 1..118: note = Synthetic
QSLEESGGDLVQPGVSLTLTCKASGFDLNNYSILWVRQAPGKGLEWIGCIYISGGSTYYASWAKGRFAISKTSSTTVTLQMTSLTAADTATYFCARDDGTTSYYLNLWGPGTLVTVSS

SEQ ID NO: 146 - protein, 118 aa, synthetic construct
  REGION 1..118: note = Synthetic
QSLEESGGDLVQPGASLTLTCKASGFDLNNYSILWVRQAPGKGLEWIGCIYISGGTTYYANWAKGRFTISRTSSTTVTLQMTSLTGADTATYFCARDDGSTSYYLNLWGPGTLVTVSS

SEQ ID NO: 147 - protein, 118 aa, synthetic construct
  REGION 1..118: note = Synthetic
QSLEESGGDLVQPGASLTLTCKASGFDLNNYSILWVRQAPGKGLEWIGCIYISGGTTYYANWAKGRFAISKTSSTTVTLQMTSLTAADTATYFCARDDGTTSYYLNLWGPGTLVTVSS

SEQ ID NO: 148 - protein, 118 aa, synthetic construct
  REGION 1..118: note = Synthetic
QSLEESGGDLVQPGASLTLTCKASGFDLNNYSILWVRQAPGKGLEWIGCIYISGGSTYYANWAKGRFAISKTSSTTVTLQMTSLTAADTATYFCARDDGTTSYYLNLWGPGTLVTVSS

SEQ ID NO: 149 - protein, 118 aa, synthetic construct
  REGION 1..118: note = Synthetic
QSLEESGGDLVQPGASLTLTCKASGFDLNNYSILWVRQAPGKGLEWIGCIYISGGSTYYANWAKGRFAVSKTSSTTVTLQMTSLTAADTATYFCARDDGTTSYYLNLWGPGTLVTVSS

SEQ ID NO: 150 - protein, 118 aa, synthetic construct
  REGION 1..118: note = Synthetic
QSLEESGGDLVQPGASLTLTCKASGFDLNNYSILWVRQAPGKGLEWIGCIYFSGGSTYYANWAKGRFAISKTSSTTVTLQMTSLTAADTATYFCARDDGTTSYYLNLWGPGTLVTVSS

SEQ ID NO: 151 - protein, 118 aa, synthetic construct; identical to SEQ ID NO: 129
  REGION 1..118: note = Synthetic

SEQ ID NO: 152 - protein, 118 aa, synthetic construct
  REGION 1..118: note = Synthetic
QSLEESGGDLVQPGTSLTLSCKASGFDLNNYSILWVRQAPGKGLEWIGCIYISGGTTYFANWAKGRFTISRPSSTTVTLQMTSLTGADTATYFCARDDGSVSYYLNLWGPGTLVIVSS

SEQ ID NO: 153 - protein, 122 aa, synthetic construct
  REGION 1..122: note = Synthetic
QQLEESGGGLVKPEGSLTLTCKGSGFDFSGTYWMWWVRQAPGKGLEWIACIYVGSGSSTYYASWAKGRFTISSTSSTAVTLQATSLTAADTATYFCARGATNNVFMNYFNLWGPGTLVTVSS

SEQ ID NO: 154 - protein, 122 aa, synthetic construct
  REGION 1..122: note = Synthetic
QQLEESGGGLVKPEGSLTLTCKGSGFDFSGTYWMWWVRQAPGKGLEWIAPIYVGSGSSTYYASWAKGRFTISSTSSTAVTLQATSLTAADTATYFCARGATNNVFMNYFNLWGPGTLVTVSS

SEQ ID NO: 155 - protein, 122 aa, synthetic construct
  REGION 1..122: note = Synthetic
QQLEESGGGLVKPEGSLTLTCKGSGFDFSGTYWMWWVRQAPGKGLEWIASIYVGSGSSTYYASWAKGRFTISSTSSTAVTLQATSLTAADTATYFCARGATNNVFMNYFNLWGPGTLVTVSS

SEQ ID NO: 156 - protein, 122 aa, synthetic construct
  REGION 1..122: note = Synthetic
QQLEESGGGLVKPEGSLTLTCKGSGFDFSGTYWMCWVRQAPGKGLEWIACIYVGSGGSTYYASWAKGRFTISSTSSTAVTLQVTSLTAADTATYFCARGATNNVFRNYFNLWGPGTLVTVSS

SEQ ID NO: 157 - protein, 118 aa, synthetic construct
  REGION 1..118: note = Synthetic
QSLEESGGGLVKPGGTLTLTCTVSGFDFSNYAMTWVRQAPGEGLEYIGFVALRGNIYYANWAKGRFTISKTSSTTVTLQMTSLTVADTATYFCARGGLYTGYSYFDLGGPGTLATVSS

SEQ ID NO: 158 - protein, 118 aa, synthetic construct
  REGION 1..118: note = Synthetic
QSLVESGGGLVQPEGSLTLTCKASGFDFNNYAVTWVRQAPGEGLEYIGFIGIRGHIYYANWAKGRFTISKTSSTTMTLQMTSLTVADTATYFCARGGLWTGNSYFDLWGPGTLVTVSS

SEQ ID NO: 159 - protein, 118 aa, synthetic construct
  REGION 1..118: note = Synthetic
QSLEESGGGLVKPGGTLTLTCTVSGFDFSNYAMTWVRQAPGKGLEYIGFIGIRGNIYYANWAKGRFTISKTSSTTVTLQMTSLTVADTATYFCARGGLWTGNSYFDLWGPGTLVTVSS

SEQ ID NO: 160 - protein, 118 aa, synthetic construct
  REGION 1..118: note = Synthetic
QSLEESGGGLVQPGASLTLTCTVSGFDFSSNAMTWVRQAPGEGLEYIGFIGIRGNIYYANWAKGRFTISKTSSTTVTLQMTSLTVADTATYFCARGGLWTGNSYFDLWGPGTLVTVSS

SEQ ID NO: 161 - protein, 118 aa, synthetic construct
  REGION 1..118: note = Synthetic
QSLEESGGGLVKPGGTLTLTCTVSGFDFSNYAVTWVRQAPGEGLEYIGFVGIYGDFYYANWAKGRFTISKTSSTTVTLQMPSLTVADTATYFCARGGLWTGNSYFDLWGPGTLVTVSS

SEQ ID NO: 162 - protein, 110 aa, synthetic construct
  REGION 1..110: note = Synthetic
DVVMTQTPASVEVAVGGTVTIKCQASEDIESYLAWYQQKPGQPPKLLIYEASKLASGVSSRFSGGGYGTEFTLTISGVECADAATYYCQQALTVGNVDNPFGGGSEVVVK

SEQ ID NO: 163 - protein, 110 aa, synthetic construct; identical to SEQ ID NO: 162
  REGION 1..110: note = Synthetic

SEQ ID NO: 164 - protein, 110 aa, synthetic construct
  REGION 1..110: note = Synthetic
DVVMTQTASPVSAAVGGTVTIKCQASQSIYSYLSWYQQKPGQPPKLLIYEASKLASGVSSRFSGSGYGTEFTLTISGVECADAATYYCQQALTVGNVDNPFGGGSEVVVK

SEQ ID NO: 165 - protein, 110 aa, synthetic construct
  REGION 1..110: note = Synthetic
DIVMTQTPASVSEPVGGTVTIKCQASQSISSYLAWYQQKPGQPPKLLIYEATKLTSGVSSRFSGSGYGTEFTLTISGVECADAATYYCQQALTVGDVDNPFGGGSEVVVK

SEQ ID NO: 166 - protein, 110 aa, synthetic construct
  REGION 1..110: note = Synthetic
DVVMTQTPASVSAAVGGTVTIKCQASEDIESYLAWYQQKPGQPPKLLIYDASTLASGVSSRFSGSGYGTEFTLTISGVECADAATYYCQQDLTVGNVDNPFGGGSEVVVE

SEQ ID NO: 167 - protein, 110 aa, synthetic construct
  REGION 1..110: note = Synthetic
DVVMTQTPASVEAAVGGTVTINCQASQSIESYLAWYQQKPGQPPKLLIYEASKLASGVSSRFSGGGYGTEFTLTISGVECADAATYYCQQALTIGNVDNPFGGGSEVVVK

SEQ ID NO: 168 - protein, 110 aa, synthetic construct
  REGION 1..110: note = Synthetic
DVVMTQTPASVEVAVGGTVTIKCQASEDIESYLAWYQQKPGQPPKLLIYEASKLASGVSSRFSGGGYGTEFTLTISGVECADAATYYCQQALTIGNVDNPFGGGSEVVVK

SEQ ID NO: 169 - protein, 110 aa, synthetic construct
  REGION 1..110: note = Synthetic
DVVMTQTPASVEVAVGGTVTIKCQASEDIESYLAWYQQKPGQPPRLLIYEASKLASGVSSRFSGGGYGTDFTLTISGVECADAATYYCHQALTIGNVDNPFGGGGEVVVK

SEQ ID NO: 170 - protein, 110 aa, synthetic construct
  REGION 1..110: note = Synthetic
DVVMTQTASPVSAAVGGTVTIKCQASEDIESYLAWYQQKPGQPPKLLIYEASKLASGVSSRFSGSGYGTEFTLTISGVECADAATYYCQQALTIGNVDNPFGGGSEVVVK

SEQ ID NO: 171 - protein, 110 aa, synthetic construct
  REGION 1..110: note = Synthetic
DVVMTQTPASVSAAVGGTVSINCQASEDIENYLAWYQQKPGQPPKLLIYEASKLASGVSSRFSGSGYGTEFTLTISGVECADAATYYCQQALTIGNVDNPFGGGSEVVVK

SEQ ID NO: 172 - protein, 110 aa, synthetic construct
  REGION 1..110: note = Synthetic
DVVMTQTPASVEVAVGGTVTLKCQASEDIESYSAWYQQKPGQPPNLLIYEASKLASGVSSRFSGSGYGTEFTLTISGVECADAATYYCQQALTIGNVDNPFGGGSEVVVR

SEQ ID NO: 173 - protein, 110 aa, synthetic construct
  REGION 1..110: note = Synthetic
DVVMTQTPASVEAAVGGTVSINCQASEDIESYLAWYQQKPGQPPKLLIYKASTLASGVSSRFKGSGSGKQFTLTISGVECADAATYYCQQVLTIGNVDNPFGGGSEVVVK

SEQ ID NO: 174 - protein, 110 aa, synthetic construct
  REGION 1..110: note = Synthetic
DVVMTQTPASVEVAVGGTVTIKCQASENIESYLAWYQQKPGQPPKLLIYDASTLASGVSSRFSGSGYGTEFTLTISGVECADAATYYCQQGLTIGNVDNPFGGGSEVVVK

SEQ ID NO: 175 - protein, 110 aa, synthetic construct
  REGION 1..110: note = Synthetic
DVVMTQTPASVEVAVGGTVTIKCQASKNIDSNLAWYQQKPGQPPKQLIYAASTLASGVSSRFSGSGYGAEFTLTISGVECADAATYYCQQALTIGNVDNPFGGGSEVVVK

SEQ ID NO: 176 - protein, 110 aa, synthetic construct
  REGION 1..110: note = Synthetic
DVVMTQTPASVEAAVGGTVTIKCQASEDIERYLAWYQQKPGQPPKLLIYEASKLPSGVSSRFSGSGYGTEFTLTISGVECADAATYYCQQALTIGYVDNPFGGGSEVVVK

SEQ ID NO: 177 - protein, 110 aa, synthetic construct; identical to SEQ ID NO: 176
  REGION 1..110: note = Synthetic

SEQ ID NO: 178 - protein, 109 aa, synthetic construct
  REGION 1..109: note = Synthetic
YDMTQTPASVSEPVGGTVTIKCQASEDIYKLLAWYQQKPGQPPKLLIYAASDLESGVPSRFKGRGSGTDYTLTISDLECADAATYYCQQAYTIGNIDNSFGGGTEVVVK

SEQ ID NO: 179 - protein, 109 aa, synthetic construct
  REGION 1..109: note = Synthetic
YDMTQTPASVSEPVGGTVTIKCQASEDIYKLLAWYQQKPGQPPKLLIYAASDLESGVPSRFKGRGSGTDYTLTISDLEAADAATYYCQQAYTIGNIDNSFGGGTEVVVK

SEQ ID NO: 180 - protein, 109 aa, synthetic construct
  REGION 1..109: note = Synthetic
YDMTQTPASVSAAVGGTVTIKCQASEDIYSLLAWYQQKPGQPPKLLIYGASNLESGVPSRFKGSGSGTEYTLTISDLECDDAATYYCQQAYTIGNIDNAFGGGTEVVVK

SEQ ID NO: 181 - protein, 109 aa, synthetic construct
  REGION 1..109: note = Synthetic
YDMTQTPASVSAAVGGTVTIKCQASEDIYSLLAWYQQKPGQPPKLLIYGASNLESGVPSRFKGSGSGTEYTLTISDLEADDAATYYCQQAYTIGNIDNAFGGGTEVVVK

SEQ ID NO: 182 - protein, 109 aa, synthetic construct
  REGION 1..109: note = Synthetic
YDMTQTPASVSAAVGGTVTIKCQASEDIYRLLAWYQQKPGQPPKLLIYGASNLESGVPSRFKGSGSGTEYTLTISDLECDDAATYYCQQAYTIGNIDNAFGGGTEVVVK

SEQ ID NO: 183 - protein, 109 aa, synthetic construct
  REGION 1..109: note = Synthetic
YDMTQTPASVEAAVGGTVTIKCQASEDIYRLLAWYQQKPGQPPKLLIYGASNLESGVPSRFKGSGSGTEYTLTISDLECDDAATYYCQQAYTIGNIDNAFGGGTEVVVK

SEQ ID NO: 184 - protein, 109 aa, synthetic construct
  REGION 1..109: note = Synthetic
YDMTQTPASVSAAVGGTVTIKCQASEDIYSLLAWYQQKPGQPPKLLIYDASDLASGVPSRFKGSGSGTEYTLTISDLECDDAATYYCQQAYTIGNIDNTFGGGTEVVVK

SEQ ID NO: 185 - protein, 109 aa, synthetic construct
  REGION 1..109: note = Synthetic
YDMTQTPASVSAAVGGTVTIKCQASEDIYSLLAWYQQKPGQPPKLLIYDASDLASGVPSRFKGSGSGTEYTLTISDLECDDAATYYCQQAYTIGNIDNAFGGGTEVVVK

SEQ ID NO: 186 - protein, 109 aa, synthetic construct
  REGION 1..109: note = Synthetic
YDMTQTPASVSAAVGGTVTIKCQASEDIYSLLAWYQQKPGQPPKLLIYDASDLASGVPSRFKGSGSGTEYTLTISALECDDAATYYCQQAYTIGNIDNTFGGGTEVVVK

SEQ ID NO: 187 - protein, 109 aa, synthetic construct; identical to SEQ ID NO: 185
  REGION 1..109: note = Synthetic

SEQ ID NO: 188 - protein, 109 aa, synthetic construct
  REGION 1..109: note = Synthetic
YDMTQTPASVSEPVGGTVTIKCQASQSIYNNFAWYQQKPGQRPKLLIYDASDLASGVPSRFKGSGSGTEYTLTISGVQCADAATYYCQQAYTIGNIDNPFGGGTEVVVK

SEQ ID NO: 189 - protein, 109 aa, synthetic construct
  REGION 1..109: note = Synthetic
YDMTQTPASVSAAVGGTVTIKCQASEDIYSLLAWYQQKPGQPPKLLIFDASDLASGVPSRFKGSGSGTEYTLTISDLECDDAATYYCQQAYTIGNIDNAFGGGTEVVVK

SEQ ID NO: 190 - protein, 109 aa, synthetic construct
  REGION 1..109: note = Synthetic
YDMTQTPASVSAAVGGTITIKCQASEDIYSLLAWYQQKPGQPPKLLIFDASTLASGVPSRFKGSGSGTEYTLTISDLECDDAATYYCQQAYTIGNIDNAFGGGTEVVVE

SEQ ID NO: 191 - protein, 109 aa, synthetic construct
  REGION 1..109: note = Synthetic
YDMTQTPASVSEPVGGTVTIKCQASEDIYKLLAWYQQKPGQPPKLLIYAASDLESGVPSRFKGSGSGTEYTLTISDLECADAATYYCQQAYTIGNIDNTFGGGTEVVVK

SEQ ID NO: 192 - protein, 109 aa, synthetic construct
  REGION 1..109: note = Synthetic
YDMTQTPASVSAAVGGTVTIKCQASEDIYSLLAWYQQKPGQPPKLLIYDASDLASGVPSRFKGSGSGTEYTLTISDLECDDAATYYCQQAYTIGNIDNSFGGGTEVVVK

SEQ ID NO: 193 - protein, 109 aa, synthetic construct
  REGION 1..109: note = Synthetic
YDMTQTPASVSEPVGGTVTIKCQASEDIYRLLAWYQQKPGQPPKLLIYDASDLASGVPSRFKGSGSGTEYTLTISGLQCADAATYYCQQGYTIGNIDNSFGGGTEVVVK

SEQ ID NO: 194 - protein, 109 aa, synthetic construct
  REGION 1..109: note = Synthetic
YDMTQTPASVSEPVGGTVTIKCQASEDIYNLLAWYQQKPGQPPKLLIYAASDLESGVPSRFKGSGSGTEYTLTISDLECDDAATYYCQQGYTIGNIDNAFGGGTEVVVK

SEQ ID NO: 195 - protein, 109 aa, synthetic construct
  REGION 1..109: note = Synthetic
YDMTQTPASVSEPVGGTVTIKCQASEDIYNLLAWYQQKPGQPPKLLIYDASTLTSGVPSRFKGSGSGTEYTLTISDLECDDAATYYCQQAYTIGNIDNTFGGGTEVVVK

SEQ ID NO: 196 - protein, 109 aa, synthetic construct
  REGION 1..109: note = Synthetic
YDMTQTPASVSEPVGGTVTIKCQASEDIYKLLAWYQQKPGQPPKLLIYAASDLESGVPSRFKGSGSGTEYTLTISDLECADAATYYCQQAYTIGNLDNAFGGGTEVVVK

SEQ ID NO: 197 - protein, 109 aa, synthetic construct; identical to SEQ ID NO: 191
  REGION 1..109: note = Synthetic

SEQ ID NO: 198 - protein, 109 aa, synthetic construct; identical to SEQ ID NO: 190
  REGION 1..109: note = Synthetic

SEQ ID NO: 199 - protein, 109 aa, synthetic construct
  REGION 1..109: note = Synthetic
YDMTQTPASVSEPVGGTVTIKCQASEDIYNLLAWYQQKPGQPPKLLIYAASDLESGVPSRFKGSGSGTEYTLTISDLECADAATYYCQQAYTVGNIDNAFGGGTEVVVK

SEQ ID NO: 200 - protein, 109 aa, synthetic construct
  REGION 1..109: note = Synthetic
YDMTQTPASVSEPVGGTVTIKCQASEDIYKLLAWYQQKPGQPPKLLIYAASDLESGVPSRFKGSGSGTDYTLTISDLECADAATYYCQQAYTIGNIDNSFGGGTEVVVK

SEQ ID NO: 201 - protein, 109 aa, synthetic construct
  REGION 1..109: note = Synthetic
YDMTQTPASVSEPVGGTVTIKCQASEDIYNLLAWYQQKPGQPPKLLIYDASTLTSGVPSRFKGGGSGTEYTLTINDLECDDAATYYCQQGYTIGNIDNTFGGGTEVVVK

SEQ ID NO: 202 - protein, 109 aa, synthetic construct
  REGION 1..109: note = Synthetic
FEMTQTPSSVSEPVGGTVTIKCQASEDISSNLGWYQQKPGQPPKLLIYGASTLASGVPSRFKGSGSGTEFTLTISDLECADAATYYCQTSYYIDDGVNGFGGGTEVVVK

SEQ ID NO: 203 - protein, 109 aa, synthetic construct
  REGION 1..109: note = Synthetic
FEMTQTPSSVSEPVGGTVTIKCQASEDISSNLGWYQQKPGQPPKLLIYGASTLASGVPSRFKGSGSGTEFTLTISDLEPADAATYYCQTSYYIDDGVNGFGGGTEVVVK

SEQ ID NO: 204 - protein, 109 aa, synthetic construct
  REGION 1..109: note = Synthetic
FEMTQTPSSVSEPVGGTVTIKCQASEDISSNLGWYQQKPGQPPKLLIYGASTLASGVPSRFKGSGSGTEFTLTISDLEAADAATYYCQTSYYIDDGVNGFGGGTEVVVK

SEQ ID NO: 205 - protein, 109 aa, synthetic construct
  REGION 1..109: note = Synthetic
FEMTQTPASVSEPVGGTVTIKCQASEDISSNLAWYQQKPGQPPKLLIYGASTLASGVPSRFKGSGSGTEFTLTISDLECADAATYYCQSSYYIDDGVNGFGGGTEVVVK

SEQ ID NO: 206 - protein, 109 aa, synthetic construct
  REGION 1..109: note = Synthetic
YDMTQTPASVEVTVGGTVTINCQASESLSSYLAWYQQKPGQPPKPLIYRAATLASGVPSRFKGSGSGTDYTLTISDLECADAATYYCQQGYGYSTVGNAFGGGTEVVVK

SEQ ID NO: 207 - protein, 109 aa, synthetic construct
  REGION 1..109: note = Synthetic
YDMTQTPASVEVAVGGTVTINCQASESISRYLAWYQQKPGQSPKPLIYRASTLASGVPSRFQGSGSGTEYTLTISDLECADAATYYCQQGYGYSTVDNAFGGGTEVVVK

SEQ ID NO: 208 - protein, 109 aa, synthetic construct
  REGION 1..109: note = Synthetic
YDMTQTPASVEVPVGGTVTINCQASENINRYLAWYQQKPGQPPKPLIYRAATLASGVPSRFKGSGSGTEYTLSISDLECADAATYYCQQGYGYSTVGNAFGGGTEVVVK

SEQ ID NO: 209 - protein, 109 aa, synthetic construct
  REGION 1..109: note = Synthetic
YDMTQTPASVSAAVGGTVTINCQASESISRYLAWYQQKPGQPPKPLIYRASTLASGVPSRFKGSGTGTDYTLTISDLECADAATYYCQQGYGYSTVGNAFGGGTEVVVK

SEQ ID NO: 210 - protein, 109 aa, synthetic construct
  REGION 1..109: note = Synthetic
YDMTQTPASVEVAVGGTVTINCQASESINRYLAWYQQKRGQPPKPLIYRAATLASGVPSRFKGSGSGTEYTLTISDLECADAATYYCQQGYGYSTVGNAFGGGTEVVVK

SEQ ID NO: 211 - protein, 12 aa, synthetic construct
  REGION 1..12: note = Synthetic
QQGYGYSTVDNA

SEQ ID NO: 212 - protein, 17 aa, synthetic construct
  REGION 1..17: note = Synthetic
CLYISGGTTYFASWATG

SEQ ID NO: 213 - protein, 12 aa, synthetic construct
  REGION 1..12: note = Synthetic
QQAYTIGNLDNA

SEQ ID NO: 214 - protein, 8 aa, synthetic construct
  REGION 1..8: note = Synthetic
GFDFNNYG

SEQ ID NO: 215 - protein, 8 aa, synthetic construct
  REGION 1..8: note = Synthetic
GFDFNKYG

SEQ ID NO: 216 - protein, 8 aa, synthetic construct
  REGION 1..8: note = Synthetic
GFDFNAYG

SEQ ID NO: 217 - protein, 8 aa, synthetic construct
  REGION 1..8: note = Synthetic
GFDFNYYG

SEQ ID NO: 218 - protein, 8 aa, synthetic construct
  REGION 1..8: note = Synthetic
GFDFNEYG

SEQ ID NO: 219 - protein, 8 aa, synthetic construct
  REGION 1..8: note = Synthetic
GFDFNSYG

SEQ ID NO: 220 - protein, 8 aa, synthetic construct
  REGION 1..8: note = Synthetic
GFDLNNYS

SEQ ID NO: 221 - protein, 8 aa, synthetic construct
  REGION 1..8: note = Synthetic
GFDLNNYA

SEQ ID NO: 222 - protein, 8 aa, synthetic construct
  REGION 1..8: note = Synthetic
GFYLNNYA

SEQ ID NO: 223 - protein, 9 aa, synthetic construct
  REGION 1..9: note = Synthetic
GFDFSGTYW

SEQ ID NO: 224 - protein, 8 aa, synthetic construct
  REGION 1..8: note = Synthetic
GFDFSNYA

SEQ ID NO: 225 - protein, 8 aa, synthetic construct
  REGION 1..8: note = Synthetic
GFDFNNYA

SEQ ID NO: 226 - protein, 8 aa, synthetic construct
  REGION 1..8: note = Synthetic
GFDFSSNA

SEQ ID NO: 227 - protein, 7 aa, synthetic construct
  REGION 1..7: note = Synthetic
IYPAYHN

SEQ ID NO: 228 - protein, 7 aa, synthetic construct
  REGION 1..7: note = Synthetic
IYPAFHN

SEQ ID NO: 229 - protein, 7 aa, synthetic construct
  REGION 1..7: note = Synthetic
IYPTYHN

SEQ ID NO: 230 - protein, 7 aa, synthetic construct
  REGION 1..7: note = Synthetic
IYPAFPN

SEQ ID NO: 231 - protein, 7 aa, synthetic construct
  REGION 1..7: note = Synthetic
IYISGGS

SEQ ID NO: 232 - protein, 7 aa, synthetic construct
  REGION 1..7: note = Synthetic
IYISGGT

SEQ ID NO: 233 - protein, 7 aa, synthetic construct
  REGION 1..7: note = Synthetic
IYISGAT

SEQ ID NO: 234 - protein, 7 aa, synthetic construct
  REGION 1..7: note = Synthetic
LYISGGT

SEQ ID NO: 235 - protein, 7 aa, synthetic construct
  REGION 1..7: note = Synthetic
IYFSGGS

SEQ ID NO: 236 - protein, 8 aa, synthetic construct
  REGION 1..8: note = Synthetic
IYVGSGSS

SEQ ID NO: 237 - protein, 8 aa, synthetic construct
  REGION 1..8: note = Synthetic
IYVGSGGS

SEQ ID NO: 238 - protein, 7 aa, synthetic construct
  REGION 1..7: note = Synthetic
VALRGNI

SEQ ID NO: 239 - protein, 7 aa, synthetic construct
  REGION 1..7: note = Synthetic
IGIRGHI

SEQ ID NO: 240 - protein, 7 aa, synthetic construct
  REGION 1..7: note = Synthetic
IGIRGNI

SEQ ID NO: 241 - protein, 7 aa, synthetic construct
  REGION 1..7: note = Synthetic
VGIYGDF

SEQ ID NO: 242 - protein, 13 aa, synthetic construct
  REGION 1..13: note = Synthetic
ARDTGSTDYNFNL

SEQ ID NO: 243 - protein, 13 aa, synthetic construct
  REGION 1..13: note = Synthetic
ARDTGTTDYNFNL

SEQ ID NO: 244 - protein, 13 aa, synthetic construct
  REGION 1..13: note = Synthetic
ARDAGTTDYNFNL

SEQ ID NO: 245 - protein, 13 aa, synthetic construct
  REGION 1..13: note = Synthetic
ARDAGSTDYKFNL

SEQ ID NO: 246 - protein, 13 aa, synthetic construct
  REGION 1..13: note = Synthetic
ARDAGGTDYNFNL

SEQ ID NO: 247 - protein, 13 aa, synthetic construct
  REGION 1..13: note = Synthetic
ARDAGNTDYNFNL

SEQ ID NO: 248 - protein, 13 aa, synthetic construct
  REGION 1..13: note = Synthetic
ARDAGSTDYNLNL

SEQ ID NO: 249 - protein, 13 aa, synthetic construct
  REGION 1..13: note = Synthetic
ARDAGGTDYNYNL

SEQ ID NO: 250 - protein, 13 aa, synthetic construct
  REGION 1..13: note = Synthetic
ARDAGGTDYAYNL

SEQ ID NO: 251 - protein, 13 aa, synthetic construct
  REGION 1..13: note = Synthetic
ARDAGSTDYYFNL

SEQ ID NO: 252 - protein, 13 aa, synthetic construct
  REGION 1..13: note = Synthetic
ARDAGSVDYNFNL

SEQ ID NO: 253 - protein, 13 aa, synthetic construct
  REGION 1..13: note = Synthetic
ARDAGSTDYNFNL

SEQ ID NO: 254 - protein, 13 aa, synthetic construct
  REGION 1..13: note = Synthetic
ARDDGTASYYLNL

SEQ ID NO: 255 - protein, 13 aa, synthetic construct
  REGION 1..13: note = Synthetic
ARDDGTTSYYLNL

SEQ ID NO: 256 - protein, 13 aa, synthetic construct
  REGION 1..13: note = Synthetic
AKNDGTTSYYLNL

SEQ ID NO: 257 - protein, 13 aa, synthetic construct
  REGION 1..13: note = Synthetic
ARDDGSTSYYLNL

SEQ ID NO: 258 - protein, 13 aa, synthetic construct
  REGION 1..13: note = Synthetic
ARNDGSTNYYLNL

SEQ ID NO: 259 - protein, 13 aa, synthetic construct
  REGION 1..13: note = Synthetic
ARDDGSVSYYLNL

SEQ ID NO: 260 - protein, 15 aa, synthetic construct
  REGION 1..15: note = Synthetic
ARGATNNVFMNYFNL

SEQ ID NO: 261 - protein, 15 aa, synthetic construct
  REGION 1..15: note = Synthetic
ARGATNNVFRNYFNL

SEQ ID NO: 262 - protein, 14 aa, synthetic construct
  REGION 1..14: note = Synthetic
ARGGLYTGYSYFDL

SEQ ID NO: 263 - protein, 14 aa, synthetic construct
  REGION 1..14: note = Synthetic
ARGGLWTGNSYFDL

SEQ ID NO: 264 - protein, 10 aa, synthetic construct
  REGION 1..10: note = Synthetic
GFDFNNYGVS

SEQ ID NO: 265 - protein, 10 aa, synthetic construct
  REGION 1..10: note = Synthetic
GFDFNKYGVS

SEQ ID NO: 266 - protein, 10 aa, synthetic construct
  REGION 1..10: note = Synthetic
GFDFNAYGVS

SEQ ID NO: 267 - protein, 10 aa, synthetic construct
  REGION 1..10: note = Synthetic
GFDFNYYGVS

SEQ ID NO: 268 - protein, 10 aa, synthetic construct
  REGION 1..10: note = Synthetic
GFDFNEYGVS

SEQ ID NO: 269 - protein, 10 aa, synthetic construct
  REGION 1..10: note = Synthetic
GFDFNSYGVS

SEQ ID NO: 270 - protein, 10 aa, synthetic construct
  REGION 1..10: note = Synthetic
GFDFNNYGVT

SEQ ID NO: 271 - protein, 10 aa, synthetic construct
  REGION 1..10: note = Synthetic
GFDFNNYGMS

SEQ ID NO: 272 - protein, 10 aa, synthetic construct
  REGION 1..10: note = Synthetic
GFDLNNYSIL

SEQ ID NO: 273 - protein, 10 aa, synthetic construct
  REGION 1..10: note = Synthetic
GFDLNNYAML

SEQ ID NO: 274 - protein, 10 aa, synthetic construct
  REGION 1..10: note = Synthetic
GFDLNNYAIL

SEQ ID NO: 275 - protein, 10 aa, synthetic construct
  REGION 1..10: note = Synthetic
GFYLNNYAML

SEQ ID NO: 276 - protein, 10 aa, synthetic construct
  REGION 1..10: note = Synthetic
GFDLNNYSML

SEQ ID NO: 277 - protein, 11 aa, synthetic construct
  REGION 1..11: note = Synthetic
GFDFSGTYWMW

SEQ ID NO: 278 - protein, 11 aa, synthetic construct
  REGION 1..11: note = Synthetic
GFDFSGTYWMC

SEQ ID NO: 279 - protein, 10 aa, synthetic construct
  REGION 1..10: note = Synthetic
GFDFSNYAMT

SEQ ID NO: 280 - protein, 10 aa, synthetic construct
  REGION 1..10: note = Synthetic
GFDFNNYAVT

SEQ ID NO: 281 - protein, 10 aa, synthetic construct
  REGION 1..10: note = Synthetic
GFDFSSNAMT

SEQ ID NO: 282 - protein, 10 aa, synthetic construct
  REGION 1..10: note = Synthetic
GFDFSNYAVT

SEQ ID NO: 283 - protein, 6 aa, synthetic construct
  REGION 1..6: note = Synthetic
EDIESY

SEQ ID NO: 284 - protein, 6 aa, synthetic construct
  REGION 1..6: note = Synthetic
QSIYSY

SEQ ID NO: 285 - protein, 6 aa, synthetic construct
  REGION 1..6: note = Synthetic
QSISSY

SEQ ID NO: 286 - protein, 6 aa, synthetic construct
  REGION 1..6: note = Synthetic
QSIESY

SEQ ID NO: 287 - protein, 6 aa, synthetic construct
  REGION 1..6: note = Synthetic
EDIENY

SEQ ID NO: 288 - protein, 6 aa, synthetic construct
  REGION 1..6: note = Synthetic
ENIESY

SEQ ID NO: 289 - protein, 6 aa, synthetic construct
  REGION 1..6: note = Synthetic
KNIDSN

SEQ ID NO: 290 - protein, 6 aa, synthetic construct
  REGION 1..6: note = Synthetic
EDIERY

SEQ ID NO: 291 - protein, 6 aa, synthetic construct
  REGION 1..6: note = Synthetic
EDIYKL

SEQ ID NO: 292 - protein, 6 aa, synthetic construct
  REGION 1..6: note = Synthetic
EDIYSL

SEQ ID NO: 293 - protein, 6 aa, synthetic construct
  REGION 1..6: note = Synthetic
EDIYRL

SEQ ID NO: 294 - protein, 6 aa, synthetic construct
  REGION 1..6: note = Synthetic
QSIYNN

SEQ ID NO: 295 - protein, 6 aa, synthetic construct
  REGION 1..6: note = Synthetic
EDIYNL

SEQ ID NO: 296 - protein, 6 aa, synthetic construct
  REGION 1..6: note = Synthetic
EDISSN

SEQ ID NO: 297 - protein, 6 aa, synthetic construct
  REGION 1..6: note = Synthetic
ESLSSY

SEQ ID NO: 298 - protein, 6 aa, synthetic construct
  REGION 1..6: note = Synthetic
ESISRY

SEQ ID NO: 299 - protein, 6 aa, synthetic construct
  REGION 1..6: note = Synthetic
ENINRY

SEQ ID NO: 300 - protein, 6 aa, synthetic construct
  REGION 1..6: note = Synthetic
ESINRY

SEQ ID NO: 301 - protein, 5 aa, synthetic construct
  REGION 1..5: note = Synthetic
  VARIANT 5: note = X is C or W
TYWMX

SEQ ID NO: 302 - protein, 8 aa, synthetic construct
  REGION 1..8: note = Synthetic
  VARIANT 6: note = X is A, E, N, K, S, or Y
GFDFNXYG

SEQ ID NO: 303 - protein, 8 aa, synthetic construct
  REGION 1..8: note = Synthetic
  VARIANT 3: note = X is D or Y
  VARIANT 8: note = X is A or S
GFXLNNYX

SEQ ID NO: 304 - protein, 8 aa, synthetic construct
  REGION 1..8: note = Synthetic
  VARIANT 8: note = X is A or S
GFDLNNYX

SEQ ID NO: 305 - protein, 8 aa, synthetic construct
  REGION 1..8: note = Synthetic
  VARIANT 5: note = X is N or S
  VARIANT 6: note = X is N or S
  VARIANT 7: note = X is N or Y
GFDFXXXA

SEQ ID NO: 306 - protein, 8 aa, synthetic construct
  REGION 1..8: note = Synthetic
  VARIANT 5: note = X is N or S
GFDFXNYA

SEQ ID NO: 307 - protein, 10 aa, synthetic construct
  REGION 1..10: note = Synthetic
  VARIANT 6: note = X is A, E, K, N, S, or Y
  VARIANT 9: note = X is M or V
  VARIANT 10: note = X S or T
GFDFNXYGXX

SEQ ID NO: 308 - protein, 10 aa, synthetic construct
  REGION 1..10: note = Synthetic
  VARIANT 6: note = X is A, E, K, N, S, or Y
  VARIANT 9: note = X is M or V
GFDFNXYGXS

SEQ ID NO: 309 - protein, 10 aa, synthetic construct
  REGION 1..10: note = Synthetic
  VARIANT 6: note = X is is A, E, K, N, S, or Y
  VARIANT 10: note = X is S or T
GFDFNXYGVX

SEQ ID NO: 310 - protein, 10 aa, synthetic construct
  REGION 1..10: note = Synthetic
  VARIANT 3: note = X is D or Y
  VARIANT 8: note = X is A or S
  VARIANT 9: note = X is I or M
GFXLNNYXXL

SEQ ID NO: 311 - protein, 10 aa, synthetic construct
  REGION 1..10: note = Synthetic
  VARIANT 8: note = X is A or S
  VARIANT 9: note = X is I or M
GFDLNNYXXL

SEQ ID NO: 312 - protein, 11 aa, synthetic construct
  REGION 1..11: note = Synthetic
  VARIANT 11: note = X is C or W
GFDFSGTYWMX

SEQ ID NO: 313 - protein, 10 aa, synthetic construct
  REGION 1..10: note = Synthetic
  VARIANT 5: note = X is N or S..
  VARIANT 6: note = X is N or S
  VARIANT 7: note = X is N or Y
  VARIANT 9: note = X is M or V
GFDFXXXAXT

SEQ ID NO: 314 - protein, 10 aa, synthetic construct
  REGION 1..10: note = Synthetic
  VARIANT 6: note = X is N or S
  VARIANT 7: note = X is N or Y
  VARIANT 9: note = X is M or V
GFDFSXXAXT

SEQ ID NO: 315 - protein, 10 aa, synthetic construct
  REGION 1..10: note = Synthetic
  VARIANT 5: note = X is N or S
  VARIANT 9: note = X is M or V
GFDFXNYAXT

SEQ ID NO: 316 - protein, 17 aa, synthetic construct
  REGION 1..17: note = Synthetic
  VARIANT 5: note = X is A or T
  VARIANT 6: note = X is F or Y
  VARIANT 7: note = X is H or P
  VARIANT 9: note = X is A, R, or T
  VARIANT 13: note = X is N, S or T
YIYPXXXNXYYAXWVNG

SEQ ID NO: 317 - protein, 17 aa, synthetic construct
  REGION 1..17: note = Synthetic
  VARIANT 6: note = X is F or Y
  VARIANT 9: note = X is A, R, or T
  VARIANT 13: note = X is N, S, or T
YIYPAXHNXYYAXWVNG

SEQ ID NO: 318 - protein, 17 aa, synthetic construct
  REGION 1..17: note = Synthetic
  VARIANT 1: note = X is C or S
  VARIANT 2: note = X is I or L
  VARIANT 4: note = X is F or I
  VARIANT 7: note = X is A or G
  VARIANT 8: note = X is S or T
  VARIANT 11: note = X is F or Y
  VARIANT 13: note = X is N or S
  VARIANT 16: note = X is K or T
XXYXSGXXTYXAXWAXG

SEQ ID NO: 319 - protein, 17 aa, synthetic construct
  REGION 1..17: note = Synthetic
  VARIANT 1: note = X is C or S
  VARIANT 8: note = X is S or T
  VARIANT 11: note = X is F or Y
  VARIANT 13: note = X is N or S
  VARIANT 16: note = X is K or T
XIYISGGXTYXAXWAXG

SEQ ID NO: 320 - protein, 18 aa, synthetic construct
  REGION 1..18: note = Synthetic
  VARIANT 1: note = X is C, P, or S
  VARIANT 8: note = X is G or S
XIYVGSGXSTYYASWAKG

SEQ ID NO: 321 - protein, 16 aa, synthetic construct
  REGION 1..16: note = Synthetic
  VARIANT 2: note = X is I or V
  VARIANT 3: note = X is A or G
  VARIANT 4: note = X is I or L
  VARIANT 5: note = X is R or Y
  VARIANT 7: note = X is D, H, or N
  VARIANT 8: note = X is F or I
FXXXXGXXYYANWAKG

SEQ ID NO: 322 - protein, 16 aa, synthetic construct
  REGION 1..16: note = Synthetic
  VARIANT 2: note = X is I or V
  VARIANT 5: note = X is R or Y
  VARIANT 7: note = X is D, N, H
  VARIANT 8: note = X is F or I
FXGIXGXXYYANWAKG

SEQ ID NO: 323 - protein, 16 aa, synthetic construct
  REGION 1..16: note = Synthetic
  VARIANT 2: note = X is I or V
  VARIANT 3: note = X is A or G
  VARIANT 4: note = X is I or L
  VARIANT 7: note = X is H or N
FXXXRGXIYYANWAKG

SEQ ID NO: 324 - protein, 16 aa, synthetic construct
  REGION 1..16: note = Synthetic
  VARIANT 7: note = X is H or N
FIGIRGXIYYANWAKG

SEQ ID NO: 325 - protein, 7 aa, synthetic construct
  REGION 1..7: note = Synthetic
  VARIANT 4: note = X is A or T
  VARIANT 5: note = X is F or Y
  VARIANT 6: note = X is H or P
IYPXXXN

SEQ ID NO: 326 - protein, 7 aa, synthetic construct
  REGION 1..7: note = Synthetic
  VARIANT 5: note = X is F or Y
  VARIANT 6: note = X is H or P
IYPAXXN

SEQ ID NO: 327 - protein, 7 aa, synthetic construct
  REGION 1..7: note = Synthetic
  VARIANT 4: note = X is A or T
  VARIANT 5: note = X is F or Y
IYPXXHN

SEQ ID NO: 328 - protein, 7 aa, synthetic construct
  REGION 1..7: note = Synthetic
  VARIANT 1: note = X is I or L
  VARIANT 6: note = X is A or G
  VARIANT 7: note = X is S or T
XYISGXX

SEQ ID NO: 329 - protein, 7 aa, synthetic construct
  REGION 1..7: note = Synthetic
  VARIANT 7: note = X is S or T
IYISGGX

SEQ ID NO: 330 - protein, 8 aa, synthetic construct
  REGION 1..8: note = Synthetic
  VARIANT 7: note = X is G or S
IYVGSGXS

SEQ ID NO: 331 - skipped

SEQ ID NO: 332 - protein, 7 aa, synthetic construct
  REGION 1..7: note = Synthetic
  VARIANT 6: note = X is H or N
IGIRGXI

SEQ ID NO: 333 - protein, 11 aa, synthetic construct
  REGION 1..11: note = Synthetic
  VARIANT 2: note = X is A or T
  VARIANT 4: note = X is G, N, S, or T
  VARIANT 5: note = X is T or V
  VARIANT 8: note = X is A, K, N, or Y
  VARIANT 9: note = X is F, L, or Y
DXGXXDYXXNL

SEQ ID NO: 334 - protein, 11 aa, synthetic construct
  REGION 1..11: note = Synthetic
  VARIANT 4: note = X is G, N, S, or T
  VARIANT 8: note = X is A, K, N, Y
  VARIANT 9: note = X is F, L or Y
DAGXTDYXXNL

SEQ ID NO: 335 - protein, 11 aa, synthetic construct
  REGION 1..11: note = Synthetic
  VARIANT 1: note = X is D or N
  VARIANT 4: note = X is S or T
  VARIANT 5: note = X is A, T or V
  VARIANT 6: note = X is N or S
XDGXXXYYLNL

SEQ ID NO: 336 - protein, 11 aa, synthetic construct
  REGION 1..11: note = Synthetic
  VARIANT 4: note = X is S or T
  VARIANT 5: note = X is A, T or V
DDGXXSYYLNL

SEQ ID NO: 337 - protein, 13 aa, synthetic construct
  REGION 1..13: note = Synthetic
  VARIANT 8: note = X is M or R
GATNNVFXNYFNL

SEQ ID NO: 338 - protein, 12 aa, synthetic construct
  REGION 1..12: note = Synthetic
  VARIANT 4: note = X is W or Y
  VARIANT 7: note = X is N or Y
GGLXTGXSYFDL

SEQ ID NO: 339 - protein, 13 aa, synthetic construct
  REGION 1..13: note = Synthetic
  VARIANT 4: note = X is A or T
  VARIANT 6: note = X is G, N, S, or T
  VARIANT 7: note = X is T or V
  VARIANT 10: note = X is A, K, N, or Y
  VARIANT 11: note = X is F, L or Y
ARDXGXXDYXXNL

SEQ ID NO: 340 - protein, 13 aa, synthetic construct
  REGION 1..13: note = Synthetic
  VARIANT 4: note = X is A or T
  VARIANT 6: note = X is G, N, S, or T
  VARIANT 10: note = X is A, K, N, or Y
  VARIANT 11: note = X is F, L, or Y
ARDXGXTDYXXNL

SEQ ID NO: 341 - protein, 13 aa, synthetic construct
  REGION 1..13: note = Synthetic
  VARIANT 2..3: note = X is K or R
  VARIANT 6: note = X is D or N
  VARIANT 7: note = X is S or T
  VARIANT 8: note = X is A, T, or V
AXXDGXXXYYLNL

SEQ ID NO: 342 - protein, 13 aa, synthetic construct
  REGION 1..13: note = Synthetic
  VARIANT 6: note = X is N or S
  VARIANT 7: note = X is S or T
ARDDGXXSYYLNL

SEQ ID NO: 343 - protein, 15 aa, synthetic construct
  REGION 1..15: note = Synthetic
  VARIANT 10: note = X is A, T, or V
ARGATNNVFXNYFNL

SEQ ID NO: 344 - protein, 14 aa, synthetic construct
  REGION 1..14: note = Synthetic
  VARIANT 6: note = X is M or R
  VARIANT 9: note = X is W or Y
ARGGLXTGXSYFDL

SEQ ID NO: 345 - protein, 11 aa, synthetic construct
  REGION 1..11: note = Synthetic
  VARIANT 4: note = X is N or Y
  VARIANT 5: note = X is E, K, or Q
  VARIANT 7: note = X is D, N, or S
  VARIANT 8: note = X is D, E, S, or Y
  VARIANT 9: note = X is N, R, or S
  VARIANT 10: note = X is N or Y
  VARIANT 11: note = X is L or S
QASXXIXXXXX

SEQ ID NO: 346 - protein, 11 aa, synthetic construct
  REGION 1..11: note = Synthetic
  VARIANT 4: note = X is A or S
  VARIANT 5: note = X is E or Q
  VARIANT 7: note = X is D, N, or S
  VARIANT 8: note = X is E, S, or Y
  VARIANT 11: note = X is N, R, or S
QASXXIXXYLX

SEQ ID NO: 347 - protein, 11 aa, synthetic construct
  REGION 1..11: note = Synthetic
  VARIANT 4: note = X is A or S
  VARIANT 5: note = X is E or Q
  VARIANT 8: note = X is D or S
  VARIANT 9: note = X is N , K, R, or S
  VARIANT 10: note = X is L or N
QASXXIYXXXA

SEQ ID NO: 348 - protein, 11 aa, synthetic construct
  REGION 1..11: note = Synthetic
  VARIANT 8: note = X is F or L
QASEDIYXLLA

SEQ ID NO: 349 - protein, 11 aa, synthetic construct
  REGION 1..11: note = Synthetic
  VARIANT 11: note = X is N, K, R, or S
QASEDISSNLX

SEQ ID NO: 350 - protein, 11 aa, synthetic construct
  REGION 1..11: note = Synthetic
  VARIANT 5: note = X is A or G
  VARIANT 6: note = X is N or S
  VARIANT 7: note = X is I or L
  VARIANT 8: note = X is N or S
QASEXXXXYLA

SEQ ID NO: 351 - protein, 11 aa, synthetic construct
  REGION 1..11: note = Synthetic
  VARIANT 5: note = X is R or S
  VARIANT 7: note = X is N or S
QASEXIXRYLA

SEQ ID NO: 352 - protein, 6 aa, synthetic construct
  REGION 1..6: note = Synthetic
  VARIANT 2: note = X is N or S
  VARIANT 5: note = X is D or N
EXIEXY

SEQ ID NO: 353 - protein, 6 aa, synthetic construct
  REGION 1..6: note = Synthetic
  VARIANT 4: note = X is N, R, or S
QSIXSY

SEQ ID NO: 354 - protein, 6 aa, synthetic construct
  REGION 1..6: note = Synthetic
  VARIANT 5: note = X is E, S, or Y
EDIYXL

SEQ ID NO: 355 - skipped

SEQ ID NO: 356 - protein, 6 aa, synthetic construct
  REGION 1..6: note = Synthetic
  VARIANT 2: note = X is R or S
  VARIANT 4: note = X is N or S
EXIXRY

SEQ ID NO: 357 - skipped

SEQ ID NO: 358 - protein, 7 aa, synthetic construct
  REGION 1..7: note = Synthetic
  VARIANT 1: note = X is A, P, or T
  VARIANT 4: note = X is A, D, E, or K
  VARIANT 6: note = X is K or T
XASXLXS

SEQ ID NO: 359 - protein, 7 aa, synthetic construct
  REGION 1..7: note = Synthetic
  VARIANT 1: note = X is A, or P
  VARIANT 4: note = X is A, D, E, or K
XASXLAS

SEQ ID NO: 360 - protein, 7 aa, synthetic construct
  REGION 1..7: note = Synthetic
  VARIANT 1: note = X is K or T
  VARIANT 4: note = X is D, N, or T
  VARIANT 6: note = X is A, E, or T
XASXLXS

SEQ ID NO: 361 - protein, 7 aa, synthetic construct
  REGION 1..7: note = Synthetic
  VARIANT 3: note = X is A or S
RAXTLAS

SEQ ID NO: 362 - protein, 12 aa, synthetic construct
  REGION 1..12: note = Synthetic
  VARIANT 1: note = X is H or Q
  VARIANT 3: note = X is A, D, G, or V
  VARIANT 6: note = X is I or V
  VARIANT 8: note = X is D, N, or Y
XQXLTXGXVDNP

SEQ ID NO: 363 - protein, 12 aa, synthetic construct
  REGION 1..12: note = Synthetic
  VARIANT 3: note = X is A, D, G, or V
  VARIANT 6: note = X is I or V
  VARIANT 8: note = X is D, N, or Y
QQXLTXGXVDNP

SEQ ID NO: 364 - protein, 12 aa, synthetic construct
  REGION 1..12: note = Synthetic
  VARIANT 3: note = X is A, or G
  VARIANT 6: note = X is I or V
  VARIANT 12: note = X is A, P, S or T
QQXYTXGNIDNX

SEQ ID NO: 365 - protein, 12 aa, synthetic construct
  REGION 1..12: note = Synthetic
  VARIANT 2: note = X is S or T
QXSYYIDDGVNG

SEQ ID NO: 366 - protein, 12 aa, synthetic construct
  REGION 1..12: note = Synthetic
  VARIANT 10: note = X is D or G
QQGYGYSTVXNA

SEQ ID NO: 367 - protein, 120 aa, synthetic construct
  REGION 1..120: note = Synthetic
  MOD_RES 1: note = Pyroglutamic acid (pE)
EEQLKESGGGLVQPGGSLTLSCKASGFDFNNYGVSWVRQAPGKGLEWIGYIYPAYHNTYYATWVNGRFTISSHNAQNTLYLQLNTLTAADTATYFCARDTGSTDYNFNLWGPGTLVTVSS

SEQ ID NO: 368 - protein, 120 aa, synthetic construct
  REGION 1..120: note = Synthetic
  MOD_RES 1: note = Pyroglutamic acid (pE)
EEQLKESGGGLGQPGGSLTLSCKASGFDFNNYGVSWVRQAPGKGLEWIGYIYPAYHNTYYATWVNGRFTISSHNAQNTLYLQLNTLTAADTATYFCARDTGTTDYNFNLWGPGTLVTVSS

SEQ ID NO: 369 - protein, 120 aa, synthetic construct
  REGION 1..120: note = Synthetic
  MOD_RES 1: note = Pyroglutamic acid (pE)
EEQLKESGGGLVQPGGSLKLSCKASGFDFNKYGVSWVRQAPGKGLEWIAYIYPAFHNTYYATWVNGRFTISSHNAQNTLYLQLNTLTAADTATYFCARDAGTTDYNFNLWGPGTLVTVSS

SEQ ID NO: 370 - protein, 120 aa, synthetic construct
  REGION 1..120: note = Synthetic
  MOD_RES 1: note = Pyroglutamic acid (pE)
EEQLKESGGGLVQPGGSLKLSCKASGFDFNAYGVSWVRQAPGKGLEWIAYIYPAFHNTYYATWVNGRFTISSHNAQNTLYLQLNTLTAADTATYFCARDAGTTDYNFNLWGPGTLVTVSS

SEQ ID NO: 371 - protein, 120 aa, synthetic construct
  REGION 1..120: note = Synthetic
  MOD_RES 1: note = Pyroglutamic acid (pE)
EEQLKESGGGLVQPGGSLKLSCKASGFDFNNYGVSWVRQAPGKGLEWIAYIYPAFHNRYYANWVNGRFTISSDNAQNTVYLQLNSLTAADTATYFCARDAGSTDYKFNLWGPGTLVTVSS

SEQ ID NO: 372 - protein, 120 aa, synthetic construct
  REGION 1..120: note = Synthetic
  MOD_RES 1: note = Pyroglutamic acid (pE)
EEQLKESGGGLVQPGGSLTLSCKASGFDFNNYGVSWVRQAPGKGLEWIAYIYPTYHNTYYATWVNGRFTISSHNAQNTLYLQLNTLTAADTATYFCARDAGGTDYNFNLWGPGTLVTVSS

SEQ ID NO: 373 - protein, 120 aa, synthetic construct
  REGION 1..120: note = Synthetic
  MOD_RES 1: note = Pyroglutamic acid (pE)
EEQLKESGGGLVQPGGSLTLSCKASGFDFNNYGVSWVRQAPGKGLEWIGYIYPAYHNTYYATWVNGRFTISSHNAQNTLYLQLNTLTAADTATYFCARDAGNTDYNFNLWGPGTLVTVSS

SEQ ID NO: 374 - protein, 120 aa, synthetic construct
  REGION 1..120: note = Synthetic
  MOD_RES 1: note = Pyroglutamic acid (pE)
EEQLKESGGGLVQPGGSLTLSCKASGFDFNYYGVSWVRQAPGKGLEWIGYIYPAYHNTYYATWVNGRFTISSHNAQNTLYLQLNTLTAADTATYFCARDAGSTDYNLNLWGPGTLVTVSS

SEQ ID NO: 375 - protein, 120 aa, synthetic construct
  REGION 1..120: note = Synthetic
  MOD_RES 1: note = Pyroglutamic acid (pE)
EEQLKESGGGLVQPGGSLKLSCKASGFDFNEYGVSWVRQAPGKGLEWIAYIYPAFHNTYYATWVNGRFTISSHNAQNTLYLQLNTLTAADTATYFCARDAGTTDYNFNLWGPGTLVTVSS

SEQ ID NO: 376 - protein, 120 aa, synthetic construct
  REGION 1..120: note = Synthetic
  MOD_RES 1: note = Pyroglutamic acid (pE)
EEQLVESGGGLVQPGGSLTLSCKGSGFDFNEYGVSWVRQAPGKGLEWIAYIYPAFHNTYYATWVNGRFTISSHNAQNTLYLQLHTLTAADTATYFCARDAGTTDYNFNLWGPGTLVTVSS

SEQ ID NO: 377 - protein, 120 aa, synthetic construct
  REGION 1..120: note = Synthetic
  MOD_RES 1: note = Pyroglutamic acid (pE)
EEQLKESGGGLVQPGGSLKLSCKASGFDFNSYGVSWVRQAPGKGLEWIAYIYPAFHNAYYANWVNGRFTISSDNAQNTVDLQLNSLTAADTATYFCARDAGGTDYNYNLWGPGTLVTVSS

SEQ ID NO: 378 - protein, 120 aa, synthetic construct
  REGION 1..120: note = Synthetic
  MOD_RES 1: note = Pyroglutamic acid (pE)
EEQLKESGGGLVQPGGSLKLSCKASGFDFNNYGVSWVRQAPGKGLEWIAYIYPAFHNTYYASWVNGRFTISSHNAQNTLYLQLNSLTAADTATYFCARDAGGTDYAYNLWGPGTLVTVSS

SEQ ID NO: 379 - protein, 120 aa, synthetic construct
  REGION 1..120: note = Synthetic
  MOD_RES 1: note = Pyroglutamic acid (pE)
EEQLKESGGGLVRLGDPLQLSCKASGFDFNNYGVTWVRQAPGKGLEWIAYIYPAFPNTYYATWVNGRFTISPHNAQNTLYLQLNSLTAADTATYFCARDAGSTDYYFNLWGPGTLATVSS

SEQ ID NO: 380 - protein, 120 aa, synthetic construct
  REGION 1..120: note = Synthetic
  MOD_RES 1: note = Pyroglutamic acid (pE)
EEQLKESGGGLVQPGGSLKLSCKASGFDFNNYGVSWVRQAPGKGLEWIAYIYPAFHNTYYATWVNGRFTISSHNAQNTLYLQLNSLTAADTATYFCARDAGSVDYNFNLWGPGTLVTVSS

SEQ ID NO: 381 - protein, 120 aa, synthetic construct
  REGION 1..120: note = Synthetic
  MOD_RES 1: note = Pyroglutamic acid (pE)
EEQLKESGGGLVQPGGSLKLSCKASGFDFNNYGMSWVRQAPGKGLEWIAYIYPAFHNTYYANWVNGRFTISSDNAQNTVDLQLNSLTAADTATYFCARDAGSTDYNFNLWGPGTLVTVSS

SEQ ID NO: 382 - protein, 118 aa, synthetic construct
  REGION 1..118: note = Synthetic
  MOD_RES 1: note = Pyroglutamic acid (pE)
ESLEESGGDLVQPGASLTLTCKASGFDLNNYSILWVRQAPGKGLEWIGCIYISGGSTYYANWAKGRFAISKTSSTTVTLQMTSLTAADTATYFCARDDGTASYYLNLWGPGTLVTVSS

SEQ ID NO: 383 - protein, 118 aa, synthetic construct
  REGION 1..118: note = Synthetic
  MOD_RES 1: note = Pyroglutamic acid (pE)
ESLEESGGDLVQPGASLTLTCKASGFDLNNYSILWVRQAPGKGLEWIGSIYISGGSTYYANWAKGRFAISKTSSTTVTLQMTSLTAADTATYFCARDDGTASYYLNLWGPGTLVTVSS

SEQ ID NO: 384 - protein, 118 aa, synthetic construct
  REGION 1..118: note = Synthetic
  MOD_RES 1: note = Pyroglutamic acid (pE)
ESLEESGGDLVQPGASLTLTCKASGFDLNNYAMLWVRQAPGKGLEWIGCIYISGGTTYFASWATGRFAISKTSSTTVTLQMTSLTAADTATYFCARDDGTTSYYLNLWGPGTLVTVSS

SEQ ID NO: 385 - protein, 118 aa, synthetic construct
  REGION 1..118: note = Synthetic
  MOD_RES 1: note = Pyroglutamic acid (pE)
ESLEESGGDLVQPGASLTLTCKASGFDLNNYAMLWVRQAPGKGLEWIGSIYISGGTTYFASWATGRFAISKTSSTTVTLQMTSLTAADTATYFCARDDGTTSYYLNLWGPGTLVTVSS

SEQ ID NO: 386 - protein, 118 aa, synthetic construct
  REGION 1..118: note = Synthetic
  MOD_RES 1: note = Pyroglutamic acid (pE)
ESLEESGGDLVQPGASLTLTCKASGFDLNNYAMLWVRQAPGKGLEWIGCIYISGGTTYFASWATGRFAISKTSSTTLTLQMTTLPPPDTATYFCAKNDGTTSYYLNLWGPGTLVTVSS

SEQ ID NO: 387 - protein, 118 aa, synthetic construct
  REGION 1..118: note = Synthetic
  MOD_RES 1: note = Pyroglutamic acid (pE)
ESLEESGGDLVQPGASLTLTCKASGFDLNNYAILWVRQAPGKGLEWIGCIYISGATTYFANWATGRFAISKTSSTTVTLQMTSLTAADTATYFCARDDGTTSYYLNLWGPGTLVTVSS

SEQ ID NO: 388 - protein, 118 aa, synthetic construct
  REGION 1..118: note = Synthetic
  MOD_RES 1: note = Pyroglutamic acid (pE)
ESLEESGGDLVQPGASLTLTCKASGFDLNNYAMLWVRQAPGKGLEWIGCIYISGGTTYFASWATGRFAISKTSSTTVTLQMTSLTAADTATYFCARDDGSTSYYLNLWGPGTLVTVSS

SEQ ID NO: 389 - protein, 118 aa, synthetic construct
  REGION 1..118: note = Synthetic
  MOD_RES 1: note = Pyroglutamic acid (pE)
ESLEESGGDLVQPGASLTLTCKASGFDLNNYAMLWVRQAPGKGLEWIGCIYISGGTTYFASWATGRFAISRTSSTTVTLQMTSLTAADTATYFCARDDGSTSYYLNLWGPGTLVTVSS

SEQ ID NO: 390 - protein, 118 aa, synthetic construct
  REGION 1..118: note = Synthetic
  MOD_RES 1: note = Pyroglutamic acid (pE)
ESLEESGGNLAHPGASLRLTCTASGFYLNNYAMLWVRQAPGKGLEWIGCLYISGGTTYFASWATGRFAISKTSSTTGTLQMTSLTAADTATYFCARNDGSTNYYLNLWGPGTLVTISS

SEQ ID NO: 391 - protein, 118 aa, synthetic construct
  REGION 1..118: note = Synthetic
  MOD_RES 1: note = Pyroglutamic acid (pE)
ESLEESGGDLVQPGASLTLTCRASGFDLNNYSMLWVRQAPGKGLEWIGCIYISGGTTYFASWATGRFAISKTSSTTVTLQMTSLTAADTATYFCARDDGSTSYYLNLWGPGTLVTVSS

SEQ ID NO: 392 - protein, 118 aa, synthetic construct
  REGION 1..118: note = Synthetic
  MOD_RES 1: note = Pyroglutamic acid (pE)
ESLEESGGDLVQPGASLTLTCRASGFDLNNYSMLWVRQAPGKGLEWIGCIYISGGTTYFASWATGRFAISKTSSTTVTLQMTILTAADTATYFCARDDGSTSYYLNLWGPGTLVTVSS

SEQ ID NO: 393 - protein, 118 aa, synthetic construct
  REGION 1..118: note = Synthetic
  MOD_RES 1: note = Pyroglutamic acid (pE)
ESLEESGGDLVQPGASLTLTCKASGFDLNNYSILWVRQAPGKGLEWIGCIYISGGTTYFASWATGRFAISKTSSTTVTLQMTSLTAADTATYFCARDDGTTSYYLNLWGPGTLVTVSS

SEQ ID NO: 394 - protein, 118 aa, synthetic construct
  REGION 1..118: note = Synthetic
  MOD_RES 1: note = Pyroglutamic acid (pE)
ESLEESGGDLVQPGASLTLTCKAAGFDLNNYSMLWVRQAPGKGLEWIGCIYISGGTTYYASWAKGRFTISKTSSTTVTLQMTSLTAADTATYFCARDDGSTSYYLNLWGPGTLVTVSS

SEQ ID NO: 395 - protein, 118 aa, synthetic construct
  REGION 1..118: note = Synthetic
  MOD_RES 1: note = Pyroglutamic acid (pE)
ESLEESGGDLVQPGVSLTLTCKASGFDLNNYSILWVRQAPGKGLEWIGCIYISGGSTYYASWAKGRFAISKTSSTTVTLQMTSLTAADTATYFCARDDGTTSYYLNLWGPGTLVTVSS

SEQ ID NO: 396 - protein, 118 aa, synthetic construct
  REGION 1..118: note = Synthetic
  MOD_RES 1: note = Pyroglutamic acid (pE)
ESLEESGGDLVQPGASLTLTCKASGFDLNNYSILWVRQAPGKGLEWIGCIYISGGTTYYANWAKGRFTISRTSSTTVTLQMTSLTGADTATYFCARDDGSTSYYLNLWGPGTLVTVSS

SEQ ID NO: 397 - protein, 118 aa, synthetic construct
  REGION 1..118: note = Synthetic
  MOD_RES 1: note = Pyroglutamic acid (pE)
ESLEESGGDLVQPGASLTLTCKASGFDLNNYSILWVRQAPGKGLEWIGCIYISGGTTYYANWAKGRFAISKTSSTTVTLQMTSLTAADTATYFCARDDGTTSYYLNLWGPGTLVTVSS

SEQ ID NO: 398 - protein, 118 aa, synthetic construct
  REGION 1..118: note = Synthetic
  MOD_RES 1: note = Pyroglutamic acid (pE)
ESLEESGGDLVQPGASLTLTCKASGFDLNNYSILWVRQAPGKGLEWIGCIYISGGSTYYANWAKGRFAISKTSSTTVTLQMTSLTAADTATYFCARDDGTTSYYLNLWGPGTLVTVSS

SEQ ID NO: 399 - protein, 118 aa, synthetic construct
  REGION 1..118: note = Synthetic
  MOD_RES 1: note = Pyroglutamic acid (pE)
ESLEESGGDLVQPGASLTLTCKASGFDLNNYSILWVRQAPGKGLEWIGCIYISGGSTYYANWAKGRFAVSKTSSTTVTLQMTSLTAADTATYFCARDDGTTSYYLNLWGPGTLVTVSS

SEQ ID NO: 400 - protein, 118 aa, synthetic construct
  REGION 1..118: note = Synthetic
  MOD_RES 1: note = Pyroglutamic acid (pE)
ESLEESGGDLVQPGASLTLTCKASGFDLNNYSILWVRQAPGKGLEWIGCIYFSGGSTYYANWAKGRFAISKTSSTTVTLQMTSLTAADTATYFCARDDGTTSYYLNLWGPGTLVTVSS

SEQ ID NO: 401 - protein, 118 aa, synthetic construct
  REGION 1..118: note = Synthetic
  MOD_RES 1: note = Pyroglutamic acid (pE)
ESLEESGGDLVQPGTSLTLSCKASGFDLNNYSILWVRQAPGKGLEWIGCIYISGGTTYFANWAKGRFTISRPSSTTVTLQMTSLTGADTATYFCARDDGSVSYYLNLWGPGTLVIVSS

SEQ ID NO: 402 - protein, 122 aa, synthetic construct
  REGION 1..122: note = Synthetic
  MOD_RES 1: note = Pyroglutamic acid (pE)
EQLEESGGGLVKPEGSLTLTCKGSGFDFSGTYWMWWVRQAPGKGLEWIACIYVGSGSSTYYASWAKGRFTISSTSSTAVTLQATSLTAADTATYFCARGATNNVFMNYFNLWGPGTLVTVSS

SEQ ID NO: 403 - protein, 122 aa, synthetic construct
  REGION 1..122: note = Synthetic
  MOD_RES 1: note = Pyroglutamic acid (pE)
EQLEESGGGLVKPEGSLTLTCKGSGFDFSGTYWMWWVRQAPGKGLEWIAPIYVGSGSSTYYASWAKGRFTISSTSSTAVTLQATSLTAADTATYFCARGATNNVFMNYFNLWGPGTLVTVSS

SEQ ID NO: 404 - protein, 122 aa, synthetic construct
  REGION 1..122: note = Synthetic
  MOD_RES 1: note = Pyroglutamic acid (pE)
EQLEESGGGLVKPEGSLTLTCKGSGFDFSGTYWMWWVRQAPGKGLEWIASIYVGSGSSTYYASWAKGRFTISSTSSTAVTLQATSLTAADTATYFCARGATNNVFMNYFNLWGPGTLVTVSS

SEQ ID NO: 405 - protein, 122 aa, synthetic construct
  REGION 1..122: note = Synthetic
  MOD_RES 1: note = Pyroglutamic acid (pE)
EQLEESGGGLVKPEGSLTLTCKGSGFDFSGTYWMCWVRQAPGKGLEWIACIYVGSGGSTYYASWAKGRFTISSTSSTAVTLQVTSLTAADTATYFCARGATNNVFRNYFNLWGPGTLVTVSS

SEQ ID NO: 406 - protein, 118 aa, synthetic construct
  REGION 1..118: note = Synthetic
  MOD_RES 1: note = Pyroglutamic acid (pE)
ESLEESGGGLVKPGGTLTLTCTVSGFDFSNYAMTWVRQAPGEGLEYIGFVALRGNIYYANWAKGRFTISKTSSTTVTLQMTSLTVADTATYFCARGGLYTGYSYFDLGGPGTLATVSS

SEQ ID NO: 407 - protein, 118 aa, synthetic construct
  REGION 1..118: note = Synthetic
  MOD_RES 1: note = Pyroglutamic acid (pE)
ESLVESGGGLVQPEGSLTLTCKASGFDFNNYAVTWVRQAPGEGLEYIGFIGIRGHIYYANWAKGRFTISKTSSTTMTLQMTSLTVADTATYFCARGGLWTGNSYFDLWGPGTLVTVSS

SEQ ID NO: 408 - protein, 118 aa, synthetic construct
  REGION 1..118: note = Synthetic
  MOD_RES 1: note = Pyroglutamic acid (pE)
ESLEESGGGLVKPGGTLTLTCTVSGFDFSNYAMTWVRQAPGKGLEYIGFIGIRGNIYYANWAKGRFTISKTSSTTVTLQMTSLTVADTATYFCARGGLWTGNSYFDLWGPGTLVTVSS

SEQ ID NO: 409 - protein, 118 aa, synthetic construct
  REGION 1..118: note = Synthetic
  MOD_RES 1: note = Pyroglutamic acid (pE)
ESLEESGGGLVQPGASLTLTCTVSGFDFSSNAMTWVRQAPGEGLEYIGFIGIRGNIYYANWAKGRFTISKTSSTTVTLQMTSLTVADTATYFCARGGLWTGNSYFDLWGPGTLVTVSS

SEQ ID NO: 410 - protein, 118 aa, synthetic construct
  REGION 1..118: note = Synthetic
  MOD_RES 1: note = Pyroglutamic acid (pE)
ESLEESGGGLVKPGGTLTLTCTVSGFDFSNYAVTWVRQAPGEGLEYIGFVGIYGDFYYANWAKGRFTISKTSSTTVTLQMPSLTVADTATYFCARGGLWTGNSYFDLWGPGTLVTVSS

SEQ ID NO: 411 - DNA, 360 bp, synthetic construct
  misc_feature 1..360: note = Synthetic
  source 1..360: mol_type = other DNA
caggaacagctgaaggagagcggcggcggcctggtgcagccaggaggaagtctgaccctgagctgcaaagccagtggattcgacttcaacaactacggcgtgagttgggtgagacaggcccccggaaaaggactggagtggattggatacatttaccccgcctaccacaacacctactacgccacctgggtgaacggcagattcacaatcagcagccacaacgcccagaacaccctgtacctgcagctgaacacactgaccgccgccgacaccgccacctacttctgcgccagggacaccggcagcaccgactacaacttcaacctgtggggaccaggcaccctggtgaccgtgtcctcc

SEQ ID NO: 412 - DNA, 360 bp, synthetic construct
  misc_feature 1..360: note = Synthetic
  source 1..360: mol_type = other DNA
caggagcagctgaaggagtccgggggaggcctgggccagcctgggggatccctgacactctcctgcaaagcctctggattcgacttcaataactatggagtgagctgggtccgccaggctccagggaaggggctggagtggatcgggtacatttatcctgcttatcataacacatactacgcgacctgggtgaatggccgattcaccatttccagccacaacgcccagaacacgctgtatctgcaactgaacactctgacagccgcggacacggccacctatttctgtgcgagagatactggtactactgattataactttaacttgtggggcccaggcaccctggtcaccgtctcctca

SEQ ID NO: 413 - DNA, 360 bp, synthetic construct
  misc_feature 1..360: note = Synthetic
  source 1..360: mol_type = other DNA
caggagcagctgaaggagtccgggggaggcctggtccagcctgggggatccctgaaactctcctgcaaagcctctggattcgacttcaataaatatggagtgagctgggtccgccaggctccagggaaggggctggagtggatcgcgtacatttatcctgcttttcataacacatactacgcgacctgggtgaatggccgattcaccatttccagccacaacgcccagaacacgctgtatctgcaactgaacactctgacagccgcggacacggccacctatttctgtgcgagagatgctggtactactgattataactttaacttgtggggcccaggcaccctggtcaccgtctcctca

SEQ ID NO: 414 - DNA, 360 bp, synthetic construct
  misc_feature 1..360: note = Synthetic
  source 1..360: mol_type = other DNA
caggaacaactcaaagagagcggcggcggcctcgtgcaacccggaggaagtctcaagctcagctgcaaggcaagcggcttcgacttcaacgcctacggcgtcagctgggtgagacaggcccccggaaagggactggagtggattgcttatatttaccctgcttttcacaacacatactacgccacctgggtgaatggcagattcaccatctcctctcacaacgcccagaacacactgtacctgcagctgaacacactgaccgccgccgacaccgccacctacttctgcgcccgggacgccggaaccaccgactacaactttaacctgtggggccccggcacactcgtcaccgtctcctcc

SEQ ID NO: 415 - DNA, 360 bp, synthetic construct
  misc_feature 1..360: note = Synthetic
  source 1..360: mol_type = other DNA
caggagcagctgaaggagtccgggggaggcctggtccagcctgggggatccctgaaactctcctgcaaagcctctggattcgacttcaataactatggagtgagctgggtccgccaggctccagggaaggggctggagtggatcgcatacatttatcctgcttttcataacagatattacgcgaactgggtgaatggccgattcaccatctccagcgacaacgcccagaacacggtgtatctgcaactgaacagtctgacagccgcggacacggccacctatttctgtgcgagagatgctggtagtactgattataaatttaacttgtggggcccaggcaccctggtcaccgtctcctca

SEQ ID NO: 416 - DNA, 360 bp, synthetic construct
  misc_feature 1..360: note = Synthetic
  source 1..360: mol_type = other DNA
caggagcagctgaaggagtccgggggaggcctggtccagcctgggggatccctgacactctcctgcaaagcctctggattcgacttcaataactatggagtgagctgggtccgccaggctccagggaaggggctggagtggatcgcgtacatttatcctacttatcataacacatactacgcgacctgggtgaatggccgattcaccatttccagccacaacgcccagaacacgctgtatctgcaactgaacactctgacagccgcggacacggccacctatttctgtgcgagagatgctggtggtactgattataactttaacttgtggggcccaggcaccctggtcaccgtctcctca

SEQ ID NO: 417 - DNA, 360 bp, synthetic construct
  misc_feature 1..360: note = Synthetic
  source 1..360: mol_type = other DNA
caggagcagctgaaggagtccgggggaggcctggtccagcctgggggatccctgacactctcctgcaaagcctctggattcgacttcaataactatggagtgagctgggtccgccaggctccagggaaggggctggagtggatcgggtacatttatcctgcttatcataacacatactacgcgacctgggtgaatggccgattcaccatttccagccacaacgcccagaacacgctgtatctgcaactgaacactctgacagccgcggacacggccacctatttctgtgcgagagatgctggtaatactgattataactttaacttgtggggcccaggcaccctggtcaccgtctcctca

SEQ ID NO: 418 - DNA, 360 bp, synthetic construct
  misc_feature 1..360: note = Synthetic
  source 1..360: mol_type = other DNA
caggaacagctgaaggagagcggcggcggcctggtgcagccaggaggaagtctgaccctgagctgcaaagccagtggattcgacttcaactactacggcgtgagttgggtgagacaggcccccggaaaaggactggagtggattggctatatttaccccgcttatcacaacacctattacgccacatgggtgaacggcagattcacaatcagcagccacaacgcccagaacacactgtacctgcagctgaacacactgaccgccgccgacaccgccacctacttctgcgccagggacgccggcagcaccgactacaacctgaacctgtggggacccggcaccctggtgaccgtgtcctcc

SEQ ID NO: 419 - DNA, 360 bp, synthetic construct
  misc_feature 1..360: note = Synthetic
  source 1..360: mol_type = other DNA
caggaacaactcaaagagagcggcggcggcctcgtgcaacccggaggaagtctcaagctcagctgcaaggcaagcggcttcgacttcaacgaatacggcgtgagctgggtgagacaggcccccggaaagggactggagtggattgcttatatttaccctgcttttcacaacacatactacgccacctgggtgaatggcagattcacaataagctctcataacgctcagaacacactgtacctgcagctgaacacactgaccgccgccgacaccgccacctacttctgcgccagggacgccggcaccaccgactacaacttcaacctgtggggccccggcaccctggtgaccgtttcctcc

SEQ ID NO: 420 - DNA, 360 bp, synthetic construct
  misc_feature 1..360: note = Synthetic
  source 1..360: mol_type = other DNA
caggagcagctggtggagtccgggggaggcctggtccagcctgggggatccctgacactctcctgcaaaggctctggattcgacttcaatgaatatggagtgagctgggtccgccaggctccagggaaggggctggagtggatcgcgtacatttatcctgcctttcataacacatactacgcgacctgggtgaatggccgattcaccatttccagccacaacgcccagaacacgctgtatctgcaactgcacactctgacagccgcggacacggccacctatttctgtgcgagagatgctggtactactgattataactttaacttgtggggcccaggcaccctggtcaccgtctcctca

SEQ ID NO: 421 - DNA, 360 bp, synthetic construct
  misc_feature 1..360: note = Synthetic
  source 1..360: mol_type = other DNA
caggaacaactcaaagagagcggcggcggcctcgtgcaacccggaggaagtctcaagctcagctgcaaggcaagcggcttcgacttcaacagctacggagtgagctgggtgagacaggcccccggcaaaggactggagtggattgcttatatttacccagccttccacaacgcctactacgccaactgggtgaatggcagattcacaatcagcagcgacaacgcccagaacaccgtggacctgcaactgaacagcctgaccgccgccgacaccgccacctacttctgcgcccgcgacgccggaggcaccgattacaactacaacctctggggccctggcaccctcgtgacagtgagctcc

SEQ ID NO: 422 - DNA, 360 bp, synthetic construct
  misc_feature 1..360: note = Synthetic
  source 1..360: mol_type = other DNA
caggaacaactcaaagagagcggcggcggcctcgtgcaacccggaggaagtctcaagctcagctgcaaggcaagcggcttcgacttcaacaactacggcgtgagctgggtgagacaggcccccggaaagggactggagtggattgcttatatttaccctgcttttcacaacacatactacgccagctgggtgaatggcagattcacaatctcctctcataacgctcagaacaccctgtacctgcaactgaacagcctgaccgccgccgacaccgccacctacttctgcgccagggacgccggcggcaccgattacgcttacaacctctggggccccggaaccctggtcaccgtgtcctcc

SEQ ID NO: 423 - DNA, 360 bp, synthetic construct
  misc_feature 1..360: note = Synthetic
  source 1..360: mol_type = other DNA
caggagcagctgaaggagagcggcggcggcctggtgcgcctgggcgatccgctgcagctctcctgcaaagcctctggattcgacttcaataactatggagtgacctgggtccgccaggctccagggaaggggctggagtggatcgcgtacatttatcctgcttttcctaacacatactacgcgacctgggtgaatggccgattcaccatctccccccacaacgcccagaacacgctgtatctgcaactgaacagtctgacagccgcggacacggccacctatttctgtgcgagagatgctggtagtactgattattactttaacttgtggggcccaggcaccctggccaccgtgtcctcc

SEQ ID NO: 424 - DNA, 360 bp, synthetic construct
  misc_feature 1..360: note = Synthetic
  source 1..360: mol_type = other DNA
caggaacaactcaaagagagcggcggcggcctcgtgcaacccggaggaagtctcaagctcagctgcaaggcaagcggcttcgacttcaacaactacggcgtgagctgggtgagacaggcccccggaaagggactggagtggattgcttatatttaccctgcttttcacaacacatactacgccacatgggtgaatggcagattcacaatatcctcccataacgctcagaacacactgtacctgcagctgaacagcctgaccgccgccgacaccgccacctacttctgcgcacgcgacgccggcagcgtggactacaacttcaacctgtggggccccggcactctcgtgaccgtctcctcc

SEQ ID NO: 425 - DNA, 360 bp, synthetic construct
  misc_feature 1..360: note = Synthetic
  source 1..360: mol_type = other DNA
caggaacaactcaaagagagcggcggcggcctcgtgcaacccggaggaagtctcaagctcagctgcaaggcaagcggcttcgacttcaacaactacggcatgagctgggtgagacaggcccccggcaaaggactggagtggattgcttatatttacccagccttccacaacacctactacgccaactgggtgaatggcagattcacaatcagcagcgacaacgcccagaacaccgtggacctgcaactgaacagcctgaccgccgccgacaccgccacctacttctgcgcccgcgacgccggaagcaccgactacaacttcaacctgtggggccccggaaccctggtcaccgtgagctcc

SEQ ID NO: 426 - DNA, 360 bp, synthetic construct
  misc_feature 1..360: note = Synthetic
  source 1..360: mol_type = other DNA
caggagcagctgaaggagtccgggggaggcctggtccagcctgggggatccctaaaactctcctgcaaagcctctggattcgacttcaataactatggaatgagctgggtccgccaggctccagggaaggggctggagtggatcgcgtacatttatcctgcttttcataatacatactacgcgaactgggtgaatggccgattcaccatctccagcgacaacgcccagaacactgtggatctgcaactgaacagtctgacagccgcggacacggccacctatttctgtgcgagagatgctggtagtactgattataactttaacttgtggggcccaggcaccctggtcaccgtctcctca

SEQ ID NO: 427 - DNA, 354 bp, synthetic construct
  misc_feature 1..354: note = Synthetic
  source 1..354: mol_type = other DNA
cagagcctggaggaaagcggcggcgacctggtgcagcccggagcttcactgaccctgacctgcaaggccagcggcttcgacctgaacaactacagcatcctgtgggtgagacaggcccccggaaaaggcctggagtggattggatgcatttacatcagtggaggctccacctactacgccaactgggccaagggcagattcgccatcagcaagacctccagcaccacagtgaccctgcagatgacaagcctgaccgccgccgacaccgccacctacttttgcgcccgcgacgacggaaccgccagctactacctgaacctgtggggacccggcaccctggtgaccgtgagctcc

SEQ ID NO: 428 - DNA, 354 bp, synthetic construct
  misc_feature 1..354: note = Synthetic
  source 1..354: mol_type = other DNA
cagagcctggaggaaagcggcggcgacctggtgcagcccggagcttcactgaccctgacctgcaaggccagcggcttcgacctgaacaactacagcatcctgtgggtgagacaggcccccggaaaaggcctggagtggattggatctatttacatcagtggaggctccacctactacgccaactgggccaagggcagattcgccatcagcaagacctccagcaccacagtgaccctgcagatgacaagcctgaccgccgctgacaccgccacctacttttgcgcccgcgacgacggaaccgccagctactacctgaacctgtggggacccggcaccctggtgaccgtgagcagc

SEQ ID NO: 429 - DNA, 354 bp, synthetic construct
  misc_feature 1..354: note = Synthetic
  source 1..354: mol_type = other DNA
cagagcctggaggaaagcggcggcgacctggtgcagcccggagcttcactgaccctgacctgcaaggccagcggcttcgacctgaacaactacgccatgctgtgggtgagacaggcccccggaaaaggcctggagtggattggatgcatttacattagcggcggcaccacctacttcgccagctgggccacaggcagattcgccatcagcaaaaccagcagcaccaccgtgaccctgcagatgacaagcctgaccgccgccgacaccgccacctacttttgcgcccgcgacgacggaaccaccagctactacctgaacctgtggggacccggcaccctggtgaccgtgagctcc

SEQ ID NO: 430 - DNA, 354 bp, synthetic construct
  misc_feature 1..354: note = Synthetic
  source 1..354: mol_type = other DNA
cagagcctggaggaaagcggcggcgacctggtgcagcccggagcttcactgaccctgacctgcaaggccagcggcttcgacctgaacaactacgccatgctgtgggtgagacaggcccccggaaaaggcctggagtggattggaagcatttacatcagtggaggcaccacctactttgccagctgggccaccggcagattcgccatcagcaaaacctcctctaccaccgtgaccctgcagatgacatctctgactgccgccgacaccgccacctacttttgcgcccgcgacgacggaaccacctcctactatctgaacctgtggggacccggcaccctggtgaccgtgagcagc

SEQ ID NO: 431 - DNA, 354 bp, synthetic construct
  misc_feature 1..354: note = Synthetic
  source 1..354: mol_type = other DNA
cagtcgttggaggagtccgggggagacctggtccagcctggggcatccctgacactcacctgcaaagcctctggattcgacctcaataactatgcaatgctctgggtccgccaggctccagggaaggggctggagtggatcggatgcatttatattagtggtggtaccacttacttcgcgagctgggcgacaggccgattcgccatctccaaaacctcgtcgaccaccttgactctgcaaatgaccacccttccccccccggacacggccacctatttctgtgcgaaaaatgatggtactactagttattatcttaacttgtggggcccaggcaccctggtcaccgtctcctca

SEQ ID NO: 432 - DNA, 354 bp, synthetic construct
  misc_feature 1..354: note = Synthetic
  source 1..354: mol_type = other DNA
cagtcgttggaggagtccgggggagacctggtccagcctggggcatccctgacactcacctgcaaggcctctggattcgacctcaataactatgcaattctctgggtccgccaggctccagggaaggggctggagtggatcggatgcatttatattagtggtgctaccacttacttcgcgaactgggcgacaggccgattcgccatctccaaaacctcgtcgaccacggtgactctgcaaatgaccagtctgacagccgcggacacggccacctatttctgtgcgagagatgatggtactactagttattatcttaacttgtggggcccaggcaccctggtcaccgtctcctca

SEQ ID NO: 433 - DNA, 354 bp, synthetic construct
  misc_feature 1..354: note = Synthetic
  source 1..354: mol_type = other DNA
cagtcgttggaggagtccgggggagacctggtccagcctggggcatccctgacactcacctgtaaagcctctggattcgacctcaataattatgcaatgctttgggtccgccaggctccagggaaggggctggagtggatcggatgcatttatattagtggtggtaccacttacttcgcgagctgggcgacaggccgattcgccatctccaaaacctcgtcgaccacggtgactctgcaaatgaccagtctgacagccgcggacacggccacctatttctgtgcgagagatgatggtaccactagttattatcttaacttgtggggcccaggcaccctggtcaccgtctcctca

SEQ ID NO: 434 - DNA, 354 bp, synthetic construct
  misc_feature 1..354: note = Synthetic
  source 1..354: mol_type = other DNA
cagtcgttggaggagtccgggggagacctggtccagcctggggcatccctgacactcacctgtaaagcctctggattcgacctcaataactatgcaatgctctgggtccgccaggctccagggaaggggctggagtggatcggatgcatttatattagtggtggtaccacttacttcgcgagctgggcgacaggccgattcgccatctccaaaacctcgtcgaccacggtgactctgcaaatgaccagtctgacagccgcggacacggccacctatttctgtgcgagagatgatggtactactagttattatcttaacttgtggggcccaggcaccctggtcaccgtctcctca

SEQ ID NO: 435 - DNA, 354 bp, synthetic construct
  misc_feature 1..354: note = Synthetic
  source 1..354: mol_type = other DNA
cagtcgttggaggagtccgggggagacctggtccagcctggggcatccctgacactcacctgtaaagcctctggattcgacctcaataactatgcaatgctctgggtccgccaggctccagggaaggggctggagtggatcggatgcatttatattagtggtggtaccacttacttcgcgagctgggcgacaggccgattcgccatctccaaaacctcgtcgaccacggtgactctgcaaatgaccagcctgacagccgcggacacggccacctatttctgtgcgagagatgatggtagtactagttattatcttaacttgtggggcccaggcaccctggtcaccgtctcctca

SEQ ID NO: 436 - DNA, 354 bp, synthetic construct
  misc_feature 1..354: note = Synthetic
  source 1..354: mol_type = other DNA
cagtcgttggaggagtccgggggagacctggtccagcctggggcatccctgacactcacctgtaaagcctctggattcgacctcaataactatgcaatgctctgggtccgccaggctccagggaaggggctggagtggatcggatgcatttatattagtggtggtaccacttacttcgcgagctgggcgacaggccgattcgccatctccagaacctcgtcgaccacggtgactctgcaaatgaccagtctgacagccgcggacacggccacctatttctgtgcgagagatgatggtagtactagttattatcttaacttgtggggcccaggcaccctggtcaccgtctcctca

SEQ ID NO: 437 - DNA, 354 bp, synthetic construct
  misc_feature 1..354: note = Synthetic
  source 1..354: mol_type = other DNA
caatcgttggaggagtccgggggaaacctggcccaccctggggcatccctgagactcacctgtacagcctctggattctacctcaataactatgcaatgctctgggtccgccaggctccagggaaggggctggagtggatcggatgcctttatattagtggtggtaccacttacttcgcgagctgggcgacaggccgattcgccatctccaaaacctcgtcaaccactgggactctgcaaatgaccagtctgacagccgcggacacggccacctatttctgtgcgagaaatgatggtagtactaattattatcttaacttgtggggcccaggcaccctggtcaccatctcctca

SEQ ID NO: 438 - DNA, 354 bp, synthetic construct
  misc_feature 1..354: note = Synthetic
  source 1..354: mol_type = other DNA
cagtcgttggaggagtccgggggagacctggtccagcctggggcatccctgacactcacctgtagagcctctggattcgacctcaataactattcaatgctctgggtccgccaggctccagggaaggggctggagtggatcggatgcatttatattagtggtggtaccacttacttcgcgagctgggcgacaggccgattcgccatctccaaaacctcgtcgaccacggtgactctgcaaatgaccagtctgacagccgcggacacggccacctatttctgtgcgagagatgatggtagtactagttattatcttaacttgtggggcccaggcaccctggtcaccgtctcctca

SEQ ID NO: 439 - DNA, 354 bp, synthetic construct
  misc_feature 1..354: note = Synthetic
  source 1..354: mol_type = other DNA
cagtcgttggaggagtccgggggagacctggtccagcctggggcatccctgacactcacctgtagagcctctggattcgacctcaataactattcaatgctctgggtccgccaggctccagggaaggggctggagtggatcggatgcatttatattagtggtggtaccacttacttcgcgagctgggcgacaggccgattcgccatctccaaaacctcgtcgaccacggtgactctgcaaatgaccattctgacagccgcggacacggccacctatttctgtgcgagagatgatggtagtactagttattatcttaacttgtggggcccaggcaccctggtcaccgtctcctca

SEQ ID NO: 440 - DNA, 354 bp, synthetic construct
  misc_feature 1..354: note = Synthetic
  source 1..354: mol_type = other DNA
cagtcgttggaggagtccgggggagacctggtccagcctggggcatccctgacactcacctgtaaagcctctggattcgacctcaacaactattcaatactatgggtccgccaggctccagggaaggggctggagtggatcggatgcatttatattagtggtggtaccacttacttcgcgagctgggcgacaggccgattcgccatctccaaaacctcgtcgaccacggtgactctgcaaatgaccagtctgacagccgcggacacggccacctatttctgtgcgagagatgatggtactactagttattatcttaacttgtggggcccaggcaccctggtcaccgtctcctca

SEQ ID NO: 441 - DNA, 354 bp, synthetic construct
  misc_feature 1..354: note = Synthetic
  source 1..354: mol_type = other DNA
cagtcgttggaggagtccgggggagacctggtccagcctggggcatccctgacactcacctgcaaagccgctggattcgacctcaataattattcaatgctctgggtccgccaggctccagggaaggggctggagtggatcggatgcatttatattagtggtggtaccacttactacgcgagctgggcgaaaggccgattcaccatctccaaaacctcgtcgaccacggtgactctgcaaatgaccagtctgacagccgcggacacggccacctatttctgtgcgagagatgatgggagtactagttattatttaaacctgtggggcccaggcaccctggtcaccgtctcctca

SEQ ID NO: 442 - DNA, 354 bp, synthetic construct
  misc_feature 1..354: note = Synthetic
  source 1..354: mol_type = other DNA
cagtcgttggaggagtccgggggagacctggtccagcctggggtatccctgacactcacctgcaaagcctctggattcgacctcaataactattcaatactctgggtccgccaggctccagggaaggggctggagtggatcggatgcatttatattagtggtggtagcacttactacgcgagctgggcgaaaggccgattcgccatctccaaaacctcgtcgaccacggtgactctgcaaatgaccagtctgacagccgcggacacggccacctatttctgtgcgagagatgatggtactactagttattatcttaacttgtggggcccaggcaccctggtcaccgtctcctca

SEQ ID NO: 443 - DNA, 354 bp, synthetic construct
  misc_feature 1..354: note = Synthetic
  source 1..354: mol_type = other DNA
cagtcgttggaggagtccgggggagacctggtccagcctggggcatccctgacactcacctgcaaagcctctggattcgacctcaataactattctatactctgggtccgccaggctccagggaaggggctggagtggatcggatgtatctatattagtggtggtaccacttactacgcgaactgggcgaaaggccgattcaccatctccagaacctcgtcgaccacggtgactctgcaaatgaccagtctgacaggcgcggacacggccacctatttctgtgcgagagatgatggtagtactagttattatttgaacttgtggggcccaggcaccctggtcaccgtctcctca

SEQ ID NO: 444 - DNA, 354 bp, synthetic construct
  misc_feature 1..354: note = Synthetic
  source 1..354: mol_type = other DNA
cagtcgttggaggagtccgggggagacctggtccagcctggggcatccctgacactcacctgcaaagcctctggattcgacctcaataactattcaatactctgggtccgccaggctccagggaaggggctggagtggatcggatgcatttatattagtggtggtaccacttactacgcgaactgggcgaaaggccgattcgccatctccaaaacctcgtcgaccacggtgactctgcaaatgaccagtctgacagccgcggacacggccacctatttctgtgcgagagatgatggtactactagttattatcttaacttgtggggcccaggcaccctggtcaccgtctcctca

SEQ ID NO: 445 - DNA, 354 bp, synthetic construct
  misc_feature 1..354: note = Synthetic
  source 1..354: mol_type = other DNA
cagtcgttggaggagtccgggggagacctggtccagcctggggcatccctgacactcacctgcaaagcctctggattcgacctcaataactattcaatactctgggtccgccaggctccagggaaggggctggagtggatcggatgcatttatattagtggtggtagcacttactacgcgaactgggcgaaaggccgattcgccatctccaaaacctcgtcgaccacggtgactctgcaaatgaccagtctgacagccgcggacacggccacctatttctgtgcgagagatgatggtactactagttattatcttaacttgtggggcccaggcaccctggtcaccgtctcctca

SEQ ID NO: 446 - DNA, 354 bp, synthetic construct
  misc_feature 1..354: note = Synthetic
  source 1..354: mol_type = other DNA
cagtcgttggaggagtccgggggagacctggtccagcctggggcatccctgacactcacctgcaaagcctctggattcgacctcaataactattcaatactctgggtccgccaggctccagggaaggggctggagtggatcggatgcatttatattagtggtggtagcacttactacgcgaactgggcgaaaggccgattcgccgtctccaaaacctcgtcgaccacggtgactctgcaaatgaccagtctgacagccgcggacacggccacctatttctgtgcgagagatgatggtactactagttattatcttaacttgtggggcccaggcaccctggtcaccgtctcctca

SEQ ID NO: 447 - DNA, 354 bp, synthetic construct
  misc_feature 1..354: note = Synthetic
  source 1..354: mol_type = other DNA
cagtcgttggaggagtccgggggagacctggtccagcctggggcatccctgacactcacctgcaaagcctctggattcgacctcaataactattcaatactctgggtccgccaggctccagggaaggggctggagtggatcggatgcatttattttagtggtggtagcacttactacgcgaactgggcgaaaggccgattcgccatctccaaaacctcgtcgaccacggtgactctgcaaatgaccagtctgacagccgcggacacggccacctatttctgtgcgagagatgatggtactactagttattatcttaacttgtggggcccaggcaccctggtcaccgtctcctca

SEQ ID NO: 448 - DNA, 354 bp, synthetic construct
  misc_feature 1..354: note = Synthetic
  source 1..354: mol_type = other DNA
cagtcgttggaggagtccgggggagacctggtccagcctggggcatccctgacactcacctgcaaagcctctggattcgacctcaataactattcaatactctgggtccgccaggctccagggaaggggctggagtggatcggatgcatttatattagtggtggtagtacttactacgcgaactgggcgaaaggccgattcgccatctccaaaacctcgtcgaccacggtgactctgcagatgaccagtctgacagccgcggacacggccacctatttctgtgcgagagatgatgggactgctagttattatcttaacttgtggggcccaggcaccctggtcaccgtctcctca

SEQ ID NO: 449 - DNA, 354 bp, synthetic construct
  misc_feature 1..354: note = Synthetic
  source 1..354: mol_type = other DNA
cagtcgttggaggagtccgggggagacctggtccagcctgggacatccctgacactctcctgcaaagcctctggattcgacctcaataactattctatactctgggtccgccaggctccagggaaggggctggaatggatcggatgcatctatattagtggtggaaccacttattttgcgaactgggcgaaaggccgattcaccatctccagaccctcgtcgaccacggtgactctgcaaatgaccagtctgacaggcgcggacacggccacttatttctgtgcgagagatgatggtagtgttagttattatttgaacttgtggggcccaggcaccctggtcatcgtctcctca

SEQ ID NO: 450 - DNA, 366 bp, synthetic construct
  misc_feature 1..366: note = Synthetic
  source 1..366: mol_type = other DNA
cagcagctggaggaaagcggcggcggcctggtgaagcccgagggaagtctgaccctgacctgcaagggcagcggcttcgatttcagcggcacctactggatgtggtgggtgagacaggcccctggcaagggactggagtggattgcttgtatctacgtgggctctggcagcagcacctattacgcttcatgggctaagggcaggtttacaatcagcagcactagcagtaccgccgtgaccctgcaggctactagtctgaccgctgccgacaccgccacatacttctgcgcccgcggcgccactaacaacgtgttcatgaactacttcaacctgtggggacccggaacactggtgaccgtgtcatcc

SEQ ID NO: 451 - DNA, 366 bp, synthetic construct
  misc_feature 1..366: note = Synthetic
  source 1..366: mol_type = other DNA
cagcagctggaggaaagcggcggcggcctggtgaagcccgagggaagtctgaccctgacctgcaagggcagcggcttcgatttcagcggcacctactggatgtggtgggtgagacaggcccctggcaagggactggagtggattgctcccatctacgtggggagcggcagcagcacctactacgcttcatgggctaagggcaggttcacaatcagcagcacctcttctaccgctgtgactctgcaggctactagtctgaccgccgccgacaccgccacctacttctgcgccagaggcgccaccaacaacgtgttcatgaactacttcaacctgtggggccccggcaccctggtgacagtgagctcc

SEQ ID NO: 452 - DNA, 366 bp, synthetic construct
  misc_feature 1..366: note = Synthetic
  source 1..366: mol_type = other DNA
cagcagctggaggaaagcggcggcggcctggtgaagcccgagggaagtctgaccctgacctgcaagggcagcggcttcgatttcagcggcacctactggatgtggtgggtgagacaggcccctggcaagggactggagtggattgctagtatctacgtggggagcggcagcagcacctactacgcttcctgggctaagggcagatttacaatcagcagcactagcagtaccgccgtgaccctgcaggccaccagcctgaccgccgctgacaccgctacatacttctgcgccagaggcgccaccaacaacgtgttcatgaactacttcaacctgtggggacccggcacactggtgaccgtgagcagc

SEQ ID NO: 453 - DNA, 366 bp, synthetic construct
  misc_feature 1..366: note = Synthetic
  source 1..366: mol_type = other DNA
cagcagctggaggagtccgggggaggcctggtcaagcctgagggatccctgacactcacctgcaaaggctctggattcgacttcagtggcacctattggatgtgctgggtccgccaggctccagggaaggggctggagtggatcgcatgcatttatgttggtagtggtggtagcacttactacgcgagctgggcgaaaggccgattcaccatctccagcacctcgtcgaccgcggtgactctgcaagtgaccagtctgacagccgcggacacggccacctatttctgtgcgagaggtgctactaataatgtgtttagaaactactttaacttgtggggcccaggcaccctggtcaccgtctcctca

SEQ ID NO: 454 - DNA, 354 bp, synthetic construct
  misc_feature 1..354: note = Synthetic
  source 1..354: mol_type = other DNA
cagagcctggaggaaagcggcggcggcctggtgaagcccggaggaacactgaccctgacctgcaccgtgagcggcttcgatttcagcaactacgccatgacctgggtgagacaggccccaggagaagggctggagtatatcggcttcgtggccctgagaggaaacatttactacgccaactgggctaagggaaggtttactatcagcaagactagcagcaccaccgtgacactgcagatgaccagtctgacagtggccgacaccgccacatacttctgcgccaggggcggcctgtacactggatacagctactttgacctgggcgggcccgggaccctggctaccgtgagctca

SEQ ID NO: 455 - DNA, 354 bp, synthetic construct
  misc_feature 1..354: note = Synthetic
  source 1..354: mol_type = other DNA
cagtcgttagtggagtccgggggaggcctggtccagcctgagggatccctgacactcacctgcaaagcctctggattcgacttcaataattatgcagtgacctgggtccgccaggctccaggggaggggctggaatacatcggattcattggtattcgtggtcatatttactacgcgaactgggcgaaaggccgattcaccatctccaaaacctcgtcgaccacgatgactctacaaatgactagtctgacagtcgcggacacggccacctatttctgtgcgagagggggcttatggactggtaatagttactttgacttgtggggcccaggcaccctggtcaccgtctcctca

SEQ ID NO: 456 - DNA, 354 bp, synthetic construct
  misc_feature 1..354: note = Synthetic
  source 1..354: mol_type = other DNA
cagtcgttggaggagtccgggggaggcctggtcaagcctggaggaaccctgacactcacctgcacagtctctggattcgacttcagtaattatgcgatgacctgggtccgccaggctccagggaaagggctggaatacatcggattcattggtattcgtggtaatatttactacgcgaactgggcgaaaggccgattcaccatctccaaaacctcgtcgaccacggtgactctgcaaatgaccagtctgacagtcgcggacacggccacctatttctgtgcgagagggggcttatggactggtaatagttattttgacttgtggggcccaggcaccctggtcaccgtctcctca

SEQ ID NO: 457 - DNA, 354 bp, synthetic construct
  misc_feature 1..354: note = Synthetic
  source 1..354: mol_type = other DNA
cagtcgttggaggagtccgggggaggcctggtccagcctggggcatccctgacactcacctgcacagtctctggattcgacttcagtagcaatgcaatgacctgggtccgccaggctccaggggaggggctggaatacatcggattcattggtattcgtggtaatatttactacgcgaactgggcgaaaggccgattcaccatctccaaaacctcgtcgaccacggtgactctgcaaatgaccagtctgacagtcgcggacacggccacgtatttctgtgcgagagggggcttatggactggtaacagttattttgacttgtggggcccaggcaccctggtcaccgtctcctca

SEQ ID NO: 458 - DNA, 354 bp, synthetic construct
  misc_feature 1..354: note = Synthetic
  source 1..354: mol_type = other DNA
cagtcgttggaggagtccgggggaggcctggtcaagcctggaggaaccctgacactcacctgcacagtctctggattcgacttcagtaattatgctgtgacctgggtccgccaggctccaggggaggggctggaatacatcggattcgttggtatttatggtgatttttactacgcgaactgggcgaaaggccgattcaccatctccaaaacctcgtcgaccacggtgactctgcaaatgcccagtctgacagtcgcggacacggccacctatttctgtgcgagagggggcttatggactggtaacagttattttgacttgtggggcccaggcaccctggtcaccgtctcctca

SEQ ID NO: 459 - DNA, 330 bp, synthetic construct
  misc_feature 1..330: note = Synthetic
  source 1..330: mol_type = other DNA
gatgtggtgatgacccagacccccgcctctgtggaagtggccgtgggcggaaccgtgaccattaaatgtcaggcctctgaagatattgagtcctatctggcctggtaccagcagaaacccggccagccccctaagctgctgatctacgaggcctccaagctggctagtggcgtgagctccagatttagcggagggggatacggcacagagttcaccctgactatcagcggcgtggagtgtgccgacgccgccacatactactgccagcaggccctgactgtgggaaatgtggacaacccattcggaggggggagcgaggtggtggtgaag

SEQ ID NO: 460 - DNA, 330 bp, synthetic construct
  misc_feature 1..330: note = Synthetic
  source 1..330: mol_type = other DNA
gacgtcgtgatgacccagactccagcctctgtggaggtagctgtgggaggcacagtcaccatcaagtgccaggccagtgaggacattgaaagctatttagcctggtatcagcagaaaccagggcagcctcccaaactcctgatctatgaagcatccaagctggcatctggggtctcatcgcggttcagcggcggtggatatgggacagagttcactctcaccatcagcggcgtggagtgtgccgatgctgccacttactactgtcaacaggctttgactgttggtaatgttgataatcctttcggcggaggaagcgaagtggtggtcaaa

SEQ ID NO: 461 - DNA, 330 bp, synthetic construct
  misc_feature 1..330: note = Synthetic
  source 1..330: mol_type = other DNA
gacgtcgtgatgacccagactgcatcccccgtgtctgcagctgtgggaggcacagtcaccatcaagtgccaggccagtcagagcatttacagctacttatcctggtatcaacagaaaccagggcagcctcccaagctcctgatctacgaagcatccaaactggcatctggggtctcatcgcggttcagcggcagtggatatgggacagagttcactctcaccatcagcggcgtggagtgtgccgatgctgccacttactactgtcaacaggctttgactgttggtaatgttgataatcctttcggcggaggaagcgaggtggtggtcaaa

SEQ ID NO: 462 - DNA, 330 bp, synthetic construct
  misc_feature 1..330: note = Synthetic
  source 1..330: mol_type = other DNA
gacattgtgatgacccagacccccgcctccgtgagcgaacccgtgggaggaaccgtgaccataaaatgccaggccagccaaagcattagcagctatctggcatggtatcagcagaaacccggacagccccccaagctgctcatatacgaggcaaccaaactgacaagcggcgtgagcagcagattcagcggcagcggctacggcaccgagttcaccctgactatcagcggcgtggaatgcgccgacgccgccacatactactgccagcaggccctgaccgtgggagacgtggacaacccctttggaggcggcagcgaggttgtcgtgaag

SEQ ID NO: 463 - DNA, 330 bp, synthetic construct
  misc_feature 1..330: note = Synthetic
  source 1..330: mol_type = other DNA
gacgtcgtgatgacccagacaccagcctccgtgtctgcagctgtgggaggcacagtcaccatcaagtgccaggccagtgaggacattgaaagctatttagcctggtatcagcagaaaccagggcagcctcccaagctcctgatctatgatgcatccactctggcatctggggtctcatcgcggttcagcggcagtggatatgggacagagttcactctcaccatcagcggcgtggagtgtgccgatgctgccacttactactgtcaacaggatttgactgttgggaatgttgataatcctttcggcggagggagcgaggtggtggtcgaa

SEQ ID NO: 464 - DNA, 330 bp, synthetic construct
  misc_feature 1..330: note = Synthetic
  source 1..330: mol_type = other DNA
gacgtcgtgatgacccagactccagcctctgtggaggcagctgtgggaggcacagtcaccatcaattgccaggccagtcagagcattgaaagctatttagcctggtatcagcagaaaccagggcagcctcccaaactcctgatctatgaagcatccaaactggcatctggggtctcatcgcggttcagcggcggtggatatgggacagagttcactctcaccatcagcggcgtggagtgtgccgatgctgccacttactactgtcaacaggctttgactattggtaatgttgataatcctttcggcggaggaagcgaggtggtggtcaaa

SEQ ID NO: 465 - DNA, 330 bp, synthetic construct
  misc_feature 1..330: note = Synthetic
  source 1..330: mol_type = other DNA
gacgtcgtgatgacccagactccagcctctgtggaggtagctgtgggaggcacagtcaccatcaagtgccaggccagtgaggacattgaaagctatttagcctggtatcagcagaaaccagggcagcctcccaaactcctgatctatgaagcatccaaactggcatctggggtctcatcgcggttcagcggcggtggatatgggacagagttcactctcaccatcagcggcgtggagtgtgccgatgctgccacttactactgtcaacaggctttgactattggtaatgttgataatcctttcggcggaggaagcgaggtggtggtcaaa

SEQ ID NO: 466 - DNA, 330 bp, synthetic construct
  misc_feature 1..330: note = Synthetic
  source 1..330: mol_type = other DNA
gatgtggtgatgacccagacccccgcctctgtggaagtggccgtgggcggaaccgtgaccattaaatgtcaggcctctgaagatattgagtcctatctggcctggtaccagcagaaacccggccagccccctagactgctgatttacgaggcctccaagctggctagtggcgtgagctccagatttagcggagggggatacggcacagacttcaccctgactatcagcggcgtggagtgcgccgacgccgctacttactactgccaccaggccctgaccattggaaatgtggacaacccattcgggggaggcggagaggtggtggtgaag

SEQ ID NO: 467 - DNA, 330 bp, synthetic construct
  misc_feature 1..330: note = Synthetic
  source 1..330: mol_type = other DNA
gacgtggtgatgacccaaacagccagccccgtgagcgccgccgtgggaggaacagtgaccattaagtgtcaggcaagcgaggacatcgagagctacctggcctggtatcagcagaaacccggccagccccccaaactcctgatctacgaggcatccaagctggccagcggcgtgagcagcagattcagcggaagcggctacggcacagaatttaccctgactatctccggcgtggaatgcgccgacgccgccacttactactgccagcaggccctgactatcggaaacgttgacaacccctttggaggaggatctgaggttgtggtgaag

SEQ ID NO: 468 - DNA, 330 bp, synthetic construct
  misc_feature 1..330: note = Synthetic
  source 1..330: mol_type = other DNA
gacgtcgtgatgacccagacaccagcctccgtgtctgcagctgtgggaggcacagtcagcatcaattgccaggccagtgaggacattgaaaactatttagcctggtatcagcagaaaccagggcagcctcccaaactcctgatctatgaagcatccaaactggcatctggggtctcatcgcggttcagcggcagtggatatgggacagagttcactctcaccatcagcggcgtggagtgtgccgatgctgccacttactactgtcaacaggctttgactattggtaatgttgataatcctttcggcggaggaagcgaggtggtggtcaaa

SEQ ID NO: 469 - DNA, 330 bp, synthetic construct
  misc_feature 1..330: note = Synthetic
  source 1..330: mol_type = other DNA
gacgtggtgatgacccaaacacccgccagcgtggaagtggccgtgggaggaacagtgaccctgaagtgccaggcaagcgaggacattgaaagctacagcgcctggtatcaacaaaaacccggccagccccccaacctgctcatctacgaggcctccaagctggcaagcggcgtgagctccagattcagcggatcaggctacggcacagaatttacactgaccatcagcggcgtggaatgcgccgacgccgccacatactactgccagcaggccctgactataggcaacgtggacaaccccttcggaggcggcagcgaggtggtggtgagg

SEQ ID NO: 470 - DNA, 330 bp, synthetic construct
  misc_feature 1..330: note = Synthetic
  source 1..330: mol_type = other DNA
gacgtggtgatgacccaaacacccgccagcgtggaagccgccgttggaggaacagttagcattaactgccaggccagcgaggacattgaaagctatctggcctggtaccagcagaagcccggacagccccccaaactgctgatttacaaggcatctacactggcaagcggcgtgtccagcagattcaaaggaagcggcagcggcaagcagttcaccctgactatcagcggcgtggaatgcgccgatgccgccacctactactgccagcaggtcctcaccataggaaacgtggacaaccccttcggaggcggaagtgaagtggtcgtcaag

SEQ ID NO: 471 - DNA, 330 bp, synthetic construct
  misc_feature 1..330: note = Synthetic
  source 1..330: mol_type = other DNA
gacgtcgtgatgacccagactccagcctctgtggaggtagctgtgggaggcacagtcaccatcaagtgccaggccagtgagaacattgaaagctatttagcctggtatcagcagaaaccagggcagcctcccaagctcctgatctatgatgcatccactctggcatctggggtctcatcgcggttcagcggcagtggatatgggacagagttcactctcaccatcagcggcgtggagtgtgccgatgctgccacttactactgtcaacagggtttgactattggtaatgttgataatcctttcggcggaggaagcgaggtggtggtcaaa

SEQ ID NO: 472 - DNA, 330 bp, synthetic construct
  misc_feature 1..330: note = Synthetic
  source 1..330: mol_type = other DNA
gacgtggtgatgacccaaacacccgccagcgtggaagtggccgtgggaggaacagtgaccattaagtgccaggccagcaagaacatagacagcaacctggcttggtatcagcaaaaacccgggcagccccccaaacaactgatatacgctgcctccacactggccagcggcgtgagcagcagattcagcggaagcggctacggagcagagttcaccctgactatcagcggcgtggaatgcgcagacgccgcaacctattactgccaacaggccctgactattggcaacgtggacaacccctttggaggcggaagtgaggtggtggtgaag

SEQ ID NO: 473 - DNA, 330 bp, synthetic construct
  misc_feature 1..330: note = Synthetic
  source 1..330: mol_type = other DNA
gacgtggtgatgacccaaacacccgccagcgtggaagccgccgttggaggaacagttaccattaaatgccaggccagcgaggacattgaaagatatttggcctggtatcagcaaaaacccggccaaccccccaaactgctgatttacgaggcctccaaactgccaagcggcgtgagcagcagattcagcggatcaggctatggcaccgaatttaccctgactatctccggcgtggaatgtgccgacgccgccacctactactgccagcaggccctgaccataggctatgtcgacaaccccttcggaggcggctctgaggttgtggtgaag

SEQ ID NO: 474 - DNA, 330 bp, synthetic construct
  misc_feature 1..330: note = Synthetic
  source 1..330: mol_type = other DNA
gacgtcgtgatgacccagactccagcctctgtggaggcagctgtgggaggcacagtcaccatcaagtgccaggccagtgaggacattgaaaggtatttagcctggtatcagcagaaaccagggcagcctcccaagctcctgatttatgaagcatccaaactgccatctggggtctcatcgcggttcagcggcagtggatatgggacagagttcactctcaccatcagcggcgtggagtgtgccgatgctgccacttactactgtcaacaggctttgactattggttacgttgataatcctttcggcggaggaagcgaggtggtggtcaaa

SEQ ID NO: 475 - DNA, 327 bp, synthetic construct
  misc_feature 1..327: note = Synthetic
  source 1..327: mol_type = other DNA
tatgatatgacccagacccccgccagtgtgtccgagcccgtgggaggaaccgtgacaattaaatgtcaggctagtgaggatatttataaactgctggcctggtaccagcagaagcccggccagcccccaaaactgctgatttacgccgccagcgacctggaaagcggcgtgccaagcagattcaagggaagaggaagcggaaccgactacaccctgactatcagcgacctggagtgtgccgacgccgccacctactactgccagcaggcctatactatcggaaatatcgacaacagcttcggaggaggaacagaagtggtggtgaag

SEQ ID NO: 476 - DNA, 327 bp, synthetic construct
  misc_feature 1..327: note = Synthetic
  source 1..327: mol_type = other DNA
tatgatatgacccagacccccgccagtgtgtccgagcccgtgggaggaaccgtgacaattaaatgtcaggctagtgaggatatttataaactgctggcctggtaccagcagaagcccggccagcccccaaaactgctgatttacgccgccagcgacctggaaagcggcgtgccaagcagattcaagggaagaggaagcggaaccgactacaccctgactatcagcgacctggaggccgccgacgccgctacatactactgtcagcaggcctatactatcggaaacattgacaacagcttcggcggaggaacagaagtggtggtgaag

SEQ ID NO: 477 - DNA, 327 bp, synthetic construct
  misc_feature 1..327: note = Synthetic
  source 1..327: mol_type = other DNA
tatgatatgacccagacccccgccagtgtgtccgccgctgtgggaggaaccgtgacaattaaatgtcaggctagtgaggatatttattccctgctggcctggtaccagcagaagcccggccagccccccaagctgctgatttacggcgcctctaatctggaatctggcgtgcccagcagatttaagggctccggcagcggcaccgagtacaccctgactatcagcgacctggaatgcgacgacgccgccacctactactgccagcaggcttacaccatcggcaacatcgacaacgccttcggaggaggcacagaagtggtggtgaag

SEQ ID NO: 478 - DNA, 327 bp, synthetic construct
  misc_feature 1..327: note = Synthetic
  source 1..327: mol_type = other DNA
tatgatatgacccagacccccgccagtgtgtccgccgctgtgggaggaaccgtgacaattaaatgtcaggctagtgaggatatttattccctgctggcctggtaccagcagaagcccggccagccccccaagctgctgatttacggcgcctctaatctggaatctggcgtgcccagcagatttaagggctccggcagcggcaccgagtacaccctgactatcagcgacctggaagctgacgacgccgccacctactactgccagcaggcttacaccatcggcaacatcgacaacgccttcggcggcggcaccgaggtggtggtgaag

SEQ ID NO: 479 - DNA, 327 bp, synthetic construct
  misc_feature 1..327: note = Synthetic
  source 1..327: mol_type = other DNA
tatgatatgacccagactccagcctccgtgtctgcagctgtgggaggcacagtcaccatcaagtgccaggccagtgaggacatttataggttattggcctggtatcagcagaaaccagggcagcctcccaagctcctgatctatggtgcatccaatctggaatctggggtcccatcgcggttcaaaggcagtggatctgggacagagtacactctcaccatcagcgacctggagtgtgacgatgctgccacttactactgtcaacaggcttatactattggtaatattgataatgctttcggcggagggaccgaggtggtggtcaaa

SEQ ID NO: 480 - DNA, 327 bp, synthetic construct
  misc_feature 1..327: note = Synthetic
  source 1..327: mol_type = other DNA
tatgatatgacccagactccagcctccgtggaggcagctgtgggaggcacagtcaccatcaagtgccaggccagtgaggacatttataggttattggcctggtatcagcagaaaccagggcagcctcccaagctcctgatctatggtgcatccaatctggaatctggggtcccatcgcggttcaaaggcagtggatctgggacagagtacactctcaccatcagcgacctggagtgtgacgatgctgccacttactactgtcaacaggcttatactattggtaatattgataatgctttcggcggagggaccgaggtggtggtcaaa

SEQ ID NO: 481 - DNA, 327 bp, synthetic construct
  misc_feature 1..327: note = Synthetic
  source 1..327: mol_type = other DNA
tatgatatgacccagactccagcctccgtgtctgcagctgtgggaggcacagtcaccatcaagtgccaggccagtgaggacatttatagcttattggcctggtatcagcagaaaccagggcagcctcccaagctcctgatctatgatgcatccgatctggcatctggggtcccatcgcggttcaaaggcagtggatctgggacagagtacactctcaccatcagcgacctggagtgtgacgatgctgccacttactactgtcaacaggcttatactattggtaatattgataatactttcggcggagggaccgaggtggtggtcaaa

SEQ ID NO: 482 - DNA, 327 bp, synthetic construct
  misc_feature 1..327: note = Synthetic
  source 1..327: mol_type = other DNA
tatgatatgacccagactccagcctccgtgtctgcagctgtgggaggcacagtcaccatcaagtgccaggccagtgaggacatttatagcttattggcctggtatcagcagaaaccagggcagcctcccaagctcctgatctatgatgcatccgatctggcatctggggtcccatcgcggttcaaaggcagtggatctgggacagagtacactctcaccatcagcgacctggagtgtgacgatgctgccacttactactgtcaacaggcttatactattggtaatattgataatgctttcggcggagggaccgaggtggtggtcaaa

SEQ ID NO: 483 - DNA, 327 bp, synthetic construct
  misc_feature 1..327: note = Synthetic
  source 1..327: mol_type = other DNA
tatgatatgacccagactccagcctccgtgtctgcagctgtgggaggcacagtcaccatcaagtgccaggccagtgaggacatttatagcttattggcctggtatcagcagaaaccagggcagcctcccaagctcctgatctatgatgcatccgatctggcatctggggtcccatcgcggttcaaaggcagtggatctgggacagagtacactctcaccatcagcgccctggagtgtgacgatgctgccacttactactgtcaacaggcttatactattggtaatattgataatactttcggcggagggaccgaggtggtggtcaaa

SEQ ID NO: 484 - DNA, 327 bp, synthetic construct
  misc_feature 1..327: note = Synthetic
  source 1..327: mol_type = other DNA
tatgatatgacccagactccagcctccgtgtctgaacctgtgggaggcacagtcaccatcaagtgccaggccagtcagagcatttacaataattttgcctggtatcagcagaaaccagggcagcgtcccaagctcctgatctatgatgcatccgatctggcatctggggtcccatcgcggttcaaaggcagtggatctgggacagagtacactctcaccatcagcggcgtgcagtgtgccgatgcagccacttactactgtcaacaggcttatactattggtaatattgataatcctttcggcggagggaccgaggtggtggtcaaa

SEQ ID NO: 485 - DNA, 327 bp, synthetic construct
  misc_feature 1..327: note = Synthetic
  source 1..327: mol_type = other DNA
tatgatatgacccagactccagcctccgtgtctgcagctgtgggaggcacagtcaccatcaagtgccaggccagtgaggacatttatagcttattggcctggtatcagcagaaaccagggcagcctcccaagctcctgatctttgatgcatccgatctggcatctggggtcccatcgcggttcaaaggcagtggatctgggacagagtacactctcaccatcagcgacctggagtgtgacgatgctgccacttactactgtcaacaggcttatactattggtaatattgataatgctttcggcggagggaccgaggtggtggtcaaa

SEQ ID NO: 486 - DNA, 327 bp, synthetic construct
  misc_feature 1..327: note = Synthetic
  source 1..327: mol_type = other DNA
tatgatatgacccagactccagcctccgtgtctgcagctgtgggaggcacaatcaccatcaagtgccaggccagtgaggacatttatagcttattggcctggtatcagcagaaaccagggcagcctcccaagctcctgatctttgatgcatccactctggcatctggggtcccatcgcggttcaaaggcagtggatctgggacagagtacactctcaccatcagcgacctggagtgtgacgatgctgccacttactactgtcaacaggcttatactattggtaatattgataatgctttcggcggagggaccgaggtggtggtcgaa

SEQ ID NO: 487 - DNA, 327 bp, synthetic construct
  misc_feature 1..327: note = Synthetic
  source 1..327: mol_type = other DNA
tatgatatgacccagactccagcctccgtgtctgaacctgtgggaggcacagtcaccatcaagtgccaggccagtgaggacatttataagttattggcctggtatcagcagaaaccagggcagcctcccaagctcctgatctatgctgcatccgatctggaatctggggtcccatcgcggttcaaaggcagtggatctgggacagagtacactctcaccatcagcgacctggagtgtgccgatgctgccacttactactgtcaacaggcttatactattggtaatattgataatactttcggcggagggaccgaggtggtggtcaaa

SEQ ID NO: 488 - DNA, 327 bp, synthetic construct
  misc_feature 1..327: note = Synthetic
  source 1..327: mol_type = other DNA
tatgatatgacccagactccagcctccgtgtctgcagctgtgggaggcacagtcaccatcaagtgccaggccagtgaggacatttatagtttattggcctggtatcagcagaaaccagggcagcctcccaagctcctgatctatgatgcatccgatctggcatctggggtcccatcgcggttcaaaggcagtggatctgggacagagtacactctcaccatcagcgacctggagtgtgacgatgctgccacttactactgtcaacaggcttatactattggtaatattgataattctttcggcggagggaccgaggtggtggtcaaa

SEQ ID NO: 489 - DNA, 327 bp, synthetic construct
  misc_feature 1..327: note = Synthetic
  source 1..327: mol_type = other DNA
tatgatatgacccagactccagcctccgtgtctgaacctgtgggaggcacagtcaccatcaagtgccaggccagtgaggacatttataggttattggcctggtatcagcagaaaccagggcagcctcccaagctcctgatctatgatgcatccgatctggcatctggggtcccatcgcggttcaaaggcagtggatctgggacagagtacactctcaccatcagcggcttgcagtgtgccgatgcagccacttactactgtcaacagggttatactattggtaatattgataattctttcggcggagggaccgaggtggtggtcaaa

SEQ ID NO: 490 - DNA, 327 bp, synthetic construct
  misc_feature 1..327: note = Synthetic
  source 1..327: mol_type = other DNA
tatgatatgacccagactccagcctccgtgtctgaacctgtgggaggcacagtcaccatcaagtgccaggccagtgaggacatttataacttattggcctggtatcagcagaaaccagggcagcctcccaagctcctgatctatgctgcatccgatctggaatctggggtcccatcgcggttcaaaggcagtggatctgggacagagtacactctcaccatcagcgacctggagtgtgacgatgctgccacttactactgtcaacagggttatactattggtaatattgataatgctttcggcggagggaccgaggtggtggtcaaa

SEQ ID NO: 491 - DNA, 327 bp, synthetic construct
  misc_feature 1..327: note = Synthetic
  source 1..327: mol_type = other DNA
tatgatatgacccagactccagcctccgtgtctgaacctgtgggaggcacagtcaccatcaagtgccaggccagtgaggacatttataacttattggcctggtatcagcagaaaccagggcagcctcccaagctcctgatctatgatgcatccactctgacatctggggtcccatcgcggttcaaaggcagtggatctgggacagagtacactctcaccatcagcgacctggagtgtgacgatgctgccacttactactgtcaacaggcttatactattggtaatattgataatactttcggcggagggaccgaggtggtggtcaaa

SEQ ID NO: 492 - DNA, 327 bp, synthetic construct
  misc_feature 1..327: note = Synthetic
  source 1..327: mol_type = other DNA
tatgatatgacccagactccagcctccgtgtctgaacctgtgggaggcacagtcaccatcaagtgccaggccagtgaggacatttataaattattggcctggtatcagcagaaaccagggcagcctcccaagctcctgatctatgctgcatctgatctggaatctggggtcccatcgcggttcaaaggcagtggatctgggacagagtatactctcaccatcagcgacctggagtgtgccgatgctgccacttactactgtcaacaggcttatactatcggtaatcttgataatgctttcggcggagggaccgaggtggtggtcaaa

SEQ ID NO: 493 - DNA, 327 bp, synthetic construct
  misc_feature 1..327: note = Synthetic
  source 1..327: mol_type = other DNA
tatgatatgacccagactccagcctccgtgtctgaacctgtgggaggcacagtcaccatcaagtgccaggccagtgaggacatttataatttattggcctggtatcagcagaaaccagggcagcctcccaagctcctgatctatgctgcatccgatctggaatctggggtcccatcgcggttcaaaggcagtggatctgggacagagtacactctcaccatcagcgacctggagtgtgccgatgctgccacttactactgtcaacaggcttatactgtcggtaatattgataatgctttcggcggcgggaccgaggtggtggtcaaa

SEQ ID NO: 494 - DNA, 327 bp, synthetic construct
  misc_feature 1..327: note = Synthetic
  source 1..327: mol_type = other DNA
tatgatatgacccagactccagcctccgtgtctgaacctgtgggaggcacagtcaccatcaagtgccaggccagtgaggacatttataagttattggcctggtatcagcagaaaccagggcagcctcccaagctcctgatctatgctgcatccgatctggaatctggggtcccatcgcggttcaaaggcagtggatctgggacagactacactctcaccatcagcgacctggagtgtgccgatgctgccacttactactgtcaacaggcttatactatcggtaatattgataattctttcggcggagggaccgaggtggtggtcaaa

SEQ ID NO: 495 - DNA, 327 bp, synthetic construct
  misc_feature 1..327: note = Synthetic
  source 1..327: mol_type = other DNA
tatgatatgacccagactccagcctccgtgtctgaacctgtgggaggcacagtcaccatcaagtgccaggccagtgaggacatttataacttattggcctggtatcagcagaaaccagggcagcctcccaagctcctgatctatgatgcgtccactctgacatctggggtcccatcgcggttcaaaggcggtggatctgggacagagtacactctcaccatcaacgacctggagtgtgacgatgctgccacttactactgtcaacagggttatactattggtaatattgataatactttcggcggagggaccgaggtggtggtcaaa

SEQ ID NO: 496 - DNA, 327 bp, synthetic construct
  misc_feature 1..327: note = Synthetic
  source 1..327: mol_type = other DNA
tttgaaatgacccagaccccctcctccgtgagcgaacccgtgggcggaaccgtgaccatcaagtgtcaggcctccgaggacatctcctccaatctgggatggtatcagcagaaacccggccagccccccaagctgctgatctatggcgccagcaccctggccagcggcgtgccttctaggtttaagggcagcggcagcggcaccgagttcaccctgactatcagcgacctggaatgcgccgacgccgccacctactactgccagaccagctactacattgacgatggcgtgaacggatttggcggaggcacagaggtggtggtgaag

SEQ ID NO: 497 - DNA, 327 bp, synthetic construct
  misc_feature 1..327: note = Synthetic
  source 1..327: mol_type = other DNA
tttgaaatgacccagaccccctcctccgtgagcgaacccgtgggcggaaccgtgaccatcaagtgtcaggcctccgaggacatctcctccaatctgggatggtatcagcagaaacccggccagccccccaagctgctgatctatggcgccagcaccctggccagcggcgtgccttctaggtttaagggcagcggcagcggcaccgagttcaccctgactatcagcgacctggaacccgccgacgccgccacttactactgccagaccagctactacattgacgatggggtgaacggatttggagggggaaccgaggtggtggtgaag

SEQ ID NO: 498 - DNA, 327 bp, synthetic construct
  misc_feature 1..327: note = Synthetic
  source 1..327: mol_type = other DNA
tttgaaatgacccagaccccctcctccgtgagcgaacccgtgggcggaaccgtgaccatcaagtgtcaggcctccgaggacatctcctccaatctgggatggtatcagcagaaacccggccagccccccaagctgctgatctatggcgccagcaccctggccagcggcgtgccttctaggtttaagggcagcggcagcggcaccgagttcaccctgactatcagcgacctggaagctgccgacgccgccacttactactgccagaccagctactacatcgacgatggggtgaatggcttcggcggaggcacagaggtggtggtgaag

SEQ ID NO: 499 - DNA, 327 bp, synthetic construct
  misc_feature 1..327: note = Synthetic
  source 1..327: mol_type = other DNA
ttcgagatgacccagactccagcctccgtgtctgaacctgtgggaggcacagtcaccatcaagtgccaggccagtgaggatattagtagtaatttagcctggtatcagcagaaaccaggacagcctcccaagctcctgatctatggtgcatccactctggcatctggggtcccatcgcggttcaaaggcagtggatctgggacagagttcactctcaccatcagcgacctggagtgtgccgatgctgccacttactactgtcaaagttcttattatattgatgatggtgtaaatggtttcggcggagggaccgaggtggtggtcaaa

SEQ ID NO: 500 - DNA, 327 bp, synthetic construct
  misc_feature 1..327: note = Synthetic
  source 1..327: mol_type = other DNA
tatgatatgacccagacccccgccagtgtggaagtgaccgtgggcggaaccgtgaccatcaattgtcaggcctccgaatccctgtcctcctatctggcctggtaccagcagaaacccggccagccccccaagcccctgatctacagagccgccaccctggccagcggcgtgcctagcagatttaagggctccggctccggcaccgactacaccctgaccatcagcgacctggaatgcgccgacgccgccacctactactgccagcagggatacggctacagcaccgtgggcaacgccttcggcggcggaacagaggtggtggtgaag

SEQ ID NO: 501 - DNA, 327 bp, synthetic construct
  misc_feature 1..327: note = Synthetic
  source 1..327: mol_type = other DNA
tatgacatgacccagactccagcctctgtggaggtagctgtgggaggcacagtcaccatcaattgccaggccagtgagagcattagtcgctacttagcctggtatcagcagaaaccagggcagtctcccaagcccctgatctacagggcttccactctggcatctggggtcccatcacgattccaaggcagtggatctgggacagaatacactctcaccatcagcgacctggagtgtgccgatgctgccacttactactgtcaacagggttatggttatagtactgttgacaatgctttcggcggagggaccgaggtggtggtcaaa

SEQ ID NO: 502 - DNA, 327 bp, synthetic construct
  misc_feature 1..327: note = Synthetic
  source 1..327: mol_type = other DNA
tatgacatgacccagactccagcctctgtggaggtacctgtgggaggcacagtcaccatcaattgccaggccagtgagaacattaatagatacttagcctggtatcagcagaaaccagggcagcctcccaagcccctgatctacagggcagccactctggcatctggggtcccatcgcggttcaaaggcagtggatctgggacagagtacactctcagcatcagcgacctggagtgtgccgatgctgccacttactactgtcaacagggttatggttatagtactgttgggaatgctttcggcggagggaccgaggtggtggtcaaa

SEQ ID NO: 503 - DNA, 327 bp, synthetic construct
  misc_feature 1..327: note = Synthetic
  source 1..327: mol_type = other DNA
tatgacatgacccagactccagcctcggtgtctgcagctgtgggaggcacagtcaccatcaattgccaggccagtgagagcattagtaggtacttagcctggtatcagcagaaaccagggcagcctcccaagcccctgatctacagggcatccactctggcatctggggtcccatcgcggttcaaaggcagtggaactgggacagactacactctcaccatcagcgacctggagtgtgccgatgctgccacttactactgtcaacagggttatggttatagtactgttgggaatgctttcggcggagggaccgaggtggtggtcaaa

SEQ ID NO: 504 - DNA, 327 bp, synthetic construct
  misc_feature 1..327: note = Synthetic
  source 1..327: mol_type = other DNA
tatgacatgacccagactccagcctctgtggaggtagctgtgggaggcacagtcaccatcaattgccaggccagtgagagcattaatagatacttagcctggtatcaacagaaacgagggcagcctcccaagcccctgatctacagggcagccactctggcatctggggtcccatcgcggttcaaaggcagtggatctgggacagagtacactctcaccatcagcgacctggagtgtgccgatgctgccacttactactgtcaacagggttatggttatagtactgttgggaatgctttcggcggagggaccgaggtggtggtcaaa

SEQ ID NO: 505 - protein, 323 aa, synthetic construct
  REGION 1..323: note = Synthetic
MARGPGLAPPPLRLPLLLLVLAAVTGHTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLTAGLIAVIVVVVVALVAGMAVLVITNRRKSGKYKKVEIKELGELRKEPSL

SEQ ID NO: 506 - protein, 323 aa, synthetic construct
  REGION 1..323: note = Synthetic
MARGPGLAPPPLRLPLLLLLLAAVTGHTAAQDNCTCPTNKMTVCSPDGPGGRCQCRALGSGVAVDCSTLTSKCLLLKARMSAPKNARTLVRPNEHALVDNDGLYDPDCDPEGRFKARQCNQTSVCWCVNSVGVRRTDKGDLSLRCDELVRTHHILIDLRHRPTASAFNHSDLDAELRRLFRERYRLHPKFVAAVHYEQPTIQIELRQNTSQKAAGDVDIGDAAYYFERDVKGESLFQGRGGLDLRVRGEPLQVERTLIYYLDEIPPKFSMKRLTAGLIAVIVVVVVALVAGVAVLVISNRRKSGKYKKVEIKELGELRKEPSL

(19) United States
(12) Patent Application Publication
(10) Pub. No.: US 2025/0206841 A1
(43) Pub. Date: Jun. 26, 2025

(54) ANTI-TROPHOBLAST CELL SURFACE ANTIGEN 2 (TROP-2) ANTIBODIES

(71) Applicants: Adept Therapeutics Inc., Beverly, MA (US); Adept Biopharmaceutical and Technology, Ltd., Shenzhen (CN)

(72) Inventors: Xinyan Zhao, Bedford, MA (US); Changyun Hu, Belmont, MA (US)

(21) Appl. No.: 18/851,745

(22) PCT Filed: Mar. 28, 2023
(86) PCT No.: PCT/US2023/016579

§ 371 Date: Sep. 27, 2024

Related U.S. Application Data
(60) Provisional application No. 63/324,225, filed on Mar. 28, 2022.

(51) Int. Cl. C07K 16/30

(52) U.S. Cl. CPC C07K 16/30; C07K 2317/732; C07K 2317/77; C07K 2317/92

(57) ABSTRACT

The present invention provides anti-Trop-2 antigen binding molecules, including antibodies and the antigen binding fragment thereof, and methods for using the same for treating a variety of diseases, including cancers.

### CROSS-REFERENCE TO RELATED APPLICATIONS

The instant application claims priority to U.S. Provisional Application No. 63/324,225, filed on Mar. 28, 2022, the entire contents of which are incorporated herein by reference.

### FIELDS

The present invention relates to antigen binding molecules, e.g., antibodies or antigen binding fragments thereof, for cancer treatment and, in particular, to anti-trophoblast cell surface antigen 2 (Trop-2) antibodies for cancer therapy.

### BACKGROUND

Human Trop-2 (trophoblast cell surface antigen 2), also known as tumor-associated calcium signal transducer 2 (TACSTD2), membrane component chromosome 1 surface marker 1 (MIS1), gastrointestinal antigen 733-1 (GA733-1), and epithelial glycoprotein-1 (EGP-1), is a transmembrane glycoprotein encoded by the Tacstd2 gene, and is structurally related to epithelial cell adhesion molecule (EpCAM). It is an intracellular calcium signal transducer that is differentially expressed in many cancers. It signals cells for self-renewal, proliferation, invasion, survival, and has a role in stem cell biology and other diseases. Trop-2 is expressed at lower level in many normal tissues, though in contrast, it is overexpressed in many cancers and the overexpression of Trop-2 is of prognostic significance. Trop-2 expression in cancer cells has been correlated with drug resistance. Drugs that can tackle Trop-2 could potentially treat many cancers with unmet need.

There is a need in the art for novel antigen binding molecules, e.g., antibodies or antigen binding fragments thereof, that specifically binds to Trop-2 for use in the treatment of diseases, e.g., cancers.

### SUMMARY

The present invention provides antigen binding molecules, e.g., anti-Trop-2 antibodies or antigen binding fragments thereof, for specifically binding to Trop-2. The Trop-2 may be on the surface of a cell, e.g., a mammalian cell, such as a tumor cell of a mammal, e.g., a mouse tumor cell, a cynomolgus tumor cell or a human tumor cell. The present invention also provides methods of using the antigen binding molecules, e.g., anti-Trop-2 antibodies or antigen binding fragments thereof, of the present invention, for specifically binding to Trop-2. The binding of the antigen binding molecules, e.g., anti-Trop-2 antibodies or antigen binding fragments thereof, of the present invention, may induce antibody-dependent cellular cytotoxicity (ADCC), complement dependent cytotoxicity (CDC), antibody-dependent cellular phagocytosis (ADCP), or internalization of antibody or a conjugate thereof into a Trop-2 expressing cell, e.g., cancer cell. The binding of the antigen binding molecules, e.g., anti-Trop-2 antibodies or antigen binding fragments thereof, of the present invention, to Trop-2 expressed on a cell surface can be used for treating a subject who would benefit from modulating, e.g., inhibiting or killing, Trop-2 expressing cells, e.g., cancer cells.

Accordingly, in one aspect, the present invention provides an isolated antigen binding molecule, e.g., an antibody or antigen-binding fragment thereof, that binds to human Trop-2. The antibody or the antigen binding fragments thereof includes a heavy chain variable (VH) domain comprising from N-terminus to C-terminus, three heavy chain complementarity-determining regions (CDRs), HCDR1, HCDR2, and HCDR3; and a light chain variable (VL) domain comprising from N-terminus to C-terminus, three light chain complementarity-determining regions (CDRs), LCDR1, LCDR2, and LCDR3; wherein (a) the HCDR1 comprises an amino acid sequence selected from the group consisting of Y-G-X1-X2 (SEQ ID NO:), Y-G-X3-S(SEQ ID NO:), and Y-G-V-X4 (SEQ ID NO:), wherein X1 is M or V, X2 is S or T, X3 is M or V, and X4 is S or T; (b) the HCDR2 comprises an amino acid sequence selected from the group consisting of Y-I-Y-P-X44-X45-X46-N-X47-Y-Y-A-X48-W-V-N-G (SEQ ID NO:), Y-I-Y-P-A-X49-H-N-X50-Y-Y-A-X51-W-V-N-G (SEQ ID NO), YIYPTYHNTYYATWVNG (SEQ ID NO:), and YIYPAFPNTYYATWVNG (SEQ ID NO:), wherein X44 is A or T, X45 is F or Y, X46 is H or P, X47 is A, R, or T, X48 is N, S or T, X49 is F or Y, X50 is A, R, or T, and X51 is N, S, or T; (c) the HCDR3 comprises an amino acid sequence D-X99-G-X100-X101-D-Y-X102-X103-N-L (SEQ ID NO:), or D-A-G-X104-T-D-Y-X105-X106-N-L (SEQ ID NO:), wherein X99 is A or T, X100 is G, N, S, or T, X101 is T or V, X102 is A, K, N, or Y, X103 is F, L, or Y, X104 is G, N, S, or T, X105 is A, K, N, Y, and X106 is F, L or Y; (d) the LCDR1 comprises an amino acid sequence selected from the group consisting of Q-A-S-X135-X136-I-X137-X138-X139-X140-X141 (SEQ ID NO:), Q-A-S-X142-X143-I-X144-X145-Y-L-X146 (SEQ ID NO:), QASEDIESYSA (SEQ ID NO:), and QASKNIDSNLA (SEQ ID NO:), wherein X135 is E, K, or Q, X136 is D, N, or S, X137 is D, E, S, or Y, X138 is N, R, or S, X139 is N or Y, X140 is L or S, X141 is A or S, X142 is E or Q, X143 is D, N, or S, X144 is E, S, or Y, X145 is N, R, or S, X146 is A or S; (e) the LCDR2 comprises an amino acid sequence selected from the group consisting of X170-A-X171-X172-L-X173-S(SEQ ID NO:), X174-A-S-X175-L-X176-S(SEQ ID NO:), X177-A-S-X178-L-A-S(SEQ ID NO:), EATKLTS (SEQ ID NO:), and EASKLPS (SEQ ID NO:), wherein X170 is A, D, E, or K, X171 is S or T, X172 is K or T, X173 is A, P, or T, X174 is A, D, E, or K, X175 is K or T, X176 is A, or P, X177 is A, D, E, or K, X178 is K or T; and (f) the LCDR3 comprises an amino acid sequence X183-Q-X184-L-T-X185-G-X186-V-D-N-P (SEQ ID NO:), or Q-Q-X187-L-T-X188-G-X189-V-D-N-P (SEQ ID NO:), wherein X183 is H or Q, X184 is A, D, G, or V, X185 is I or V, X186 is D, N, or Y, X187 is A, D, G, or V, X188 is I or V, and X189 is D, N, or Y.

In one embodiment, the HCDR1 comprises an amino acid sequence selected from the group consisting of SEQ ID NOs: 1-3; (b) the HCDR2 comprises an amino acid sequence selected from the group consisting of SEQ ID NOs: 13-20; (c) the HCDR3 comprises an amino acid sequence selected from the group consisting of SEQ ID NOs: 39-50; (d) the LCDR1 comprises an amino acid sequence selected from the group consisting of SEQ ID NOs: 61-69; (e) the LCDR2 comprises an amino acid sequence selected from the group consisting of SEQ ID NOs: 81-86; and (f) the LCDR3 comprises an amino acid sequence as set forth in SEQ ID NO: 94-101.

In another embodiment, the antibody, or the antigen binding fragment thereof, includes: (a) the HCDR1 comprising an amino acid sequence set forth in SEQ ID NO: 1, the HCDR2 comprising an amino acid sequence set forth in SEQ ID NO: 13, the HCDR3 comprising an amino acid sequence set forth in SEQ ID NO: 39, the LCDR1 comprising an amino acid sequence set forth in SEQ ID NO: 61, the LCDR2 comprising an amino acid sequence set forth in SEQ ID NO: 81, and the LCDR3 comprising an amino acid sequence set forth in SEQ ID NO: 94; (b) the HCDR1 comprising an amino acid sequence set forth in SEQ ID NO: 1, the HCDR2 comprising an amino acid sequence set forth in SEQ ID NO: 13, the HCDR3 comprising an amino acid sequence set forth in SEQ ID NO: 40, the LCDR1 comprising an amino acid sequence set forth in SEQ ID NO: 61, the LCDR2 comprising an amino acid sequence set forth in SEQ ID NO: 81, and the LCDR3 comprising an amino acid sequence set forth in SEQ ID NO: 94; (c) the HCDR1 comprising an amino acid sequence set forth in SEQ ID NO: 1, the HCDR2 comprising an amino acid sequence set forth in SEQ ID NO: 14, the HCDR3 comprising an amino acid sequence set forth in SEQ ID NO: 41, the LCDR1 comprising an amino acid sequence set forth in SEQ ID NO: 62, the LCDR2 comprising an amino acid sequence set forth in SEQ ID NO: 81, and the LCDR3 comprising an amino acid sequence set forth in SEQ ID NO: 94; (d) the HCDR1 comprising an amino acid sequence set forth in SEQ ID NO: 1, the HCDR2 comprising an amino acid sequence set forth in SEQ ID NO: 14, the HCDR3 comprising an amino acid sequence set forth in SEQ ID NO: 41, the LCDR1 comprising an amino acid sequence set forth in SEQ ID NO: 63, the LCDR2 comprising an amino acid sequence set forth in SEQ ID NO: 82, and the LCDR3 comprising an amino acid sequence set forth in SEQ ID NO: 95; (e) the HCDR1 comprising an amino acid sequence set forth in SEQ ID NO: 1, the HCDR2 comprising an amino acid sequence set forth in SEQ ID NO: 15, the HCDR3 comprising an amino acid sequence set forth in SEQ ID NO: 42, the LCDR1 comprising an amino acid sequence set forth in SEQ ID NO: 61, the LCDR2 comprising an amino acid sequence set forth in SEQ ID NO: 83, and the LCDR3 comprising an amino acid sequence set forth in SEQ ID NO: 96; (f) the HCDR1 comprising an amino acid sequence set forth in SEQ ID NO: 1, the HCDR2 comprising an amino acid sequence set forth in SEQ ID NO: 16, the HCDR3 comprising an amino acid sequence set forth in SEQ ID NO: 43, the LCDR1 comprising an amino acid sequence set forth in SEQ ID NO: 64, the LCDR2 comprising an amino acid sequence set forth in SEQ ID NO: 81, and the LCDR3 comprising an amino acid sequence set forth in SEQ ID NO: 97; (g) the HCDR1 comprising an amino acid sequence set forth in SEQ ID NO: 1, the HCDR2 comprising an amino acid sequence set forth in SEQ ID NO: 13, the HCDR3 comprising an amino acid sequence set forth in SEQ ID NO: 44, the LCDR1 comprising an amino acid sequence set forth in SEQ ID NO: 61, the LCDR2 comprising an amino acid sequence set forth in SEQ ID NO: 81, and the LCDR3 comprising an amino acid sequence set forth in SEQ ID NO: 97; (h) the HCDR1 comprising an amino acid sequence set forth in SEQ ID NO: 1, the HCDR2 comprising an amino acid sequence set forth in SEQ ID NO: 13, the HCDR3 comprising an amino acid sequence set forth in SEQ ID NO: 45, the LCDR1 comprising an amino acid sequence set forth in SEQ ID NO: 61, the LCDR2 comprising an amino acid sequence set forth in SEQ ID NO: 81, and the LCDR3 comprising an amino acid sequence set forth in SEQ ID NO: 98; (i) the HCDR1 comprising an amino acid sequence set forth in SEQ ID NO: 1, the HCDR2 comprising an amino acid sequence set forth in SEQ ID NO: 14, the HCDR3 comprising an amino acid sequence set forth in SEQ ID NO: 41, the LCDR1 comprising an amino acid sequence set forth in SEQ ID NO: 61, the LCDR2 comprising an amino acid sequence set forth in SEQ ID NO: 81, and the LCDR3 comprising an amino acid sequence set forth in SEQ ID NO: 97; (j) the HCDR1 comprising an amino acid sequence set forth in SEQ ID NO: 1, the HCDR2 comprising an amino acid sequence set forth in SEQ ID NO: 14, the HCDR3 comprising an amino acid sequence set forth in SEQ ID NO: 41, the LCDR1 comprising an amino acid sequence set forth in SEQ ID NO: 65, the LCDR2 comprising an amino acid sequence set forth in SEQ ID NO: 81, and the LCDR3 comprising an amino acid sequence set forth in SEQ ID NO: 97; (k) the HCDR1 comprising an amino acid sequence set forth in SEQ ID NO: 1, the HCDR2 comprising an amino acid sequence set forth in SEQ ID NO: 17, the HCDR3 comprising an amino acid sequence set forth in SEQ ID NO: 46, the LCDR1 comprising an amino acid sequence set forth in SEQ ID NO: 66, the LCDR2 comprising an amino acid sequence set forth in SEQ ID NO: 81, and the LCDR3 comprising an amino acid sequence set forth in SEQ ID NO: 97; (1) the HCDR1 comprising an amino acid sequence set forth in SEQ ID NO: 1, the HCDR2 comprising an amino acid sequence set forth in SEQ ID NO: 18, the HCDR3 comprising an amino acid sequence set forth in SEQ ID NO: 47, the LCDR1 comprising an amino acid sequence set forth in SEQ ID NO: 61, the LCDR2 comprising an amino acid sequence set forth in SEQ ID NO: 84, and the LCDR3 comprising an amino acid sequence set forth in SEQ ID NO: 99; (m) the HCDR1 comprising an amino acid sequence set forth in SEQ ID NO: 2, the HCDR2 comprising an amino acid sequence set forth in SEQ ID NO: 19, the HCDR3 comprising an amino acid sequence set forth in SEQ ID NO: 48, the LCDR1 comprising an amino acid sequence set forth in SEQ ID NO: 67, the LCDR2 comprising an amino acid sequence set forth in SEQ ID NO: 83, and the LCDR3 comprising an amino acid sequence set forth in SEQ ID NO: 100; (n) the HCDR1 comprising an amino acid sequence set forth in SEQ ID NO: 1, the HCDR2 comprising an amino acid sequence set forth in SEQ ID NO: 14, the HCDR3 comprising an amino acid sequence set forth in SEQ ID NO: 49, the LCDR1 comprising an amino acid sequence set forth in SEQ ID NO: 68, the LCDR2 comprising an amino acid sequence set forth in SEQ ID NO: 85, and the LCDR3 comprising an amino acid sequence set forth in SEQ ID NO: 97; or (o) the HCDR1 comprising an amino acid sequence set forth in SEQ ID NO: 3, the HCDR2 comprising an amino acid sequence set forth in SEQ ID NO: 20, the HCDR3 comprising an amino acid sequence set forth in SEQ ID NO: 50, the LCDR1 comprising an amino acid sequence set forth in SEQ ID NO: 69, the LCDR2 comprising an amino acid sequence set forth in SEQ ID NO: 86, and the LCDR3 comprising an amino acid sequence set forth in SEQ ID NO: 101.

In still another embodiment, the isolated antibody, or the antigen binding fragment thereof, comprises: (a) a heavy chain variable region (HCVR) comprising an amino acid sequence selected from the group consisting of SEQ ID NOs: 113-127; and (b) a light chain variable region (LCVR) comprising an amino acid sequence selected from the group consisting of SEQ ID NOs: 162-176.

In yet another embodiment, the isolated antibody, or the antigen binding fragment thereof, comprises: (a) the HCVR comprising an amino acid sequence set forth in SEQ ID NO: 113, and the LCVR comprising an amino acid sequence set forth in SEQ ID NO: 162; (b) the HCVR comprising an amino acid sequence set forth in SEQ ID NO: 114, and the LCVR comprising an amino acid sequence set forth in SEQ ID NO: 163; (c) the HCVR comprising an amino acid sequence set forth in SEQ ID NO: 115, and the LCVR comprising an amino acid sequence set forth in SEQ ID NO: 164; (d) the HCVR comprising an amino acid sequence set forth in SEQ ID NO: 116, and the LCVR comprising an amino acid sequence set forth in SEQ ID NO: 165; (e) the HCVR comprising an amino acid sequence set forth in SEQ ID NO: 117, and the LCVR comprising an amino acid sequence set forth in SEQ ID NO: 166; (f) the HCVR comprising an amino acid sequence set forth in SEQ ID NO: 118, and the LCVR comprising an amino acid sequence set forth in SEQ ID NO: 167; (g) the HCVR comprising an amino acid sequence set forth in SEQ ID NO: 119, and the LCVR comprising an amino acid sequence set forth in SEQ ID NO: 168; (h) the HCVR comprising an amino acid sequence set forth in SEQ ID NO: 120, and the LCVR comprising an amino acid sequence set forth in SEQ ID NO: 169; (i) the HCVR comprising an amino acid sequence set forth in SEQ ID NO: 121, and the LCVR comprising an amino acid sequence set forth in SEQ ID NO: 170; (j) the HCVR comprising an amino acid sequence set forth in SEQ ID NO: 122, and the LCVR comprising an amino acid sequence set forth in SEQ ID NO: 171; (k) the HCVR comprising an amino acid sequence set forth in SEQ ID NO: 123, and the LCVR comprising an amino acid sequence set forth in SEQ ID NO: 172; (1) the HCVR comprising an amino acid sequence set forth in SEQ ID NO: 124, and the LCVR comprising an amino acid sequence set forth in SEQ ID NO: 173; (m) the HCVR comprising an amino acid sequence set forth in SEQ ID NO: 125, and the LCVR comprising an amino acid sequence set forth in SEQ ID NO: 174; (n) the HCVR comprising an amino acid sequence set forth in SEQ ID NO: 126, and the LCVR comprising an amino acid sequence set forth in SEQ ID NO: 175; or (o) the HCVR comprising an amino acid sequence set forth in SEQ ID NO: 127, and the LCVR comprising an amino acid sequence set forth in SEQ ID NO: 176.

In another aspect, the present invention provides an isolated antigen binding molecule, e.g., an antibody, or an antigen binding fragment thereof, that binds to human Trop-2. The antibody, or the antigen binding fragment thereof, includes a heavy chain variable (VH) domain comprising from N-terminus to C-terminus, three heavy chain complementarity-determining regions (CDRs), HCDR1, HCDR2, and HCDR3; and a light chain variable (VL) domain comprising from N-terminus to C-terminus, three light chain complementarity-determining regions (CDRs), LCDR1, LCDR2, and LCDR3; wherein (a) the HCDR1 comprises an amino acid sequence that is about 80%, 85%, 90%, 95%, 96%, 97%, 98%, 99% to about 100% identical to an amino acid sequence selected from the group consisting of SEQ ID NOs: 1-3; (b) the HCDR2 comprises an amino acid sequence that is about 80%, 85%, 90%, 95%, 96%, 97%, 98%, 99% to about 100% identical to an amino acid sequence selected from the group consisting of SEQ ID NOs: 13-20; (c) the HCDR3 comprises an amino acid sequence that is about 80%, 85%, 90%, 95%, 96%, 97%, 98%, 99% to about 100% identical to an amino acid sequence selected from the group consisting of SEQ ID NO: 39-50; (d) the LCDR1 comprises an amino acid sequence that is about 80%, 85%, 90%, 95%, 96%, 97%, 98%, 99% to about 100% identical to an amino acid sequence selected from the group consisting of SEQ ID NOs: 61-69; (e) the LCDR2 comprises an amino acid sequence that is about 80%, 85%, 90%, 95%, 96%, 97%, 98%, 99% to about 100% identical to an amino acid sequence selected from the group consisting of SEQ ID NOs: 81-86, (f) the LCDR3 comprises an amino acid sequence that is about 80%, 85%, 90%, 95%, 96%, 97%, 98%, 99% to about 100% identical to an amino acid sequence selected from the group consisting of SEQ ID NO: 94-101.

In still another embodiment, the present invention provides an isolated antigen binding molecule, e.g., an antibody or an antigen binding fragment thereof, that binds human Trop-2. The antibody or the antigen binding fragment thereof comprises; (a) a heavy chain variable region (HCVR) comprising an amino acid sequence that is about 80%, 85%, 90%, 95%, 96%, 97%, 98%, 99% to about 100% identical to an amino acid sequence selected from the group consisting of SEQ ID NOs: 113-127; and (b) a light chain variable region (LCVR) comprising an amino acid sequence that is about 80%, 85%, 90%, 95%, 96%, 97%, 98%, 99% to about 100% identical to an amino acid sequence selected from the group consisting of SEQ ID NOs: 162-176.

In one aspect, the present invention provides an isolated antigen binding molecule, e.g., an antibody, or an antigen binding fragment thereof. The antibody, or the antigen-binding fragment thereof, includes a heavy chain variable (V_{H}) domain comprising from N-terminus to C-terminus, three heavy chain complementarity-determining regions (CDRs), HCDR1, HCDR2, and HCDR3; and a light chain variable (V_{L}) domain comprising from N-terminus to C-terminus, three light chain complementarity-determining regions (CDRs), LCDR1, LCDR2, and LCDR3; wherein (a) the HCDR1 comprises an amino acid sequence selected from the group consisting of Y-X5-X6-L, Y-A-X7-L (SEQ ID NO:), and Y-S-X8-L (SEQ ID NO:) wherein X5 is A or S, X6 is I or M, X7 is I or M, and X8 is I or M; (b) the HCDR2 comprises an amino acid sequence X52-X53-Y-X54-S-G-X55-X56-T-Y-X57-A-X58-W-A-X59-G (SEQ ID NO:), or X60-I-Y-I-S-G-G-X61-T-Y-X62-A-X63-W-A-X64-G (SEQ ID NO:), wherein X52 is C or S, X53 is I or L, X54 is F or I, X55 is A or G, X56 is S or T, X57 is F or Y, X58 is N or S, X59 is K or T, X60 is C or S, X61 is S or T, X62 is F or Y, X63 is N or S, and X64 is K or T; (c) the HCDR3 comprises an amino acid sequence X107-D-G-X108-X109-X110-Y-Y-L-N-L (SEQ ID NO:), or D-D-G-X111-X112-S-Y-Y-L-N-L (SEQ ID NO:), wherein X107 is D or N, X108 is S or T, X109 is A, T or V, X110 is N or S, X111 is S or T, and X112 is A, T or V; (d) the LCDR1 comprises an amino acid sequence Q-A-S-X147-X148-I-Y-X149-X150-X151-A (SEQ ID NO:), or Q-A-S-E-D-I-Y-X152-L-L-A (SEQ ID NO:), wherein X147 is E or Q, X148 is D or S, X149 is N, K, R, or S, X150 is L or N, X151 is F or L, and X152 is N, K, R, or S; (e) the LCDR2 comprises an amino acid sequence X179-A-S-X180-L-X181-S(SEQ ID NO:); wherein X179 is A, D, or G, X180 is D, N, or T, X181 is A, E, or T; and (f) the LCDR3 comprises an amino acid sequence Q-Q-X190-Y-T-X191-G-N-I-D-N-X192 (SEQ ID NO:), wherein X190 is A, or G, X191 is I or V, X192 is A, P, S or T.

In one embodiment, the HCDR1 comprises an amino acid sequence selected from the group consisting of SEQ ID NOs: 4-7; (b) the HCDR2 comprises an amino acid sequence selected from the group consisting of SEQ ID NOs: 21-30, and 212; (c) the HCDR3 comprises an amino acid sequence selected from the group consisting of SEQ ID NO: 51-56; (d) the LCDR1 comprises an amino acid sequence selected from the group consisting of SEQ ID NOs: 70-74; (e) the LCDR2 comprises an amino acid sequence selected from the group consisting of SEQ ID NOs: 87-90; and (f) the LCDR3 comprises an amino acid sequence as selected from the group consisting of SEQ ID NOs: 102-109.

In another embodiment, the antibody, or the antigen binding fragment thereof, of claim 9, wherein the antibody comprises: (a) the HCDR1 comprising an amino acid sequence set forth in SEQ ID NO: 4, the HCDR2 comprising an amino acid sequence set forth in SEQ ID NO: 21, the HCDR3 comprising an amino acid sequence set forth in SEQ ID NO: 51, the LCDR1 comprising an amino acid sequence set forth in SEQ ID NO: 70, the LCDR2 comprising an amino acid sequence set forth in SEQ ID NO: 87, and the LCDR3 comprising an amino acid sequence set forth in SEQ ID NO: 102; (b) the HCDR1 comprising an amino acid sequence set forth in SEQ ID NO: 4, the HCDR2 comprising an amino acid sequence set forth in SEQ ID NO: 22, the HCDR3 comprising an amino acid sequence set forth in SEQ ID NO: 51, the LCDR1 comprising an amino acid sequence set forth in SEQ ID NO: 70, the LCDR2 comprising an amino acid sequence set forth in SEQ ID NO: 87, and the LCDR3 comprising an amino acid sequence set forth in SEQ ID NO: 102; (c) the HCDR1 comprising an amino acid sequence set forth in SEQ ID NO: 5, the HCDR2 comprising an amino acid sequence set forth in SEQ ID NO: 23, the HCDR3 comprising an amino acid sequence set forth in SEQ ID NO: 52, the LCDR1 comprising an amino acid sequence set forth in SEQ ID NO: 71, the LCDR2 comprising an amino acid sequence set forth in SEQ ID NO: 88, and the LCDR3 comprising an amino acid sequence set forth in SEQ ID NO: 103; (d) the HCDR1 comprising an amino acid sequence set forth in SEQ ID NO: 5, the HCDR2 comprising an amino acid sequence set forth in SEQ ID NO: 24, the HCDR3 comprising an amino acid sequence set forth in SEQ ID NO: 52, the LCDR1 comprising an amino acid sequence set forth in SEQ ID NO: 71, the LCDR2 comprising an amino acid sequence set forth in SEQ ID NO: 88, and the LCDR3 comprising an amino acid sequence set forth in SEQ ID NO: 103; (e) the HCDR1 comprising an amino acid sequence set forth in SEQ ID NO: 5, the HCDR2 comprising an amino acid sequence set forth in SEQ ID NO: 23, the HCDR3 comprising an amino acid sequence set forth in SEQ ID NO: 53, the LCDR1 comprising an amino acid sequence set forth in SEQ ID NO: 72, the LCDR2 comprising an amino acid sequence set forth in SEQ ID NO: 88, and the LCDR3 comprising an amino acid sequence set forth in SEQ ID NO: 103; (f) the HCDR1 comprising an amino acid sequence set forth in SEQ ID NO: 6, the HCDR2 comprising an amino acid sequence set forth in SEQ ID NO: 25, the HCDR3 comprising an amino acid sequence set forth in SEQ ID NO: 52, the LCDR1 comprising an amino acid sequence set forth in SEQ ID NO: 72, the LCDR2 comprising an amino acid sequence set forth in SEQ ID NO: 88, and the LCDR3 comprising an amino acid sequence set forth in SEQ ID NO: 103; (g) the HCDR1 comprising an amino acid sequence set forth in SEQ ID NO: 5, the HCDR2 comprising an amino acid sequence set forth in SEQ ID NO: 23, the HCDR3 comprising an amino acid sequence set forth in SEQ ID NO: 52, the LCDR1 comprising an amino acid sequence set forth in SEQ ID NO: 71, the LCDR2 comprising an amino acid sequence set forth in SEQ ID NO: 89, and the LCDR3 comprising an amino acid sequence set forth in SEQ ID NO: 104; (h) the HCDR1 comprising an amino acid sequence set forth in SEQ ID NO: 5, the HCDR2 comprising an amino acid sequence set forth in SEQ ID NO: 23, the HCDR3 comprising an amino acid sequence set forth in SEQ ID NO: 52, the LCDR1 comprising an amino acid sequence set forth in SEQ ID NO: 71, the LCDR2 comprising an amino acid sequence set forth in SEQ ID NO: 89, and the LCDR3 comprising an amino acid sequence set forth in SEQ ID NO: 103; (i) the HCDR1 comprising an amino acid sequence set forth in SEQ ID NO: 5, the HCDR2 comprising an amino acid sequence set forth in SEQ ID NO: 23, the HCDR3 comprising an amino acid sequence set forth in SEQ ID NO: 54, the LCDR1 comprising an amino acid sequence set forth in SEQ ID NO: 71, the LCDR2 comprising an amino acid sequence set forth in SEQ ID NO: 89, and the LCDR3 comprising an amino acid sequence set forth in SEQ ID NO: 104; (j) the HCDR1 comprising an amino acid sequence set forth in SEQ ID NO: 5, the HCDR2 comprising an amino acid sequence set forth in SEQ ID NO: 23, the HCDR3 comprising an amino acid sequence set forth in SEQ ID NO: 54, the LCDR1 comprising an amino acid sequence set forth in SEQ ID NO: 71, the LCDR2 comprising an amino acid sequence set forth in SEQ ID NO: 89, and the LCDR3 comprising an amino acid sequence set forth in SEQ ID NO: 103; (k) the HCDR1 comprising an amino acid sequence set forth in SEQ ID NO: 5, the HCDR2 comprising an amino acid sequence set forth in SEQ ID NO: 212, the HCDR3 comprising an amino acid sequence set forth in SEQ ID NO: 55, the LCDR1 comprising an amino acid sequence set forth in SEQ ID NO: 73, the LCDR2 comprising an amino acid sequence set forth in SEQ ID NO: 89, and the LCDR3 comprising an amino acid sequence set forth in SEQ ID NO: 105; (1) the HCDR1 comprising an amino acid sequence set forth in SEQ ID NO: 7, the HCDR2 comprising an amino acid sequence set forth in SEQ ID NO: 23, the HCDR3 comprising an amino acid sequence set forth in SEQ ID NO: 54, the LCDR1 comprising an amino acid sequence set forth in SEQ ID NO: 71, the LCDR2 comprising an amino acid sequence set forth in SEQ ID NO: 89, and the LCDR3 comprising an amino acid sequence set forth in SEQ ID NO: 103; (m) the HCDR1 comprising an amino acid sequence set forth in SEQ ID NO: 7, the HCDR2 comprising an amino acid sequence set forth in SEQ ID NO: 23, the HCDR3 comprising an amino acid sequence set forth in SEQ ID NO: 54, the LCDR1 comprising an amino acid sequence set forth in SEQ ID NO: 71, the LCDR2 comprising an amino acid sequence set forth in SEQ ID NO: 83, and the LCDR3 comprising an amino acid sequence set forth in SEQ ID NO: 103; (n) the HCDR1 comprising an amino acid sequence set forth in SEQ ID NO: 7, the HCDR2 comprising an amino acid sequence set forth in SEQ ID NO: 23, the HCDR3 comprising an amino acid sequence set forth in SEQ ID NO: 54, the LCDR1 comprising an amino acid sequence set forth in SEQ ID NO: 70, the LCDR2 comprising an amino acid sequence set forth in SEQ ID NO: 87, and the LCDR3 comprising an amino acid sequence set forth in SEQ ID NO: 104; (o) the HCDR1 comprising an amino acid sequence set forth in SEQ ID NO: 4, the HCDR2 comprising an amino acid sequence set forth in SEQ ID NO: 23, the HCDR3 comprising an amino acid sequence set forth in SEQ ID NO: 52, the LCDR1 comprising an amino acid sequence set forth in SEQ ID NO: 71, the LCDR2 comprising an amino acid sequence set forth in SEQ ID NO: 89, and the LCDR3 comprising an amino acid sequence set forth in SEQ ID NO: 102; (p) the HCDR1 comprising an amino acid sequence set forth in SEQ ID NO: 7, the HCDR2 comprising an amino acid sequence set forth in SEQ ID NO: 26, the HCDR3 comprising an amino acid sequence set forth in SEQ ID NO: 54, the LCDR1 comprising an amino acid sequence set forth in SEQ ID NO: 72, the LCDR2 comprising an amino acid sequence set forth in SEQ ID NO: 89, and the LCDR3 comprising an amino acid sequence set forth in SEQ ID NO: 106; (q) the HCDR1 comprising an amino acid sequence set forth in SEQ ID NO: 4, the HCDR2 comprising an amino acid sequence set forth in SEQ ID NO: 27, the HCDR3 comprising an amino acid sequence set forth in SEQ ID NO: 52, the LCDR1 comprising an amino acid sequence set forth in SEQ ID NO: 74, the LCDR2 comprising an amino acid sequence set forth in SEQ ID NO: 87, and the LCDR3 comprising an amino acid sequence set forth in SEQ ID NO: 107; (r) the HCDR1 comprising an amino acid sequence set forth in SEQ ID NO: 4, the HCDR2 comprising an amino acid sequence set forth in SEQ ID NO: 28, the HCDR3 comprising an amino acid sequence set forth in SEQ ID NO: 54, the LCDR1 comprising an amino acid sequence set forth in SEQ ID NO: 74, the LCDR2 comprising an amino acid sequence set forth in SEQ ID NO: 90, and the LCDR3 comprising an amino acid sequence set forth in SEQ ID NO: 104; (s) the HCDR1 comprising an amino acid sequence set forth in SEQ ID NO: 4, the HCDR2 comprising an amino acid sequence set forth in SEQ ID NO: 28, the HCDR3 comprising an amino acid sequence set forth in SEQ ID NO: 52, the LCDR1 comprising an amino acid sequence set forth in SEQ ID NO: 70, the LCDR2 comprising an amino acid sequence set forth in SEQ ID NO: 87, and the LCDR3 comprising an amino acid sequence set forth in SEQ ID NO: 103; (t) the HCDR1 comprising an amino acid sequence set forth in SEQ ID NO: 4, the HCDR2 comprising an amino acid sequence set forth in SEQ ID NO: 21, the HCDR3 comprising an amino acid sequence set forth in SEQ ID NO: 52, the LCDR1 comprising an amino acid sequence set forth in SEQ ID NO: 70, the LCDR2 comprising an amino acid sequence set forth in SEQ ID NO: 87, and the LCDR3 comprising an amino acid sequence set forth in SEQ ID NO: 104; (u) the HCDR1 comprising an amino acid sequence set forth in SEQ ID NO: 4, the HCDR2 comprising an amino acid sequence set forth in SEQ ID NO: 21, the HCDR3 comprising an amino acid sequence set forth in SEQ ID NO: 52, the LCDR1 comprising an amino acid sequence set forth in SEQ ID NO: 71, the LCDR2 comprising an amino acid sequence set forth in SEQ ID NO: 83, and the LCDR3 comprising an amino acid sequence set forth in SEQ ID NO: 103; (v) the HCDR1 comprising an amino acid sequence set forth in SEQ ID NO: 4, the HCDR2 comprising an amino acid sequence set forth in SEQ ID NO: 29, the HCDR3 comprising an amino acid sequence set forth in SEQ ID NO: 52, the LCDR1 comprising an amino acid sequence set forth in SEQ ID NO: 74, the LCDR2 comprising an amino acid sequence set forth in SEQ ID NO: 87, and the LCDR3 comprising an amino acid sequence set forth in SEQ ID NO: 108; (w) the HCDR1 comprising an amino acid sequence set forth in SEQ ID NO: 4, the HCDR2 comprising an amino acid sequence set forth in SEQ ID NO: 21, the HCDR3 comprising an amino acid sequence set forth in SEQ ID NO: 51, the LCDR1 comprising an amino acid sequence set forth in SEQ ID NO: 70, the LCDR2 comprising an amino acid sequence set forth in SEQ ID NO: 87, and the LCDR3 comprising an amino acid sequence set forth in SEQ ID NO: 102; or (x) the HCDR1 comprising an amino acid sequence set forth in SEQ ID NO: 4, the HCDR2 comprising an amino acid sequence set forth in SEQ ID NO: 30, the HCDR3 comprising an amino acid sequence set forth in SEQ ID NO: 56, the LCDR1 comprising an amino acid sequence set forth in SEQ ID NO: 74, the LCDR2 comprising an amino acid sequence set forth in SEQ ID NO: 90, and the LCDR3 comprising an amino acid sequence set forth in SEQ ID NO: 109.

In still another embodiment, the antibody or the antigen binding fragment thereof, comprises: (a) a heavy chain variable region (HCVR) comprising an amino acid sequence selected from the group consisting of SEQ ID NOs: 129-152; (b) a light chain variable region (LCVR) comprising an amino acid sequence selected from the group consisting of SEQ ID NOs: 178-201.

In yet another embodiment, isolated antibody, or the antigen binding fragment thereof, comprises: (a) the HCVR comprising an amino acid sequence set forth in SEQ ID NO: 129, and the LCVR comprising an amino acid sequence set forth in SEQ ID NO: 178; (b) the HCVR comprising an amino acid sequence set forth in SEQ ID NO: 130, and the LCVR comprising an amino acid sequence set forth in SEQ ID NO: 179; (c) the HCVR comprising an amino acid sequence set forth in SEQ ID NO: 131, and the LCVR comprising an amino acid sequence set forth in SEQ ID NO: 180; (d) the HCVR comprising an amino acid sequence set forth in SEQ ID NO: 132, and the LCVR comprising an amino acid sequence set forth in SEQ ID NO: 181; (e) the HCVR comprising an amino acid sequence set forth in SEQ ID NO: 133, and the LCVR comprising an amino acid sequence set forth in SEQ ID NO: 182; (f) the HCVR comprising an amino acid sequence set forth in SEQ ID NO: 134, and the LCVR comprising an amino acid sequence set forth in SEQ ID NO: 183; (g) the HCVR comprising an amino acid sequence set forth in SEQ ID NO: 135, and the LCVR comprising an amino acid sequence set forth in SEQ ID NO: 184; (h) the HCVR comprising an amino acid sequence set forth in SEQ ID NO: 136, and the LCVR comprising an amino acid sequence set forth in SEQ ID NO: 185; (i) the HCVR comprising an amino acid sequence set forth in SEQ ID NO: 137, and the LCVR comprising an amino acid sequence set forth in SEQ ID NO: 186; (j) the HCVR comprising an amino acid sequence set forth in SEQ ID NO: 138, and the LCVR comprising an amino acid sequence set forth in SEQ ID NO: 187; (k) the HCVR comprising an amino acid sequence set forth in SEQ ID NO: 139, and the LCVR comprising an amino acid sequence set forth in SEQ ID NO: 188; (1) the HCVR comprising an amino acid sequence set forth in SEQ ID NO: 140, and the LCVR comprising an amino acid sequence set forth in SEQ ID NO: 189; (m) the HCVR comprising an amino acid sequence set forth in SEQ ID NO: 141, and the LCVR comprising an amino acid sequence set forth in SEQ ID NO: 190; (n) the HCVR comprising an amino acid sequence set forth in SEQ ID NO: 142, and the LCVR comprising an amino acid sequence set forth in SEQ ID NO: 191; (o) the HCVR comprising an amino acid sequence set forth in SEQ ID NO: 143, and the LCVR comprising an amino acid sequence set forth in SEQ ID NO: 192; (p) the HCVR comprising an amino acid sequence set forth in SEQ ID NO: 144, and the LCVR comprising an amino acid sequence set forth in SEQ ID NO: 193; (q) the HCVR comprising an amino acid sequence set forth in SEQ ID NO: 145, and the LCVR comprising an amino acid sequence set forth in SEQ ID NO: 194; (r) the HCVR comprising an amino acid sequence set forth in SEQ ID NO: 146, and the LCVR comprising an amino acid sequence set forth in SEQ ID NO: 195; (s) the HCVR comprising an amino acid sequence set forth in SEQ ID NO: 147, and the LCVR comprising an amino acid sequence set forth in SEQ ID NO: 196; (t) the HCVR comprising an amino acid sequence set forth in SEQ ID NO: 148, and the LCVR comprising an amino acid sequence set forth in SEQ ID NO: 197; (u) the HCVR comprising an amino acid sequence set forth in SEQ ID NO: 149, and the LCVR comprising an amino acid sequence set forth in SEQ ID NO: 198; (v) the HCVR comprising an amino acid sequence set forth in SEQ ID NO: 150, and the LCVR comprising an amino acid sequence set forth in SEQ ID NO: 199; (w) the HCVR comprising an amino acid sequence set forth in SEQ ID NO: 151, and the LCVR comprising an amino acid sequence set forth in SEQ ID NO: 200; or (x) the HCVR comprising an amino acid sequence set forth in SEQ ID NO: 152, and the LCVR comprising an amino acid sequence set forth in SEQ ID NO: 201; In one aspect, the present invention provides an isolated antigen binding fragment, e.g., an antibody or an antigen binding fragment thereof. The antibody comprises a heavy chain variable (VH) domain comprising from N-terminus to C-terminus, three heavy chain complementarity-determining regions (CDRs), HCDR1, HCDR2, and HCDR3; and a light chain variable (VL) domain comprising from N-terminus to C-terminus, three light chain complementarity-determining regions (CDRs), LCDR1, LCDR2, and LCDR3; wherein (a) the HCDR1 comprises an amino acid sequence that is about 80%, 85%, 90%, 95%, 96%, 97%, 98%, 99% to about 100% identical to an amino acid sequence selected from the group consisting of SEQ ID NOs: 4-7; (b) the HCDR2 comprises an amino acid sequence that is about 80%, 85%, 90%, 95%, 96%, 97%, 98%, 99% to about 100% identical to an amino acid sequence selected from the group consisting of SEQ ID NOs: 21-30, and 212; (c) the HCDR3 comprises an amino acid sequence that is about 80%, 85%, 90%, 95%, 96%, 97%, 98%, 99% to about 100% identical to an amino acid sequence selected from the group consisting of: 51-56; (d) the LCDR1 comprises an amino acid sequence that is about 80%, 85%, 90%, 95%, 96%, 97%, 98%, 99% to about 100% identical to an amino acid sequence selected from the group consisting of SEQ ID NOs: 70-74; (e) the LCDR2 comprises an amino acid sequence that is about 80%, 85%, 90%, 95%, 96%, 97%, 98%, 99% to about 100% identical to an amino acid sequence selected from the group consisting of SEQ ID NOs: 87-90; and (f) the LCDR3 comprises an amino acid sequence that is about 80%, 85%, 90%, 95%, 96%, 97%, 98%, 99% to about 100% identical to an amino acid sequence selected from the group consisting of SEQ ID NOs: 102-109.

In another aspect, the present invention provides an isolated antigen binding molecule, e.g., an antibody, or an antigen binding fragment thereof, that binds human Trop-2. The antibody comprises (a) a heavy chain variable region (HCVR) comprising an amino acid sequence that is about 80%, 85%, 90%, 95%, 96%, 97%, 98%, 99% to about 100% identical to an amino acid sequence selected from the group consisting of SEQ ID NOs: 129-152; and (b) a light chain variable region (LCVR) comprising an amino acid sequence that is about 80%, 85%, 90%, 95%, 96%, 97%, 98%, 99% to about 100% identical to an amino acid sequence selected from the group consisting of SEQ ID NOs: 178-201.

In one aspect, the present invention provides an isolated antigen binding molecule, e.g., an antibody or an antigen binding fragment thereof. The antibody comprises a heavy chain variable (V_{H}) domain comprising from N-terminus to C-terminus, three heavy chain complementarity-determining regions (CDRs), HCDR1, HCDR2, and HCDR3; and a light chain variable (V_{L}) domain comprising from N-terminus to C-terminus, three light chain complementarity-determining regions (CDRs), LCDR1, LCDR2, and LCDR3; wherein (a) the HCDR1 comprises an amino acid sequence T-Y-W-M-W (SEQ ID NO:) or T-Y-W-M-C(SEQ ID NO:); (b) the HCDR2 comprises an amino acid sequence X65-I-Y-V-G-S-G-X66-S-T-Y-Y-A-S-W-A-K-G (SEQ ID NO:), wherein X65 is C, P, or S, and X66 is G or S; (c) the HCDR3 comprises an amino acid sequence G-A-T-N-N-V-F-M-N-Y-F-N-L (SEQ ID NO:), or G-A-T-N-N-V-F-R-N-Y-F-N-L (SEQ ID NO:); (d) the LCDR1 comprises an amino acid sequence Q-A-S-E-D-I-S-S-N-L-A (SEQ ID NO:) or Q-A-S-E-D-I-S-S-N-L-G (SEQ ID NO:); (e) the LCDR2 comprises an amino acid sequence G-A-S-T-L-A-S(SEQ ID NO:); and (f) the LCDR3 comprises an amino acid sequence Q-S-S-Y-Y-I-D-D-G-V-N-G (SEQ ID NO:) or Q-T-S-Y-Y-I-D-D-G-V-N-G (SEQ ID NO:).

In one embodiment, (a) the HCDR1 comprises an amino acid sequence set forth in SEQ ID NO: 8 or 9; (b) the HCDR2 comprises an amino acid sequence selected from the group consisting of SEQ ID NOs: 31-34; (c) the HCDR3 comprises an amino acid sequence selected from the group consisting of SEQ ID NOs: 57 and 58; (d) the LCDR1 comprises an amino acid sequence selected from the group consisting of SEQ ID NOs: 75 and 76; (e) the LCDR2 comprises an amino acid sequence as set forth in SEQ ID NO: 91; and (f) the LCDR3 comprises an amino acid sequence as selected from the group consisting of SEQ ID NOs: 110-111.

In one embodiment, (a) the HCDR1 comprises an amino acid sequence set forth in SEQ ID NO: 8, the HCDR2 comprising an amino acid sequence set forth in SEQ ID NO: 31, the HCDR3 comprising an amino acid sequence set forth in SEQ ID NO: 57, the LCDR1 comprising an amino acid sequence set forth in SEQ ID NO: 75, the LCDR2 comprising an amino acid sequence set forth in SEQ ID NO: 91, and the LCDR3 comprising an amino acid sequence set forth in SEQ ID NO: 110; (b) the HCDR1 comprises an amino acid sequence set forth in SEQ ID NO: 8, the HCDR2 comprising an amino acid sequence set forth in SEQ ID NO: 32, the HCDR3 comprising an amino acid sequence set forth in SEQ ID NO: 57, the LCDR1 comprising an amino acid sequence set forth in SEQ ID NO: 75, the LCDR2 comprising an amino acid sequence set forth in SEQ ID NO: 91, and the LCDR3 comprising an amino acid sequence set forth in SEQ ID NO: 110; (c) the HCDR1 comprises an amino acid sequence set forth in SEQ ID NO: 8, the HCDR2 comprising an amino acid sequence set forth in SEQ ID NO: 33, the HCDR3 comprising an amino acid sequence set forth in SEQ ID NO: 57, the LCDR1 comprising an amino acid sequence set forth in SEQ ID NO: 75, the LCDR2 comprising an amino acid sequence set forth in SEQ ID NO: 91, and the LCDR3 comprising an amino acid sequence set forth in SEQ ID NO: 110; or (d) the HCDR1 comprises an amino acid sequence set forth in SEQ ID NO: 9, the HCDR2 comprising an amino acid sequence set forth in SEQ ID NO: 34, the HCDR3 comprising an amino acid sequence set forth in SEQ ID NO: 58, the LCDR1 comprising an amino acid sequence set forth in SEQ ID NO: 76, the LCDR2 comprising an amino acid sequence set forth in SEQ ID NO: 91, and the LCDR3 comprising an amino acid sequence set forth in SEQ ID NO: 111.

In another embodiment, the antibody, or the antigen binding fragment thereof, comprises: (a) a heavy chain variable region (HCVR) comprising an amino acid sequence selected from the group consisting of SEQ ID NOs: 153-156; and (b) a light chain variable region (LCVR) comprising an amino acid sequence selected from the group consisting of SEQ ID NOs: 202-205.

In still another embodiment, the antibody comprises: (a) the HCVR comprising an amino acid sequence set forth in SEQ ID NO: 153, and the LCVR comprising an amino acid sequence set forth in SEQ ID NO: 202; (b) the HCVR comprising an amino acid sequence set forth in SEQ ID NO: 154, and the LCVR comprising an amino acid sequence set forth in SEQ ID NO: 203; (c) the HCVR comprising an amino acid sequence set forth in SEQ ID NO: 155, and the LCVR comprising an amino acid sequence set forth in SEQ ID NO: 204; or (b) the HCVR comprising an amino acid sequence set forth in SEQ ID NO: 156, and the LCVR comprising an amino acid sequence set forth in SEQ ID NO: 205.

In another aspect, the present invention provides an antigen binding molecule, e.g., an antibody, or antigen binding fragment thereof, that binds to human Trop-2. The antibody comprises a heavy chain variable (VH) domain comprising from N-terminus to C-terminus, three heavy chain complementarity-determining regions (CDRs), HCDR1, HCDR2, and HCDR3; and a light chain variable (VL) domain comprising from N-terminus to C-terminus, three light chain complementarity-determining regions (CDRs), LCDR1, LCDR2, and LCDR3; wherein (a) the HCDR1 comprises an amino acid sequence that is about 80%, 85%, 90%, 95%, 96%, 97%, 98%, 99% to about 100% identical to an amino acid sequence selected from the group consisting of SEQ ID NOs: 8 and 9; (b) the HCDR2 comprises an amino acid sequence that is about 80%, 85%, 90%, 95%, 96%, 97%, 98%, 99% to about 100% identical to an amino acid sequence selected from the group consisting of SEQ ID NOs: 31-34; (c) the HCDR3 comprises an amino acid sequence that is about 80%, 85%, 90%, 95%, 96%, 97%, 98%, 99% to about 100% identical to an amino acid sequence set forth in SEQ ID NO: 57 or 58; (d) the LCDR1 comprises an amino acid sequence that is about 80%, 85%, 90%, 95%, 96%, 97%, 98%, 99% to about 100% identical to an amino acid sequence selected from the group consisting of SEQ ID NOs: 75 and 76; (e) the LCDR2 comprises an amino acid sequence that is about 80%, 85%, 90%, 95%, 96%, 97%, 98%, 99% to about 100% identical to an amino acid sequence set forth in SEQ ID NO: 91; and (f) the LCDR3 comprises an amino acid sequence that is about 80%, 85%, 90%, 95%, 96%, 97%, 98%, 99% to about 100% identical to an amino acid sequence as set forth in SEQ ID NO: 110 or 111.

In still another aspect, the present invention provides an antigen binding molecule, e.g., an antibody or an antigen binding fragment thereof. The antibody comprises (a) a heavy chain variable region (HCVR) comprising an amino acid sequence that is about 80%, 85%, 90%, 95%, 96%, 97%, 98%, 99% to about 100% identical to an amino acid sequence selected from the group consisting of SEQ ID NOs: 153-156; and (b) a light chain variable region (LCVR) comprising an amino acid sequence that is about 80%, 85%, 90%, 95%, 96%, 97%, 98%, 99% to about 100% identical to an amino acid sequence selected from the group consisting of SEQ ID NOs: 202-205.

In one aspect, the present invention provides an antigen binding molecule, e.g., an antibody or an antigen binding fragment thereof, that binds human Trop-2. The antibody comprises: a heavy chain variable (V_{H}) domain comprising from N-terminus to C-terminus, three heavy chain complementarity-determining regions (CDRs), HCDR1, HCDR2, and HCDR3; and a light chain variable (V_{L}) domain comprising from N-terminus to C-terminus, three light chain complementarity-determining regions (CDRs), LCDR1, LCDR2, and LCDR3; wherein (a) the HCDR1 comprises an amino acid sequence selected from the group consisting of X10-A-X11-T (SEQ ID NO:), Y-A-X12-T (SEQ ID NO:), and X13-A-M-T (SEQ ID NO:), wherein X10 is N or Y, X1I is M or V, X12 is M or V, and X13 is N or Y; (b) the HCDR2 comprises an amino acid sequence selected from the group consisting of F-X67-X68-X69-X70-G-X71-X72-Y-Y-A-N-W-A-K-G (SEQ ID NO:), F-X73-G-I-X74-G-X75-X76-Y-Y-A-N-W-A-K-G (SEQ ID NO:), F-X77-X78-X79-R-G-X80-I-Y-Y-A-N-W-A-K-G (SEQ ID NO:) and F-I-G-I-R-G-X81-I-Y-Y-A-N-W-A-K-G (SEQ ID NO:) wherein X67 is I or V, X68 is A or G, X69 is I or L, X70 is R or Y, X71 is D, H, or N, X72 is I or F, X73 is I or V, X74 is R or Y, X75 is D, N, H, X76 is I or F, X77 is I or V, X78 is A or G, X79 is I or L, X80 is H or N, and X81 is H or N; (c) the HCDR3 comprises an amino acid sequence G-G-L-X114-T-G-X115-S-Y-F-D-L (SEQ ID NO:), wherein X114 is W or Y, and X115 is N or Y; (d) the LCDR1 comprises an amino acid sequence selected from the group consisting of Q-A-S-E-X154-X155-X156-X157-Y-L-A (SEQ ID NO:), Q-A-S-E-X158-I-X159-R-Y-L-A or Q-A-S-E-S-L-S-S-Y-L-A, and QASESLSSYLA (SEQ ID NO:) wherein X154 is N or S, X155 is I or L, X156 is N or S, X157 is R or S, X158 is N or S, and X159 is N or S; (e) the LCDR2 comprises an amino acid sequence R-A-A-T-L-A-S(SEQ ID NO:) or R-A-S-T-L-A-S(SEQ ID NO:); and (f) the LCDR3 comprises an amino acid sequence Q-Q-G-Y-G-Y-S-T-V-D-N-A (SEQ ID NO:) or Q-Q-G-Y-G-Y-S-T-V-G-N-A (SEQ ID NO:).

In one embodiment, (a) the HCDR1 comprises an amino acid sequence selected from the group consisting of SEQ ID NOs: 10-12; (b) the HCDR2 comprises an amino acid sequence selected from the group consisting of SEQ ID NOs: 35-38; (c) the HCDR3 comprises an amino acid sequence as set forth in SEQ ID NO: 59 or 60; (d) the LCDR1 comprises an amino acid sequence selected from the group consisting of 77-80; (e) the LCDR2 comprises an amino acid sequence as set forth in SEQ ID NO: 92 or 93; and (f) the LCDR3 comprises an amino acid sequence as set forth in SEQ ID NO: 112 or 211.

In another embodiment, the antibody comprises (a) the HCDR1 comprising an amino acid sequence set forth in SEQ ID NO: 10, the HCDR2 comprising an amino acid sequence set forth in SEQ ID NO: 35, the HCDR3 comprising an amino acid sequence set forth in SEQ ID NO: 59, the LCDR1 comprising an amino acid sequence set forth in SEQ ID NO: 77, the LCDR2 comprising an amino acid sequence set forth in SEQ ID NO: 92, and the LCDR3 comprising an amino acid sequence set forth in SEQ ID NO: 112; (b) the HCDR1 comprising an amino acid sequence set forth in SEQ ID NO: 11, the HCDR2 comprising an amino acid sequence set forth in SEQ ID NO: 36, the HCDR3 comprising an amino acid sequence set forth in SEQ ID NO: 60, the LCDR1 comprising an amino acid sequence set forth in SEQ ID NO: 78, the LCDR2 comprising an amino acid sequence set forth in SEQ ID NO: 93, and the LCDR3 comprising an amino acid sequence set forth in SEQ ID NO: 211; (c) the HCDR1 comprising an amino acid sequence set forth in SEQ ID NO: 10, the HCDR2 comprising an amino acid sequence set forth in SEQ ID NO: 37, the HCDR3 comprising an amino acid sequence set forth in SEQ ID NO: 60, the LCDR1 comprising an amino acid sequence set forth in SEQ ID NO: 79, the LCDR2 comprising an amino acid sequence set forth in SEQ ID NO: 92, and the LCDR3 comprising an amino acid sequence set forth in SEQ ID NO: 112; (d) the HCDR1 comprising an amino acid sequence set forth in SEQ ID NO: 12, the HCDR2 comprising an amino acid sequence set forth in SEQ ID NO: 37, the HCDR3 comprising an amino acid sequence set forth in SEQ ID NO: 60, the LCDR1 comprising an amino acid sequence set forth in SEQ ID NO: 78, the LCDR2 comprising an amino acid sequence set forth in SEQ ID NO: 93, and the LCDR3 comprising an amino acid sequence set forth in SEQ ID NO: 112; or (e) the HCDR1 comprising an amino acid sequence set forth in SEQ ID NO: 11, the HCDR2 comprising an amino acid sequence set forth in SEQ ID NO: 38, the HCDR3 comprising an amino acid sequence set forth in SEQ ID NO: 60, the LCDR1 comprising an amino acid sequence set forth in SEQ ID NO: 80, the LCDR2 comprising an amino acid sequence set forth in SEQ ID NO: 92, and the LCDR3 comprising an amino acid sequence set forth in SEQ ID NO: 112.

In one embodiment, the antibody comprises: (a) a heavy chain variable region (HCVR) comprising an amino acid sequence selected from the group consisting of SEQ ID NOs: 157-161; and (b) a light chain variable region (LCVR) comprising an amino acid sequence selected from the group consisting of SEQ ID NOs: 206-210.

In another embodiment, the antibody comprises: (a) the HCVR comprising an amino acid sequence set forth in SEQ ID NO: 157, and the LCVR comprising an amino acid sequence set forth in SEQ ID NO: 206; (b) the HCVR comprising an amino acid sequence set forth in SEQ ID NO: 158, and the LCVR comprising an amino acid sequence set forth in SEQ ID NO: 207; (c) the HCVR comprising an amino acid sequence set forth in SEQ ID NO: 159, and the LCVR comprising an amino acid sequence set forth in SEQ ID NO: 208; (d) the HCVR comprising an amino acid sequence set forth in SEQ ID NO: 160, and the LCVR comprising an amino acid sequence set forth in SEQ ID NO: 209; or (e) the HCVR comprising an amino acid sequence set forth in SEQ ID NO: 161, and the LCVR comprising an amino acid sequence set forth in SEQ ID NO: 210.

In another aspect, the present invention provides an antigen binding molecule, e.g., an antibody or an antigen binding fragment thereof. The antibody comprises a heavy chain variable (VH) domain comprising from N-terminus to C-terminus, three heavy chain complementarity-determining regions (CDRs), HCDR1, HCDR2, and HCDR3; and a light chain variable (VL) domain comprising from N-terminus to C-terminus, three light chain complementarity-determining regions (CDRs), LCDR1, LCDR2, and LCDR3; wherein (a) the HCDR1 comprises an amino acid sequence that is about 80%, 85%, 90%, 95%, 96%, 97%, 98%, 99% to about 100% identical to an amino acid sequence selected from the group consisting of SEQ ID NOs: 10-12; (b) the HCDR2 comprises an amino acid sequence that is about 80%, 85%, 90%, 95%, 96%, 97%, 98%, 99% to about 100% identical to an amino acid sequence selected from the group consisting of SEQ ID NOs: 35-38; (c) the HCDR3 comprises an amino acid sequence that is about 80%, 85%, 90%, 95%, 96%, 97%, 98%, 99% to about 100% identical to an amino acid sequence set forth in SEQ ID NO: 59 or 60; (d) the LCDR1 comprises an amino acid sequence that is about 80%, 85%, 90%, 95%, 96%, 97%, 98%, 99% to about 100% identical to an amino acid sequence selected from the group consisting of SEQ ID NOs: 77-80; (e) the LCDR2 comprises an amino acid sequence that is about 80%, 85%, 90%, 95%, 96%, 97%, 98%, 99% to about 100% identical to an amino acid sequence as set forth in SEQ ID NO: 92 or 93; and (f) the LCDR3 comprises an amino acid sequence that is about 80%, 85%, 90%, 95%, 96%, 97%, 98%, 99% to about 100% identical to an amino acid sequence as set forth in SEQ ID NO: 112 or 211.

In still another aspect, the present invention provides an antigen binding molecule, e.g., an antibody or an antigen binding fragment thereof. The antibody comprises (a) a heavy chain variable region (HCVR) comprising an amino acid sequence that is about 80%, 85%, 90%, 95%, 96%, 97%, 98%, 99% to about 100% identical to an amino acid sequence selected from the group consisting of SEQ ID NOs: 157-161; and (b) a light chain variable region (LCVR) comprising an amino acid sequence that is about 80%, 85%, 90%, 95%, 96%, 97%, 98%, 99% to about 100% identical to an amino acid sequence selected from the group consisting of SEQ ID NOs: 206-210.

In various aspects of the invention and embodiments thereof, the antibody, or the antigen-binding fragment thereof, is an antigen binding fragment of the antibody. In various aspects of the invention and embodiments thereof, the human Trop-2 comprises a sequence as set forth in SEQ ID NO:.

In one embodiment of various aspects of the invention, the N-terminus of the heavy chain and/or light chain of the antibody or the antigen binding fragment thereof is a pyroglutamate (pE) residue.

In one embodiment of various aspects of the invention, the antibody or the antigen binding fragment thereof, or a conjugate thereof, (i) competes for binding to human Trop-2 with a monoclonal antibody of selected from the group consisting of 9F7, 2H3, 69H10, 65D5, 3E9, 9A5, 12G6, 1D10, 62E3, 3A4, 63H3, 66A1, 2E12, 72F12, 74F11/15F5, 9D7, 9D7-2, 10A9, 10A9-2, 62A2, 74E4, 66A6, 10E12, 8H2, 74H11, 74A1, 10F12, 65E11, 69C2, 61F12, 10D3, 67H4, 64G9, 72G12, 65G8, 69D8, 2H5, 64A6, 62B10, 5D4, 5D4-1, 5D4-2, 70G2, 4F2, 62A10, 70E2, 71F10, and 73C2; (ii) specifically binds to a human and/or a cynomolgus Trop-2; (iii) specifically binds to a cell surface human Trop-2; (iv) induces ADCC; (v) induces ADCP; (vi) induces CDC; or (vii) induces internalization of the antibody, or the antigen binding fragment thereof, or a conjugate thereof.

In one aspect, the present invention provides an antigen binding molecule, e.g., an antibody or an antigen binding fragment thereof. The antibody competes for binding to human Trop-2 with an antibody of any above aspects and the various embodiments thereof.

In one embodiment, the isolated antigen binding molecule, e.g., the antibody or antigen binding fragment thereof, specifically binds to human or cynomolgus Trop-2 with an equilibrium dissociation constant (K_{D}) of about 0.01 nM to about 5 nM, as measured by a biolayer interferometry method described in Example 1, and 3 or a substantially similar method. In another embodiment, the binding of the antibody to a Trop-2, or a cell surface Trop-2, is determined using ELISA-based assays or flow cytometry-based assays as described in Examples 3, and 4, or substantial similar assays thereof.

In another embodiment, the isolated antigen binding molecule, e.g., the antibody or antigen binding fragment thereof, may induce ADCC, ADCP, CDC, or internalization of the antibody or antigen binding fragment thereof or a conjugate thereof, or any combination thereof.

In still another embodiment, the antibody specifically binds to human Trop-2 and/or cynomolgus Trop-2. In yet another embodiment, the antibody specifically binds to human Trop-2 and/or cynomolgus Trop-2 with similar affinity. In one embodiment, the antibody does not bind to non-primate Trop-2 or binds to non-primate Trop-2 with an affinity that is significantly lower than that of human Trop-2 and/or cynomolgus Trop-2. In various aspects of the invention and embodiments thereof, the cynomolgus Trop-2 comprises a sequence as set forth in SEQ ID NO:.

In one aspect, the present invention provides an isolated antigen binding molecule, e.g., an antibody, or an antigen-binding fragment thereof, that competes for binding to human Trop-2 with an antibody of any aspect.

In one embodiment, the antigen binding molecule, e.g., the antibody, or the antigen binding fragment thereof, is a humanized antibody or a chimeric antibody. In another embodiment, the antibody comprises a heavy chain constant region of a class selected from IgA, IgD, IgE, IgG, or IgM.

In still another embodiment, the antibody comprises a heavy chain constant region of the class IgG, and wherein the IgG is selected from the group consisting of IgG4, IgG1, IgG2, and IgG3. In yet another embodiment, the antibody comprises a human heavy chain constant region.

In one aspect, the present invention provides a conjugate, e.g., an antibody conjugate. The conjugate includes an antigen binding molecule, e.g., an antibody or antigen binding fragment thereof, of the present invention, conjugated to an agent. In certain embodiment, the agent is a therapeutic agent for the treatment of cancer.

In one aspect, the present invention provides a fusion protein. The fusion protein includes an antigen binding molecule, e.g., an antibody or antigen binding fragment thereof, of the present invention and a different protein or peptide. In one embodiment, the different protein or peptide is a cytokine, chemokine or a functional domain of thereof. In another embodiment, the cytokine is selected from the group consisting of IL-2, IL-5, IL-7, IL-12, IL-15, IL-21, and GM-CSF. In still another embodiment, the different protein or peptide is a trap protein or peptide that blocks the binding of a ligand to a receptor that may result in suppression of immune response of an immune cell. In yet another embodiment, the trap protein or peptide is an extracellular domain of transforming growth factor beta receptor. In one embodiment, the different protein or peptide is operably linked to the antigen binding molecule, e.g., the antibody or antigen binding fragment thereof, of the present invention. In another embodiment, the different protein or peptide is operably linked to a C-terminus of the antigen binding molecule, e.g., the antibody or antigen binding fragment thereof. In still another embodiment, the different protein or peptide is operably linked to an N-terminus of the antigen binding molecule, e.g., the antibody or antigen binding fragment thereof. In yet another embodiment, more than one different protein or peptide is operably linked to the antigen binding molecule, e.g., the antibody or antigen binding fragment thereof, of the present invention.

In another aspect, the present invention provides an isolated polynucleotide encoding the antigen binding molecule, e.g., the antibody of any aspects and the various embodiments thereof, an HCVR thereof, an LCVR thereof, a light chain thereof, a heavy chain thereof, or an antigen binding fragment thereof.

In still another aspect, the present invention provides an expression vector that includes comprising the polynucleotide.

In yet another aspect, the present invention provides a recombinant cell that includes the polynucleotide or the expression vector.

In one aspect, the present invention provides a method of producing the antigen binding molecule, e.g., the antibody of any aspects and the various embodiments thereof. The method includes expressing the antibody in the recombinant cell and isolating the expressed antibody.

In one aspect, the present invention provides a pharmaceutical composition. The pharmaceutical composition includes the antigen binding molecule, e.g., the antibody or antigen binding fragment thereof, of any aspects and various embodiments thereof, and a pharmaceutically acceptable carrier or diluent.

In one embodiment, the antigen binding molecule, e.g., the antibody or antigen binding fragment thereof, or a conjugate thereof, in the pharmaceutical composition is in an amount effective to (a) specifically bind to a cell surface human or cynomolgus Trop-2; (b) induce ADCC; (c) induce ADCP; (d) induce CDC; (e) induce internalization of the or the antigen binding fragment thereof, or a conjugate thereof; or (f) any combination of (a)-(e), in a subject.

In one aspect, the present invention provides a method of killing a cancer cell by antibody-dependent cellular-cytotoxicity (ADCC). The method includes contacting the cancer cell with an isolated antibody, or an antigen binding fragment thereof, of any aspect of the present invention and any embodiment thereof, or a pharmaceutical composition of any aspect of the present invention and any embodiment thereof, thereby killing the cancer cell, wherein the cancer cell comprises Trop-2 expressed on the cell surface.

In another aspect, the present invention provides a method of inducing internalization of an antibody, or an antigen binding fragment thereof, or a conjugate thereof, into a Trop-2 expressing cell, comprising contacting the cell with an isolated antibody, or an antigen binding fragment thereof, of any aspect of the present invention and any embodiment thereof, or a pharmaceutical composition of any aspect of the present invention and any embodiment thereof, thereby inducing the internalization of the antibody or the antigen binding fragment thereof into the Trop-2 expressing cell. In one embodiment, the antibody or the antigen binding fragment thereof is conjugated to an agent, optionally the agent is a therapeutically active agent. In another embodiment, the cell is a cancer cell.

In one embodiment, the cell is inside a subject, optionally the subject is a human. In another embodiment, the method is used for the treatment of a cancer in a subject.

In still another aspect, the present invention provides a method of inhibiting growth of a tumor in a subject. The method includes administering an isolated antibody of any aspect or the pharmaceutical composition of any aspect to the subject, thereby inhibiting growth of the tumor.

In yet another aspect, the present invention provides a method of treating cancer in a subject, comprising administering an isolated antibody of any aspect or the pharmaceutical composition of aspect, thereby treating the cancer. In one embodiment, the cancer is any cancer described herein. In one particular embodiment, the cancer is selected from the group of triple negative breast cancer (TNBC), pancreatic ductal adenocarcinoma (PDAC), metastatic castration-resistant prostate (mCRPC), renal cell carcinoma (RCC), multiple myeloma, colorectal cancer (CRC), esophageal cancer (EC), non-small cell lung cancer (NSCLC), small cell lung cancer (SCLC), ovarian epithelial cancer (OEC), head and neck cancer (HNC), cervical cancer (CC), follicular thyroid cancer (FTC), glioblastoma (GBM), and other therapy-resistant solid tumor that is advanced/metastatic or has no available therapeutic options.

In another embodiment, the method of any of above aspect further includes administering an additional therapeutic agent. In one embodiment, the additional therapeutic agent includes any therapeutic agent described herein. In another embodiment, the additional therapeutic agent comprises an anti-tumor agent, radiotherapy, a chemotherapeutic agent, a surgery, a cancer vaccine, an agonist to a stimulatory receptor of an immune cell, a cytokine, a cell therapy, or a checkpoint inhibitor. In one embodiment, the additional therapeutic agent is an antibody, including multi-specific antibody, e.g., bispecific antibody.

In still another embodiment, checkpoint inhibitor is an agent that inhibits an immune checkpoint protein selected from the group consisting of A2aR, CTLA-4, PD-1, PD-L1, PD-L2, TIGIT, LAG-3, TIM-3, B7-H3, B7-H4, A2aR, CD73, PVRIG/PVRL2, neuritin, BTLA, CECAM-1, CECAM-5, CECAM6, IL-1R8, VISTA, LAIRI, LILRBI, LILRB2, LILRB3, LILRB4, LILRB5, CD47, SIRPa, CD200R, CD96, CD112R, 2B4, TGFβ-R, KIR, NKG2A, SEMA4D, Axl, MerTK, GAS6, TNFR2, GARP, CCR8, IDO, NOX2, SIGLEC7, SIGLEC15, and any combination thereof. In yet another embodiment, the checkpoint inhibitor is an agent that inhibits the interaction between PD-1 and PD-L1 and is selected from the group consisting of pembrolizumab, nivolumab, atezolizumab, avelumab, durvalumab, BMS-936559, sintilimab, toripalimab, tislelizumab, camrelizumab, envafolimab, sugemalimab, penpulimab, cadonilimab, sulfamonomethoxine, and sulfamethizole. In one embodiment, the CTLA inhibitor is selected from the group consisting of ipilimumab, cadonilimab, YH001 (Encure Biopharma), ADGI16 (Adagene), and ADG126 (Adagene).

In yet another embodiment, the additional therapeutic agent is an agonist to a stimulatory receptor of an immune cell selected from OX40, CD2, CD3, CD7, CD16, CD27, CD28, CD30, CD40, ICAM-i, LFA-i, ICOS (CD278), 4-1 BB (CD137), GITR, BAFFR, HVEM, LIGHT, NKG2C, NKG2D, SLAMF7, NKp46, NKp80, CD160, and any combination thereof.

In one embodiment, the additional therapeutic agent is formulated in the same pharmaceutical composition as the antibody. In another embodiment, the additional therapeutic agent is formulated in a different pharmaceutical composition from the antibody.

In still another embodiment, the additional therapeutic agent is administered prior to the antigen binding molecule, e.g., antibody, of various aspects. In yet another embodiment, the additional therapeutic agent is administered subsequent to the antigen biding molecule, e.g., antibody, subsequently to administering the antibody. In another embodiment, the additional therapeutic agent is administered concurrently with the antigen binding molecule, e.g., the antibody.

In one aspect, the present invention provides a kit. The kit includes the pharmaceutical composition of any aspect. In one embodiment, the pharmaceutical composition further comprises any one or more of the additional therapeutic agents described herein.

### DETAILED DESCRIPTION

The invention and accompanying drawings will now be discussed to enable one skilled in the art to practice the present invention. The skilled artisan will understand, however, that the inventions described below can be practiced without employing these specific details, or that they can be used for purposes other than those described herein. Indeed, they can be modified and can be used in conjunction with products and techniques known to those of skill in the art considering the present disclosure. The drawings and descriptions are intended to be exemplary of various aspects of the invention and are not intended to narrow the scope of the appended claims. Furthermore, it will be appreciated that the drawings may show aspects of the invention in isolation and the elements in one figure may be used in conjunction with elements shown in other figures.

It will be appreciated that reference throughout this specification to aspects, features, advantages, or similar language does not imply that all the aspects and advantages may be realized with the present invention should be or are in any single embodiment of the invention. Rather, language referring to the aspects and advantages is understood to mean that a specific aspect, feature, advantage, or characteristic described in connection with an embodiment is included in at least one embodiment of the present invention. Thus, discussion of the aspects and advantages, and similar language, throughout this specification may, but do not necessarily, refer to the same embodiment.

The described aspects, features, advantages, and characteristics of the invention may be combined in any suitable manner in one or more further embodiments. Furthermore, one skilled in the relevant art will recognize that the invention may be practiced without one or more of the specific aspects or advantages of a particular embodiment. In other instances, additional aspects, features, and advantages may be recognized and claimed in certain embodiments that may not be present in all embodiments of the invention.

Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present invention. The described aspects and embodiments of the application are not limited to the methods and materials described.

Further, with respect to the teachings in the present invention, any cited references, any issued patent or patent application publication described in this application is expressly incorporated by reference herein.

### I. Definitions

In order that the present invention may be more readily understood, certain terms are first defined. In addition, it should be noted that whenever a value or range of values of a parameter are recited, it is intended that values and ranges intermediate to the recited values are also intended to be part of this invention.

The use of the terms “a” and “an” and “the” and similar referents in the context of describing the invention (especially in the context of the following claims) are to be construed to cover both the singular and the plural (i.e., one or more), unless otherwise indicated herein or clearly contradicted by context. The terms “comprising, “having,” “including,” and “containing” are to be construed as open-ended terms (i.e., meaning “including, but not limited to”) unless otherwise noted. Recitation of ranges of values herein are merely intended to serve as a shorthand method of referring individually to each separate value recited or falling within the range, unless otherwise indicated herein, and each separate value is incorporated into the specification as if it were individually recited.

Where the phrases “in one embodiment,” “in another embodiment,” “in other embodiments,” “in some embodiments,” or “in certain embodiments” are used, the present disclosure should be construed as embracing combinations of any of the features defining the different embodiments described therein, unless the features are not combinable with one another, are mutually exclusive, or are expressly disclaimed herein.

The term “about” or “approximately,” as applied to one or more values provided herein, refers to a value that is similar to a stated reference value. In some embodiments, the term “about” or “approximately” refers to a range of values that fall within and include 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context. In some embodiments, “about” or “approximately” can be understood as about 2 standard deviations from the mean. In some embodiments, “about” or “approximately” means up to and including ±10% (e.g., ±10%, ±9%, ±8%, ±7%, ±6%, ±5%, ±4%, ±3%, ±2%, 1%, or less). In some embodiments, “about” or “approximately” means±5%. When “about” or “approximately” is present before a series of numbers or a range, it is understood that it can modify each of the numbers in the series or range.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about”, it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

The term “administering” or “administration of,” as used herein, means to provide an agent, e.g., an antibody to a subject. In some embodiments, “administering” or “administration of” means to provide an antibody or antibody conjugate to a subject in a manner that is physiologically and/or (e.g., and) pharmacologically useful (e.g., to treat a condition in the subject). Non-limiting examples of routes of administration include intravenous, intramuscular, intraperitoneal, intracerebrospinal, subcutaneous, intra-articular, intrasynovial, or intrathecal routes. In some embodiments, the route of administration is subcutaneous.

As used herein, the term “agent” is used with reference to any substance, compound (e.g., molecule), supramolecular complex, material, or combination or mixture thereof. A compound may be any agent that can be represented by a chemical formula, chemical structure, or sequence.

Example of agents, include, e.g., small molecules, polypeptides, nucleic acids (e.g., RNAi agents, antisense oligonucleotide, aptamers), lipids, polysaccharides, etc. In general, agents may be obtained using any suitable method known in the art. The ordinary skilled artisan will select an appropriate method based, e.g., on the nature of the agent. An agent may be at least partly purified. In some embodiments an agent may be provided as part of a composition, which may contain, e.g., a counter-ion, aqueous or non-aqueous diluent or carrier, buffer, preservative, or other ingredient, in addition to the agent, in various embodiments. In some embodiments an agent may be provided as a salt, ester, hydrate, or solvate. In some embodiments an agent is cell-permeable, e.g., within the range of typical agents that are taken up by cells and acts intracellularly, e.g., within mammalian cells, to produce a biological effect. Certain compounds may exist in particular geometric or stereoisomeric forms.

Such compounds, including cis- and trans-isomers, E- and Z-isomers, R- and S-enantiomers, diastereomers, (D)-isomers, (L)-isomers, (−)—and (+)-isomers, racemic mixtures thereof, and other mixtures thereof are encompassed by this disclosure in various embodiments unless otherwise indicated. Certain compounds may exist in a variety or protonation states, may have a variety of configurations, may exist as solvates (e.g., with water (i.e., hydrates) or common solvents) and/or may have different crystalline forms (e.g., polymorphs) or different tautomeric forms. Embodiments exhibiting such alternative protonation states, configurations, solvates, and forms are encompassed by the present disclosure where applicable.

In certain embodiments and depending on the context, an “agent” also includes a method of treatment, such as radiotherapy, chemotherapy, or surgery.

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gin; Q), Glycine (Gly; G); histidine (His; H), isoleucine (He; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

The term “antagonist” or “inhibitor” refers to a substance that prevents, blocks, inhibits, neutralizes, or reduces a biological activity or effect of another molecule, such as a receptor.

The term “agonist” refers to a substance which promotes (i.e., induces, causes, enhances, or increases) the biological activity or effect of another molecule. The term agonist encompasses substances which bind receptor, such as an antibody, and substances which promote receptor function without binding thereto (e.g., by activating an associated protein).

The term “antibody”, as used herein, means any antigen binding molecule or molecular complex comprising at least one complementarity determining region (CDR) that specifically binds to or interacts with a particular antigen (e.g., Trop-2). The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H} or VH (V_{H} and VH are used interchangeably herein unless the context indicates otherwise)) and a heavy chain constant region. The heavy chain constant region comprises three domains, C_{H}1, C_{H}2 and C_{H}3 (C_{H} and C_{H} are used interchangeably herein unless the context indicates otherwise). Each light chain comprises a light chain variable region (abbreviated herein as LCVR or V_{L} or VL (V_{L} and VL are used interchangeably herein unless the context indicates otherwise)) and a light chain constant region. The light chain constant region comprises one domain (C_{L}) (C_{L} and CL are used interchangeably herein unless the context indicates otherwise). The VH and V_{L} regions can be further subdivided into regions of hypervariability, termed complementarity determining regions (CDRs), interspersed with regions that are more conserved, termed framework regions (FR). Each VH and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of the anti-Trop-2 antibody (or antigen binding fragment thereof) may be identical to the murine, rabbit, rat, or human germ line sequences, or may be naturally or artificially modified. An amino acid consensus sequence may be defined based on a side-by-side analysis of two or more CDRs.

The term “antibody”, as used herein, also includes antigen binding fragments of full antibody molecules. The terms “antigen binding portion” of an antibody, “antigen binding fragment” of an antibody, and the like, as used herein, include any naturally occurring, enzymatically obtainable, synthetic, or genetically engineered polypeptide or glycoprotein that specifically binds an antigen to form a complex. Antigen binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen binding fragment,” as used herein.

An antigen binding fragment of an antibody will typically comprise at least one variable domain. The variable domain may be of any size or amino acid composition and will generally comprise at least one CDR which is adjacent to or in frame with one or more framework sequences.

In antigen binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen binding fragment of an antibody may contain a monomeric V_{H} or V_{L} domain.

In certain embodiments, an antigen binding fragment of an antibody may contain at least one variable domain covalently linked to at least one constant domain. Non-limiting, exemplary configurations of variable and constant domains that may be found within an antigen binding fragment of an antibody of the present invention include: (i) V_{H}-C_{H}1; (ii) V_{H}-C_{H}2; (iii) V_{H}-C_{H}3; (iv) VH-C_{H}1-C_{H}2; (v) V_{H}-C_{H}1—C_{H}2-C_{H}3; (vi) V_{H}-C_{H}2-C_{H}3; (vii) V_{H}-C_{L}; (viii) V_{L}-C_{H}1; (iX) V_{L}-C_{H}2; (X) V_{L}-C_{H}3; (Xi) V_{L}-C_{H}1—C_{H}2; (Xii) V_{L}-C_{H}1—C_{H}2-C_{H}3; (Xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may comprise at least 2 (e.g., 5, 10, 15, 20, 40, 60 or more) amino acids which result in a flexible or semi-flexible linkage between adjacent variable and/or constant domains in a single polypeptide molecule. Moreover, an antigen binding fragment may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)). When describing polypeptide domain arrangements with hyphens between individual domains (e.g., C_{H}2-C_{H}3), it should be understood that the order of the listed domains is from the N-terminus to the C-terminus.

As with full antibody molecules, antigen binding fragments may be monospecific or multispecific (e.g., bispecific). A multispecific antigen binding fragment of an antibody will typically comprise at least two different variable domains, wherein each variable domain is capable of specifically binding to a separate antigen or to a different epitope on the same antigen. Any multispecific antibody format may be adapted for use in the context of an antigen binding fragment of an antibody of the present invention using routine techniques available in the art.

The antibodies of the invention may be isolated antibodies. An “isolated” molecule, such as an isolated antibody or an isolated polypeptide, as used herein, means a molecule, e.g., an antibody, that has been identified and separated and/or recovered from at least one component of its natural environment. For example, a molecule, e.g., an antibody, that has been separated or removed from at least one component of an organism, or from a tissue or cell in which the antibody naturally exists or is naturally produced, is an “isolated” molecule, e.g., antibody, for purposes of the present invention. An isolated molecule, e.g., an antibody also includes a molecule, e.g., an antibody in situ within a recombinant cell. In certain embodiments, isolated molecules, e.g., antibodies, are molecules, e.g., antibodies that have been subjected to at least one purification or isolation step. According to certain embodiments, an isolated molecule, e.g., antibody may be substantially free of other cellular material and/or chemicals.

The present invention also includes one-arm antibodies that bind Trop-2. As used herein, a “one-arm antibody” means an antigen binding molecule comprising a single antibody heavy chain and a single antibody light chain. The one-arm antibodies of the present invention may comprise any of the HCVR/LCVR or CDR amino acid sequences as set forth in Tables 1-19.

The anti-Trop-2 antibodies herein, or the antigen binding fragments thereof, may comprise one or more amino acid substitutions, insertions and/or deletions in the framework and/or CDR regions of the heavy and light chain variable domains as compared to the corresponding germline sequences from which the antigen binding molecules, e.g., antibodies or antigen binding fragments were derived. Such mutations can be readily ascertained by comparing the amino acid sequences disclosed herein to germline sequences available from, for example, public antibody sequence databases. The present invention includes antibodies, and the antigen binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can produce numerous antibodies and antigen binding fragments, which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the frameworks and/or CDR residue(s) are mutated to the corresponding residue(s) of a different germline sequence (i.e., a germline sequence that is different from the germline sequence from which the antibody was originally derived). Furthermore, the antibodies, or the antigen binding domains thereof, of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies, or the antigen binding fragments thereof, that contain one or more germline mutations can be easily tested for one or more desired property such as, improved binding specificity, increased binding affinity, reduced immunogenicity, etc. Antibodies, or the antigen binding fragments thereof, obtained in this general manner are encompassed within the present invention.

The present invention also includes anti-Trop-2 antibodies comprising variants of any of the HCVR, LCVR, and/or CDR amino acid sequences disclosed herein. Exemplary variants included within this aspect of the invention include variants of any of the HCVR, LCVR, and/or CDR amino acid sequences disclosed herein having one or more conservative substitutions. For example, the present invention includes anti-Trop-2 antibodies and antigen binding proteins having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences set forth in the Tables herein.

Light chains are classified as either kappa or lambda (K, X). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

As used herein, the term “light chain constant region” or “C_{L}” are used interchangeably herein with reference to amino acid sequences derived from an antibody light chain. Preferably, the light chain constant region comprises at least one of a constant kappa domain or constant lambda domain.

As used herein, the term “heavy chain constant region” includes amino acid sequences derived from an immunoglobulin heavy chain. A polypeptide comprising a heavy chain constant region comprises at least one of: a C_{H}1 domain, a hinge (e.g., upper, middle, and/or lower hinge region) domain, a C_{H}2 domain, a C_{H}3 domain, or a variant or fragment thereof. For example, an antigen binding polypeptide for use in the disclosure may comprise a polypeptide chain comprising a C_{H}1 domain; a polypeptide chain comprising a C_{H}1 domain, at least a portion of a hinge domain, and a C_{H}2 domain; a polypeptide chain comprising a C_{H}1 domain and a C_{H}3 domain; a polypeptide chain comprising a C_{H}1 domain, at least a portion of a hinge domain, and a C_{H}3 domain, or a polypeptide chain comprising a C_{H}1 domain, at least a portion of a hinge domain, a C_{H}2 domain, and a C_{H}3 domain. In some embodiments, a polypeptide of the disclosure comprises a polypeptide chain comprising a C_{H}3 domain. Further, an antibody for use in the disclosure may lack at least a portion of a C_{H}2 domain (e.g., all or part of a C_{H}2 domain). It should be understood that the heavy chain constant region may be modified such that they vary in amino acid sequence from the naturally occurring immunoglobulin molecule.

The heavy chain constant region of an antibody disclosed herein may be derived from different immunoglobulin molecules. For example, a heavy chain constant region of a polypeptide may comprise a C_{H}1 domain derived from an IgG₁ molecule and a hinge region derived from an IgG₃ molecule. In another example, a heavy chain constant region can comprise a hinge region derived, in part, from an IgG₁ molecule and, in part, from an IgG₃ molecule. In another example, a heavy chain portion can comprise a chimeric hinge derived, in part, from an IgG₁ molecule and, in part, from an IgG₄ molecule.

A “light chain-heavy chain pair” refers to the collection of a light chain and heavy chain that can form a dimer through a disulfide bond between the C_{L} domain of the light chain and the C_{H}i domain of the heavy chain.

The subunit structures and three-dimensional configurations of the constant regions of the various immunoglobulin classes are well known. As used herein, the term “V_{H} domain” includes the N terminal variable domain of an immunoglobulin heavy chain and the term “C_{H}1 domain” includes the first (most N terminal) constant region domain of an immunoglobulin heavy chain. The C_{H}1 domain is adjacent to the V_{H} domain and is N-terminal to the hinge region of an immunoglobulin heavy chain molecule.

As used herein the term “C_{H}2 domain” includes the portion of a heavy chain molecule that extends, e.g., from about residue 244 to residue 360 of an antibody using conventional numbering schemes (residues 244 to 360, Kabat numbering system; and residues 231-340, EU numbering system). The C_{H}2 domain is unique in that it is not closely paired with another domain. Rather, two N-linked branched carbohydrate chains are interposed between the two C_{H}2 domains of an intact native IgG molecule. The C_{H}3 domain extends from the C_{H}2 domain to the C-terminal of the IgG molecule and comprises approximately 108 residues.

As used herein, the term “hinge region” includes the portion of a heavy chain molecule that joins the C_{H}1 domain to the C_{H}2 domain. This hinge region comprises approximately 25 residues and is flexible, thus allowing the two N-terminal antigen binding regions to move independently. Hinge regions can be subdivided into three distinct domains: upper, middle, and lower hinge domains.

As used herein the term “disulfide bond” includes a covalent bond formed between two sulfur atoms. The amino acid cysteine comprises a thiol group that can form a disulfide bond or bridge with a second thiol group. In most naturally occurring IgG molecules, the C_{H}1 and C_{L} regions are linked by a disulfide bond and the two heavy chains are linked by two disulfide bonds at positions corresponding to 239 and 242 using the Kabat numbering system (position 226 or 229, EU numbering system).

The term “antibody” also encompasses various broad classes of polypeptides that can be distinguished biochemically. Those skilled in the art will appreciate that heavy chains are classified as alpha, delta, epsilon, gamma, and mu, or α, δ, ε, γ and μ) with some subclasses among them (e.g., γ1-γ4). It is the nature of this chain that determines the “class” of the antibody as IgG, IgM, IgA IgD, or IgE, respectively. The immunoglobulin subclasses (isotypes) e.g., IgG1, IgG2, IgG3, IgG4, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are within the scope of the present disclosure, the following discussion will generally be directed to the IgG class of immunoglobulin molecules.

Antibodies of the disclosure include, but are not limited to, polyclonal, monoclonal, multispecific, bispecific, trispecific, human, humanized, primatized, chimeric and single chain antibodies. Antibodies disclosed herein may be from any animal origin, including birds and mammals. Preferably, the antibodies are human, murine, rat, donkey, rabbit, goat, guinea pig, camel, llama, horse, or chicken antibodies. In some embodiments, the variable region may be condricthoid in origin (e.g., from sharks).

The term “humanized antibody” as used herein, refers to a genetically engineered non-human antibody, which contains human antibody constant domains and non-human variable domains modified to contain a high level of sequence homology to human variable domains. This can be achieved by grafting the six non-human antibody complementarity-determining regions (CDRs), which together form the antigen binding site, onto a homologous human acceptor framework region (FR). In order to reconstitute the binding affinity and specificity of the parental antibody, the substitution of framework residues from the parental antibody (i.e., the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

As used herein, the phrase “chimeric antibody,” refers to an antibody where the immunoreactive region or site is obtained or derived from a first species and the constant region (which may be intact, partial or modified in accordance with the instant disclosure) is obtained from a second species. In certain embodiments the target binding region or site will be from a non-human source (e.g., mouse, rat, rabbit, or primate) and the constant region is human.

A “single-chain fragment variable” or “scFv” refers to a fusion protein of the variable regions of the heavy (VH) and light chains (VL) of immunoglobulins. In some aspects, the regions are connected with a short linker peptide of ten to about 25 amino acids. The linker can be rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker.

With regard to IgGs, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration where the light chains bracket the heavy chains starting at the mouth of the “Y” and continuing through the variable region.

The term “biological activity” means any biological property of a molecule, whether present naturally in vivo, or provided or enabled by recombinant means. Biological activities include, but are not limited to, binding to a receptor, inducing cell proliferation, inhibiting cell growth, inducing other cytokines, inducing apoptosis, and enzymatic activity.

The term “conjugate” or “antibody conjugate” refers to an antibody, or the antigent binding fragment thereof, linked to one or more agents. The antibody, or the antigent binding fragment thereof, can be covalently linked to the agent via a covalent bond or a linker. In certain embodiments, the linker is covalently bonded to the antibody, or the antigent binding fragment thereof, and also covalently bonded to the agent. In certain embodiments, the linker is linked to the antibody, or the antigent binding fragment thereof, and/or the agent via non-covalent means. In certain embodiments, the linker is linked to the agent via a covalent bond and linked to the antibody, or the antigent binding fragment thereof, via specifical binding. In certain embodiments, the linker is a moiety that can specifically binds to the antibody, e.g., an antibody that binds to the Fc region of the antibody. In certain embodiments, a conjugate comprises an antibody linked to one or more agents via non-covalent means. In certain embodiments, the agent may be a therapeutic agent, a cytotoxic agent, or diagnostic agent. In certain embodiments, the agent may be an oligonucleotide, an oligopeptide, or a small molecule drug. In certain embodiments, the agent can be attached, for example to reduced SH groups and/or to carbohydrate side chains.

The term “control” or “reference,” when referring to a substance, means a composition used as a standard or a point of comparison against which other test results are measured. In some embodiments, a “control” or “reference” is a composition known to not contain analyte (“negative control”) or to contain analyte (“positive control”). A positive control can comprise a known concentration of analyte. “Control,” and “positive control,” may be used to refer to a composition comprising a known concentration of analyte. A “positive control” can be used to establish assay performance characteristics and is a useful indicator of the integrity of reagents (e.g., analytes). In some embodiments, an appropriate “control” or “reference” is where only one element is changed in order to determine the effect of the one element. In some embodiments, a control is a level of a target gene (e.g., in a cell or in a subject) before treatment (e.g., with an RNAi agent described herein).

The term “control” or “reference” also means a baseline level of a measurement depending upon the context, in which the term is used. A baseline level of a measurement is a standard or a point of comparison against which the measurement is compared. In some embodiments, a “control” or a “reference” refers to a level of a measurement for certain biological activity or substance in a cell, a tissue, an organ, or a subject, e.g., the expression level of a gene, copy number of mRNA for such gene, or level of protein encoded by such gene, without treatment of the cell, the tissue, the organ, or the subject, with an agent, e.g., an RNAi agent. In some embodiments, a “control” or a “reference” refers to a level of an average measurement for a certain biological activity or substance in a cell, a tissue, an organ, or a subject, e.g., certain enzyme activity of the liver, among a group of healthy subjects, e.g., the general population within certain geographic or demographic limits or any other limits that may be appropriate for the study of certain disease or disorder, that does not have certain disease or disorder, e.g., liver disease.

The term “control” may be used in “control individual,” who is an individual with similar condition, e.g., an individual afflicted with the same cell proliferative disorder as the individual being treated, who is about the same age as the individual being treated (to ensure that the stages of the disease in the treated individual and the control individual(s) are comparable). The individual (also referred to as “patient” or “subject”) being treated may be a fetus, infant, child, adolescent, or adult human with a cell proliferative disorder.

The term “reference” may also be used in “reference sequence.” The term “reference sequence” refers to a sequence, e.g., a nucleic acid sequence or an amino acid sequence, used as a basis for sequence comparison.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstances, an epitope may include moieties of saccharides, phosphoryl groups, or sulfonyl groups on the antigen.

The term “immunoconjugate” refers to an antibody which is fused by covalent linkage to a peptide or small molecule drug. The peptide or small molecule drug can be linked to the C-terminus of a constant heavy chain or to the N-terminus of a variable light and/or heavy chain.

The terms, “improve,” “increase,” or “reduce,” as used in this context, indicate values or parameters relative to a baseline/control/reference measurement, such as a measurement in a cell or a tissue prior to initiation of the treatment described herein, or a measurement in a cell or a tissue in the absence of the treatment described herein, a measurement in the same individual prior to initiation of the treatment described herein, or a measurement in a control individual (or a standard measurement derived from multiple control individuals, such as the average value of the multiple control individuals) in the absence of the treatment described herein.

The term “inhibit,” “inhibition,” “reduce,” and “reduction,” in the context of the level of activity of an agent, refers to a statistically significant decrease in such level. The decrease can be, for example, at least 10%, 20%, 25%, 30%, 35%, 40%, 45%, 50%, 55%, 60%, 65%, 70%, 75%, 80%, 85%, 90%, or 95%, or below the level of detection for the detection method.

The term “prevent” refers to a decrease in the occurrence of disease symptoms in a patient. As indicated above, the prevention may be complete (no detectable symptoms) or partial, such that fewer symptoms are observed than would likely occur absent treatment.

As used herein, the term “recombinant,” when in connection with polypeptides or polynucleotides, refers to polypeptides or polynucleotides that do not exist naturally and which may be created by combining polynucleotides or polypeptides in arrangements that would not normally occur together. The term “recombinant cell,” as used herein, refers to a non-naturally occurring host cell comprising one or more (e.g., two, several) heterologous polynucleotides. The term “host cell” means any cell type that is susceptible to transformation, transfection, transduction, and the like with a nucleic acid construct or expression vector.

The phrase “small molecule drug” refers to a molecular entity, often organic or organometallic, that is not a polymer, that has medicinal activity, and that has a molecular weight less than about 2 kDa, less than about 1 kDa, less than about 900 Da, less than about 800 Da or less than about 700 Da. The term encompasses most medicinal compounds termed “drugs” other than protein or nucleic acids, although a small peptide or nucleic acid analog can be considered a small molecule drug. Examples include chemotherapeutic anticancer drugs and enzymatic inhibitors. Small molecules drugs can be derived synthetically, semi-synthetically (i.e., from naturally occurring precursors), or biologically.

As used herein, the terms “specific binding” or “specifically binds” refer to an ability to discriminate between possible binding partners in the environment in which binding is to occur. In some embodiments, an antibody that interacts, e.g., preferentially interacts, with one particular antigen when other potential antibodies are present is said to “bind specifically” to the antigen with which it interacts. In some embodiments, specific binding is assessed by detecting or determining the degree of association between the antibody and its targeted antigen; in some embodiments, specific binding is assessed by detecting or determining degree of dissociation of an antibody-antigen complex. In some embodiments, specific binding is assessed by detecting or determining the ability of the antibody to compete with an alternative interaction between its target and another antibody. In some embodiments, specific binding is assessed by performing such detections or determinations across a range of concentrations. In general, an antibody binds to an epitope via its antigen binding domain, and that the binding entails some complementarity between the antigen binding domain and the epitope. Thus, an antibody is said to “specifically bind” to an epitope when it binds to that epitope via its antigen binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.” In some embodiments, an antibody or an antibody fragment “has specificity to” an antigen if the antibody or the antigen binding fragment thereof forms a complex with the antigen with an equilibrium dissociation constant (K_{D}) of 10⁻⁶M or less, 10⁻⁷M or less, 10⁻⁸ M or less, 10⁻⁹M or less, 10¹⁰ M or less, 10¹¹ M or less, or 10⁻²M or less. In certain embodiments, the specific binding of the antigen binding molecules, e.g., anti-human Trop-2 antibodies or antigen binding fragment thereof, can be shown by the preferential binding of the antigen binding molecules to human Trop-2 expressed on a cell surface using assays described in Examples 3 and 4, or substantially similar methods.

The term “subject,” as used herein, refers to a mammal. In some embodiments, a subject is non-human primate, or rodent. In some embodiments, a subject is a human. In some embodiments, a subject is a patient, e.g., a human patient that has or is suspected of having a disease. In some embodiments, the subject is a human patient who has or is suspected of having a Trop-2 disease or Trop-2-associated disease.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 85%, 80%, or 95%, and more preferably at least about 96%, 97%, 98% or 99% of the nucleotide bases, as measured by any well-known algorithm of sequence identity, such as FASTA, BLAST or Gap, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

The present invention also includes antigen-binding molecules comprising an antigen binding domain with an HCVR, LCVR, and/or CDR amino acid sequence that is substantially identical to any of the HCVR, LCVR, and/or CDR amino acid sequences disclosed herein. As applied to polypeptides, the term “substantial similarity” or “substantially similar” means that two peptide sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et a. (1992) Science 256: 1443-1445. A “moderately conservative” replacement is any change having a nonnegative value in the PAM250 log-likelihood matrix.

“Sequence similarity” for polypeptides or polynucleotides, which is also referred to as “sequence identity,” is typically measured using sequence analysis software. Protein or nucleic acid analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215:403-410 and Altschul et a/. (1997) Nucleic Acids Res. 25:3389-402.

The term “sequence identity” also refers to a comparison between pairs of nucleic acid or polypeptide molecules, i.e., the relatedness between two nucleotide sequences. In general, the sequences are aligned so that the highest order match is obtained. Methods for determining sequence identity are known and can be determined by commercially available computer programs that can calculate the percentage of identity between two or more sequences. A typical example of such a computer program is BLAST, or CLUSTAL.

The term “treat,” “treatment,” as used herein, mean the methods or steps taken to provide relief from or alleviation of the number, severity, and/or frequency of one or more symptoms of a disease in a subject. As used herein, “treat” and treatment” may include the prevention, management, prophylactic treatment, and/or inhibition of the number, severity, and/or frequency of one or more symptoms of a disease in a subject. The terms “treat” and “treatment” refer to any indicia of success in the therapy or amelioration of an injury, disease, pathology or condition, including prevention or delay of the onset of one or more symptoms of the disease or disorder; lessening of the severity or frequency of one or more symptoms of the disease or disorder; any objective or subjective parameter such as abatement; remission; diminishing of symptoms or making the injury, pathology or condition more tolerable to the patient; slowing in the rate of degeneration or decline; making the final point of degeneration less debilitating; and/or improving a patient's physical or mental well-being.

The phrases “to a patient in need thereof,” “to a patient in need of treatment” or “a subject in need of treatment” includes subjects, such as mammalian subjects, that would benefit from administration of the antibodies of the present disclosure for treatment of a cell proliferative disorder.

The terms “therapeutically effective amount,” “pharmacologically effective amount,” “physiologically effective amount,” and “effective amount” are used interchangeably to mean the amount of an active agent sufficient to ameliorate at least one symptom of the disease or disorder. For example, for the given parameter, a therapeutically effective amount will show an increase or decrease of at least 5%, 10%, 15%, 20%, 25%, 40%, 50%, 60%, 75%, 80%, 90%, 95%, 99%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control. The precise amount will depend upon numerous factors, e.g., the particular active agent, the components and physical characteristics of the composition, intended patient population, patient considerations, including weight, sex and the like, and can readily be determined by one skilled in the art, based upon the information provided herein or otherwise available in the relevant literature.

The term “variant,” as used herein, refers to a polypeptide, e.g., an antibody, or a polynucleotide, that is derived by incorporation of one or more amino acid or nucleotide insertions, substitutions, or deletions in a precursor polypeptide or polynucleotide (e.g., “parent” polypeptide or polynucleotide). In certain embodiments, a variant polypeptide or polynucleotide has at least about 85% amino acid or nucleotide sequence identity, e.g., about 90%, about 91%, about 92%, about 93%, about 94%, about 95%, about 96%, about 97%, about 98%, about 99%, or about 100%, amino acid or nucleotide sequence identity to the entire amino acid or nucleotide sequence of a parent polypeptide or polynucleotide. A variant of a protein or peptide maintains substantially the structures, functions or activities of the protein. For example, a variant of an antibody maintains the function or activities of specifically binding to its antigen and/or modulates, e.g., inhibits, the activities of the antigen. In the case of a polynucleotide, a variant thereof maintains its function or activities of the parent polynucleotide. For example, a variant polynucleotide may encode a protein or peptide that has similar functions or activities of the polypeptide encoded by the parent polynucleotide.

As used herein, the term “Trop-2” refers to trophoblast cell surface antigen 2. Unless indicated otherwise, such as by specific reference to human Trop-2, the term “Trop-2” includes all mammalian species of native Trop-2 from, e.g., human, primate, rodent, canine, feline, equine, and bovine. The nucleotide and amino acid sequence of Trop-2 is known and may be found in, for example, GenBank Accession Nos. NP_002344.2, P09758, XP_005543292.2, XP_001114599.1, NP_001009540.2, Q8BGV3.1, XP 852842.5, the entire contents of each of which are incorporated herein by reference. The following is an exemplary human Trop-2 amino acid sequence:

(SEQ ID NO:)
MARGPGLAPPPLRLPLLLLVLAAVTGHTAAQDNCTCPTNKMTVCSPDGP
GGRCQCRALGSGMAVDCSTLTSKCLLLKARMSAPKNARTLVRPSEHALV
DNDGLYDPDCDPEGRFKARQCNQTSVCWCVNSVGVRRTDKGDLSLRCDE
LVRTHHILIDLRHRPTAGAFNHSDLDAELRRLFRERYRLHPKFVAAVHY
EQPTIQIELRQNTSQKAAGDVDIGDAAYYFERDIKGESLFQGRGGLDLR
VRGEPLQVERTLIYYLDEIPPKFSMKRLTAGLIAVIVVVVVALVAGMAV
LVITNRRKSGKYKKVEIKELGELRKEPSL

An exemplary cynomolgus Trop-2 amino acid sequence is shown below:

(SEQ ID NO:)
MARGPGLAPPPLRLPLLLLLLAAVTGHTAAQDNCTCPTNKMTVCSPDGPG
GRCQCRALGSGVAVDCSTLTSKCLLLKARMSAPKNARTLVRPNEHALVDN
DGLYDPDCDPEGRFKARQCNQTSVCWCVNSVGVRRTDKGDLSLRCDELVR
THHILIDLRHRPTASAFNHSDLDAELRRLFRERYRLHPKFVAAVHYEQPT
IQIELRQNTSQKAAGDVDIGDAAYYFERDVKGESLFQGRGGLDLRVRGEP
LQVERTLIYYLDEIPPKFSMKRLTAGLIAVIVVVVVALVAGVAVLVISNR
RKSGKYKKVEIKELGELRKEPSL

The term “anti-Trop-2 antibody,” or “Trop-2 antibody” refers to an antibody or polypeptide that specifically binds to Trop-2. In certain embodiments, the anti-Trop-2 antibody is able to induce ADCC, CDC, ADCP, and/or internalization of the antibody or a conjugate thereof into a Trop-2 expressing cells, e.g., cancer cells. Anti-Trop-2 antibodies encompass antibodies or polypeptides contain one or more antigen binding domains in the form of CDRs or variable regions.

As used herein, the term “Trop2 disease” or “Trop2-associated disease,” is a disease or disorder that is caused by, or associated with, Trop2 expression and/or activity. The term “Trop2 disease” or “Trop2-associated disease” includes a disease, disorder or condition that would benefit from a modulation, e.g., inhibition, in Trop2 gene expression, replication, or protein activity. In certain embodiments, a Trop2 disease or Trop2-associated disease is a cancer.

### II. Trop-2 Antibodies and Antigen Binding Proteins

The present invention provides Trop-2 antigen binding molecules that bind specifically to Trop-2. As used herein, the term “antigen binding molecule” refers to a protein, polypeptide or molecular complex comprising or consisting of at least one complementarity determining region (CDR) that alone, or in combination with one or more additional CDRs and/or framework regions (FRs), specifically binds to a particular antigen. In certain embodiments, an antigen binding molecule is an antibody or an antigen binding fragment thereof, as those terms are defined elsewhere herein. In certain embodiments, the antigen binding molecules of the present invention induce ADCC, CDC, ADCP, and/or internalization of the antibody or a conjugate thereof into Trop-2 expressing cells, e.g., cancer cells.

In certain embodiments, the Trop-2 is a human Trop-2. An exemplary human Trop-2 has the amino acid sequence as set forth in SEQ ID NO:. In some embodiments, the Trop-2 is a cynomolgus Trop-2. An exemplary cynomolgus Trop-2 has the amino acid sequence as set forth in SEQ ID NO:.

### 1. Exemplary Antigen Binding Molecules

The Trop-2 antigen binding molecules may be in the form of monoclonal antibodies; one or more polypeptide fragment(s) containing one or more Trop-2 antigen binding domains; or one or more nucleic acids encoding one or more Trop-2 binding domains.

In various exemplary embodiments of the present invention, an antigen binding molecules, e.g., an anti-Trop-2 antibodies or antigen binding fragments thereof, includes (1) a heavy chain variable region, wherein the heavy chain variable region comprises three complementarity determining regions (HCDRs): HCDR1, HCDR2 and HCDR3 and (2) a light chain variable region, wherein the light chain variable region comprises three complementarity determining regions (LCDRs): LCDR1, LCDR2 and LCDR3; wherein the antigen binding molecules binds specifically to human Trop-2. Exemplary HCDR- and LCDR amino acid sequences corresponding to the exemplary anti-human Trop-2 monoclonal antibodies disclosed in the present invention are shown in Tables 1-5.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol, 2001, 309:657-70 (“AHo” numbering scheme); each of which is incorporated by reference in its entirety. Tables 1 and 2 show the sequences of heavy chain CDRs of exemplary antibodies of the invention according to Kabat numbering scheme and IMGT numbering scheme, respectively. Table 3 shows the sequences of heavy chain CDRs of exemplary antibodies of the invention, in which the CDR sequences are defined by combining the CDRs based on Kabat and IMGT numbering schemes. Tables 4 and 5 show the sequences of light chain CDRs of exemplary antibodies of the invention according to Kabat numbering scheme and IMGT numbering scheme.

In certain embodiments, the present invention includes antigen binding molecules, e.g., anti-Trop-2 antibodies or antigen binding fragments thereof, comprising CDRs which are defined based on Kabat and IMGT numbering scheme, or the combination thereof. Accordingly, in certain embodiments, the present invention includes antigen binding molecules, e.g., anti-Trop-2 antibodies or antigen binding fragments thereof, comprising (1) a HCDR1 having an amino acid sequence that is about 80%, 85%, 90%, 95%, 96%, 97%, 98%, 99% to about 100% identical to or comprises one or two mutations to an amino acid sequence selected from the HCDR1 sequences listed in Tables 1 and 6; (2) a HCDR2 having an amino acid sequence that is about 80%, 85%, 90%, 95%, 96%, 97%, 98%, 99% to about 100% identical to or comprises 1, 2, 3, 4, 5, 6, 7, or 8 mutations to an amino acid sequence selected from the HCDR2 sequences listed in Tables 1 and 9; (3) a HCDR3 having an amino acid sequence that is about 80%, 85%, 90%, 95%, 96%, 97%, 98%, 99% to about 100% identical to or comprises 1, 2, 3, 4, or 5 mutations to an amino acid sequence selected from the HCDR3 sequences listed in Tables 1 and 11; (4) a LCDR1 having an amino acid sequence that is about 80%, 85%, 90%, 95%, 96%, 97%, 98%, 99% to about 100% identical to or comprises 1, 2, 3, 4, 5, 6, or 7 mutations an amino acid sequence selected from the LCDR1 sequences listed in Tables 4 and 13; (5) a LCDR2 having an amino acid sequence that is about 80%, 85%, 90%, 95%, 96%, 97%, 98%, 99% to about 100% identical or comprises 1, 2, 3, or 4 mutations to an amino acid sequence selected from the LCDR2 sequences listed in Tables 4 and 15; and (6) a LCDR3 having an amino acid sequence that is about 80%, 85%, 90%, 95%, 96%, 97%, 98%, 99% to about 100% identical to or comprises 1, 2, 3, 4, or 5 mutations to an amino acid sequence selected from the LCDR3 sequences listed in Tables 4 and 16. As used herein, the term “mutation” refers to a change of or difference in the amino acid sequence (substitution, insertion or deletion) compared to a reference. In certain embodiments, a mutation is a conservative substitution.

In certain embodiments, the present invention includes antigen binding molecules, e.g., anti-Trop-2 antibodies or antigen binding fragments thereof, comprising (1) a HCDR1 having an amino acid sequence selected from the HCDR1 sequences that is about 80%, 85%, 90%, 95%, 96%, 97%, 98%, 99% to about 100% identical to or comprises 1, 2, or 3 mutations to an amino acid sequence listed in Tables 2 and 7; (2) a HCDR2 having an amino acid sequence that is about 80%, 85%, 90%, 95%, 96%, 97%, 98%, 99% to about 100% identical to or comprises 1, 2, 3, or 4 mutations an amino acid sequence selected from the HCDR2 sequences listed in Tables 2 and 10; (3) a HCDR3 having an amino acid sequence that is about 80%, 85%, 90%, 95%, 96%, 97%, 98%, 99% to about 100% identical to or comprises 1, 2, 3, 4, or 5 mutations to an amino acid sequence selected from the HCDR3 sequences listed in Tables 2 and 12; (4) a LCDR1 having an amino acid sequence that is about 80%, 85%, 90%, 95%, 96%, 97%, 98%, 99% to about 100% identical to or comprises 1, 2, or 3 mutations to an amino acid sequence selected from the LCDR1 sequences listed in Table 5 and 14; (5) a LCDR2 having an amino acid sequence that is about 80%, 85%, 90%, 95%, 96%, 97%, 98%, 99% to about 100% identical to or comprises one mutation to an amino acid sequence selected from the LCDR2 sequences listed in Table 5; and (6) a LCDR3 having an amino acid sequence that is about 80%, 85%, 90%, 95%, 96%, 97%, 98%, 99% to about 100% identical to or comprises 1, 2, 3, or 4 mutations to an amino acid sequence selected from the LCDR3 sequences listed in Tables 5 and 16.

In certain embodiments, the present invention includes antigen binding molecules, e.g., anti-Trop-2 antibodies or antigen binding fragments thereof, comprising (1) a HCDR1 having an amino acid sequence that is about 80%, 85%, 90%, 95%, 96%, 97%, 98%, 99% to about 100% identical to or comprises 1, 2, 3, or 4 mutations to an amino acid sequence selected from the HCDR1 sequences listed in Tables 3 and 8; (2) a HCDR2 having an amino acid sequence that is about 80%, 85%, 90%, 95%, 96%, 97%, 98%, 99% to about 100% identical to or comprises 1, 2, 3, 4, 5, 6, 7, or 8 mutations to an amino acid sequence selected from the HCDR2 sequences listed in Tables 3 and 9; (3) a HCDR3 having an amino acid sequence that is about 80%, 85%, 90%, 95%, 96%, 97%, 98%, 99% to about 100% identical to or comprises 1, 2, 3, 4, or 5 mutations to an amino acid sequence selected from the HCDR3 sequences listed in Tables 3 and 12; (4) a LCDR1 having an amino acid sequence that is about 80%, 85%, 90%, 95%, 96%, 97%, 98%, 99% to about 100% identical to or comprises 1, 2, 3, 4, 5, 6, or 7 mutations to an amino acid sequence selected from the LCDR1 sequences listed in Tables 4 and 13; (5) a LCDR2 having an amino acid sequence that is about 80%, 85%, 90%, 95%, 96%, 97%, 98%, 99% to about 100% identical to or comprises 1, 2, 3, or 4 mutations to an amino acid sequence selected from the LCDR2 sequences listed in Tables 4 and 15; and (6) a LCDR3 having an amino acid sequence that is about 80%, 85%, 90%, 95%, 96%, 97%, 98%, 99% to about 100% identical to or comprises 1, 2, 3, 4, or 5 mutations to an amino acid sequence selected from the LCDR3 sequences listed in Tables 4 and 16.

As used herein, a “position” in a CDR refers to the amino acid counted from the N-terminus of the CDR. For example, position 1 or the 1^{st} position in HCDR1 refers to the first amino acid in HCDR1. Accordingly, in clone 9F7, position 1 of HCDR1 based on Kabat numbering scheme is a tyrosine (Y).

As shown in Tables 6-16, certain positions in the CDRs are more conservative. For example, for clones 9F7, 2H3, 69H10, 65D5, 3E9, 9A5, 12G6, 1D10, 62E3, 3A4, 63H3, 66A1, 2E12, 72F12, 74F11, 15F5, the first position of HCDR1 based on Kabat numbering scheme is highly conservative, which contains a “Y.” On the other hand, certain positions in the CDRs are less conservative. For example, for the same group of clones, the third and fourth position is less conservative, which can be “M” or “V,” and “S” or “T,” respectively. In certain embodiments, the mutations occur at one or more less conservative positions in the CDRs.

TABLE 1
Amino Acid Sequences of Heavy Chain CDRs of Exemplary Antibodies
(Kabat Numbering Scheme)

| Clone | HCDR1 | SEQ ID NO: | HCDR2 | SEQ ID NO: | HCDR3 | SEQ ID NO: |
|---|---|---|---|---|---|---|
| 9F7 | YGVS | 1 | YIYPAYHNTYYATWVNG | 13 | DIGSTDYNFNL | 39 |
| 2H3 | YGVS | 1 | YIYPAYHNTYYATWVNG | 13 | DTGTTDYNFNL | 40 |
| 69H10 | YGVS | 1 | YIYPAFHNTYYATWVNG | 14 | DAGTTDYNFNL | 41 |
| 65D5 | YGVS | 1 | YIYPAFHNTYYATWVNG | 14 | DAGTTDYNFNL | 41 |
| 3E9 | YGVS | 1 | YIYPAFHNRYYANWVNG | 15 | DAGSTDYKFNL | 42 |
| 9A5 | YGVS | 1 | YIYPTYHNTYYATWVNG | 16 | DAGGTDYNFNL | 43 |
| 12G6 | YGVS | 1 | YIYPAYHNTYYATWVNG | 13 | DAGNTDYNFNL | 44 |
| 1D10 | YGVS | 1 | YIYPAYHNTYYATWVNG | 13 | DAGSTDYNLNL | 45 |
| 62E3 | YGVS | 1 | YIYPAFHNTYYATWVNG | 14 | DAGTTDYNFNL | 41 |
| 3A4 | YGVS | 1 | YIYPAFHNTYYATWVNG | 14 | DAGTTDYNFNL | 41 |
| 63H3 | YGVS | 1 | YIYPAFHNAYYANWVNG | 17 | DAGGTDYNYNL | 46 |
| 66A1 | YGVS | 1 | YIYPAFHNTYYASWVNG | 18 | DAGGTDYAYNL | 47 |
| 2E12 | YGVT | 2 | YIYPAFPNTYYATWVNG | 19 | DAGSTDYYFNL | 48 |
| 72F12 | YGVS | 1 | YIYPAFHNTYYATWVNG | 14 | DAGSVDYNFNL | 49 |
| 74F11/15F5 | YGMS | 3 | YIYPAFHNTYYANWVNG | 20 | DAGSTDYNFNL | 50 |
| 9D7 | YSIL | 4 | CIYISGGSTYYANWAKG | 21 | DDGTASYYLNL | 51 |
| 9D7-2 | YSIL | 4 | SIYISGGSTYYANWAKG | 22 | DDGTASYYLNL | 51 |
| 10A9 | YAML | 5 | CIYISGGTTYFASWATG | 23 | DDGTTSYYLNL | 52 |
| 10A9-2 | YAML | 5 | SIYISGGTTYFASWATG | 24 | DDGTTSYYLNL | 52 |
| 62A2 | YAML | 5 | CIYISGGTTYFASWATG | 23 | NDGTTSYYLNL | 53 |
| 74E4 | YAIL | 6 | CIYISGATTYFANWATG | 25 | DDGTTSYYLNL | 52 |
| 66A6 | YAML | 5 | CIYISGGTTYFASWATG | 23 | DDGTTSYYLNL | 52 |
| 10E12 | YAML | 5 | CIYISGGTTYFASWATG | 23 | DDGTTSYYLNL | 52 |
| 8H2 | YAML | 5 | CIYISGGTTYFASWATG | 23 | DDGSTSYYLNL | 54 |
| 74H11 | YAML | 5 | CIYISGGTTYFASWATG | 23 | DDGSTSYYLNL | 54 |
| 74A1 | YAML | 5 | CLYISGGTTYFASWATG | 212 | NDGSTNYYLNL | 55 |
| 10F12 | YSML | 7 | CIYISGGTTYFASWATG | 23 | DDGSTSYYLNL | 54 |
| 65E11 | YSML | 7 | CIYISGGTTYFASWATG | 23 | DDGSTSYYLNL | 54 |
| 69C2 | YSML | 7 | CIYISGGTTYFASWATG | 23 | DDGSTSYYLNL | 54 |
| 61F12 | YSIL | 4 | CIYISGGTTYFASWATG | 23 | DDGTTSYYLNL | 52 |
| 10D3 | YSML | 7 | CIYISGGTTYYASWAKG | 26 | DDGSTSYYLNL | 54 |
| 67H4 | YSIL | 4 | CIYISGGSTYYASWAKG | 27 | DDGTTSYYLNL | 52 |
| 64G9 | YSIL | 4 | CIYISGGTTYYANWAKG | 28 | DDGSTSYYLNL | 54 |
| 72G12 | YSIL | 4 | CIYISGGTTYYANWAKG | 28 | DDGTTSYYLNL | 52 |
| 65G8 | YSIL | 4 | CIYISGGSTYYANWAKG | 21 | DDGTTSYYLNL | 52 |
| 69D8 | YSIL | 4 | CIYISGGSTYYANWAKG | 21 | DDGTTSYYLNL | 52 |
| 2H5 | YSIL | 4 | CIYFSGGSTYYANWAKG | 29 | DDGTTSYYLNL | 52 |
| 64A6 | YSIL | 4 | CIYISGGSTYYANWAKG | 21 | DDGTASYYLNL | 51 |
| 62B10 | YSIL | 4 | CIYISGGTTYFANWAKG | 30 | DDGSVSYYLNL | 56 |
| 5D4 | TYWMW | 8 | CIYVGSGSSTYYASWAKG | 31 | GATNNVFMNYFNL | 57 |
| 5D4-1 | TYWMW | 8 | PIYVGSGSSTYYASWAKG | 32 | GATNNVFMNYFNL | 57 |
| 5D4-2 | TYWMW | 8 | SIYVGSGSSTYYASWAKG | 33 | GATNNVFMNYFNL | 57 |
| 70G2 | TYWMC | 9 | CIYVGSGGSTYYASWAKG | 34 | GATNNVFRNYFNL | 58 |
| 4F2 | YAMT | 10 | FVALRGNIYYANWAKG | 35 | GGLYTGYSYFDL | 59 |
| 62A10 | YAVT | 11 | FIGIRGHIYYANWAKG | 36 | GGLWTGNSYFDL | 60 |
| 70E2 | YAMT | 10 | FIGIRGNIYYANWAKG | 37 | GGLWTGNSYFDL | 60 |
| 71F10 | NAMT | 12 | FIGIRGNIYYANWAKG | 37 | GGLWTGNSYFDL | 60 |
| 73C2 | YAVT | 11 | FVGIYGDFYYANWAKG | 38 | GGLWTGNSYFDL | 60 |

TABLE 2
Amino Acid Sequences of Heavy Chain CDRs of Exemplary Antibodies
(IMGT Numbering Scheme)

| Clone | HCDR1 | SEQ ID NO | HCDR2 | SEQ ID NO | HCDR3 | SEQ ID NO |
|---|---|---|---|---|---|---|
| 9F7 | GFDFNNYG | | IYPAYHN | | ARDTGSTDYNFNL | |
| 2H3 | GFDFNNYG | | IYPAYHN | | ARDTGTTDYNFNL | |
| 69H10 | GFDFNKYG | | IYPAFHN | | ARDAGTTDYNFNL | |
| 65D5 | GFDFNAYG | | IYPAFHN | | ARDAGTTDYNFNL | |
| 3E9 | GFDFNNYG | | IYPAFHN | | ARDAGSTDYKFNL | |
| 9A5 | GFDFNNYG | | IYPTYHN | | ARDAGGTDYNFNL | |
| 12G6 | GFDFNNYG | | IYPAYHN | | ARDAGNTDYNFNL | |
| 1D10 | GFDFNYYG | | IYPAYHN | | ARDAGSTDYNLNL | |
| 62E3 | GFDFNEYG | | IYPAFHN | | ARDAGTTDYNFNL | |
| 3A4 | GFDFNEYG | | IYPAFHN | | ARDAGTTDYNFNL | |
| 63H3 | GFDFNSYG | | IYPAFHN | | ARDAGGTDYNYNL | |
| 66A1 | GFDFNNYG | | IYPAFHN | | ARDAGGTDYAYNL | |
| 2E12 | GFDFNNYG | | IYPAFPN | | ARDAGSTDYYFNL | |
| 72F12 | GFDFNNYG | | IYPAFHN | | ARDAGSVDYNFNL | |
| 74F11/15F5 | GFDFNNYG | | IYPAFHN | | ARDAGSTDYNFNL | |
| 9D7 | GFDLNNYS | | IYISGGS | | ARDDGTASYYLNL | |
| 9D7-2 | GFDLNNYS | | IYISGGS | | ARDDGTASYYLNL | |
| 10A9 | GFDLNNYA | | IYISGGT | | ARDDGTTSYYLNL | |
| 10A9-2 | GFDLNNYA | | IYISGGT | | ARDDGTTSYYLNL | |
| 62A2 | GFDLNNYA | | IYISGGT | | AKNDGTTSYYLNL | |
| 74E4 | GFDLNNYA | | IYISGAT | | ARDDGTTSYYLNL | |
| 66A6 | GFDLNNYA | | IYISGGT | | ARDDGTTSYYLNL | |
| 10E12 | GFDLNNYA | | IYISGGT | | ARDDGTTSYYLNL | |
| 8H2 | GFDLNNYA | | IYISGGT | | ARDDGSTSYYLNL | |
| 74H11 | GFDLNNYA | | IYISGGT | | ARDDGSTSYYLNL | |
| 74A1 | GFYLNNYA | | LYISGGT | | ARNDGSTNYYLNL | |
| 10F12 | GFDLNNYS | | IYISGGT | | ARDDGSTSYYLNL | |
| 65E11 | GFDLNNYS | | IYISGGT | | ARDDGSTSYYLNL | |
| 69C2 | GFDLNNYS | | IYISGGT | | ARDDGSTSYYLNL | |
| 61F12 | GFDLNNYS | | IYISGGT | | ARDDGTTSYYLNL | |
| 10D3 | GFDLNNYS | | IYISGGT | | ARDDGSTSYYLNL | |
| 67H4 | GFDLNNYS | | IYISGGS | | ARDDGTTSYYLNL | |
| 64G9 | GFDLNNYS | | IYISGGT | | ARDDGSTSYYLNL | |
| 72G12 | GFDLNNYS | | IYISGGT | | ARDDGTTSYYLNL | |
| 65G8 | GFDLNNYS | | IYISGGS | | ARDDGTTSYYLNL | |
| 69D8 | GFDLNNYS | | IYISGGS | | ARDDGTTSYYLNL | |
| 2H5 | GFDLNNYS | | IYFSGGS | | ARDDGTTSYYLNL | |
| 64A6 | GFDLNNYS | | IYISGGS | | ARDDGTASYYLNL | |
| 62B10 | GFDLNNYS | | IYISGGT | | ARDDGSVSYYLNL | |
| 5D4 | GFDFSGTYW | | IYVGSGSS | | ARGATNNVFMNYFNL | |
| 5D4-1 | GFDFSGTYW | | IYVGSGSS | | ARGATNNVFMNYFNL | |
| 5D4-2 | GFDFSGTYW | | IYVGSGSS | | ARGATNNVFMNYFNL | |
| 70G2 | GFDFSGTYW | | IYVGSGGS | | ARGATNNVFRNYFNL | |
| 4F2 | GFDFSNYA | | VALRGNI | | ARGGLYTGYSYFDL | |
| 62A10 | GFDFNNYA | | IGIRGHI | | ARGGLWTGNSYFDL | |
| 70E2 | GFDFSNYA | | IGIRGNI | | ARGGLWTGNSYFDL | |
| 71F10 | GFDFSSNA | | IGIRGNI | | ARGGLWTGNSYFDL | |
| 73C2 | GFDFSNYA | | VGIYGDF | | ARGGLWTGNSYFDL | |

TABLE 3
Amino Acid Sequences of Heavy Chain CDRs of Exemplary Antibodies
(Combining Kabat and IMGT Numbering Scheme)

| Clone | HCDR1 | SEQ ID NO | HCDR2 | SEQ ID NO | HCDR3 | SEQ ID NO |
|---|---|---|---|---|---|---|
| 9F7 | GFDFNNYGVS | | YIYPAYHNTYYATWVNG | | ARDTGSTDYNFNL | |
| 2H3 | GFDFNNYGVS | | YIYPAYHNTYYATWVNG | | ARDTGTTDYNFNL | |
| 69H10 | GFDFNKYGVS | | YIYPAFHNTYYATWVNG | | ARDAGTTDYNFNL | |
| 65D5 | GFDFNAYGVS | | YIYPAFHNTYYATWVNG | | ARDAGTTDYNFNL | |
| 3E9 | GFDFNNYGVS | | YIYPAFHNRYYANWVNG | | ARDAGSTDYKFNL | |
| 9A5 | GFDFNNYGVS | | YIYPTYHNTYYATWVNG | | ARDAGGTDYNFNL | |
| 12G6 | GFDFNNYGVS | | YIYPAYHNTYYATWVNG | | ARDAGNTDYNFNL | |
| 1D10 | GFDFNYYGVS | | YIYPAYHNTYYATWVNG | | ARDAGSTDYNLNL | |
| 62E3 | GFDFNEYGVS | | YIYPAFHNTYYATWVNG | | ARDAGTTDYNFNL | |
| 3A4 | GFDFNEYGVS | | YIYPAFHNTYYATWVNG | | ARDAGTTDYNFNL | |
| 63H3 | GFDFNSYGVS | | YIYPAFHNAYYANWVNG | | ARDAGGTDYNYNL | |
| 66A1 | GFDFNNYGVS | | YIYPAFHNTYYASWVNG | | ARDAGGTDYAYNL | |
| 2E12 | GFDFNNYGVT | | YIYPAFPNTYYATWVNG | | ARDAGSTDYYFNL | |
| 72F12 | GFDFNNYGVS | | YIYPAFHNTYYATWVNG | | ARDAGSVDYNFNL | |
| 74F11/15F5 | GFDFNNYGMS | | YIYPAFHNTYYANWVNG | | ARDAGSTDYNFNL | |
| 9D7 | GFDLNNYSIL | | CIYISGGSTYYANWAKG | | ARDDGTASYYLNL | |
| 9D7-2 | GFDLNNYSIL | | SIYISGGSTYYANWAKG | | ARDDGTASYYLNL | |
| 10A9 | GFDLNNYAML | | CIYISGGTTYFASWATG | | ARDDGTTSYYLNL | |
| 10A9-2 | GFDLNNYAML | | SIYISGGTTYFASWATG | | ARDDGTTSYYLNL | |
| 62A2 | GFDLNNYAML | | CIYISGGTTYFASWATG | | AKNDGTTSYYLNL | |
| 74E4 | GFDLNNYAIL | | CIYISGATTYFANWATG | | ARDDGTTSYYLNL | |
| 66A6 | GFDLNNYAML | | CIYISGGTTYFASWATG | | ARDDGTTSYYLNL | |
| 10E12 | GFDLNNYAML | | CIYISGGTTYFASWATG | | ARDDGTTSYYLNL | |
| 8H2 | GFDLNNYAML | | CIYISGGTTYFASWATG | | ARDDGSTSYYLNL | |
| 74H11 | GFDLNNYAML | | CIYISGGTTYFASWATG | | ARDDGSTSYYLNL | |
| 74A1 | GFYLNNYAML | | CLYISGGTTYFASWATG | | ARNDGSTNYYLNL | |
| 10F12 | GFDLNNYSML | | CIYISGGTTYFASWATG | | ARDDGSTSYYLNL | |
| 65E11 | GFDLNNYSML | | CIYISGGTTYFASWATG | | ARDDGSTSYYLNL | |
| 69C2 | GFDLNNYSML | | CIYISGGTTYFASWATG | | ARDDGSTSYYLNL | |
| 61F12 | GFDLNNYSIL | | CIYISGGTTYFASWATG | | ARDDGTTSYYLNL | |
| 10D3 | GFDLNNYSML | | CIYISGGTTYYASWAKG | | ARDDGSTSYYLNL | |
| 67H4 | GFDLNNYSIL | | CIYISGGSTYYASWAKG | | ARDDGTTSYYLNL | |
| 64G9 | GFDLNNYSIL | | CIYISGGTTYYANWAKG | | ARDDGSTSYYLNL | |
| 72G12 | GFDLNNYSIL | | CIYISGGTTYYANWAKG | | ARDDGTTSYYLNL | |
| 65G8 | GFDLNNYSIL | | CIYISGGSTYYANWAKG | | ARDDGTTSYYLNL | |
| 69D8 | GFDLNNYSIL | | CIYISGGSTYYANWAKG | | ARDDGTTSYYLNL | |
| 2H5 | GFDLNNYSIL | | CIYFSGGSTYYANWAKG | | ARDDGTTSYYLNL | |
| 64A6 | GFDLNNYSIL | | CIYISGGSTYYANWAKG | | ARDDGTASYYLNL | |
| 62B10 | GFDLNNYSIL | | CIYISGGTTYFANWAKG | | ARDDGSVSYYLNL | |
| 5D4 | GFDFSGTYWMW | | CIYVGSGSSTYYASWAKG | | ARGATNNVFMNYFNL | |
| 5D4-1 | GFDFSGTYWMW | | PIYVGSGSSTYYASWAKG | | ARGATNNVFMNYFNL | |
| 5D4-2 | GFDFSGTYWMW | | SIYVGSGSSTYYASWAKG | | ARGATNNVFMNYFNL | |
| 70G2 | GFDFSGTYWMC | | CIYVGSGGSTYYASWAKG | | ARGAINNVFRNYFNL | |
| 4F2 | GFDFSNYAMT | | FVALRGNIYYANWAKG | | ARGGLYTGYSYFDL | |
| 62A10 | GFDFNNYAVT | | FIGIRGHIYYANWAKG | | ARGGLWTGNSYFDL | |
| 70E2 | GFDFSNYAMT | | FIGIRGNIYYANWAKG | | ARGGLWTGNSYFDL | |
| 71F10 | GFDFSSNAMT | | FIGIRGNIYYANWAKG | | ARGGLWTGNSYFDL | |
| 73C2 | GFDFSNYAVT | | FVGIYGDFYYANWAKG | | ARGGLWTGNSYFDL | |

TABLE 4
Amino Acid Sequences Light Chain CDRs of Exemplary Antigen Binding Molecules
(Kabat Numbering Scheme)

| Clone | LCDR1 | SEQ ID NO | LCDR2 | SEQ ID NO | LCDR3 | SEQ ID NO |
|---|---|---|---|---|---|---|
| 9F7 | QASEDIESYLA | 61 | EASKLAS | 81 | QQALTVGNVDNP | 94 |
| 2H3 | QASEDIESYLA | 61 | EASKLAS | 81 | QQALTVGNVDNP | 94 |
| 69H10 | QASQSIYSYLS | 62 | EASKLAS | 81 | QQALTVGNVDNP | 94 |
| 65D5 | QASQSISSYLA | 63 | EATKLIS | 82 | QQALTVGDVDNP | 95 |
| 3E9 | QASEDIESYLA | 61 | DASTLAS | 83 | QQDLTVGNVDNP | 96 |
| 9A5 | QASQSIESYLA | 64 | EASKLAS | 81 | QQALTIGNVDNP | 97 |
| 12G6 | QASEDIESYLA | 61 | EASKLAS | 81 | QQALTIGNVDNP | 97 |
| 1D10 | QASEDIESYLA | 61 | EASKLAS | 81 | HQALTIGNVDNP | 98 |
| 62E3 | QASEDIESYLA | 61 | EASKLAS | 81 | QQALTIGNVDNP | 97 |
| 3A4 | QASEDIENYLA | 65 | EASKLAS | 81 | QQALTIGNVDNP | 97 |
| 63H3 | QASEDIESYSA | 66 | EASKLAS | 81 | QQALTIGNVDNP | 97 |
| 66A1 | QASEDIESYLA | 61 | KASTLAS | 84 | QQVLTIGNVDNP | 99 |
| 2E12 | QASENIESYLA | 67 | DASTLAS | 83 | QQGLTIGNVDNP | 100 |
| 72F12 | QASKNIDSNLA | 68 | AASTLAS | 85 | QQALTIGNVDNP | 97 |
| 74F11/15F5 | QASEDIERYLA | 69 | EASKLPS | 86 | QQALTIGYVDNP | 101 |
| 9D7 | QASEDIYKLLA | 70 | AASDLES | 87 | QQAYTIGNIDNS | 102 |
| 9D7-2 | QASEDIYKLLA | 70 | AASDLES | 87 | QQAYTIGNIDNS | 102 |
| 10A9 | QASEDIYSLLA | 71 | GASNLES | 88 | QQAYTIGNIDNA | 103 |
| 10A9-2 | QASEDIYSLLA | 71 | GASNLES | 88 | QQAYTIGNIDNA | 103 |
| 62A2 | QASEDIYRLLA | 72 | GASNLES | 88 | QQAYTIGNIDNA | 103 |
| 74E4 | QASEDIYRLLA | 72 | GASNLES | 88 | QQAYTIGNIDNA | 103 |
| 66A6 | QASEDIYSLLA | 71 | DASDLAS | 89 | QQAYTIGNIDNT | 104 |
| 10E12 | QASEDIYSLLA | 71 | DASDLAS | 89 | QQAYTIGNIDNA | 103 |
| 8H2 | QASEDIYSLLA | 71 | DASDLAS | 89 | QQAYTIGNIDNT | 104 |
| 74H11 | QASEDIYSLLA | 71 | DASDLAS | 89 | QQAYTIGNIDNA | 103 |
| 74A1 | QASQSIYNNFA | 73 | DASDLAS | 89 | QQAYTIGNIDNP | 105 |
| 10F12 | QASEDIYSLLA | 71 | DASDLAS | 89 | QQAYTIGNIDNA | 103 |
| 65E11 | QASEDIYSLLA | 71 | DASTLAS | 83 | QQAYTIGNIDNA | 103 |
| 69C2 | QASEDIYKLLA | 70 | AASDLES | 87 | QQAYTIGNIDNT | 104 |
| 61F12 | QASEDIYSLLA | 71 | DASDLAS | 89 | QQAYTIGNIDNS | 102 |
| 10D3 | QASEDIYRLLA | 72 | DASDLAS | 89 | QQGYTIGNIDNS | 106 |
| 67H4 | QASEDIYNLLA | 74 | AASDLES | 87 | QQGYTIGNIDNA | 107 |
| 64G9 | QASEDIYNLLA | 74 | DASTLTS | 90 | QQAYTIGNIDNT | 104 |
| 72G12 | QASEDIYKLLA | 70 | AASDLES | 87 | QQAYTIGNLDNA | 103 |
| 65G8 | QASEDIYKLLA | 70 | AASDLES | 87 | QQAYTIGNIDNT | 104 |
| 69D8 | QASEDIYSLLA | 71 | DASTLAS | 83 | QQAYTIGNIDNA | 103 |
| 2H5 | QASEDIYNLLA | 74 | AASDLES | 87 | QQAYTVGNIDNA | 108 |
| 64A6 | QASEDIYKLLA | 70 | AASDLES | 87 | QQAYTIGNIDNS | 102 |
| 62B10 | QASEDIYNLLA | 74 | DASTLTS | 90 | QQGYTIGNIDNT | 109 |
| 5D4 | QASEDISSNLG | 75 | GASTLAS | 91 | QTSYYIDDGVNG | 110 |
| 5D4-1 | QASEDISSNLG | 75 | GASTLAS | 91 | QTSYYIDDGVNG | 110 |
| 5D4-2 | QASEDISSNLG | 75 | GASTLAS | 91 | QTSYYIDDGVNG | 110 |
| 70G2 | QASEDISSNLA | 76 | GASTLAS | 91 | QSSYYIDDGVNG | 111 |
| 4F2 | QASESLSSYLA | 77 | RAATLAS | 92 | QQGYGYSTVGNA | 112 |
| 62A10 | QASESISRYLA | 78 | RASTLAS | 93 | QQGYGYSTVDNA | 211 |
| 70E2 | QASENINRYLA | 79 | RAATLAS | 92 | QQGYGYSTVGNA | 112 |
| 71F10 | QASESISRYLA | 78 | RASTLAS | 93 | QQGYGYSTVGNA | 112 |
| 73C2 | QASESINRYLA | 80 | RAATLAS | 92 | QQGYGYSTVGNA | 112 |

TABLE 5
Amino Acid Sequences Light Chain CDRs of Exemplary Antigen Binding Molecules
(IMGT Numbering Scheme)

| Clone | LCDR1 | SEQ ID NO | LCDR2 | SEQ ID NO | LCDR3 | SEQ ID NO |
|---|---|---|---|---|---|---|
| 9F7 | EDIESY | | EAS | | QQALTVGNVDNP | |
| 2H3 | EDIESY | | EAS | | QQALTVGNVDNP | |
| 69H10 | QSIYSY | | EAS | | QQALTVGNVDNP | |
| 65D5 | QSISSY | | EAT | | QQALTVGDVDNP | |
| 3E9 | EDIESY | | DAS | | QQDLTVGNVDNP | |
| 9A5 | QSIESY | | EAS | | QQALTIGNVDNP | |
| 12G6 | EDIESY | | EAS | | QQALTIGNVDNP | |
| 1D10 | EDIESY | | EAS | | HQALTIGNVDNP | |
| 62E3 | EDIESY | | EAS | | QQALTIGNVDNP | |
| 3A4 | EDIENY | | EAS | | QQALTIGNVDNP | |
| 63H3 | EDIESY | | EAS | | QQALTIGNVDNP | |
| 66A1 | EDIESY | | KAS | | QQVLTIGNVDNP | |
| 2E12 | ENIESY | | DAS | | QQGLTIGNVDNP | |
| 72F12 | KNIDSN | | AAS | | QQALTIGNVDNP | |
| 74F11/15F5 | EDIERY | | EAS | | QQALTIGYVDNP | |
| 9D7 | EDIYKL | | AAS | | QQAYTIGNIDNS | |
| 9D7-2 | EDIYKL | | AAS | | QQAYTIGNIDNS | |
| 10A9 | EDIYSL | | GAS | | QQAYTIGNIDNA | |
| 10A9-2 | EDIYSL | | GAS | | QQAYTIGNIDNA | |
| 62A2 | EDIYRL | | GAS | | QQAYTIGNIDNA | |
| 74E4 | EDIYRL | | GAS | | QQAYTIGNIDNA | |
| 66A6 | EDIYSL | | DAS | | QQAYTIGNIDNT | |
| 10E12 | EDIYSL | | DAS | | QQAYTIGNIDNA | |
| 8H2 | EDIYSL | | DAS | | QQAYTIGNIDNT | |
| 74H11 | EDIYSL | | DAS | | QQAYTIGNIDNA | |
| 74A1 | QSIYNN | | DAS | | QQAYTIGNIDNP | |
| 10F12 | EDIYSL | | DAS | | QQAYTIGNIDNA | |
| 65E11 | EDIYSL | | DAS | | QQAYTIGNIDNA | |
| 69C2 | EDIYKL | | AAS | | QQAYTIGNIDNT | |
| 61F12 | EDIYSL | | DAS | | QQAYTIGNIDNS | |
| 10D3 | EDIYRL | | DAS | | QQGYTIGNIDNS | |
| 67H4 | EDIYNL | | AAS | | QQGYTIGNIDNA | |
| 64G9 | EDIYNL | | DAS | | QQAYTIGNIDNT | |
| 72G12 | EDIYKL | | AAS | | QQAYTIGNLDNA | |
| 65G8 | EDIYKL | | AAS | | QQAYTIGNIDNT | |
| 69D8 | EDIYSL | | DAS | | QQAYTIGNIDNA | |
| 2H5 | EDIYNL | | AAS | | QQAYTVGNIDNA | |
| 64A6 | EDIYKL | | AAS | | QQAYTIGNIDNS | |
| 62B10 | EDIYNL | | DAS | | QQGYTIGNIDNT | |
| 5D4 | EDISSN | | GAS | | QTSYYIDDGVNG | |
| 5D4-1 | EDISSN | | GAS | | QTSYYIDDGVNG | |
| 5D4-2 | EDISSN | | GAS | | QTSYYIDDGVNG | |
| 70G2 | EDISSN | | GAS | | QSSYYIDDGVNG | |
| 4F2 | ESLSSY | | RAA | | QQGYGYSTVGNA | |
| 62A10 | ESISRY | | RAS | | QQGYGYSTVDNA | |
| 70E2 | ENINRY | | RAA | | QQGYGYSTVGNA | |
| 71F10 | ESISRY | | RAS | | QQGYGYSTVGNA | |
| 73C2 | ESINRY | | RAA | | QQGYGYSTVGNA | |

Table 4 also shows the amino acid sequences of LCDRs of the exemplary antibodies combining Kabat and IMGT numbering scheme (same as Kabat).

Tables 6-16 show the consensus amino acid sequences for several CDRs in several exemplary antibodies of the present disclosure.

TABLE 6
Consensus Sequences of Heavy Chain CDR1 in Exemplary Antigen Binding Molecules
(Kabat Numbering Scheme)

| Clone | Sequence | SEQ ID NO: |
|---|---|---|
| 9F7 | YGVS | |
| 2H3 | YGVS | |
| 69H10 | YGVS | |
| 65D5 | YGVS | |
| 3E9 | YGVS | |
| 9A5 | YGVS | |
| 12G6 | YGVS | |
| 1D10 | YGVS | |
| 62E3 | YGVS | |
| 3A4 | YGVS | |
| 63H3 | YGVS | |
| 66A1 | YGVS | |
| 2E12 | YGVT | |
| 72F12 | YGVS | |
| 74F11/15F5 | YGMS | |
| N/A | Y-G-X1-X2 (SEQ ID NO: ) or Y-G-X3-S (SEQ ID | |
| | NO: ) (excluding clone 2E12) or Y-G-V-X4 (SEQ ID NO: ) | |
| | (excluding clones 74F11/15F5) | |
| 9D7 | YSIL | |
| 9D7-2 | YSIL | |
| 10A9 | YAML | |
| 10A9-2 | YAML | |
| 62A2 | YAML | |
| 74E4 | YAIL | |
| 66A6 | YAML | |
| 10E12 | YAML | |
| 8H2 | YAML | |
| 74H11 | YAML | |
| 74A1 | YAML | |
| 10F12 | YSML | |
| 65E11 | YSML | |
| 69C2 | YSML | |
| 61F12 | YSIL | |
| 10D3 | YSML | |
| 67H4 | YSIL | |
| 64G9 | YSIL | |
| 72G12 | YSIL | |
| 65G8 | YSIL | |
| 69D8 | YSIL | |
| 2H5 | YSIL | |
| 64A6 | YSIL | |
| 62B10 | YSIL | |
| N/A | Y-X5-X6-L (SEQ ID NO: ) or Y-A-X7-L (SEQ ID | |
| | NO: ) (clones with 2nd position being an A) or | |
| | Y-S-X8-L (SEQ ID NO: ) (clones with 2^{nd} | |
| | position being an S) | |
| 5D4 | TYWMW | |
| 5D4-1 | TYWMW | |
| 5D4-2 | TYWMW | |
| 70G2 | TYWMC | |
| N/A | T-Y-W-M-X9 | |
| 4F2 | YAMT | |
| 62A10 | YAVT | |
| 70E2 | YAMT | |
| 71F10 | NAMT | |
| 73C2 | YAVT | |
| N/A | X10-A-X11-T (SEQ ID NO: ) or Y-A-X12-T (SEQ | |
| | ID NO: ) (excluding clone 71F10) or X13-A-M-T | |
| | (SEQ ID NO: ) (clones 4F2 and 71F10) | |

As used herein, X1 is M or V, X2 is S or T, X3 is M or V, X4 is S or T, X5 is A or S, X6 is J or M, X7 is J or M, X8 is J or M, X9 is C or W, X10 is N or Y, X11 is M or V, X12 is M or V, and X13 is Nor Y.

TABLE 7
Consensus Sequences of Heavy Chain CDR1 in Exemplary Antigen Binding Molecules
(IMGT Numbering Scheme)

| Clone | Sequence | SEQ ID NO: |
|---|---|---|
| 9F7 | GFDFNNYG | |
| 2H3 | GFDFNNYG | |
| 69H10 | GFDFNKYG | |
| 65D5 | GFDFNAYG | |
| 3E9 | GFDFNNYG | |
| 9A5 | GFDFNNYG | |
| 12G6 | GFDFNNYG | |
| 1D10 | GFDFNYYG | |
| 62E3 | GFDFNEYG | |
| 3A4 | GFDFNEYG | |
| 63H3 | GFDFNSYG | |
| 66A1 | GFDFNNYG | |
| 2E12 | GFDFNNYG | |
| 72F12 | GFDFNNYG | |
| 74F11/15F5 | GFDFNNYG | |
| N/A | G-F-D-F-N-X14-Y-G | |
| 9D7 | GFDLNNYS | |
| 9D7-2 | GFDLNNYS | |
| 10A9 | GFDLNNYA | |
| 10A9-2 | GFDLNNYA | |
| 62A2 | GFDLNNYA | |
| 74E4 | GFDLNNYA | |
| 66A6 | GFDLNNYA | |
| 10E12 | GFDLNNYA | |
| 8H2 | GFDLNNYA | |
| 74H11 | GFDLNNYA | |
| 74A1 | GFYLNNYA | |
| 10F12 | GFDLNNYS | |
| 65E11 | GFDLNNYS | |
| 69C2 | GFDLNNYS | |
| 61F12 | GFDLNNYS | |
| 10D3 | GFDLNNYS | |
| 67H4 | GFDLNNYS | |
| 64G9 | GFDLNNYS | |
| 72G12 | GFDLNNYS | |
| 65G8 | GFDLNNYS | |
| 69D8 | GFDLNNYS | |
| 2H5 | GFDLNNYS | |
| 64A6 | GFDLNNYS | |
| 62B10 | GFDLNNYS | |
| N/A | G-F-X15-L-N-N-Y-X16 (SEQ ID NO: ) or G-F-D-L-N-N-Y-X17 (SEQ ID NO: ) | |
| | (excluding 74A1) | |
| 4F2 | GFDFSNYA | |
| 62A10 | GFDFNNYA | |
| 70E2 | GFDFSNYA | |
| 71F10 | GFDFSSNA | |
| 73C2 | GFDFSNYA | |
| N/A | G-F-D-F-X18-X19-X20-A (SEQ ID NO: ) or G-F-D-F-X21-N-Y-A (SEQ ID NO: ) | |
| | (excluding clone 71F10) | |

As used herein, X14 is A, E, N, K, 5, or Y, X15 is D or Y, X16 is A or 5, X17 is A or S, X18 is N or 5, X19 is N or 5, X20 is N or Y, and X21 is Nor S.

TABLE 8
Consensus Sequences of Heavy Chain CDR1 in Exemplary Antigen Binding Molecules
(Combing Kabat and IMGT Numbering Scheme)

| Clone | Sequence | SEQ ID NO |
|---|---|---|
| 9F7 | GFDFNNYGVS | |
| 2H3 | GFDFNNYGVS | |
| 69H10 | GFDFNKYGVS | |
| 65D5 | GFDFNAYGVS | |
| 3E9 | GFDFNNYGVS | |
| 9A5 | GFDFNNYGVS | |
| 12G6 | GFDFNNYGVS | |
| 1D10 | GFDFNYYGVS | |
| 62E3 | GFDFNEYGVS | |
| 3A4 | GFDFNEYGVS | |
| 63H3 | GFDFNSYGVS | |
| 66A1 | GFDFNNYGVS | |
| 2E12 | GFDFNNYGVT | |
| 72F12 | GFDFNNYGVS | |
| 74F11/15F5 | GFDFNNYGMS | |
| N/A | G-F-D-F-N-X22-Y-G-X23-X24 (SEQ ID NO: ) or G-F- | |
| | D-F-N-X25-Y-G-X26-S (SEQ ID NO: ) (excluding | |
| | clone 2E12) or G-F-D-F-N-X27-Y-G-V-X28 (SEQ ID | |
| | NO: ) (excluding clones 74F11/15F5) | |
| 9D7 | GFDLNNYSIL | |
| 9D7-2 | GFDLNNYSIL | |
| 10A9 | GFDLNNYAML | |
| 10A9-2 | GFDLNNYAML | |
| 62A2 | GFDLNNYAML | |
| 74E4 | GFDLNNYAIL | |
| 66A6 | GFDLNNYAML | |
| 10E12 | GFDLNNYAML | |
| 8H2 | GFDLNNYAML | |
| 74H11 | GFDLNNYAML | |
| 74A1 | GFYLNNYAML | |
| 10F12 | GFDLNNYSML | |
| 65E11 | GFDLNNYSML | |
| 69C2 | GFDLNNYSML | |
| 61F12 | GFDLNNYSIL | |
| 10D3 | GFDLNNYSML | |
| 67H4 | GFDLNNYSIL | |
| 64G9 | GFDLNNYSIL | |
| 72G12 | GFDLNNYSIL | |
| 65G8 | GFDLNNYSIL | |
| 69D8 | GFDLNNYSIL | |
| 2H5 | GFDLNNYSIL | |
| 64A6 | GFDLNNYSIL | |
| 62B10 | GFDLNNYSIL | |
| N/A | G-F-X29-L-N-N-Y-X30-X31-L (SEQ ID NO: ) or G-F- | |
| | D-L-N-N-Y-X32-X33-L (SEQ ID NO: ) (excluding | |
| | clone 74A1) | |
| 5D4 | GFDFSGTYWMW | |
| 5D4-1 | GFDFSGTYWMW | |
| 5D4-2 | GFDFSGTYWMW | |
| 70G2 | GFDFSGTYWMC | |
| N/A | G-F-D-F-S-G-T-Y-W-M-X34 | |
| 4F2 | GFDFSNYAMT | |
| 62A10 | GFDFNNYAVT | |
| 70E2 | GFDFSNYAMT | |
| 71F10 | GFDFSSNAMT | |
| 73C2 | GFDFSNYAVT | |
| N/A | G-F-D-F-X35-X36-X37-A-X38-T (SEQ ID NO: ) or G- | |
| | F-D-F-S-X39-X40-A-X41-T (SEQ ID NO: ) (excluding | |
| | clone 62A10) or G-F-D-F-X42-N-Y-A-X43-T (SEQ ID | |
| | NO: ) (excluding clone 71F10) | |

As used herein, X22 is A, E, K, N, 5, or Y, X23 is M or V, X24 S or T, X25 is A, E, K, N, S, or Y, X26 is M or V, X27is is A, E, K, N, S, or Y, X28 is S or T, X29 is D or Y, X3 O is A or S, X31 is I or M, X32 is A or 5, X33 is I or M, X34 is C or W, X35 is N or 5, X36 is N or 5, X37 is N or Y, X38 is M or V, X39 is N or S, X40O is Nor Y, X41is M or V, X42 is Nor S, and X43 is M or V.

TABLE 9
Consensus Sequences of Heavy Chain CDR2 in Exemplary Antigen Binding Molecules
(Kabat Numbering Scheme)

| Clone | Sequences | SEQ ID NO |
|---|---|---|
| 9F7 | YIYPAYHNTYYATWVNG | |
| 2H3 | YIYPAYHNTYYATWVNG | |
| 69H10 | YIYPAFHNTYYATWVNG | |
| 65D5 | YIYPAFHNTYYATWVNG | |
| 3E9 | YIYPAFHNRYYANWVNG | |
| 9A5 | YIYPTYHNTYYATWVNG | |
| 12G6 | YIYPAYHNTYYATWVNG | |
| 1D10 | YIYPAYHNTYYATWVNG | |
| 62E3 | YIYPAFHNTYYATWVNG | |
| 3A4 | YIYPAFHNTYYATWVNG | |
| 63H3 | YIYPAFHNAYYANWVNG | |
| 66A1 | YIYPAFHNTYYASWVNG | |
| 2E12 | YIYPAFPNTYYATWVNG | |
| 72F12 | YIYPAFHNTYYATWVNG | |
| 74F11/15F5 | YIYPAFHNTYYANWVNG | |
| N/A | Y-I-Y-P-X44-X45-X46-N-X47-Y-Y-A-X48-W-V-N-G (SEQ ID NO: ) | |
| | or Y-I-Y-P-A-X49-H-N-X50-Y-Y-A-X51-W-V-N-G (SEQ ID NO: ) | |
| | (excluding clones 9A5 and 2E12) | |
| 9D7 | CIYISGGSTYYANWAKG | |
| 9D7-2 | SIYISGGSTYYANWAKG | |
| 10A9 | CIYISGGTTYFASWATG | |
| 10A9-2 | SIYISGGTTYFASWATG | |
| 62A2 | CIYISGGTTYFASWATG | |
| 74E4 | CIYISGATTYFANWATG | |
| 66A6 | CIYISGGTTYFASWATG | |
| 10E12 | CIYISGGTTYFASWATG | |
| 8H2 | CIYISGGTTYFASWATG | |
| 74H11 | CIYISGGTTYFASWATG | |
| 74A1 | CLYISGGTTYFASWATG | |
| 10F12 | CIYISGGTTYFASWATG | |
| 65E11 | CIYISGGTTYFASWATG | |
| 69C2 | CIYISGGTTYFASWATG | |
| 61F12 | CIYISGGTTYFASWATG | |
| 10D3 | CIYISGGTTYYASWAKG | |
| 67H4 | CIYISGGSTYYASWAKG | |
| 64G9 | CIYISGGTTYYANWAKG | |
| 72G12 | CIYISGGTTYYANWAKG | |
| 65G8 | CIYISGGSTYYANWAKG | |
| 69D8 | CIYISGGSTYYANWAKG | |
| 2H5 | CIYFSGGSTYYANWAKG | |
| 64A6 | CIYISGGSTYYANWAKG | |
| 62B10 | CIYISGGTTYFANWAKG | |
| N/A | X52-X53-Y-X54-S-G-X55-X56-T-Y-X57-A-X58-W-A-X59-G (SEQ ID NO: ) | |
| | or X60-I-Y-I-S-G-G-X61-T-Y-X62-A-X63-W-A-X64-G (SEQ ID NO: ) | |
| | (excluding clones 74A1, 74E4, and 2H5) | |
| 5D4 | CIYVGSGSSTYYASWAKG | |
| 5D4-1 | PIYVGSGSSTYYASWAKG | |
| 5D4-2 | SIYVGSGSSTYYASWAKG | |
| 70G2 | CIYVGSGGSTYYASWAKG | |
| N/A | X65-I-Y-V-G-S-G-X66-S-T-Y-Y-A-S-W-A-K-G | |
| 4F2 | FVALRGNIYYANWAKG | |
| 62A10 | FIGIRGHIYYANWAKG | |
| 70E2 | FIGIRGNIYYANWAKG | |
| 71F10 | FIGIRGNIYYANWAKG | |
| 73C2 | FVGIYGDFYYANWAKG | |
| N/A | F-X67-X68-X69-X70-G-X71-X72-Y-Y-A-N-W-A-K-G (SEQ ID NO: ) or | |
| | F-X73-G-I-X74-G-X75-X76-Y-Y-A-N-W-A-K-G (SEQ ID NO: ) | |
| | (excluding clone 4F2) or F-X77-X78-X79-R-G-X80-I-Y-Y-A-N-W-A- | |
| | K-G (SEQ ID NO: ) (excluding clone 73C2) or F-I-G-I-R-G-X81-I- | |
| | Y-Y-A-N-W-A-K-G (SEQ ID NO: ) (excluding clones 4F2 and 73C2) | |

As used herein, X44 is A or T, X45 is F or Y, X46 is H or P, X47 is A, R, or T, X48 is N, S or T, X49 is F or Y, X50 is A, R, or T, X51 is N, 5, or T, X52 is C or 5, X53 is I or L, X54 is F or I, X55 is A or G, X56 is S or T, X57 is F or Y, X58 is N or 5, X59 is K or T, X60 is C or 5, X61 is S or T, X62 is F or Y, X63 is N or S, X64 is K or T, X65 is C, P, or S, X66 is G or S, X67 is I or V, X6S is A or G, X69 is I or L, X70 is R or Y, X71 is D, H, or N, X72 is F or I, X73 is I or V, X74 is R or Y, X75 is D, N, H, X76 is F or I, X77 is I or V, X78 is A or G, X79 is I or L, X80 O is H or N, and X81 is H or N.

TABLE 10
Consensus Sequences of Heavy Chain CDR2 in Exemplary Antigen Binding Molecules
(IMGT Numbering Scheme)

| Clone | Sequences | SEQ ID NO: |
|---|---|---|
| 9F7 | IYPAYHN | |
| 2H3 | IYPAYHN | |
| 69H10 | IYPAFHN | |
| 65D5 | IYPAFHN | |
| 3E9 | IYPAFHN | |
| 9A5 | IYPTYHN | |
| 12G6 | IYPAYHN | |
| 1D10 | IYPAYHN | |
| 62E3 | IYPAFHN | |
| 3A4 | IYPAFHN | |
| 63H3 | IYPAFHN | |
| 66A1 | IYPAFHN | |
| 2E12 | IYPAFPN | |
| 72F12 | IYPAFHN | |
| 74F11/15F5 | IYPAFHN | |
| N/A | I-Y-P-X82-X83-X84-N (SEQ ID NO: ) or I-Y-P-A- | |
| | X85-X86-N (SEQ ID NO: ) (excluding clone 9A5) or | |
| | I-Y-P-X87-X88-H-N (SEQ ID NO: ) (excluding clone 2E12) | |
| 9D7 | IYISGGS | |
| 9D7-2 | IYISGGS | |
| 10A9 | IYISGGT | |
| 10A9-2 | IYISGGT | |
| 62A2 | IYISGGT | |
| 74E4 | IYISGAT | |
| 66A6 | IYISGGT | |
| 10E12 | IYISGGT | |
| 8H2 | IYISGGT | |
| 74H11 | IYISGGT | |
| 74A1 | LYISGGT | |
| 10F12 | IYISGGT | |
| 65E11 | IYISGGT | |
| 69C2 | IYISGGT | |
| 61F12 | IYISGGT | |
| 10D3 | IYISGGT | |
| 67H4 | IYISGGS | |
| 64G9 | IYISGGT | |
| 72G12 | IYISGGT | |
| 65G8 | IYISGGS | |
| 69D8 | IYISGGS | |
| 2H5 | IYFSGGS | |
| 64A6 | IYISGGS | |
| 62B10 | IYISGGT | |
| N/A | X89-Y-I-S-G-X90-X91 (SEQ ID NO: ) or I-Y-I-S-G- | |
| | G-X92 (SEQ ID NO: ) (excluding clones 74A1 and 74E4) | |
| 5D4 | IYVGSGSS | |
| 5D4-1 | IYVGSGSS | |
| 5D4-2 | IYVGSGSS | |
| 70G2 | IYVGSGGS | |
| N/A | I-Y-V-G-S-G-X93-S | |
| 4F2 | VALRGNI | |
| 62A10 | IGIRGHI | |
| 70E2 | IGIRGNI | |
| 71F10 | IGIRGNI | |
| 73C2 | VGIYGDF | |
| N/A | X94-G-I-X95-G-X96-X97 (SEQ ID NO: ) (excluding | |
| | clone 4F2) or I-G-I-R-G-X98-I (SEQ ID NO: ) | |
| | (excluding clones 4F2 and 73C2) | |

As used herein, X82 is A or T, X83 is F or Y, X84 is H or P, X85 is F or Y, X86 is H or P, X87 is A or T, X88 is F or Y, X89 is I or L, X90 is A or G, X91 is S or T, X92 is S or T, X93 is G or S, X94 is I or V, X95 is R or Y, X96 is D, H, or N, X97 is I or F, and X98 is H or N..

Table 9 also shows several consensus sequences of heavy chain CDR2 of several exemplary antibodies combining Kabat and IMGT numbering scheme (same as Kabat).

TABLE 11
Consensus Sequences of Heavy Chain CDR3 in Exemplary Antigen Binding Molecules
(Kabat Numbering Scheme)

| Clone | Sequences | SEQ ID NO |
|---|---|---|
| 9F7 | DTGSTDYNFNL | |
| 2H3 | DTGTTDYNFNL | |
| 69H10 | DAGTTDYNFNL | |
| 65D5 | DAGTIDYNFNL | |
| 3E9 | DAGSTDYKFNL | |
| 9A5 | DAGGTDYNFNL | |
| 12G6 | DAGNTDYNFNL | |
| 1D10 | DAGSTDYNLNL | |
| 62E3 | DAGTTDYNFNL | |
| 3A4 | DAGTTDYNFNL | |
| 63H3 | DAGGTDYNYNL | |
| 66A1 | DAGGTDYAYNL | |
| 2E12 | DAGSTDYYFNL | |
| 72F12 | DAGSVDYNFNL | |
| 74F11/15F5 | DAGSTDYNFNL | |
| N/A | D-X99-G-X100-X101-D-Y-X102-X103-N-L (SEQ ID NO: ) or D-A-G-X104- | |
| | T-D-Y-X105-X106-N-L (SEQ ID NO: ) | |
| | (excluding clones 9F7, 2H3, and 72F12) | |
| 9D7 | DDGTASYYLNL | |
| 9D7-2 | DDGTASYYLNL | |
| 10A9 | DDGTTSYYLNL | |
| 10A9-2 | DDGTTSYYLNL | |
| 62A2 | NDGTTSYYLNL | |
| 74E4 | DDGTTSYYLNL | |
| 66A6 | DDGTTSYYLNL | |
| 10E12 | DDGTTSYYLNL | |
| 8H2 | DDGSTSYYLNL | |
| 74H11 | DDGSTSYYLNL | |
| 74A1 | NDGSTNYYLNL | |
| 10F12 | DDGSTSYYLNL | |
| 65E11 | DDGSTSYYLNL | |
| 69C2 | DDGSTSYYLNL | |
| 61F12 | DDGTTSYYLNL | |
| 10D3 | DDGSTSYYLNL | |
| 67H4 | DDGTTSYYLNL | |
| 64G9 | DDGSTSYYLNL | |
| 72G12 | DDGTTSYYLNL | |
| 65G8 | DDGTTSYYLNL | |
| 69D8 | DDGTTSYYLNL | |
| 2H5 | DDGTTSYYLNL | |
| 64A6 | DDGTASYYLNL | |
| 62B10 | DDGSVSYYLNL | |
| N/A | X107-D-G-X108-X109-X110-Y-Y-L-N-L (SEQ ID NO: ) or D-D-G-X111- | |
| | X112-S-Y-Y-L-N-L (SEQ ID NO: ) | |
| | (excluding clones 62A2 and 74A1) | |
| 5D4 | GATNNVFMNYFNL | |
| 5D4-1 | GATNNVFMNYFNL | |
| 5D4-2 | GATNNVFMNYFNL | |
| 70G2 | GATNNVFRNYFNL | |
| N/A | G-A-T-N-N-V-F-X113-N-Y-F-N-L | |
| 4F2 | GGLYTGYSYFDL | |
| 62A10 | GGLWTGNSYFDL | |
| 70E2 | GGLWTGNSYFDL | |
| 71F10 | GGLWTGNSYFDL | |
| 73C2 | GGLWTGNSYFDL | |
| N/A | G-G-L-X114-T-G-X115-S-Y-F-D-L | |

As used herein, X99 is A or T, X100 is G, N, 5, or T, X1O1 is T or V, X102 is A, K, N, or Y, X103 is F, L, or Y, X104 is G, N, 5, or T, X105 is A, K, N, Y, X106 is F, L or Y, X107 is D or N, X1OS is S or T, X109 is A, T or V, X111O is N or 5, XI II is S or T, X112 is A, T or V, X113 is M or R, X114 is W or Y, and X115 is N or Y.

TABLE 12
Consensus Sequences of Heavy Chain CDR3 in Exemplary Antigen Binding Molecules
(IMGT Numbering Scheme)

| Clone | Sequences | SEQ ID NO |
|---|---|---|
| 9F7 | ARDTGSTDYNFNL | |
| 2H3 | ARDTGTTDYNFNL | |
| 69H10 | ARDAGTTDYNFNL | |
| 65D5 | ARDAGTTDYNFNL | |
| 3E9 | ARDAGSTDYKENL | |
| 9A5 | ARDAGGTDYNFNL | |
| 12G6 | ARDAGNTDYNFNL | |
| 1D10 | ARDAGSTDYNLNL | |
| 62E3 | ARDAGTTDYNFNL | |
| 3A4 | ARDAGTTDYNFNL | |
| 63H3 | ARDAGGTDYNYNL | |
| 66A1 | ARDAGGTDYAYNL | |
| 2E12 | ARDAGSTDYYFNL | |
| 72F12 | ARDAGSVDYNFNL | |
| 74F11/15F5 | ARDAGSTDYNFNL | |
| N/A | A-R-D-X116-G-X117-X118-D-Y-X119-X120-N-L (SEQ ID | |
| | NO: ) or A-R-D-X121-G-X122-T-D-Y-X123-X124-N-L | |
| | (SEQ ID NO: ) (excluding clone 72F12) | |
| 9D7 | ARDDGTASYYLNL | |
| 9D7-2 | ARDDGTASYYLNL | |
| 10A9 | ARDDGTTSYYLNL | |
| 10A9-2 | ARDDGTTSYYLNL | |
| 62A2 | AKNDGTTSYYLNL | |
| 74E4 | ARDDGTTSYYLNL | |
| 66A6 | ARDDGTTSYYLNL | |
| 10E12 | ARDDGTTSYYLNL | |
| 8H2 | ARDDGSTSYYLNL | |
| 74H11 | ARDDGSTSYYLNL | |
| 74A1 | ARNDGSTNYYLNL | |
| 10F12 | ARDDGSTSYYLNL | |
| 65E11 | ARDDGSTSYYLNL | |
| 69C2 | ARDDGSTSYYLNL | |
| 61F12 | ARDDGTTSYYLNL | |
| 10D3 | ARDDGSTSYYLNL | |
| 67H4 | ARDDGTTSYYLNL | |
| 64G9 | ARDDGSTSYYLNL | |
| 72G12 | ARDDGTTSYYLNL | |
| 65G8 | ARDDGTTSYYLNL | |
| 69D8 | ARDDGTTSYYLNL | |
| 2H5 | ARDDGTTSYYLNL | |
| 64A6 | ARDDGTASYYLNL | |
| 62B10 | ARDDGSVSYYLNL | |
| N/A | A-X125-X126-D-G-X127-X128-X129-Y-Y-L-N-L (SEQ ID | |
| | NO: ) or A-R-D-D-G-X130-X131-S-Y-Y-L-N-L (SEQ ID | |
| | NO: ) (excluding clones 62A2 and 74A1) | |
| 5D4 | ARGATNNVFMNYFNL | |
| 5D4-1 | ARGATNNVFMNYFNL | |
| 5D4-2 | ARGATNNVEMNYFNL | |
| 70G2 | ARGATNNVFRNYFNL | |
| N/A | A-R-G-A-T-N-N-V-F-X132-N-Y-F-N-L | |
| 4F2 | ARGGLYTGYSYFDL | |
| 62A10 | ARGGLWTGNSYFDL | |
| 70E2 | ARGGLWTGNSYFDL | |
| 71F10 | ARGGLWTGNSYFDL | |
| 73C2 | ARGGLWTGNSYFDL | |
| N/A | A-R-G-G-L-X133-T-G-X134-S-Y-F-D-L | |

As used herein, X16 is A or T, X117 is G, N, S, or T, X118 is T or V, X119 is A, K, N, or Y, X120 is F, L or Y, X121 is A or T, X122 is G, N, S, or T, X123 is A, K, N, or Y, X124 is F, L, or Y, X125 is K or R, X126 is D or N, X127 is S or T, X128 is A, T, or V, X129 is N or S, X130 is S or T, X131 is A, T, or V, X132 is M or R, X133 is W or Y, and X134 is N or Y.

Table 12 also shows several consensus sequences of heavy chain CDR3 of several exemplary antibodies combining Kabat and IMGT numbering scheme (same as IMGT).

TABLE 13
Consensus Sequences of Light Chain CDR1 in Exemplary Antigen Binding Molecules
(Kabat Numbering Scheme)

| Clone | Sequences | SEQ ID NO |
|---|---|---|
| 9F7 | QASEDIESYLA | |
| 2H3 | QASEDIESYLA | |
| 69H10 | QASQSIYSYLS | |
| 65D5 | QASQSISSYLA | |
| 3E9 | QASEDIESYLA | |
| 9A5 | QASQSIESYLA | |
| 12G6 | QASEDIESYLA | |
| 1D10 | QASEDIESYLA | |
| 62E3 | QASEDIESYLA | |
| 3A4 | QASEDIENYLA | |
| 63H3 | QASEDIESYSA | |
| 66A1 | QASEDIESYLA | |
| 2E12 | QASENIESYLA | |
| 72F12 | QASKNIDSNLA | |
| 74F11/15F5 | QASEDIERYLA | |
| N/A | Q-A-S-X135-X136-I-X137-X138-X139-X140-X141 (SEQ ID NO: ) or | |
| | Q-A-S-X142-X143-I-X144-X145-Y-L-X146 | |
| | (SEQ ID NO: ) (excluding clones 63H3 and 72F12) | |
| 9D7 | QASEDIYKLLA | |
| 9D7-2 | QASEDIYKLLA | |
| 10A9 | QASEDIYSLLA | |
| 10A9-2 | QASEDIYSLLA | |
| 62A2 | QASEDIYRLLA | |
| 74E4 | QASEDIYRLLA | |
| 66A6 | QASEDIYSLLA | |
| 10E12 | QASEDIYSLLA | |
| 8H2 | QASEDIYSLLA | |
| 74H11 | QASEDIYSLLA | |
| 74A1 | QASQSIYNNFA | |
| 10F12 | QASEDIYSLLA | |
| 65E11 | QASEDIYSLLA | |
| 69C2 | QASEDIYKLLA | |
| 61F12 | QASEDIYSLLA | |
| 10D3 | QASEDIYRLLA | |
| 67H4 | QASEDIYNLLA | |
| 64G9 | QASEDIYNLLA | |
| 72G12 | QASEDIYKLLA | |
| 65G8 | QASEDIYKLLA | |
| 69D8 | QASEDIYSLLA | |
| 2H5 | QASEDIYNLLA | |
| 64A6 | QASEDIYKLLA | |
| 62B10 | QASEDIYNLLA | |
| N/A | Q-A-S-X147-X148-I-Y-X149-X150-X151-A (SEQ ID NO: ) or Q-A-S-E- | |
| | D-I-Y-X152-L-L-A (SEQ ID NO: ) | |
| | (excluding clone 74A1) | |
| 5D4 | QASEDISSNLG | |
| 5D4-1 | QASEDISSNLG | |
| 5D4-2 | QASEDISSNLG | |
| 70G2 | QASEDISSNLA | |
| N/A | Q-A-S-E-D-I-S-S-N-L-X153 | |
| 4F2 | QASESLSSYLA | |
| 62A10 | QASESISRYLA | |
| 70E2 | QASENINRYLA | |
| 71F10 | QASESISRYLA | |
| 73C2 | QASESINRYLA | |
| N/A | Q-A-S-E-X154-X155-X156-X157-Y-L-A (SEQ ID NO: ) or Q-A-S-E- | |
| | X158-I-X159-R-Y-L-A (SEQ ID NO: ) | |
| | (excluding clone 4F2) | |

As used herein, X135 is E, K, or Q, X136 is D, N, or 5, X137 is D, E, 5, or Y, X138 is N, R, or 5, X139 is N or Y, X140 is L or 5, X141 is A or 5, X142 is E or Q, X143 is D, N, or 5, X144 is E, 5, or Y, X145 is N, R, or 5, X146 is A or 5, X147 is E or Q, X148 is D or 5, X149 is N, K, R, or 5, X150 O is L or N, X151 is F or L, X152 is N, K, R, or S, X153 is A or G, X154 is Nor S, X155 is I or L, X156 is N or 5, X157 is R or 5, X158 is N or S, and X159 is N or S.

TABLE 14
Consensus Sequences of Light Chain CDR1 in Exemplary Antigen Binding Molecules
(IMGT Numbering Scheme)

| Clone | Sequences | SEQ ID NO |
|---|---|---|
| 9F7 | EDIESY | |
| 2H3 | EDIESY | |
| 69H10 | QSIYSY | |
| 65D5 | QSISSY | |
| 3E9 | EDIESY | |
| 9A5 | QSIESY | |
| 12G6 | EDIESY | |
| 1D10 | EDIESY | |
| 62E3 | EDIESY | |
| 3A4 | EDIENY | |
| 63H3 | EDIESY | |
| 66A1 | EDIESY | |
| 2E12 | ENIESY | |
| 72F12 | KNIDSN | |
| 74F11/15F5 | EDIERY | |
| N/A | E-X160-I-E-X161-Y (SEQ ID NO: ) (clones starting | |
| | with an E) or Q-S-I-X162-S-Y (clones starting | |
| | with QS); excluding clone 72F12 | |
| 9D7 | EDIYKL | |
| 9D7-2 | EDIYKL | |
| 10A9 | EDIYSL | |
| 10A9-2 | EDIYSL | |
| 62A2 | EDIYRL | |
| 74E4 | EDIYRL | |
| 66A6 | EDIYSL | |
| 10E12 | EDIYSL | |
| 8H2 | EDIYSL | |
| 74H11 | EDIYSL | |
| 74A1 | QSIYNN | |
| 10F12 | EDIYSL | |
| 65E11 | EDIYSL | |
| 69C2 | EDIYKL | |
| 61F12 | EDIYSL | |
| 10D3 | EDIYRL | |
| 67H4 | EDIYNL | |
| 64G9 | EDIYNL | |
| 72G12 | EDIYKL | |
| 65G8 | EDIYKL | |
| 69D8 | EDIYSL | |
| 2H5 | EDIYNL | |
| 64A6 | EDIYKL | |
| 62B10 | EDIYNL | |
| N/A | E-D-I-Y-X163-L (SEQ ID NO: ) (clones starting | |
| | with an “E”) | |
| 4F2 | ESLSSY | |
| 62A10 | ESISRY | |
| 70E2 | ENINRY | |
| 71F10 | ESISRY | |
| 73C2 | ESINRY | |
| N/A | E-X164-X165-X166-X167-Y (SEQ ID NO: ) or E-X168- | |
| | I-X169-R-Y (SEQ ID NO: ) (excluding clone 4F2) | |

As used herein, X160 is D or N, X161 is N, R, or S, X162 is E, S, or Y, X163 is K, N, R, or S, X164 is N or S, X165 is I or L, X166 is N or S, X167 is R or S, X168 is N or S, and X169 is N or S.

Table 13 also shows several consensus sequences of light chain CDR1 of several exemplary antibodies combining Kabat and IMGT numbering scheme (same as Kabat).

TABLE 15
Consensus Sequences of Light Chain CDR2 in
Exemplary Antigen Binding Molecules
(Kabat Numbering Scheme)

| | | SEQ |
|---|---|---|
| | | ID |
| Clone | Sequences | NO |
| 9F7 | EASKLAS | |
| 2H3 | EASKLAS | |
| 69H10 | EASKLAS | |
| 65D5 | EATKLTS | |
| 3E9 | DASTLAS | |
| 9A5 | EASKLAS | |
| 12G6 | EASKLAS | |
| 1D10 | EASKLAS | |
| 62E3 | EASKLAS | |
| 3A4 | EASKLAS | |
| 63H3 | EASKLAS | |
| 66A1 | KASTLAS | |
| 2E12 | DASTLAS | |
| 72F12 | AASTLAS | |
| 74F11/15F5 | EASKLPS | |
| N/A | X170-A-X171- | |
| | X172-L-X173-S | |
| | (SEQ ID NO: ) | |
| | or | |
| | X174-A-S-X175- | |
| | L-X176-S | |
| | (SEQ ID NO: ) | |
| | (excluding | |
| | clone 65D5) | |
| | or X177-A-S- | |
| | X178-L-A-S | |
| | (SEQ ID NO: ) | |
| | excluding | |
| | clones 65D5, | |
| | 74F11/15F5) | |
| 9D7 | AASDLES | |
| 9D7-2 | AASDLES | |
| 10A9 | GASNLES | |
| 10A9-2 | GASNLES | |
| 62A2 | GASNLES | |
| 74E4 | GASNLES | |
| 66A6 | DASDLAS | |
| 10E12 | DASDLAS | |
| 8H2 | DASDLAS | |
| 74H11 | DASDLAS | |
| 74A1 | DASDLAS | |
| 10F12 | DASDLAS | |
| 65E11 | DASTLAS | |
| 69C2 | AASDLES | |
| 61F12 | DASDLAS | |
| 10D3 | DASDLAS | |
| 67H4 | AASDLES | |
| 64G9 | DASTLTS | |
| 72G12 | AASDLES | |
| 65G8 | AASDLES | |
| 69D8 | DASTLAS | |
| 2H5 | AASDLES | |
| 64A6 | AASDLES | |
| 62B10 | DASTLTS | |
| N/A | X179-A-S- | |
| | X180-L- | |
| | X181-S | |
| 4F2 | RAATLAS | |
| 62A10 | RASTLAS | |
| 70E2 | RAATLAS | |
| 71F10 | RASTLAS | |
| 73C2 | RAATLAS | |
| N/A | R-A-X182- | |
| | T-L-A-S | |

As used herein, X170 is A, D, E, or K, X171 is S or T, X172 is K or T, X173 is A, P, or T, X174 is A, D, E, or K, X175 is K or T, X176 is A, or P, X177 is A, D, E, or K, X178 is K or T, X179 is A, D, or G, X180 is D, N, or T, X181 is A, E, or T, and X182 is A or S.

Consensus sequences of light chain CDR2 using IMGT numbering scheme of the exemplary antibodies of the present disclosure were not generated as the light chain CDR2 using IMGT numbering scheme only contains three (3) amino acids.

Table 15 also shows several consensus sequences of light chain CDR2 of several exemplary antibodies combining Kabat and IMGT numbering scheme (same as Kabat).

TABLE 16
Consensus Sequences of Light Chain CDR3
in Exemplary Antigen Binding Molecules
(Kabat Numbering Scheme)

| | | SEQ |
|---|---|---|
| Clone | Sequences | ID NO |
| 9F7 | QQALTVGNVDNP | |
| 2H3 | QQALTVGNVDNP | |
| 69H10 | QQALTVGNVDNP | |
| 65D5 | QQALTVGDVDNP | |
| 3E9 | QQDLTVGNVDNP | |
| 9A5 | QQALTIGNVDNP | |
| 12G6 | QQALTIGNVDNP | |
| 1D10 | HQALTIGNVDNP | |
| 62E3 | QQALTIGNVDNP | |
| 3A4 | QQALTIGNVDNP | |
| 63H3 | QQALTIGNVDNP | |
| 66A1 | QQVLTIGNVDNP | |
| 2E12 | QQGLTIGNVDNP | |
| 72F12 | QQALTIGNVDNP | |
| 74F11/15F5 | QQALTIGYVDNP | |
| N/A | X183-Q-X184- | |
| | L-T-X185-G- | |
| | X186-V-D-N- | |
| | P (SEQ ID NO:) | |
| | or Q-Q-X187- | |
| | L-T-X188-G- | |
| | X189-V-D-N- | |
| | P (SEQ ID | |
| | NO: ) | |
| | (excluding | |
| | clones 1D10) | |
| 9D7 | QQAYTIGNIDNS | |
| 9D7-2 | QQAYTIGNIDNS | |
| 10A9 | QQAYTIGNIDNA | |
| 10A9-2 | QQAYTIGNIDNA | |
| 62A2 | QQAYTIGNIDNA | |
| 74E4 | QQAYTIGNIDNA | |
| 66A6 | QQAYTIGNIDNT | |
| 10E12 | QQAYTIGNIDNA | |
| 8H2 | QQAYTIGNIDNT | |
| 74H11 | QQAYTIGNIDNA | |
| 74A1 | QQAYTIGNIDNP | |
| 10F12 | QQAYTIGNIDNA | |
| 65E11 | QQAYTIGNIDNA | |
| 69C2 | QQAYTIGNIDNT | |
| 61F12 | QQAYTIGNIDNS | |
| 10D3 | QQGYTIGNIDNS | |
| 67H4 | QQGYTIGNIDNA | |
| 64G9 | QQAYTIGNIDNT | |
| 72G12 | QQAYTIGNLDNA | |
| 65G8 | QQAYTIGNIDNT | |
| 69D8 | QQAYTIGNIDNA | |
| 2H5 | QQAYTVGNIDNA | |
| 64A6 | QQAYTIGNIDNS | |
| 62B10 | QQGYTIGNIDNT | |
| N/A | Q-Q-X190-Y-T- | |
| | X191-G-N-I- | |
| | D-N-X192 | |
| 5D4 | QTSYYIDDGVNG | |
| 5D4-1 | QTSYYIDDGVNG | |
| 5D4-2 | QTSYYIDDGVNG | |
| 70G2 | QSSYYIDDGVNG | |
| N/A | Q-X193-S-Y-Y- | |
| | I-D-D-G-V-N- | |
| | G | |
| 4F2 | QQGYGYSTVGNA | |
| 62A10 | QQGYGYSTVDNA | |
| 70E2 | QQGYGYSTVGNA | |
| 71F10 | QQGYGYSTVGNA | |
| 73C2 | QQGYGYSTVGNA | |
| N/A | Q-Q-G-Y-G-Y- | |
| | S-T-V-X194- | |
| | N-A | |

As used herein, X83 is H or Q, X184 is A, D, G, or V, X185 is I or V, X186 is D, N, or Y, X187 is A, D, G, or V, X188 is I or V, X189 is D, N, or Y, X190 is A, or G, X191 is I or V, X192 is A, P, S or T, X193 is S or T, and X194 is D or G.

Table 16 also shows several consensus sequences of light chain CDR3 of several exemplary antibodies using IMGT numbering scheme or combining Kabat and IMGT numbering scheme (same as Kabat).

In some embodiments, the antibody, or the antigen binding fragment thereof, comprises: (1) a heavy chain variable region (HCVR) having an amino acid sequence that is about 80%, 85%, 90%, 95%, 96%, 97%, 98%, 99% to about 100% identical to an amino acid sequence selected from the HCVR sequence listed in Tables 17 and 18; and (2) a light chain variable region (LCVR) having an amino acid sequence that is about 80%, 85%, 90%, 95%, 96%, 97%, 98%, 99% to about 100% identical to an amino acid sequence selected from the LCVR sequence listed in Table 19, wherein the antibody, or the antigen binding fragment thereof, binds specifically to human Trop-2. Exemplary HCVR- and LCVR amino acid sequences corresponding to the exemplary anti-human Trop-2 monoclonal antibodies disclosed in the present invention are shown in Tables 17-19.

TABLE 17
Amino Acid Sequences of HCVRs of Exemplary Antigen Binding Molecules

| | | SEQ |
|---|---|---|
| | | ID |
| Clone | Sequences | NO: |
| 9F7 | QEQLKESGGGLVQPGGSLTLSCKASGFDENNYGVSWVRQAPGKGLEWIGYIYPAYHNTYYATWVNGRFTISSHNAQNT | 113 |
| | LYLQLNTLTAADTATYFCARDTGSTDYNFNLWGPGTLVTVSS | |
| 2H3 | QEQLKESGGGLGQPGGSLTLSCKASGFDFNNYGVSWVRQAPGKGLEWIGYIYPAYHNTYYATWVNGRFTISSHNAQNT | 114 |
| | LYLQLNTLTAADTATYFCARDTGTTDYNFNLWGPGTLVTVSS | |
| 69H10 | QEQLKESGGGLVQPGGSLKLSCKASGFDFNKYGVSWVRQAPGKGLEWIAYIYPAFHNTYYATWVNGRFTISSHNAQNT | 115 |
| | LYLQLNTLTAADTATYFCARDAGTTDYNFNLWGPGTLVTVSS | |
| 65D5 | QEQLKESGGGLVQPGGSLKLSCKASGFDFNAYGVSWVRQAPGKGLEWIAYIYPAFHNTYYATWVNGRFTISSHNAQNT | 116 |
| | LYLQLNTLTAADTATYFCARDAGTTDYNENLWGPGTLVTVSS | |
| 3E9 | QEQLKESGGGLVQPGGSLKLSCKASGFDENNYGVSWVRQAPGKGLEWIAYIYPAFHNRYYANWVNGRFTISSDNAQNT | 117 |
| | VYLQLNSLTAADTATYFCARDAGSTDYKFNLWGPGTLVTVSS | |
| 9A5 | QEQLKESGGGLVQPGGSLTLSCKASGFDENNYGVSWVRQAPGKGLEWIAYIYPTYHNTYYATWVNGRFTISSHNAQNT | 118 |
| | LYLQLNTLTAADTATYFCARDAGGTDYNFNLWGPGTLVTVSS | |
| 12G6 | QEQLKESGGGLVQPGGSLTLSCKASGFDENNYGVSWVRQAPGKGLEWIGYIYPAYHNTYYATWVNGRFTISSHNAQNT | 119 |
| | LYLQLNTLTAADTATYFCARDAGNTDYNFNLWGPGTLVTVSS | |
| 1D10 | QEQLKESGGGLVQPGGSLTLSCKASGFDFNYYGVSWVRQAPGKGLEWIGYIYPAYHNTYYATWVNGRFTISSHNAQNT | 120 |
| | LYLQLNTLTAADTATYFCARDAGSTDYNLNLWGPGTLVTVSS | |
| 62E3 | QEQLKESGGGLVQPGGSLKLSCKASGFDENEYGVSWVRQAPGKGLEWIAYIYPAFHNTYYATWVNGRFTISSHNAQNT | 121 |
| | LYLQLNTLTAADTATYFCARDAGTTDYNFNLWGPGTLVTVSS | |
| 3A4 | QEQLVESGGGLVQPGGSLTLSCKGSGFDFNEYGVSWVRQAPGKGLEWIAYIYPAFHNTYYATWVNGRFTISSHNAQNT | 122 |
| | LYLQLHTLTAADTATYFCARDAGTTDYNENLWGPGTLVTVSS | |
| 63H3 | QEQLKESGGGLVQPGGSLKLSCKASGFDFNSYGVSWVRQAPGKGLEWIAYIYPAFHNAYYANWVNGRFTISSDNAQNT | 123 |
| | VDLQLNSLTAADTATYFCARDAGGTDYNYNLWGPGTLVTVSS | |
| 66A1 | QEQLKESGGGLVQPGGSLKLSCKASGFDENNYGVSWVRQAPGKGLEWIAYIYPAFHNTYYASWVNGRFTISSHNAQNT | 124 |
| | LYLQLNSLTAADTATYFCARDAGGTDYAYNLWGPGTLVTVSS | |
| 2E12 | QEQLKESGGGLVRLGDPLQLSCKASGFDENNYGVTWVRQAPGKGLEWIAYIYPAFPNTYYATWVNGRFTISPHNAQNT | 125 |
| | LYLQLNSLTAADTATYFCARDAGSTDYYFNLWGPGTLATVSS | |
| 72F12 | QEQLKESGGGLVQPGGSLKLSCKASGFDENNYGVSWVRQAPGKGLEWIAYIYPAFHNTYYATWVNGRFTISSHNAQNT | 126 |
| | LYLQLNSLTAADTATYFCARDAGSVDYNENLWGPGTLVTVSS | |
| 74F11 | QEQLKESGGGLVQPGGSLKLSCKASGFDFNNYGMSWVRQAPGKGLEWIAYIYPAFHNTYYANWVNGRFTISSDNAQNT | 127 |
| | VDLQLNSLTAADTATYFCARDAGSTDYNFNLWGPGTLVTVSS | |
| 15F5 | QEQLKESGGGLVQPGGSLKLSCKASGFDENNYGMSWVRQAPGKGLEWIAYIYPAFHNTYYANWVNGRFTISSDNAQNT | |
| | VDLQLNSLTAADTATYFCARDAGSTDYNFNLWGPGTLVTVSS | |
| 9D7 | QSLEESGGDLVQPGASLTLTCKASGFDLNNYSILWVRQAPGKGLEWIGCIYISGGSTYYANWAKGRFAISKTSSTTVT | 129 |
| | LQMTSLTAADTATYFCARDDGTASYYLNLWGPGTLVTVSS | |
| 9D7-2 | QSLEESGGDLVQPGASLTLTCKASGFDLNNYSILWVRQAPGKGLEWIGSIYISGGSTYYANWAKGRFAISKTSSTTVT | 130 |
| | LQMTSLTAADTATYFCARDDGTASYYLNLWGPGTLVTVSS | |
| 10A9 | QSLEESGGDLVQPGASLTLTCKASGFDLNNYAMLWVRQAPGKGLEWIGCIYISGGTTYFASWATGRFAISKTSSTTVT | 131 |
| | LQMTSLTAADTATYFCARDDGTTSYYLNLWGPGTLVTVSS | |
| 10A9-2 | QSLEESGGDLVQPGASLTLTCKASGFDLNNYAMLWVRQAPGKGLEWIGSIYISGGTTYFASWATGRFAISKTSSTTVT | 132 |
| | LQMTSLTAADTATYFCARDDGTTSYYLNLWGPGTLVTVSS | |
| 62A2 | QSLEESGGDLVQPGASLTLTCKASGFDLNNYAMLWVRQAPGKGLEWIGCIYISGGTTYFASWATGRFAISKTSSTTLT | 133 |
| | LQMTTLPPPDTATYFCAKNDGTTSYYLNLWGPGTLVTVSS | |
| 74E4 | QSLEESGGDLVQPGASLTLTCKASGFDLNNYAILWVRQAPGKGLEWIGCIYISGATTYFANWATGRFAISKTSSTIVT | 134 |
| | LQMTSLTAADTATYFCARDDGTTSYYLNLWGPGTLVTVSS | |
| 66A6 | QSLEESGGDLVQPGASLTLTCKASGFDLNNYAMLWVRQAPGKGLEWIGCIYISGGTTYFASWATGRFAISKTSSTTVT | 135 |
| | LQMTSLTAADTATYFCARDDGTTSYYLNLWGPGTLVTVSS | |
| 10E12 | QSLEESGGDLVQPGASLTLTCKASGFDLNNYAMLWVRQAPGKGLEWIGCIYISGGTTYFASWATGRFAISKTSSTTVT | 136 |
| | LQMTSLTAADTATYFCARDDGTTSYYLNLWGPGTLVTVSS | |
| 8H2 | QSLEESGGDLVQPGASLTLTCKASGFDLNNYAMLWVRQAPGKGLEWIGCIYISGGTTYFASWATGRFAISKTSSTTVT | 137 |
| | LQMTSLTAADTATYFCARDDGSTSYYLNLWGPGTLVTVSS | |
| 74H11 | QSLEESGGDLVQPGASLTLTCKASGFDLNNYAMLWVRQAPGKGLEWIGCIYISGGTTYFASWATGRFAISRTSSTTVT | 138 |
| | LQMTSLTAADTATYFCARDDGSTSYYLNLWGPGTLVTVSS | |
| 74A1 | QSLEESGGNLAHPGASLRLTCTASGFYLNNYAMLWVRQAPGKGLEWIGCLYISGGTTYFASWATGRFAISKTSSTIGT | 139 |
| | LQMTSLTAADTATYFCARNDGSTNYYLNLWGPGTLVTISS | |
| 10F12 | QSLEESGGDLVQPGASLTLTCRASGFDLNNYSMLWVRQAPGKGLEWIGCIYISGGTTYFASWATGRFAISKTSSTTVT | 140 |
| | LQMTSLTAADTATYFCARDDGSTSYYLNLWGPGTLVTVSS | |
| 65E11 | QSLEESGGDLVQPGASLTLTCRASGFDLNNYSMLWVRQAPGKGLEWIGCIYISGGTTYFASWATGRFAISKTSSTTVT | 141 |
| | LQMTILTAADTATYFCARDDGSTSYYLNLWGPGTLVTVSS | |
| 69C2 | QSLEESGGDLVQPGASLTLTCRASGFDLNNYSMLWVRQAPGKGLEWIGCIYISGGTTYFASWATGRFAISKTSSTTVT | 142 |
| | LQMTILTAADTATYFCARDDGSTSYYLNLWGPGTLVTVSS | |
| 61F12 | QSLEESGGDLVQPGASLTLTCKASGFDLNNYSILWVRQAPGKGLEWIGCIYISGGTTYFASWATGRFAISKTSSTTVT | 143 |
| | LQMTSLTAADTATYFCARDDGTTSYYLNLWGPGTLVTVSS | |
| 10D3 | QSLEESGGDLVQPGASLTLTCKAAGFDLNNYSMLWVRQAPGKGLEWIGCIYISGGTTYYASWAKGRFTISKTSSTTVT | 144 |
| | LQMTSLTAADTATYFCARDDGSTSYYLNLWGPGTLVTVSS | |
| 67H4 | QSLEESGGDLVQPGVSLTLTCKASGFDLNNYSILWVRQAPGKGLEWIGCIYISGGSTYYASWAKGRFAISKTSSTTVT | 145 |
| | LQMTSLTAADTATYFCARDDGTTSYYLNLWGPGTLVTVSS | |
| 64G9 | QSLEESGGDLVQPGASLTLTCKASGFDLNNYSILWVRQAPGKGLEWIGCIYISGGTTYYANWAKGRFTISRTSSTTVT | 146 |
| | LQMTSLTGADTATYFCARDDGSTSYYLNLWGPGTLVTVSS | |
| 72G12 | QSLEESGGDLVQPGASLTLTCKASGFDLNNYSILWVRQAPGKGLEWIGCIYISGGTTYYANWAKGRFAISKTSSTTVT | 147 |
| | LQMTSLTAADTATYFCARDDGTTSYYLNLWGPGTLVTVSS | |
| 65G8 | QSLEESGGDLVQPGASLTLTCKASGFDLNNYSILWVRQAPGKGLEWIGCIYISGGSTYYANWAKGRFAISKTSSTTVT | 148 |
| | LQMTSLTAADTATYFCARDDGTTSYYLNLWGPGTLVTVSS | |
| 69D8 | QSLEESGGDLVQPGASLTLICKASGFDLNNYSILWVRQAPGKGLEWIGCIYISGGSTYYANWAKGRFAVSKTSSTTVT | 149 |
| | LQMTSLTAADTATYFCARDDGTTSYYLNLWGPGTLVTVSS | |
| 2H5 | QSLEESGGDLVQPGASLTLTCKASGFDLNNYSILWVRQAPGKGLEWIGCIYFSGGSTYYANWAKGRFAISKTSSTTVT | 150 |
| | LQMTSLTAADTATYFCARDDGTTSYYLNLWGPGTLVTVSS | |
| 64A6 | QSLEESGGDLVQPGASLTLTCKASGFDLNNYSILWVRQAPGKGLEWIGCIYISGGSTYYANWAKGRFAISKTSSTTVT | 151 |
| | LQMTSLTAADTATYFCARDDGTASYYLNLWGPGTLVTVSS | |
| 62B10 | QSLEESGGDLVQPGTSLILSCKASGFDLNNYSILWVRQAPGKGLEWIGCIYISGGTTYFANWAKGRFTISRPSSTTVT | 152 |
| | LQMTSLTGADTATYFCARDDGSVSYYLNLWGPGTLVIVSS | |
| 5D4 | QQLEESGGGLVKPEGSLTLTCKGSGFDFSGTYWMWWVRQAPGKGLEWIACIYVGSGSSTYYASWAKGRFTISSTSSTA | 153 |
| | VTLQATSLTAADTATYFCARGATNNVFMNYFNLWGPGTLVTVSS | |
| 5D4-1 | QQLEESGGGLVKPEGSLTLTCKGSGFDFSGTYWMWWVRQAPGKGLEWIAPIYVGSGSSTYYASWAKGRFTISSTSSTA | 154 |
| | VTLQATSLTAADTATYFCARGATNNVFMNYFNLWGPGTLVTVSS | |
| 5D4-2 | QQLEESGGGLVKPEGSLTLTCKGSGFDFSGTYWMWWVRQAPGKGLEWIASIYVGSGSSTYYASWAKGRFTISSTSSTA | 155 |
| | VTLQATSLTAADTATYFCARGATNNVFMNYFNLWGPGTLVTVSS | |
| 70G2 | QQLEESGGGLVKPEGSLTLTCKGSGFDFSGTYWMCWVRQAPGKGLEWIACIYVGSGGSTYYASWAKGRFTISSTSSTA | 156 |
| | VTLQVTSLTAADTATYFCARGAINNVERNYFNLWGPGTLVTVSS | |
| 4F2 | QSLEESGGGLVKPGGTLTLTCTVSGFDFSNYAMTWVRQAPGEGLEYIGFVALRGNIYYANWAKGRFTISKTSSTTVTL | 157 |
| | QMTSLTVADTATYFCARGGLYTGYSYFDLGGPGTLATVSS | |
| 62A10 | QSLVESGGGLVQPEGSLTLTCKASGFDENNYAVTWVRQAPGEGLEYIGFIGIRGHIYYANWAKGRFTISKTSSTTMTL | 158 |
| | QMTSLTVADTATYFCARGGLWTGNSYFDLWGPGTLVTVSS | |
| 70E2 | QSLEESGGGLVKPGGTLTLTCTVSGFDFSNYAMTWVRQAPGKGLEYIGFIGIRGNIYYANWAKGRFTISKTSSTTVTL | 159 |
| | QMTSLTVADTATYFCARGGLWTGNSYFDLWGPGTLVTVSS | |
| 71F10 | QSLEESGGGLVQPGASLTLTCTVSGFDFSSNAMTWVRQAPGEGLEYIGFIGIRGNIYYANWAKGRFTISKTSSTTVTL | 160 |
| | QMTSLTVADTATYFCARGGLWTGNSYFDLWGPGTLVTVSS | |
| 73C2 | QSLEESGGGLVKPGGTLTLTCTVSGFDFSNYAVTWVRQAPGEGLEYIGFVGIYGDFYYANWAKGRFTISKTSSTTVTL | 161 |
| | QMPSLTVADTATYFCARGGLWTGNSYFDLWGPGTLVTVSS | |

TABLE 18
Amino Acid Sequences of Pyroglutamylated HCVRs
of Exemplary Antigen Binding Molecules

| | | SEQ |
|---|---|---|
| | | ID |
| Clone | Sequence | NO: |
| 9F7 | PEEQLKESGGGLVQPGGSLTLSCKASGFDENNYGVSWVRQAPGKGLEWIGYIYPAYHNTYYATWVNGRFTISSHNAQNT | |
| | LYLQLNTLTAADTATYFCARDTGSTDYNFNLWGPGTLVTVSS | |
| 2H3 | PEEQLKESGGGLGQPGGSLTLSCKASGFDENNYGVSWVRQAPGKGLEWIGYIYPAYHNTYYATWVNGRFTISSHNAQNT | |
| | LYLQLNTLTAADTATYFCARDTGTTDYNFNLWGPGTLVTVSS | |
| 69H10 | pEEQLKESGGGLVQPGGSLKLSCKASGFDFNKYGVSWVRQAPGKGLEWIAYIYPAFHNTYYATWVNGRFTISSHNAQNT | |
| | LYLQLNTLTAADTATYFCARDAGTTDYNFNLWGPGTLVTVSS | |
| 65D5 | pEEQLKESGGGLVQPGGSLKLSCKASGFDFNAYGVSWVRQAPGKGLEWIAYIYPAFHNTYYATWVNGRFTISSHNAQNT | |
| | LYLQLNTLTAADTATYFCARDAGTTDYNFNLWGPGTLVTVSS | |
| 3E9 | pEEQLKESGGGLVQPGGSLKLSCKASGFDENNYGVSWVRQAPGKGLEWIAYIYPAFHNRYYANWVNGRFTISSDNAQNT | |
| | VYLQLNSLTAADTATYFCARDAGSTDYKFNLWGPGTLVTVSS | |
| 9A5 | PEEQLKESGGGLVQPGGSLTLSCKASGFDENNYGVSWVRQAPGKGLEWIAYIYPTYHNTYYATWVNGRFTISSHNAQNT | |
| | LYLQLNTLTAADTATYFCARDAGGTDYNFNLWGPGTLVTVSS | |
| 12G6 | PEEQLKESGGGLVQPGGSLTLSCKASGFDFNNYGVSWVRQAPGKGLEWIGYIYPAYHNTYYATWVNGRFTISSHNAQNT | |
| | LYLQLNTLTAADTATYFCARDAGNTDYNFNLWGPGTLVTVSS | |
| 1D10 | PEEQLKESGGGLVQPGGSLTLSCKASGFDFNYYGVSWVRQAPGKGLEWIGYIYPAYHNTYYATWVNGRFTISSHNAQNT | |
| | LYLQLNTLTAADTATYFCARDAGSTDYNLNLWGPGTLVTVSS | |
| 62E3 | PEEQLKESGGGLVQPGGSLKLSCKASGFDFNEYGVSWVRQAPGKGLEWIAYIYPAFHNTYYATWVNGRFTISSHNAQNT | |
| | LYLQLNTLTAADTATYFCARDAGTTDYNFNLWGPGTLVTVSS | |
| 3A4 | pEEQLVESGGGLVQPGGSLTLSCKGSGFDFNEYGVSWVRQAPGKGLEWIAYIYPAFHNTYYATWVNGRFTISSHNAQNT | |
| | LYLQLHTLTAADTATYFCARDAGTTDYNFNLWGPGTLVTVSS | |
| 63H3 | pEEQLKESGGGLVQPGGSLKLSCKASGFDFNSYGVSWVRQAPGKGLEWIAYIYPAFHNAYYANWVNGRFTISSDNAQNT | |
| | VDLQLNSLTAADTATYFCARDAGGTDYNYNLWGPGTLVTVSS | |
| 66A1 | PEEQLKESGGGLVQPGGSLKLSCKASGFDENNYGVSWVRQAPGKGLEWIAYIYPAFHNTYYASWVNGRFTISSHNAQNT | |
| | LYLQLNSLTAADTATYFCARDAGGTDYAYNLWGPGTLVTVSS | |
| 2E12 | pEEQLKESGGGLVRLGDPLQLSCKASGFDENNYGVTWVRQAPGKGLEWIAYIYPAFPNTYYATWVNGRFTISPHNAQNT | |
| | LYLQLNSLTAADTATYFCARDAGSTDYYFNLWGPGTLATVSS | |
| 72F12 | pEEQLKESGGGLVQPGGSLKLSCKASGFDENNYGVSWVRQAPGKGLEWIAYIYPAFHNTYYATWVNGRFTISSHNAQNT | |
| | LYLQLNSLTAADTATYFCARDAGSVDYNFNLWGPGTLVTVSS | |
| 74F11 | PEEQLKESGGGLVQPGGSLKLSCKASGFDENNYGMSWVRQAPGKGLEWIAYIYPAFHNTYYANWVNGRFTISSDNAQNT | |
| | VDLQLNSLTAADTATYFCARDAGSTDYNFNLWGPGTLVTVSS | |
| 15F5 | PEEQLKESGGGLVQPGGSLKLSCKASGFDENNYGMSWVRQAPGKGLEWIAYIYPAFHNTYYANWVNGRFTISSDNAQNT | |
| | VDLQLNSLTAADTATYFCARDAGSTDYNFNLWGPGTLVTVSS | |
| 9D7 | pESLEESGGDLVQPGASLTLTCKASGFDLNNYSILWVRQAPGKGLEWIGCIYISGGSTYYANWAKGRFAISKTSSTTVT | |
| | LQMTSLTAADTATYFCARDDGTASYYLNLWGPGTLVTVSS | |
| 9D7-2 | pESLEESGGDLVQPGASLTLTCKASGFDLNNYSILWVRQAPGKGLEWIGSIYISGGSTYYANWAKGRFAISKTSSTTVT | |
| | LQMTSLTAADTATYFCARDDGTASYYLNLWGPGTLVTVSS | |
| 10A9 | pESLEESGGDLVQPGASLTLTCKASGFDLNNYAMLWVRQAPGKGLEWIGCIYISGGTTYFASWATGRFAISKTSSTTVT | |
| | LQMTSLTAADTATYFCARDDGTTSYYLNLWGPGTLVTVSS | |
| 10A9-2 | pESLEESGGDLVQPGASLTLTCKASGFDLNNYAMLWVRQAPGKGLEWIGSIYISGGTTYFASWATGRFAISKTSSTTVT | |
| | LQMTSLTAADTATYFCARDDGTTSYYLNLWGPGTLVTVSS | |
| 62A2 | pESLEESGGDLVQPGASLTLTCKASGFDLNNYAMLWVRQAPGKGLEWIGCIYISGGTTYFASWATGRFAISKTSSTTLT | |
| | LQMTTLPPPDTATYFCAKNDGTTSYYLNLWGPGTLVTVSS | |
| 74E4 | pESLEESGGDLVQPGASLTLTCKASGFDLNNYAILWVRQAPGKGLEWIGCIYISGATTYFANWATGRFAISKTSSTTVT | |
| | LQMTSLTAADTATYFCARDDGTTSYYLNLWGPGTLVTVSS | |
| 66A6 | pESLEESGGDLVQPGASLTLTCKASGFDLNNYAMLWVRQAPGKGLEWIGCIYISGGTTYFASWATGRFAISKTSSTIVT | |
| | LQMTSLTAADTATYFCARDDGTTSYYLNLWGPGTLVTVSS | |
| 10E12 | pESLEESGGDLVQPGASLTLTCKASGFDLNNYAMLWVRQAPGKGLEWIGCIYISGGTTYFASWATGRFAISKTSSTTVT | |
| | LQMTSLTAADTATYFCARDDGTTSYYLNLWGPGTLVTVSS | |
| 8H2 | pESLEESGGDLVQPGASLTLTCKASGFDLNNYAMLWVRQAPGKGLEWIGCIYISGGTTYFASWATGRFAISKTSSTTVT | |
| | LQMTSLTAADTATYFCARDDGSTSYYLNLWGPGTLVTVSS | |
| 74H11 | pESLEESGGDLVQPGASLTLTCKASGFDLNNYAMLWVRQAPGKGLEWIGCIYISGGTTYFASWATGRFAISRTSSTTVT | |
| | LQMTSLTAADTATYFCARDDGSTSYYLNLWGPGTLVTVSS | |
| 74A1 | PESLEESGGNLAHPGASLRLTCTASGFYLNNYAMLWVRQAPGKGLEWIGCLYISGGTTYFASWATGRFAISKTSSTTGT | |
| | LQMTSLTAADTATYFCARNDGSTNYYLNLWGPGTLVTISS | |
| 10F12 | pESLEESGGDLVQPGASLTLTCRASGFDLNNYSMLWVRQAPGKGLEWIGCIYISGGTTYFASWATGRFAISKTSSTTVT | |
| | LQMTSLTAADTATYFCARDDGSTSYYLNLWGPGTLVTVSS | |
| 65E11 | pESLEESGGDLVQPGASLTLTCRASGFDLNNYSMLWVRQAPGKGLEWIGCIYISGGTTYFASWATGRFAISKTSSTTVT | |
| | LQMTILTAADTATYFCARDDGSTSYYLNLWGPGTLVTVSS | |
| 69C2 | pESLEESGGDLVQPGASLTLTCRASGFDLNNYSMLWVRQAPGKGLEWIGCIYISGGTTYFASWATGRFAISKTSSTTVT | |
| | LQMTILTAADTATYFCARDDGSTSYYLNLWGPGTLVTVSS | |
| 61F12 | pESLEESGGDLVQPGASLTLTCKASGFDLNNYSILWVRQAPGKGLEWIGCIYISGGTTYFASWATGRFAISKTSSTTVT | |
| | LQMTSLTAADTATYFCARDDGTTSYYLNLWGPGTLVTVSS | |
| 10D3 | pESLEESGGDLVQPGASLTLTCKAAGFDLNNYSMLWVRQAPGKGLEWIGCIYISGGTTYYASWAKGRFTISKTSSTTVT | |
| | LQMTSLTAADTATYFCARDDGSTSYYLNLWGPGTLVTVSS | |
| 67H4 | pESLEESGGDLVQPGVSLTLTCKASGFDLNNYSILWVRQAPGKGLEWIGCIYISGGSTYYASWAKGRFAISKTSSTTVT | |
| | LQMTSLTAADTATYFCARDDGTTSYYLNLWGPGTLVTVSS | |
| 64G9 | pESLEESGGDLVQPGASLTLTCKASGFDLNNYSILWVRQAPGKGLEWIGCIYISGGTTYYANWAKGRFTISRTSSTTVT | |
| | LQMTSLTGADTATYFCARDDGSTSYYLNLWGPGTLVTVSS | |
| 72G12 | pESLEESGGDLVQPGASLTLTCKASGFDLNNYSILWVRQAPGKGLEWIGCIYISGGTTYYANWAKGRFAISKTSSTTVT | |
| | LQMTSLTAADTATYFCARDDGTTSYYLNLWGPGTLVTVSS | |
| 65G8 | pESLEESGGDLVQPGASLTLTCKASGFDLNNYSILWVRQAPGKGLEWIGCIYISGGSTYYANWAKGRFAISKTSSTTVT | |
| | LQMTSLTAADTATYFCARDDGTTSYYLNLWGPGTLVTVSS | |
| 69D8 | pESLEESGGDLVQPGASLTLTCKASGFDLNNYSILWVRQAPGKGLEWIGCIYISGGSTYYANWAKGRFAVSKTSSTTVT | |
| | LQMTSLTAADTATYFCARDDGTTSYYLNLWGPGTLVTVSS | |
| 2H5 | pESLEESGGDLVQPGASLTLTCKASGFDLNNYSILWVRQAPGKGLEWIGCIYFSGGSTYYANWAKGRFAISKTSSTTVT | |
| | LQMTSLTAADTATYFCARDDGTTSYYLNLWGPGTLVTVSS | |
| 64A6 | pESLEESGGDLVQPGASLTLTCKASGFDLNNYSILWVRQAPGKGLEWIGCIYISGGSTYYANWAKGRFAISKTSSTTVT | |
| | LQMTSLTAADTATYFCARDDGTASYYLNLWGPGTLVTVSS | |
| 62B10 | pESLEESGGDLVQPGTSLTLSCKASGFDLNNYSILWVRQAPGKGLEWIGCIYISGGTTYFANWAKGRFTISRPSSTTVT | |
| | LQMTSLTGADTATYFCARDDGSVSYYLNLWGPGTLVIVSS | |
| 5D4 | pEQLEESGGGLVKPEGSLTLTCKGSGFDFSGTYWMWWVRQAPGKGLEWIACIYVGSGSSTYYASWAKGRFTISSTSSTA | |
| | VTLQATSLTAADTATYFCARGAINNVEMNYFNLWGPGTLVTVSS | |
| 5D4-1 | pEQLEESGGGLVKPEGSLTLTCKGSGFDFSGTYWMWWVRQAPGKGLEWIAPIYVGSGSSTYYASWAKGRFTISSTSSTA | |
| | VTLQATSLTAADTATYFCARGATNNVFMNYFNLWGPGTLVTVSS | |
| 5D4-2 | pEQLEESGGGLVKPEGSLTLTCKGSGFDFSGTYWMWWVRQAPGKGLEWIASIYVGSGSSTYYASWAKGRFTISSTSSTA | |
| | VTLQATSLTAADTATYFCARGAINNVEMNYFNLWGPGTLVTVSS | |
| 70G2 | pEQLEESGGGLVKPEGSLTLTCKGSGFDFSGTYWMCWVRQAPGKGLEWIACIYVGSGGSTYYASWAKGRFTISSTSSTA | |
| | VTLQVTSLTAADTATYFCARGATNNVFRNYFNLWGPGTLVTVSS | |
| 4F2 | pESLEESGGGLVKPGGTLTLTCTVSGFDFSNYAMTWVRQAPGEGLEYIGFVALRGNIYYANWAKGRFTISKTSSTTVTL | |
| | QMTSLTVADTATYFCARGGLYTGYSYFDLGGPGTLATVSS | |
| 62A10 | pESLVESGGGLVQPEGSLTLTCKASGFDENNYAVTWVRQAPGEGLEYIGFIGIRGHIYYANWAKGRFTISKTSSTTMTL | |
| | QMTSLTVADTATYFCARGGLWTGNSYFDLWGPGTLVTVSS | |
| 70E2 | pESLEESGGGLVKPGGTLTLTCTVSGFDFSNYAMTWVRQAPGKGLEYIGFIGIRGNIYYANWAKGRFTISKTSSTTVIL | |
| | QMTSLTVADTATYFCARGGLWTGNSYFDLWGPGTLVTVSS | |
| 71F10 | pESLEESGGGLVQPGASLTLTCTVSGFDFSSNAMTWVRQAPGEGLEYIGFIGIRGNIYYANWAKGRFTISKTSSTTVIL | |
| | QMTSLTVADTATYFCARGGLWTGNSYFDLWGPGTLVTVSS | |
| 73C2 | pESLEESGGGLVKPGGTLTLTCTVSGFDFSNYAVTWVRQAPGEGLEYIGFVGIYGDFYYANWAKGRFTISKTSSTTVTL | |
| | QMPSLTVADTATYFCARGGLWTGNSYFDLWGPGTLVTVSS | |

TABLE 19
Amino Acid Sequences of LCVRs of Exemplary Antigen Binding Molecules

| | | SEQ |
|---|---|---|
| | | ID |
| Clone | Sequences | NO: |
| 9F7 | DVVMTQTPASVEVAVGGTVTIKCQASEDIESYLAWYQQKPGQPPKLLIYEASKLASGVSSRFSGGGYGTEFTLTISG | 162 |
| | VECADAATYYCQQALTVGNVDNPFGGGSEVVVK | |
| 2H3 | DVVMTQTPASVEVAVGGTVTIKCQASEDIESYLAWYQQKPGQPPKLLIYEASKLASGVSSRFSGGGYGTEFTLTISG | 163 |
| | VECADAATYYCQQALTVGNVDNPFGGGSEVVVK | |
| 69H10 | DVVMTQTASPVSAAVGGTVTIKCQASQSIYSYLSWYQQKPGQPPKLLIYEASKLASGVSSRFSGSGYGTEFTLTISG | 164 |
| | VECADAATYYCQQALTVGNVDNPFGGGSEVVVK | |
| 65D5 | DIVMTQTPASVSEPVGGTVTIKCQASQSISSYLAWYQQKPGQPPKLLIYEATKLTSGVSSRFSGSGYGTEFTLTISG | 165 |
| | VECADAATYYCQQALTVGDVDNPFGGGSEVVVK | |
| 3E9 | DVVMTQTPASVSAAVGGTVTIKCQASEDIESYLAWYQQKPGQPPKLLIYDASTLASGVSSRFSGSGYGTEFTLTISG | 166 |
| | VECADAATYYCQQDLTVGNVDNPFGGGSEVVVE | |
| 9A5 | DVVMTQTPASVEAAVGGTVTINCQASQSIESYLAWYQQKPGQPPKLLIYEASKLASGVSSRFSGGGYGTEFTLTISG | 167 |
| | VECADAATYYCQQALTIGNVDNPFGGGSEVVVK | |
| 12G6 | DVVMTQTPASVEVAVGGTVTIKCQASEDIESYLAWYQQKPGQPPKLLIYEASKLASGVSSRFSGGGYGTEFTLTISG | 168 |
| | VECADAATYYCQQALTIGNVDNPFGGGSEVVVK | |
| 1D10 | DVVMTQTPASVEVAVGGTVTIKCQASEDIESYLAWYQQKPGQPPRLLIYEASKLASGVSSRFSGGGYGTDFTLTISG | 169 |
| | VECADAATYYCHQALTIGNVDNPFGGGGEVVVK | |
| 62E3 | DVVMTQTASPVSAAVGGTVTIKCQASEDIESYLAWYQQKPGQPPKLLIYEASKLASGVSSRFSGSGYGTEFTLTISG | 170 |
| | VECADAATYYCQQALTIGNVDNPFGGGSEVVVK | |
| 3A4 | DVVMTQTPASVSAAVGGTVSINCQASEDIENYLAWYQQKPGQPPKLLIYEASKLASGVSSRFSGSGYGTEFTLTISG | 171 |
| | VECADAATYYCQQALTIGNVDNPFGGGSEVVVK | |
| 63H3 | DVVMTQTPASVEVAVGGTVTLKCQASEDIESYSAWYQQKPGQPPNLLIYEASKLASGVSSRFSGSGYGTEFTLTISG | 172 |
| | VECADAATYYCQQALTIGNVDNPFGGGSEVVVR | |
| 66A1 | DVVMTQTPASVEAAVGGTVSINCQASEDIESYLAWYQQKPGQPPKLLIYKASTLASGVSSRFKGSGSGKQFTLTISG | 173 |
| | VECADAATYYCQQVLTIGNVDNPFGGGSEVVVK | |
| 2E12 | DVVMTQTPASVEVAVGGTVTIKCQASENIESYLAWYQQKPGQPPKLLIYDASTLASGVSSRFSGSGYGTEFTLTISG | 174 |
| | VECADAATYYCQQGLTIGNVDNPFGGGSEVVVK | |
| 72F12 | DVVMTQTPASVEVAVGGTVTIKCQASKNIDSNLAWYQQKPGQPPKQLIYAASTLASGVSSRFSGSGYGAEFTLTISG | 175 |
| | VECADAATYYCQQALTIGNVDNPFGGGSEVVVK | |
| 74F11 | DVVMTQTPASVEAAVGGTVTIKCQASEDIERYLAWYQQKPGQPPKLLIYEASKLPSGVSSRFSGSGYGTEFTLTISG | 176 |
| | VECADAATYYCQQALTIGYVDNPFGGGSEVVVK | |
| 15F5 | DVVMTQTPASVEAAVGGTVTIKCQASEDIERYLAWYQQKPGQPPKLLIYEASKLPSGVSSRFSGSGYGTEFTLTISG | |
| | VECADAATYYCQQALTIGYVDNPFGGGSEVVVK | |
| 9D7 | YDMTQTPASVSEPVGGTVTIKCQASEDIYKLLAWYQQKPGQPPKLLIYAASDLESGVPSRFKGRGSGTDYTLTISDL | 178 |
| | ECADAATYYCQQAYTIGNIDNSFGGGTEVVVK | |
| 9D7-2 | YDMTQTPASVSEPVGGTVTIKCQASEDIYKLLAWYQQKPGQPPKLLIYAASDLESGVPSRFKGRGSGTDYTLTISDL | 179 |
| | EAADAATYYCQQAYTIGNIDNSFGGGTEVVVK | |
| 10A9 | YDMTQTPASVSAAVGGTVTIKCQASEDIYSLLAWYQQKPGQPPKLLIYGASNLESGVPSRFKGSGSGTEYTLTISDL | 180 |
| | ECDDAATYYCQQAYTIGNIDNAFGGGTEVVVK | |
| 10A9-2 | YDMTQTPASVSAAVGGTVTIKCQASEDIYSLLAWYQQKPGQPPKLLIYGASNLESGVPSRFKGSGSGTEYTLTISDL | 181 |
| | EADDAATYYCQQAYTIGNIDNAFGGGTEVVVK | |
| 62A2 | YDMTQTPASVSAAVGGTVTIKCQASEDIYRLLAWYQQKPGQPPKLLIYGASNLESGVPSRFKGSGSGTEYTLTISDL | 182 |
| | ECDDAATYYCQQAYTIGNIDNAFGGGTEVVVK | |
| 74E4 | YDMTQTPASVEAAVGGTVTIKCQASEDIYRLLAWYQQKPGQPPKLLIYGASNLESGVPSRFKGSGSGTEYTLTISDL | 183 |
| | ECDDAATYYCQQAYTIGNIDNAFGGGTEVVVK | |
| 66A6 | YDMTQTPASVSAAVGGTVTIKCQASEDIYSLLAWYQQKPGQPPKLLIYDASDLASGVPSRFKGSGSGTEYTLTISDL | 184 |
| | ECDDAATYYCQQAYTIGNIDNTFGGGTEVVVK | |
| 10E12 | YDMTQTPASVSAAVGGTVTIKCQASEDIYSLLAWYQQKPGQPPKLLIYDASDLASGVPSRFKGSGSGTEYTLTISDL | 185 |
| | ECDDAATYYCQQAYTIGNIDNAFGGGTEVVVK | |
| 8H2 | YDMTQTPASVSAAVGGTVTIKCQASEDIYSLLAWYQQKPGQPPKLLIYDASDLASGVPSRFKGSGSGTEYTLTISAL | 186 |
| | ECDDAATYYCQQAYTIGNIDNTFGGGTEVVVK | |
| 74H11 | YDMTQTPASVSAAVGGTVTIKCQASEDIYSLLAWYQQKPGQPPKLLIYDASDLASGVPSRFKGSGSGTEYTLTISDL | 187 |
| | ECDDAATYYCQQAYTIGNIDNAFGGGTEVVVK | |
| 74A1 | YDMTQTPASVSEPVGGTVTIKCQASQSIYNNFAWYQQKPGQRPKLLIYDASDLASGVPSRFKGSGSGTEYTLTISGV | 188 |
| | QCADAATYYCQQAYTIGNIDNPFGGGTEVVVK | |
| 10F12 | YDMTQTPASVSAAVGGTVTIKCQASEDIYSLLAWYQQKPGQPPKLLIFDASDLASGVPSRFKGSGSGTEYTLTISDL | 189 |
| | ECDDAATYYCQQAYTIGNIDNAFGGGTEVVVK | |
| 65E11 | YDMTQTPASVSAAVGGTITIKCQASEDIYSLLAWYQQKPGQPPKLLIFDASTLASGVPSRFKGSGSGTEYTLTISDL | 190 |
| | ECDDAATYYCQQAYTIGNIDNAFGGGTEVVVE | |
| 69C2 | YDMTQTPASVSEPVGGTVTIKCQASEDIYKLLAWYQQKPGQPPKLLIYAASDLESGVPSRFKGSGSGTEYTLTISDL | 191 |
| | ECADAATYYCQQAYTIGNIDNTFGGGTEVVVK | |
| 61F12 | YDMTQTPASVSAAVGGTVTIKCQASEDIYSLLAWYQQKPGQPPKLLIYDASDLASGVPSRFKGSGSGTEYTLTISDL | 192 |
| | ECDDAATYYCQQAYTIGNIDNSFGGGTEVVVK | |
| 10D3 | YDMTQTPASVSEPVGGTVTIKCQASEDIYRLLAWYQQKPGQPPKLLIYDASDLASGVPSRFKGSGSGTEYTLTISGL | 193 |
| | QCADAATYYCQQGYTIGNIDNSFGGGTEVVVK | |
| 67H4 | YDMTQTPASVSEPVGGTVTIKCQASEDIYNLLAWYQQKPGQPPKLLIYAASDLESGVPSRFKGSGSGTEYTLTISDL | 194 |
| | ECDDAATYYCQQGYTIGNIDNAFGGGTEVVVK | |
| 64G9 | YDMTQTPASVSEPVGGTVTIKCQASEDIYNLLAWYQQKPGQPPKLLIYDASTLTSGVPSRFKGSGSGTEYTLTISDL | 195 |
| | ECDDAATYYCQQAYTIGNIDNTFGGGTEVVVK | |
| 72G12 | YDMTQTPASVSEPVGGTVTIKCQASEDIYKLLAWYQQKPGQPPKLLIYAASDLESGVPSRFKGSGSGTEYTLTISDL | 196 |
| | ECADAATYYCQQAYTIGNLDNAFGGGTEVVVK | |
| 65G8 | YDMTQTPASVSEPVGGTVTIKCQASEDIYKLLAWYQQKPGQPPKLLIYAASDLESGVPSRFKGSGSGTEYTLTISDL | 197 |
| | ECADAATYYCQQAYTIGNIDNTFGGGTEVVVK | |
| 69D8 | YDMTQTPASVSAAVGGTITIKCQASEDIYSLLAWYQQKPGQPPKLLIFDASTLASGVPSRFKGSGSGTEYTLTISDL | 198 |
| | ECDDAATYYCQQAYTIGNIDNAFGGGTEVVVE | |
| 2H5 | YDMTQTPASVSEPVGGTVTIKCQASEDIYNLLAWYQQKPGQPPKLLIYAASDLESGVPSRFKGSGSGTEYTLTISDL | 199 |
| | ECADAATYYCQQAYTVGNIDNAFGGGTEVVVK | |
| 64A6 | YDMTQTPASVSEPVGGTVTIKCQASEDIYKLLAWYQQKPGQPPKLLIYAASDLESGVPSRFKGSGSGTDYTLTISDL | 200 |
| | ECADAATYYCQQAYTIGNIDNSFGGGTEVVVK | |
| 62B10 | YDMTQTPASVSEPVGGTVTIKCQASEDIYNLLAWYQQKPGQPPKLLIYDASTLTSGVPSRFKGGGSGTEYTLTINDL | 201 |
| | ECDDAATYYCQQGYTIGNIDNTFGGGTEVVVK | |
| 5D4 | FEMTQTPSSVSEPVGGTVTIKCQASEDISSNLGWYQQKPGQPPKLLIYGASTLASGVPSRFKGSGSGTEFTLTISDL | 202 |
| | ECADAATYYCQTSYYIDDGVNGFGGGTEVVVK | |
| 5D4-1 | FEMTQTPSSVSEPVGGTVTIKCQASEDISSNLGWYQQKPGQPPKLLIYGASTLASGVPSRFKGSGSGTEFTLTISDL | 203 |
| | EPADAATYYCQTSYYIDDGVNGFGGGTEVVVK | |
| 5D4-2 | FEMTQTPSSVSEPVGGTVTIKCQASEDISSNLGWYQQKPGQPPKLLIYGASTLASGVPSRFKGSGSGTEFTLTISDL | 204 |
| | EAADAATYYCQTSYYIDDGVNGFGGGTEVVVK | |
| 70G2 | FEMTQTPASVSEPVGGTVTIKCQASEDISSNLAWYQQKPGQPPKLLIYGASTLASGVPSRFKGSGSGTEFTLTISDL | 205 |
| | ECADAATYYCQSSYYIDDGVNGFGGGTEVVVK | |
| 4F2 | YDMTQTPASVEVTVGGTVTINCQASESLSSYLAWYQQKPGQPPKPLIYRAATLASGVPSRFKGSGSGTDYTLTISDL | 206 |
| | ECADAATYYCQQGYGYSTVGNAFGGGTEVVVK | |
| 62A10 | YDMTQTPASVEVAVGGTVTINCQASESISRYLAWYQQKPGQSPKPLIYRASTLASGVPSRFQGSGSGTEYTLTISDL | 207 |
| | ECADAATYYCQQGYGYSTVDNAFGGGTEVVVK | |
| 70E2 | YDMTQTPASVEVPVGGTVTINCQASENINRYLAWYQQKPGQPPKPLIYRAATLASGVPSRFKGSGSGTEYTLSISDL | 208 |
| | ECADAATYYCQQGYGYSTVGNAFGGGTEVVVK | |
| 71F10 | YDMTQTPASVSAAVGGTVTINCQASESISRYLAWYQQKPGQPPKPLIYRASTLASGVPSRFKGSGTGTDYTLTISDL | 209 |
| | ECADAATYYCQQGYGYSTVGNAFGGGTEVVVK | |
| 73C2 | YDMTQTPASVEVAVGGTVTINCQASESINRYLAWYQQKRGQPPKPLIYRAATLASGVPSRFKGSGSGTEYTLTISDL | 210 |
| | ECADAATYYCQQGYGYSTVGNAFGGGTEVVVK | |

TABLE 20
Nucleic Acid Sequences of HCVR Encoding Genes

| | | SEQ |
|---|---|---|
| | | ID |
| Clone | Sequences | NO: |
| 9F7 | CAGGAACAGCTGAAGGAGAGCGGCGGCGGCCTGGTGCAGCCAGGAGGAAGTCTGACCCTGAGCTGCAAAGCCAGTGGATTCGACTTC | |
| | AACAACTACGGCGTGAGTTGGGTGAGACAGGCCCCCGGAAAAGGACTGGAGTGGATTGGATACATTTACCCCGCCTACCACAACACC | |
| | TACTACGCCACCTGGGTGAACGGCAGATTCACAATCAGCAGCCACAACGCCCAGAACACCCTGTACCTGCAGCTGAACACACTGACC | |
| | GCCGCCGACACCGCCACCTACTTCTGCGCCAGGGACACCGGCAGCACCGACTACAACTTCAACCTGTGGGGACCAGGCACCCTGGTG | |
| | ACCGTGTCCTCC | |
| 2H3 | CAGGAGCAGCTGAAGGAGTCCGGGGGAGGCCTGGGCCAGCCTGGGGGATCCCTGACACTCTCCTGCAAAGCCTCTGGATTCGACTTC | |
| | AATAACTATGGAGTGAGCTGGGTCCGCCAGGCTCCAGGGAAGGGGCTGGAGTGGATCGGGTACATTTATCCTGCTTATCATAACACA | |
| | TACTACGCGACCTGGGTGAATGGCCGATTCACCATTTCCAGCCACAACGCCCAGAACACGCTGTATCTGCAACTGAACACTCTGACA | |
| | GCCGCGGACACGGCCACCTATTTCTGTGCGAGAGATACTGGTACTACTGATTATAACTTTAACTTGTGGGGCCCAGGCACCCTGGTC | |
| | ACCGTCTCCTCA | |
| 69H10 | CAGGAGCAGCTGAAGGAGTCCGGGGGAGGCCTGGTCCAGCCTGGGGGATCCCTGAAACTCTCCTGCAAAGCCTCTGGATTCGACTTC | |
| | AATAAATATGGAGTGAGCTGGGTCCGCCAGGCTCCAGGGAAGGGGCTGGAGTGGATCGCGTACATTTATCCTGCTTTTCATAACACA | |
| | TACTACGCGACCTGGGTGAATGGCCGATTCACCATTTCCAGCCACAACGCCCAGAACACGCTGTATCTGCAACTGAACACTCTGACA | |
| | GCCGCGGACACGGCCACCTATTTCTGTGCGAGAGATGCTGGTACTACTGATTATAACTTTAACTTGTGGGGCCCAGGCACCCTGGTC | |
| | ACCGTCTCCTCA | |
| 65D5 | CAGGAACAACTCAAAGAGAGCGGCGGCGGCCTCGTGCAACCCGGAGGAAGTCTCAAGCTCAGCTGCAAGGCAAGCGGCTTCGACTTC | |
| | AACGCCTACGGCGTCAGCTGGGTGAGACAGGCCCCCGGAAAGGGACTGGAGTGGATTGCTTATATTTACCCTGCTTTTCACAACACA | |
| | TACTACGCCACCTGGGTGAATGGCAGATTCACCATCTCCTCTCACAACGCCCAGAACACACTGTACCTGCAGCTGAACACACTGACC | |
| | GCCGCCGACACCGCCACCTACTTCTGCGCCCGGGACGCCGGAACCACCGACTACAACTTTAACCTGTGGGGCCCCGGCACACTCGTC | |
| | ACCGTCTCCTCC | |
| 3E9 | CAGGAGCAGCTGAAGGAGTCCGGGGGAGGCCTGGTCCAGCCTGGGGGATCCCTGAAACTCTCCTGCAAAGCCTCTGGATTCGACTTC | |
| | AATAACTATGGAGTGAGCTGGGTCCGCCAGGCTCCAGGGAAGGGGCTGGAGTGGATCGCATACATTTATCCTGCTTTTCATAACAGA | |
| | TATTACGCGAACTGGGTGAATGGCCGATTCACCATCTCCAGCGACAACGCCCAGAACACGGTGTATCTGCAACTGAACAGTCTGACA | |
| | GCCGCGGACACGGCCACCTATTTCTGTGCGAGAGATGCTGGTAGTACTGATTATAAATTTAACTTGTGGGGCCCAGGCACCCTGGTC | |
| | ACCGTCTCCTCA | |
| 9A5 | CAGGAGCAGCTGAAGGAGTCCGGGGGAGGCCTGGTCCAGCCTGGGGGATCCCTGACACTCTCCTGCAAAGCCTCTGGATTCGACTTC | |
| | AATAACTATGGAGTGAGCTGGGTCCGCCAGGCTCCAGGGAAGGGGCTGGAGTGGATCGCGTACATTTATCCTACTTATCATAACACA | |
| | TACTACGCGACCTGGGTGAATGGCCGATTCACCATTTCCAGCCACAACGCCCAGAACACGCTGTATCTGCAACTGAACACTCTGACA | |
| | GCCGCGGACACGGCCACCTATTTCTGTGCGAGAGATGCTGGTGGTACTGATTATAACTTTAACTTGTGGGGCCCAGGCACCCTGGTC | |
| | ACCGTCTCCTCA | |
| 12G6 | CAGGAGCAGCTGAAGGAGTCCGGGGGAGGCCTGGTCCAGCCTGGGGGATCCCTGACACTCTCCTGCAAAGCCTCTGGATTCGACTTC | |
| | AATAACTATGGAGTGAGCTGGGTCCGCCAGGCTCCAGGGAAGGGGCTGGAGTGGATCGGGTACATTTATCCTGCTTATCATAACACA | |
| | TACTACGCGACCTGGGTGAATGGCCGATTCACCATTTCCAGCCACAACGCCCAGAACACGCTGTATCTGCAACTGAACACTCTGACA | |
| | GCCGCGGACACGGCCACCTATTTCTGTGCGAGAGATGCTGGTAATACTGATTATAACTTTAACTTGTGGGGCCCAGGCACCCTGGTC | |
| | ACCGTCTCCTCA | |
| 1D10 | CAGGAACAGCTGAAGGAGAGCGGCGGCGGCCTGGTGCAGCCAGGAGGAAGTCTGACCCTGAGCTGCAAAGCCAGTGGATTCGACTTC | |
| | AACTACTACGGCGTGAGTTGGGTGAGACAGGCCCCCGGAAAAGGACTGGAGTGGATTGGCTATATTTACCCCGCTTATCACAACACC | |
| | TATTACGCCACATGGGTGAACGGCAGATTCACAATCAGCAGCCACAACGCCCAGAACACACTGTACCTGCAGCTGAACACACTGACC | |
| | GCCGCCGACACCGCCACCTACTTCTGCGCCAGGGACGCCGGCAGCACCGACTACAACCTGAACCTGTGGGGACCCGGCACCCTGGTG | |
| | ACCGTGTCCTCC | |
| 62E3 | CAGGAACAACTCAAAGAGAGCGGCGGCGGCCTCGTGCAACCCGGAGGAAGTCTCAAGCTCAGCTGCAAGGCAAGCGGCTTCGACTTC | |
| | AACGAATACGGCGTGAGCTGGGTGAGACAGGCCCCCGGAAAGGGACTGGAGTGGATTGCTTATATTTACCCTGCTTTTCACAACACA | |
| | TACTACGCCACCTGGGTGAATGGCAGATTCACAATAAGCTCTCATAACGCTCAGAACACACTGTACCTGCAGCTGAACACACTGACC | |
| | GCCGCCGACACCGCCACCTACTTCTGCGCCAGGGACGCCGGCACCACCGACTACAACTTCAACCTGTGGGGCCCCGGCACCCTGGTG | |
| | ACCGTTTCCTCC | |
| 3A4 | CAGGAGCAGCTGGTGGAGTCCGGGGGAGGCCTGGTCCAGCCTGGGGGATCCCTGACACTCTCCTGCAAAGGCTCTGGATTCGACTTC | |
| | AATGAATATGGAGTGAGCTGGGTCCGCCAGGCTCCAGGGAAGGGGCTGGAGTGGATCGCGTACATTTATCCTGCCTTTCATAACACA | |
| | TACTACGCGACCTGGGTGAATGGCCGATTCACCATTTCCAGCCACAACGCCCAGAACACGCTGTATCTGCAACTGCACACTCTGACA | |
| | GCCGCGGACACGGCCACCTATTTCTGTGCGAGAGATGCTGGTACTACTGATTATAACTTTAACTTGTGGGGCCCAGGCACCCTGGTC | |
| | ACCGTCTCCTCA | |
| 63H3 | CAGGAACAACTCAAAGAGAGCGGCGGCGGCCTCGTGCAACCCGGAGGAAGTCTCAAGCTCAGCTGCAAGGCAAGCGGCTTCGACTTC | |
| | AACAGCTACGGAGTGAGCTGGGTGAGACAGGCCCCCGGCAAAGGACTGGAGTGGATTGCTTATATTTACCCAGCCTTCCACAACGCC | |
| | TACTACGCCAACTGGGTGAATGGCAGATTCACAATCAGCAGCGACAACGCCCAGAACACCGTGGACCTGCAACTGAACAGCCTGACC | |
| | GCCGCCGACACCGCCACCTACTTCTGCGCCCGCGACGCCGGAGGCACCGATTACAACTACAACCTCTGGGGCCCTGGCACCCTCGTG | |
| | ACAGTGAGCTCC | |
| 66A1 | CAGGAACAACTCAAAGAGAGCGGCGGCGGCCTCGTGCAACCCGGAGGAAGTCTCAAGCTCAGCTGCAAGGCAAGCGGCTTCGACTTC | |
| | AACAACTACGGCGTGAGCTGGGTGAGACAGGCCCCCGGAAAGGGACTGGAGTGGATTGCTTATATTTACCCTGCTTTTCACAACACA | |
| | TACTACGCCAGCTGGGTGAATGGCAGATTCACAATCTCCTCTCATAACGCTCAGAACACCCTGTACCTGCAACTGAACAGCCTGACC | |
| | GCCGCCGACACCGCCACCTACTTCTGCGCCAGGGACGCCGGCGGCACCGATTACGCTTACAACCTCTGGGGCCCCGGAACCCTGGTC | |
| | ACCGTGTCCTCC | |
| 2E12 | CAGGAGCAGCTGAAGGAGAGCGGCGGCGGCCTGGTGCGCCTGGGCGATCCGCTGCAGCTCTCCTGCAAAGCCTCTGGATTCGACTTC | |
| | AATAACTATGGAGTGACCTGGGTCCGCCAGGCTCCAGGGAAGGGGCTGGAGTGGATCGCGTACATTTATCCTGCTTTTCCTAACACA | |
| | TACTACGCGACCTGGGTGAATGGCCGATTCACCATCTCCCCCCACAACGCCCAGAACACGCTGTATCTGCAACTGAACAGTCTGACA | |
| | GCCGCGGACACGGCCACCTATTTCTGTGCGAGAGATGCTGGTAGTACTGATTATTACTTTAACTTGTGGGGCCCAGGCACCCTGGCC | |
| | ACCGTGTCCTCC | |
| 72F12 | CAGGAACAACTCAAAGAGAGCGGCGGCGGCCTCGTGCAACCCGGAGGAAGTCTCAAGCTCAGCTGCAAGGCAAGCGGCTTCGACTTC | |
| | AACAACTACGGCGTGAGCTGGGTGAGACAGGCCCCCGGAAAGGGACTGGAGTGGATTGCTTATATTTACCCTGCTTTTCACAACACA | |
| | TACTACGCCACATGGGTGAATGGCAGATTCACAATATCCTCCCATAACGCTCAGAACACACTGTACCTGCAGCTGAACAGCCTGACC | |
| | GCCGCCGACACCGCCACCTACTTCTGCGCACGCGACGCCGGCAGCGTGGACTACAACTTCAACCTGTGGGGCCCCGGCACTCTCGTG | |
| | ACCGTCTCCTCC | |
| 74F11 | CAGGAACAACTCAAAGAGAGCGGCGGCGGCCTCGTGCAACCCGGAGGAAGTCTCAAGCTCAGCTGCAAGGCAAGCGGCTTCGACTTC | |
| | AACAACTACGGCATGAGCTGGGTGAGACAGGCCCCCGGCAAAGGACTGGAGTGGATTGCTTATATTTACCCAGCCTTCCACAACACC | |
| | TACTACGCCAACTGGGTGAATGGCAGATTCACAATCAGCAGCGACAACGCCCAGAACACCGTGGACCTGCAACTGAACAGCCTGACC | |
| | GCCGCCGACACCGCCACCTACTTCTGCGCCCGCGACGCCGGAAGCACCGACTACAACTTCAACCTGTGGGGCCCCGGAACCCTGGTC | |
| | ACCGTGAGCTCC | |
| 15F5 | CAGGAGCAGCTGAAGGAGTCCGGGGGAGGCCTGGTCCAGCCTGGGGGATCCCTAAAACTCTCCTGCAAAGCCTCTGGATTCGACTTC | |
| | AATAACTATGGAATGAGCTGGGTCCGCCAGGCTCCAGGGAAGGGGCTGGAGTGGATCGCGTACATTTATCCTGCTTTTCATAATACA | |
| | TACTACGCGAACTGGGTGAATGGCCGATTCACCATCTCCAGCGACAACGCCCAGAACACTGTGGATCTGCAACTGAACAGTCTGACA | |
| | GCCGCGGACACGGCCACCTATTTCTGTGCGAGAGATGCTGGTAGTACTGATTATAACTTTAACTTGTGGGGCCCAGGCACCCTGGTC | |
| | ACCGTCTCCTCA | |
| 9D7 | CAGAGCCTGGAGGAAAGCGGCGGCGACCTGGTGCAGCCCGGAGCTTCACTGACCCTGACCTGCAAGGCCAGCGGCTTCGACCTGAAC | |
| | AACTACAGCATCCTGTGGGTGAGACAGGCCCCCGGAAAAGGCCTGGAGTGGATTGGATGCATTTACATCAGTGGAGGCTCCACCTAC | |
| | TACGCCAACTGGGCCAAGGGCAGATTCGCCATCAGCAAGACCTCCAGCACCACAGTGACCCTGCAGATGACAAGCCTGACCGCCGCC | |
| | GACACCGCCACCTACTTTTGCGCCCGCGACGACGGAACCGCCAGCTACTACCTGAACCTGTGGGGACCCGGCACCCTGGTGACCGTG | |
| | AGCTCC | |
| 9D7-2 | CAGAGCCTGGAGGAAAGCGGCGGCGACCTGGTGCAGCCCGGAGCTTCACTGACCCTGACCTGCAAGGCCAGCGGCTTCGACCTGAAC | |
| | AACTACAGCATCCTGTGGGTGAGACAGGCCCCCGGAAAAGGCCTGGAGTGGATTGGATCTATTTACATCAGTGGAGGCTCCACCTAC | |
| | TACGCCAACTGGGCCAAGGGCAGATTCGCCATCAGCAAGACCTCCAGCACCACAGTGACCCTGCAGATGACAAGCCTGACCGCCGCT | |
| | GACACCGCCACCTACTTTTGCGCCCGCGACGACGGAACCGCCAGCTACTACCTGAACCTGTGGGGACCCGGCACCCTGGTGACCGTG | |
| | AGCAGC | |
| 10A9 | CAGAGCCTGGAGGAAAGCGGCGGCGACCTGGTGCAGCCCGGAGCTTCACTGACCCTGACCTGCAAGGCCAGCGGCTTCGACCTGAAC | |
| | AACTACGCCATGCTGTGGGTGAGACAGGCCCCCGGAAAAGGCCTGGAGTGGATTGGATGCATTTACATTAGCGGCGGCACCACCTAC | |
| | TTCGCCAGCTGGGCCACAGGCAGATTCGCCATCAGCAAAACCAGCAGCACCACCGTGACCCTGCAGATGACAAGCCTGACCGCCGCC | |
| | GACACCGCCACCTACTTTTGCGCCCGCGACGACGGAACCACCAGCTACTACCTGAACCTGTGGGGACCCGGCACCCTGGTGACCGTG | |
| | AGCTCC | |
| 10A9-2 | CAGAGCCTGGAGGAAAGCGGCGGCGACCTGGTGCAGCCCGGAGCTTCACTGACCCTGACCTGCAAGGCCAGCGGCTTCGACCTGAAC | |
| | AACTACGCCATGCTGTGGGTGAGACAGGCCCCCGGAAAAGGCCTGGAGTGGATTGGAAGCATTTACATCAGTGGAGGCACCACCTAC | |
| | TTTGCCAGCTGGGCCACCGGCAGATTCGCCATCAGCAAAACCTCCTCTACCACCGTGACCCTGCAGATGACATCTCTGACTGCCGCC | |
| | GACACCGCCACCTACTTTTGCGCCCGCGACGACGGAACCACCTCCTACTATCTGAACCTGTGGGGACCCGGCACCCTGGTGACCGTG | |
| | AGCAGC | |
| 62A2 | CAGTCGTTGGAGGAGTCCGGGGGAGACCTGGTCCAGCCTGGGGCATCCCTGACACTCACCTGCAAAGCCTCTGGATTCGACCTCAAT | |
| | AACTATGCAATGCTCTGGGTCCGCCAGGCTCCAGGGAAGGGGCTGGAGTGGATCGGATGCATTTATATTAGTGGTGGTACCACTTAC | |
| | TTCGCGAGCTGGGCGACAGGCCGATTCGCCATCTCCAAAACCTCGTCGACCACCTTGACTCTGCAAATGACCACCCTTCCCCCCCCG | |
| | GACACGGCCACCTATTTCTGTGCGAAAAATGATGGTACTACTAGTTATTATCTTAACTTGTGGGGCCCAGGCACCCTGGTCACCGTC | |
| | TCCTCA | |
| 74E4 | CAGTCGTTGGAGGAGTCCGGGGGAGACCTGGTCCAGCCTGGGGCATCCCTGACACTCACCTGCAAGGCCTCTGGATTCGACCTCAAT | |
| | AACTATGCAATTCTCTGGGTCCGCCAGGCTCCAGGGAAGGGGCTGGAGTGGATCGGATGCATTTATATTAGTGGTGCTACCACTTAC | |
| | TTCGCGAACTGGGCGACAGGCCGATTCGCCATCTCCAAAACCTCGTCGACCACGGTGACTCTGCAAATGACCAGTCTGACAGCCGCG | |
| | GACACGGCCACCTATTTCTGTGCGAGAGATGATGGTACTACTAGTTATTATCTTAACTTGTGGGGCCCAGGCACCCTGGTCACCGTC | |
| | TCCTCA | |
| 66A6 | CAGTCGTTGGAGGAGTCCGGGGGAGACCTGGTCCAGCCTGGGGCATCCCTGACACTCACCTGTAAAGCCTCTGGATTCGACCTCAAT | |
| | AATTATGCAATGCTTTGGGTCCGCCAGGCTCCAGGGAAGGGGCTGGAGTGGATCGGATGCATTTATATTAGTGGTGGTACCACTTAC | |
| | TTCGCGAGCTGGGCGACAGGCCGATTCGCCATCTCCAAAACCTCGTCGACCACGGTGACTCTGCAAATGACCAGTCTGACAGCCGCG | |
| | GACACGGCCACCTATTTCTGTGCGAGAGATGATGGTACCACTAGTTATTATCTTAACTTGTGGGGCCCAGGCACCCTGGTCACCGTC | |
| | TCCTCA | |
| 10E12 | CAGTCGTTGGAGGAGTCCGGGGGAGACCTGGTCCAGCCTGGGGCATCCCTGACACTCACCTGTAAAGCCTCTGGATTCGACCTCAAT | |
| | AACTATGCAATGCTCTGGGTCCGCCAGGCTCCAGGGAAGGGGCTGGAGTGGATCGGATGCATTTATATTAGTGGTGGTACCACTTAC | |
| | TTCGCGAGCTGGGCGACAGGCCGATTCGCCATCTCCAAAACCTCGTCGACCACGGTGACTCTGCAAATGACCAGTCTGACAGCCGCG | |
| | GACACGGCCACCTATTTCTGTGCGAGAGATGATGGTACTACTAGTTATTATCTTAACTTGTGGGGCCCAGGCACCCTGGTCACCGTC | |
| | TCCTCA | |
| 8H2 | CAGTCGTTGGAGGAGTCCGGGGGAGACCTGGTCCAGCCTGGGGCATCCCTGACACTCACCTGTAAAGCCTCTGGATTCGACCTCAAT | |
| | AACTATGCAATGCTCTGGGTCCGCCAGGCTCCAGGGAAGGGGCTGGAGTGGATCGGATGCATTTATATTAGTGGTGGTACCACTTAC | |
| | TTCGCGAGCTGGGCGACAGGCCGATTCGCCATCTCCAAAACCTCGTCGACCACGGTGACTCTGCAAATGACCAGCCTGACAGCCGCG | |
| | GACACGGCCACCTATTTCTGTGCGAGAGATGATGGTAGTACTAGITATTATCTTAACTTGTGGGGCCCAGGCACCCTGGTCACCGTC | |
| | TCCTCA | |
| 74H11 | CAGTCGTTGGAGGAGTCCGGGGGAGACCTGGTCCAGCCTGGGGCATCCCTGACACTCACCTGTAAAGCCTCTGGATTCGACCTCAAT | |
| | AACTATGCAATGCTCTGGGTCCGCCAGGCTCCAGGGAAGGGGCTGGAGTGGATCGGATGCATTTATATTAGTGGTGGTACCACTTAC | |
| | TTCGCGAGCTGGGCGACAGGCCGATTCGCCATCTCCAGAACCTCGTCGACCACGGTGACTCTGCAAATGACCAGTCTGACAGCCGCG | |
| | GACACGGCCACCTATTTCTGTGCGAGAGATGATGGTAGTACTAGTTATTATCTTAACTTGTGGGGCCCAGGCACCCTGGTCACCGTC | |
| | TCCTCA | |
| 74A1 | CAATCGTTGGAGGAGTCCGGGGGAAACCTGGCCCACCCTGGGGCATCCCTGAGACTCACCTGTACAGCCTCTGGATTCTACCTCAAT | |
| | AACTATGCAATGCTCTGGGTCCGCCAGGCTCCAGGGAAGGGGCTGGAGTGGATCGGATGCCITTATATTAGTGGTGGTACCACTTAC | |
| | TTCGCGAGCTGGGCGACAGGCCGATTCGCCATCTCCAAAACCTCGTCAACCACTGGGACTCTGCAAATGACCAGTCTGACAGCCGCG | |
| | GACACGGCCACCTATTTCTGTGCGAGAAATGATGGTAGTACTAATTATTATCTTAACTTGTGGGGCCCAGGCACCCTGGTCACCATC | |
| | TCCTCA | |
| 10F12 | CAGTCGTTGGAGGAGTCCGGGGGAGACCTGGTCCAGCCTGGGGCATCCCTGACACTCACCTGTAGAGCCTCTGGATTCGACCTCAAT | |
| | AACTATTCAATGCTCTGGGTCCGCCAGGCTCCAGGGAAGGGGCTGGAGTGGATCGGATGCATTTATATTAGTGGTGGTACCACTTAC | |
| | TTCGCGAGCTGGGCGACAGGCCGATTCGCCATCTCCAAAACCTCGTCGACCACGGTGACTCTGCAAATGACCAGTCTGACAGCCGCG | |
| | GACACGGCCACCTATTTCTGTGCGAGAGATGATGGTAGTACTAGTTATTATCTTAACTTGTGGGGCCCAGGCACCCTGGTCACCGTC | |
| | TCCTCA | |
| 65E11 | CAGTCGTTGGAGGAGTCCGGGGGAGACCTGGTCCAGCCTGGGGCATCCCTGACACTCACCTGTAGAGCCTCTGGATTCGACCTCAAT | |
| | AACTATTCAATGCTCTGGGTCCGCCAGGCTCCAGGGAAGGGGCTGGAGTGGATCGGATGCATTTATATTAGTGGTGGTACCACTTAC | |
| | TTCGCGAGCTGGGCGACAGGCCGATTCGCCATCTCCAAAACCTCGTCGACCACGGTGACTCTGCAAATGACCATTCTGACAGCCGCG | |
| | GACACGGCCACCTATTTCTGTGCGAGAGATGATGGTAGTACTAGTTATTATCTTAACTTGTGGGGCCCAGGCACCCTGGTCACCGTC | |
| | TCCTCA | |
| 69C2 | CAGTCGTTGGAGGAGTCCGGGGGAGACCTGGTCCAGCCTGGGGCATCCCTGACACTCACCTGTAGAGCCTCTGGATTCGACCTCAAT | |
| | AACTATTCAATGCTCTGGGTCCGCCAGGCTCCAGGGAAGGGGCTGGAGTGGATCGGATGCATTTATATTAGTGGTGGTACCACTTAC | |
| | TTCGCGAGCTGGGCGACAGGCCGATTCGCCATCTCCAAAACCTCGTCGACCACGGTGACTCTGCAAATGACCATTCTGACAGCCGCG | |
| | GACACGGCCACCTATTTCTGTGCGAGAGATGATGGTAGTACTAGITATTATCTTAACTTGTGGGGCCCAGGCACCCTGGTCACCGTC | |
| | TCCTCA | |
| 61F12 | CAGTCGTTGGAGGAGTCCGGGGGAGACCTGGTCCAGCCTGGGGCATCCCTGACACTCACCTGTAAAGCCTCTGGATTCGACCTCAAC | |
| | AACTATTCAATACTATGGGTCCGCCAGGCTCCAGGGAAGGGGCTGGAGTGGATCGGATGCATTTATATTAGTGGTGGTACCACTTAC | |
| | TTCGCGAGCTGGGCGACAGGCCGATTCGCCATCTCCAAAACCTCGTCGACCACGGTGACTCTGCAAATGACCAGTCTGACAGCCGCG | |
| | GACACGGCCACCTATTTCTGTGCGAGAGATGATGGTACTACTAGTTATTATCTTAACTTGTGGGGCCCAGGCACCCTGGTCACCGTC | |
| | TCCTCA | |
| 10D3 | CAGTCGTTGGAGGAGTCCGGGGGAGACCTGGTCCAGCCTGGGGCATCCCTGACACTCACCTGCAAAGCCGCTGGATTCGACCTCAAT | |
| | AATTATTCAATGCTCTGGGTCCGCCAGGCTCCAGGGAAGGGGCTGGAGTGGATCGGATGCATTTATATTAGTGGTGGTACCACTTAC | |
| | TACGCGAGCTGGGCGAAAGGCCGATTCACCATCTCCAAAACCTCGTCGACCACGGTGACTCTGCAAATGACCAGTCTGACAGCCGCG | |
| | GACACGGCCACCTATTTCTGTGCGAGAGATGATGGGAGTACTAGTTATTATTTAAACCTGTGGGGCCCAGGCACCCTGGTCACCGTC | |
| | TCCTCA | |
| 67H4 | CAGTCGTTGGAGGAGTCCGGGGGAGACCTGGTCCAGCCTGGGGTATCCCTGACACTCACCTGCAAAGCCTCTGGATTCGACCTCAAT | |
| | AACTATTCAATACTCTGGGTCCGCCAGGCTCCAGGGAAGGGGCTGGAGTGGATCGGATGCATTTATATTAGTGGTGGTAGCACTTAC | |
| | TACGCGAGCTGGGCGAAAGGCCGATTCGCCATCTCCAAAACCTCGTCGACCACGGTGACTCTGCAAATGACCAGTCTGACAGCCGCG | |
| | GACACGGCCACCTATTTCTGTGCGAGAGATGATGGTACTACTAGTTATTATCTTAACTTGTGGGGCCCAGGCACCCTGGTCACCGTC | |
| | TCCTCA | |
| 64G9 | CAGTCGTTGGAGGAGTCCGGGGGAGACCTGGTCCAGCCTGGGGCATCCCTGACACTCACCTGCAAAGCCTCTGGATTCGACCTCAAT | |
| | AACTATTCTATACTCTGGGTCCGCCAGGCTCCAGGGAAGGGGCTGGAGTGGATCGGATGTATCTATATTAGTGGTGGTACCACTTAC | |
| | TACGCGAACTGGGCGAAAGGCCGATTCACCATCTCCAGAACCTCGTCGACCACGGTGACTCTGCAAATGACCAGTCTGACAGGCGCG | |
| | GACACGGCCACCTATTTCTGTGCGAGAGATGATGGTAGTACTAGTTATTATTTGAACTTGTGGGGCCCAGGCACCCTGGTCACCGTC | |
| | TCCTCA | |
| 72G12 | CAGTCGTTGGAGGAGTCCGGGGGAGACCTGGTCCAGCCTGGGGCATCCCTGACACTCACCTGCAAAGCCTCTGGATTCGACCTCAAT | |
| | AACTATTCAATACTCTGGGTCCGCCAGGCTCCAGGGAAGGGGCTGGAGTGGATCGGATGCATTTATATTAGTGGTGGTACCACTTAC | |
| | TACGCGAACTGGGCGAAAGGCCGATTCGCCATCTCCAAAACCTCGTCGACCACGGTGACTCTGCAAATGACCAGTCTGACAGCCGCG | |
| | GACACGGCCACCTATTTCTGTGCGAGAGATGATGGTACTACTAGTTATTATCTTAACTTGTGGGGCCCAGGCACCCTGGTCACCGTC | |
| | TCCTCA | |
| 65G8 | CAGTCGTTGGAGGAGTCCGGGGGAGACCTGGTCCAGCCTGGGGCATCCCTGACACTCACCTGCAAAGCCTCTGGATTCGACCTCAAT | |
| | AACTATTCAATACTCTGGGTCCGCCAGGCTCCAGGGAAGGGGCTGGAGTGGATCGGATGCATTTATATTAGTGGTGGTAGCACTTAC | |
| | TACGCGAACTGGGCGAAAGGCCGATTCGCCATCTCCAAAACCTCGTCGACCACGGTGACTCTGCAAATGACCAGTCTGACAGCCGCG | |
| | GACACGGCCACCTATTTCTGTGCGAGAGATGATGGTACTACTAGTTATTATCTTAACTTGTGGGGCCCAGGCACCCTGGTCACCGTC | |
| | TCCTCA | |
| 69D8 | CAGTCGTTGGAGGAGTCCGGGGGAGACCTGGTCCAGCCTGGGGCATCCCTGACACTCACCTGCAAAGCCTCTGGATTCGACCTCAAT | |
| | AACTATTCAATACTCTGGGTCCGCCAGGCTCCAGGGAAGGGGCTGGAGTGGATCGGATGCATTTATATTAGTGGTGGTAGCACTTAC | |
| | TACGCGAACTGGGCGAAAGGCCGATTCGCCGTCTCCAAAACCTCGTCGACCACGGTGACTCTGCAAATGACCAGTCTGACAGCCGCG | |
| | GACACGGCCACCTATTTCTGTGCGAGAGATGATGGTACTACTAGTTATTATCTTAACTTGTGGGGCCCAGGCACCCTGGTCACCGTC | |
| | TCCTCA | |
| 2H5 | CAGTCGTTGGAGGAGTCCGGGGGAGACCTGGTCCAGCCTGGGGCATCCCTGACACTCACCTGCAAAGCCTCTGGATTCGACCTCAAT | |
| | AACTATTCAATACTCTGGGTCCGCCAGGCTCCAGGGAAGGGGCTGGAGTGGATCGGATGCATTTATTTTAGTGGTGGTAGCACTTAC | |
| | TACGCGAACTGGGCGAAAGGCCGATTCGCCATCTCCAAAACCTCGTCGACCACGGTGACTCTGCAAATGACCAGTCTGACAGCCGCG | |
| | GACACGGCCACCTATTTCTGTGCGAGAGATGATGGTACTACTAGITATTATCTTAACTTGTGGGGCCCAGGCACCCTGGTCACCGTC | |
| | TCCTCA | |
| 64A6 | CAGTCGTTGGAGGAGTCCGGGGGAGACCTGGTCCAGCCTGGGGCATCCCTGACACTCACCTGCAAAGCCTCTGGATTCGACCTCAAT | |
| | AACTATTCAATACTCTGGGTCCGCCAGGCTCCAGGGAAGGGGCTGGAGTGGATCGGATGCATTTATATTAGTGGTGGTAGTACTTAC | |
| | TACGCGAACTGGGCGAAAGGCCGATTCGCCATCTCCAAAACCTCGTCGACCACGGTGACTCTGCAGATGACCAGTCTGACAGCCGCG | |
| | GACACGGCCACCTATTTCTGTGCGAGAGATGATGGGACTGCTAGTTATTATCTTAACTTGTGGGGCCCAGGCACCCTGGTCACCGTC | |
| | TCCTCA | |
| 62B10 | CAGTCGTTGGAGGAGTCCGGGGGAGACCTGGTCCAGCCTGGGACATCCCTGACACTCTCCTGCAAAGCCTCTGGATTCGACCTCAAT | |
| | AACTATTCTATACTCTGGGTCCGCCAGGCTCCAGGGAAGGGGCTGGAATGGATCGGATGCATCTATATTAGTGGTGGAACCACTTAT | |
| | TTTGCGAACTGGGCGAAAGGCCGATTCACCATCTCCAGACCCTCGTCGACCACGGTGACTCTGCAAATGACCAGTCTGACAGGCGCG | |
| | GACACGGCCACTTATTTCTGTGCGAGAGATGATGGTAGTGTTAGTTATTATTTGAACTTGTGGGGCCCAGGCACCCTGGTCATCGTC | |
| | TCCTCA | |
| 5D4 | CAGCAGCTGGAGGAAAGCGGCGGCGGCCTGGTGAAGCCCGAGGGAAGTCTGACCCTGACCTGCAAGGGCAGCGGCTTCGATTTCAGC | |
| | GGCACCTACTGGATGTGGTGGGTGAGACAGGCCCCTGGCAAGGGACTGGAGTGGATTGCTTGTATCTACGTGGGCTCTGGCAGCAGC | |
| | ACCTATTACGCTTCATGGGCTAAGGGCAGGTTTACAATCAGCAGCACTAGCAGTACCGCCGTGACCCTGCAGGCTACTAGTCTGACC | |
| | GCTGCCGACACCGCCACATACTTCTGCGCCCGCGGCGCCACTAACAACGTGTTCATGAACTACTTCAACCTGTGGGGACCCGGAACA | |
| | CTGGTGACCGTGTCATCC | |
| 5D4-1 | CAGCAGCTGGAGGAAAGCGGCGGCGGCCTGGTGAAGCCCGAGGGAAGTCTGACCCTGACCTGCAAGGGCAGCGGCTTCGATTTCAGC | |
| | GGCACCTACTGGATGTGGTGGGTGAGACAGGCCCCTGGCAAGGGACTGGAGTGGATTGCTCCCATCTACGTGGGGAGCGGCAGCAGC | |
| | ACCTACTACGCTTCATGGGCTAAGGGCAGGTTCACAATCAGCAGCACCTCTTCTACCGCTGTGACTCTGCAGGCTACTAGTCTGACC | |
| | GCCGCCGACACCGCCACCTACTTCTGCGCCAGAGGCGCCACCAACAACGTGTTCATGAACTACTTCAACCTGTGGGGCCCCGGCACC | |
| | CTGGTGACAGTGAGCTCC | |
| 5D4-2 | CAGCAGCTGGAGGAAAGCGGCGGCGGCCTGGTGAAGCCCGAGGGAAGTCTGACCCTGACCTGCAAGGGCAGCGGCTTCGATTTCAGC | |
| | GGCACCTACTGGATGTGGTGGGTGAGACAGGCCCCTGGCAAGGGACTGGAGTGGATTGCTAGTATCTACGTGGGGAGCGGCAGCAGC | |
| | ACCTACTACGCTTCCTGGGCTAAGGGCAGATTTACAATCAGCAGCACTAGCAGTACCGCCGTGACCCTGCAGGCCACCAGCCTGACC | |
| | GCCGCTGACACCGCTACATACTTCTGCGCCAGAGGCGCCACCAACAACGTGTTCATGAACTACTTCAACCTGTGGGGACCCGGCACA | |
| | CTGGTGACCGTGAGCAGC | |
| 70G2 | CAGCAGCTGGAGGAGTCCGGGGGAGGCCTGGTCAAGCCTGAGGGATCCCTGACACTCACCTGCAAAGGCTCTGGATTCGACTTCAGT | |
| | GGCACCTATTGGATGTGCTGGGTCCGCCAGGCTCCAGGGAAGGGGCTGGAGTGGATCGCATGCATTTATGTTGGTAGTGGTGGTAGC | |
| | ACTTACTACGCGAGCTGGGCGAAAGGCCGATTCACCATCTCCAGCACCTCGTCGACCGCGGTGACTCTGCAAGTGACCAGTCTGACA | |
| | GCCGCGGACACGGCCACCTATTTCTGTGCGAGAGGTGCTACTAATAATGTGTTTAGAAACTACTTTAACTTGTGGGGCCCAGGCACC | |
| | CTGGTCACCGTCTCCTCA | |
| 4F2 | CAGAGCCTGGAGGAAAGCGGCGGCGGCCTGGTGAAGCCCGGAGGAACACTGACCCTGACCTGCACCGTGAGCGGCTTCGATTTCAGC | |
| | AACTACGCCATGACCTGGGTGAGACAGGCCCCAGGAGAAGGGCTGGAGTATATCGGCTTCGTGGCCCTGAGAGGAAACATTTACTAC | |
| | GCCAACTGGGCTAAGGGAAGGTTTACTATCAGCAAGACTAGCAGCACCACCGTGACACTGCAGATGACCAGTCTGACAGTGGCCGAC | |
| | ACCGCCACATACTTCTGCGCCAGGGGCGGCCTGTACACTGGATACAGCTACTTTGACCTGGGCGGGCCCGGGACCCTGGCTACCGTG | |
| | AGCTCA | |
| 62A10 | CAGTCGTTAGTGGAGTCCGGGGGAGGCCTGGTCCAGCCTGAGGGATCCCTGACACTCACCTGCAAAGCCTCTGGATTCGACTTCAAT | |
| | AATTATGCAGTGACCTGGGTCCGCCAGGCTCCAGGGGAGGGGCTGGAATACATCGGATTCATTGGTATTCGTGGTCATATTTACTAC | |
| | GCGAACTGGGCGAAAGGCCGATTCACCATCTCCAAAACCTCGTCGACCACGATGACTCTACAAATGACTAGTCTGACAGTCGCGGAC | |
| | ACGGCCACCTATTTCTGTGCGAGAGGGGGCTTATGGACTGGTAATAGTTACTTTGACTTGTGGGGCCCAGGCACCCTGGTCACCGTC | |
| | TCCTCA | |
| 70E2 | CAGTCGTTGGAGGAGTCCGGGGGAGGCCTGGTCAAGCCTGGAGGAACCCTGACACTCACCTGCACAGTCTCTGGATTCGACTTCAGT | |
| | AATTATGCGATGACCTGGGTCCGCCAGGCTCCAGGGAAAGGGCTGGAATACATCGGATTCATTGGTATTCGTGGTAATATTTACTAC | |
| | GCGAACTGGGCGAAAGGCCGATTCACCATCTCCAAAACCTCGTCGACCACGGTGACTCTGCAAATGACCAGTCTGACAGTCGCGGAC | |
| | ACGGCCACCTATTTCTGTGCGAGAGGGGGCTTATGGACTGGTAATAGTTATTTTGACTTGTGGGGCCCAGGCACCCTGGTCACCGTC | |
| | TCCTCA | |
| 71F10 | CAGTCGTTGGAGGAGTCCGGGGGAGGCCTGGTCCAGCCTGGGGCATCCCTGACACTCACCTGCACAGTCTCTGGATTCGACTTCAGT | |
| | AGCAATGCAATGACCTGGGTCCGCCAGGCTCCAGGGGAGGGGCTGGAATACATCGGATTCATTGGTATTCGTGGTAATATTTACTAC | |
| | GCGAACTGGGCGAAAGGCCGATTCACCATCTCCAAAACCTCGTCGACCACGGTGACTCTGCAAATGACCAGTCTGACAGTCGCGGAC | |
| | ACGGCCACGTATTTCTGTGCGAGAGGGGGCTTATGGACTGGTAACAGTTATTTTGACTTGTGGGGCCCAGGCACCCTGGTCACCGTC | |
| | TCCTCA | |
| 73C2 | CAGTCGTTGGAGGAGTCCGGGGGAGGCCTGGTCAAGCCTGGAGGAACCCTGACACTCACCTGCACAGTCTCTGGATTCGACTTCAGT | |
| | AATTATGCTGTGACCTGGGTCCGCCAGGCTCCAGGGGAGGGGCTGGAATACATCGGATTCGTTGGTATTTATGGTGATTTTTACTAC | |
| | GCGAACTGGGCGAAAGGCCGATTCACCATCTCCAAAACCTCGTCGACCACGGTGACTCTGCAAATGCCCAGTCTGACAGTCGCGGAC | |
| | ACGGCCACCTATTTCTGTGCGAGAGGGGGCTTATGGACTGGTAACAGTTATTTTGACTTGTGGGGCCCAGGCACCCTGGTCACCGTC | |
| | TCCTCA | |

TABLE 21
Nucleic Acid Sequences of LCVR Encoding Genes

| | | SEQ |
|---|---|---|
| | | ID |
| Clone | Sequences | NO |
| 9F7 | GATGTGGTGATGACCCAGACCCCCGCCTCTGTGGAAGTGGCCGTGGGCGGAACCGTGACCATTAAATGTCAGGCCTCTGAAGATATT | |
| | GAGTCCTATCTGGCCTGGTACCAGCAGAAACCCGGCCAGCCCCCTAAGCTGCTGATCTACGAGGCCTCCAAGCTGGCTAGTGGCGTG | |
| | AGCTCCAGATTTAGCGGAGGGGGATACGGCACAGAGTTCACCCTGACTATCAGCGGCGTGGAGTGTGCCGACGCCGCCACATACTAC | |
| | TGCCAGCAGGCCCTGACTGTGGGAAATGTGGACAACCCATTCGGAGGGGGGAGCGAGGTGGTGGTGAAG | |
| 2H3 | GACGTCGTGATGACCCAGACTCCAGCCTCTGTGGAGGTAGCTGTGGGAGGCACAGTCACCATCAAGTGCCAGGCCAGTGAGGACATT | |
| | GAAAGCTATTTAGCCTGGTATCAGCAGAAACCAGGGCAGCCTCCCAAACTCCTGATCTATGAAGCATCCAAGCTGGCATCTGGGGTC | |
| | TCATCGCGGTTCAGCGGCGGTGGATATGGGACAGAGTTCACTCTCACCATCAGCGGCGTGGAGTGTGCCGATGCTGCCACTTACTAC | |
| | TGTCAACAGGCTTTGACTGTTGGTAATGTTGATAATCCTTTCGGCGGAGGAAGCGAAGTGGTGGTCAAA | |
| 69H10 | GACGTCGTGATGACCCAGACTGCATCCCCCGTGTCTGCAGCTGTGGGAGGCACAGTCACCATCAAGTGCCAGGCCAGTCAGAGCATT | |
| | TACAGCTACTTATCCTGGTATCAACAGAAACCAGGGCAGCCTCCCAAGCTCCTGATCTACGAAGCATCCAAACTGGCATCTGGGGTC | |
| | TCATCGCGGTTCAGCGGCAGTGGATATGGGACAGAGTTCACTCTCACCATCAGCGGCGTGGAGTGTGCCGATGCTGCCACTTACTAC | |
| | TGTCAACAGGCTTTGACTGTTGGTAATGTTGATAATCCTTTCGGCGGAGGAAGCGAGGTGGTGGTCAAA | |
| 65D5 | GACATTGTGATGACCCAGACCCCCGCCTCCGTGAGCGAACCCGTGGGAGGAACCGTGACCATAAAATGCCAGGCCAGCCAAAGCATT | |
| | AGCAGCTATCTGGCATGGTATCAGCAGAAACCCGGACAGCCCCCCAAGCTGCTCATATACGAGGCAACCAAACTGACAAGCGGCGTG | |
| | AGCAGCAGATTCAGCGGCAGCGGCTACGGCACCGAGTTCACCCTGACTATCAGCGGCGTGGAATGCGCCGACGCCGCCACATACTAC | |
| | TGCCAGCAGGCCCTGACCGTGGGAGACGTGGACAACCCCTTTGGAGGCGGCAGCGAGGTTGTCGTGAAG | |
| 3E9 | GACGTCGTGATGACCCAGACACCAGCCTCCGTGTCTGCAGCTGTGGGAGGCACAGTCACCATCAAGTGCCAGGCCAGTGAGGACATT | |
| | GAAAGCTATTTAGCCTGGTATCAGCAGAAACCAGGGCAGCCTCCCAAGCTCCTGATCTATGATGCATCCACTCTGGCATCTGGGGTC | |
| | TCATCGCGGTTCAGCGGCAGTGGATATGGGACAGAGTTCACTCTCACCATCAGCGGCGTGGAGTGTGCCGATGCTGCCACTTACTAC | |
| | TGTCAACAGGATTTGACTGTTGGGAATGTTGATAATCCTTTCGGCGGAGGGAGCGAGGTGGTGGTCGAA | |
| 9A5 | GACGTCGTGATGACCCAGACTCCAGCCTCTGTGGAGGCAGCTGTGGGAGGCACAGTCACCATCAATTGCCAGGCCAGTCAGAGCATT | |
| | GAAAGCTATTTAGCCTGGTATCAGCAGAAACCAGGGCAGCCTCCCAAACTCCTGATCTATGAAGCATCCAAACTGGCATCTGGGGTC | |
| | TCATCGCGGTTCAGCGGCGGTGGATATGGGACAGAGTTCACTCTCACCATCAGCGGCGTGGAGTGTGCCGATGCTGCCACTTACTAC | |
| | TGTCAACAGGCTTTGACTATTGGTAATGTTGATAATCCTTTCGGCGGAGGAAGCGAGGTGGTGGTCAAA | |
| 12G6 | GACGTCGTGATGACCCAGACTCCAGCCTCTGTGGAGGTAGCTGTGGGAGGCACAGTCACCATCAAGTGCCAGGCCAGTGAGGACATT | |
| | GAAAGCTATTTAGCCTGGTATCAGCAGAAACCAGGGCAGCCTCCCAAACTCCTGATCTATGAAGCATCCAAACTGGCATCTGGGGTC | |
| | TCATCGCGGTTCAGCGGCGGTGGATATGGGACAGAGTTCACTCTCACCATCAGCGGCGTGGAGTGTGCCGATGCTGCCACTTACTAC | |
| | TGTCAACAGGCTTTGACTATTGGTAATGTTGATAATCCTTTCGGCGGAGGAAGCGAGGTGGTGGTCAAA | |
| 1D10 | GATGTGGTGATGACCCAGACCCCCGCCTCTGTGGAAGTGGCCGTGGGCGGAACCGTGACCATTAAATGTCAGGCCTCTGAAGATATT | |
| | GAGTCCTATCTGGCCTGGTACCAGCAGAAACCCGGCCAGCCCCCTAGACTGCTGATTTACGAGGCCTCCAAGCTGGCTAGTGGCGTG | |
| | AGCTCCAGATTTAGCGGAGGGGGATACGGCACAGACTTCACCCTGACTATCAGCGGCGTGGAGTGCGCCGACGCCGCTACTTACTAC | |
| | TGCCACCAGGCCCTGACCATTGGAAATGTGGACAACCCATTCGGGGGAGGCGGAGAGGTGGTGGTGAAG | |
| 62E3 | GACGTGGTGATGACCCAAACAGCCAGCCCCGTGAGCGCCGCCGTGGGAGGAACAGTGACCATTAAGTGTCAGGCAAGCGAGGACATC | |
| | GAGAGCTACCTGGCCTGGTATCAGCAGAAACCCGGCCAGCCCCCCAAACTCCTGATCTACGAGGCATCCAAGCTGGCCAGCGGCGTG | |
| | AGCAGCAGATTCAGCGGAAGCGGCTACGGCACAGAATTTACCCTGACTATCTCCGGCGTGGAATGCGCCGACGCCGCCACTTACTAC | |
| | TGCCAGCAGGCCCTGACTATCGGAAACGTTGACAACCCCTTTGGAGGAGGATCTGAGGTTGTGGTGAAG | |
| 3A4 | GACGTCGTGATGACCCAGACACCAGCCTCCGTGTCTGCAGCTGTGGGAGGCACAGTCAGCATCAATTGCCAGGCCAGTGAGGACATT | |
| | GAAAACTATTTAGCCTGGTATCAGCAGAAACCAGGGCAGCCTCCCAAACTCCTGATCTATGAAGCATCCAAACTGGCATCTGGGGTC | |
| | TCATCGCGGTTCAGCGGCAGTGGATATGGGACAGAGTTCACTCTCACCATCAGCGGCGTGGAGTGTGCCGATGCTGCCACTTACTAC | |
| | TGTCAACAGGCTTTGACTATTGGTAATGTTGATAATCCTTTCGGCGGAGGAAGCGAGGTGGTGGTCAAA | |
| 63H3 | GACGTGGTGATGACCCAAACACCCGCCAGCGTGGAAGTGGCCGTGGGAGGAACAGTGACCCTGAAGTGCCAGGCAAGCGAGGACATT | |
| | GAAAGCTACAGCGCCTGGTATCAACAAAAACCCGGCCAGCCCCCCAACCTGCTCATCTACGAGGCCTCCAAGCTGGCAAGCGGCGTG | |
| | AGCTCCAGATTCAGCGGATCAGGCTACGGCACAGAATTTACACTGACCATCAGCGGCGTGGAATGCGCCGACGCCGCCACATACTAC | |
| | TGCCAGCAGGCCCTGACTATAGGCAACGTGGACAACCCCTTCGGAGGCGGCAGCGAGGTGGTGGTGAGG | |
| 66A1 | GACGTGGTGATGACCCAAACACCCGCCAGCGTGGAAGCCGCCGTTGGAGGAACAGTTAGCATTAACTGCCAGGCCAGCGAGGACATT | |
| | GAAAGCTATCTGGCCTGGTACCAGCAGAAGCCCGGACAGCCCCCCAAACTGCTGATTTACAAGGCATCTACACTGGCAAGCGGCGTG | |
| | TCCAGCAGATTCAAAGGAAGCGGCAGCGGCAAGCAGTTCACCCTGACTATCAGCGGCGTGGAATGCGCCGATGCCGCCACCTACTAC | |
| | TGCCAGCAGGTCCTCACCATAGGAAACGTGGACAACCCCTTCGGAGGCGGAAGTGAAGTGGTCGTCAAG | |
| 2E12 | GACGTCGTGATGACCCAGACTCCAGCCTCTGTGGAGGTAGCTGTGGGAGGCACAGTCACCATCAAGTGCCAGGCCAGTGAGAACATT | |
| | GAAAGCTATTTAGCCTGGTATCAGCAGAAACCAGGGCAGCCTCCCAAGCTCCTGATCTATGATGCATCCACTCTGGCATCTGGGGTC | |
| | TCATCGCGGTTCAGCGGCAGTGGATATGGGACAGAGTTCACTCTCACCATCAGCGGCGTGGAGTGTGCCGATGCTGCCACTTACTAC | |
| | TGTCAACAGGGTTTGACTATTGGTAATGTTGATAATCCTTTCGGCGGAGGAAGCGAGGTGGTGGTCAAA | |
| 72F12 | GACGTGGTGATGACCCAAACACCCGCCAGCGTGGAAGTGGCCGTGGGAGGAACAGTGACCATTAAGTGCCAGGCCAGCAAGAACATA | |
| | GACAGCAACCTGGCTTGGTATCAGCAAAAACCCGGGCAGCCCCCCAAACAACTGATATACGCTGCCTCCACACTGGCCAGCGGCGTG | |
| | AGCAGCAGATTCAGCGGAAGCGGCTACGGAGCAGAGTTCACCCTGACTATCAGCGGCGTGGAATGCGCAGACGCCGCAACCTATTAC | |
| | TGCCAACAGGCCCTGACTATTGGCAACGTGGACAACCCCTTTGGAGGCGGAAGTGAGGTGGTGGTGAAG | |
| 74F11 | GACGTGGTGATGACCCAAACACCCGCCAGCGTGGAAGCCGCCGTTGGAGGAACAGTTACCATTAAATGCCAGGCCAGCGAGGACATT | |
| | GAAAGATATTTGGCCTGGTATCAGCAAAAACCCGGCCAACCCCCCAAACTGCTGATTTACGAGGCCTCCAAACTGCCAAGCGGCGTG | |
| | AGCAGCAGATTCAGCGGATCAGGCTATGGCACCGAATTTACCCTGACTATCTCCGGCGTGGAATGTGCCGACGCCGCCACCTACTAC | |
| | TGCCAGCAGGCCCTGACCATAGGCTATGTCGACAACCCCTTCGGAGGCGGCTCTGAGGTTGTGGTGAAG | |
| 15F5 | GACGTCGTGATGACCCAGACTCCAGCCTCTGTGGAGGCAGCTGTGGGAGGCACAGTCACCATCAAGTGCCAGGCCAGTGAGGACATT | |
| | GAAAGGTATTTAGCCTGGTATCAGCAGAAACCAGGGCAGCCTCCCAAGCTCCTGATTTATGAAGCATCCAAACTGCCATCTGGGGTC | |
| | TCATCGCGGTTCAGCGGCAGTGGATATGGGACAGAGTTCACTCTCACCATCAGCGGCGTGGAGTGTGCCGATGCTGCCACTTACTAC | |
| | TGTCAACAGGCTTTGACTATTGGTTACGTTGATAATCCTTTCGGCGGAGGAAGCGAGGTGGTGGTCAAA | |
| 9D7 | TATGATATGACCCAGACCCCCGCCAGTGTGTCCGAGCCCGTGGGAGGAACCGTGACAATTAAATGTCAGGCTAGTGAGGATATTTAT | |
| | AAACTGCTGGCCTGGTACCAGCAGAAGCCCGGCCAGCCCCCAAAACTGCTGATTTACGCCGCCAGCGACCTGGAAAGCGGCGTGCCA | |
| | AGCAGATTCAAGGGAAGAGGAAGCGGAACCGACTACACCCTGACTATCAGCGACCTGGAGTGTGCCGACGCCGCCACCTACTACTGC | |
| | CAGCAGGCCTATACTATCGGAAATATCGACAACAGCTTCGGAGGAGGAACAGAAGTGGTGGTGAAG | |
| 9D7-2 | TATGATATGACCCAGACCCCCGCCAGTGTGTCCGAGCCCGTGGGAGGAACCGTGACAATTAAATGTCAGGCTAGTGAGGATATTTAT | |
| | AAACTGCTGGCCTGGTACCAGCAGAAGCCCGGCCAGCCCCCAAAACTGCTGATTTACGCCGCCAGCGACCTGGAAAGCGGCGTGCCA | |
| | AGCAGATTCAAGGGAAGAGGAAGCGGAACCGACTACACCCTGACTATCAGCGACCTGGAGGCCGCCGACGCCGCTACATACTACTGT | |
| | CAGCAGGCCTATACTATCGGAAACATTGACAACAGCTTCGGCGGAGGAACAGAAGTGGTGGTGAAG | |
| 10A9 | TATGATATGACCCAGACCCCCGCCAGTGTGTCCGCCGCTGTGGGAGGAACCGTGACAATTAAATGTCAGGCTAGTGAGGATATTTAT | |
| | TCCCTGCTGGCCTGGTACCAGCAGAAGCCCGGCCAGCCCCCCAAGCTGCTGATTTACGGCGCCTCTAATCTGGAATCTGGCGTGCCC | |
| | AGCAGATTTAAGGGCTCCGGCAGCGGCACCGAGTACACCCTGACTATCAGCGACCTGGAATGCGACGACGCCGCCACCTACTACTGC | |
| | CAGCAGGCTTACACCATCGGCAACATCGACAACGCCTTCGGAGGAGGCACAGAAGTGGTGGTGAAG | |
| 10A9-2 | TATGATATGACCCAGACCCCCGCCAGTGTGTCCGCCGCTGTGGGAGGAACCGTGACAATTAAATGTCAGGCTAGTGAGGATATTTAT | |
| | TCCCTGCTGGCCTGGTACCAGCAGAAGCCCGGCCAGCCCCCCAAGCTGCTGATTTACGGCGCCTCTAATCTGGAATCTGGCGTGCCC | |
| | AGCAGATTTAAGGGCTCCGGCAGCGGCACCGAGTACACCCTGACTATCAGCGACCTGGAAGCTGACGACGCCGCCACCTACTACTGC | |
| | CAGCAGGCTTACACCATCGGCAACATCGACAACGCCTTCGGCGGCGGCACCGAGGTGGTGGTGAAG | |
| 62A2 | TATGATATGACCCAGACTCCAGCCTCCGTGTCTGCAGCTGTGGGAGGCACAGTCACCATCAAGTGCCAGGCCAGTGAGGACATTTAT | |
| | AGGTTATTGGCCTGGTATCAGCAGAAACCAGGGCAGCCTCCCAAGCTCCTGATCTATGGTGCATCCAATCTGGAATCTGGGGTCCCA | |
| | TCGCGGTTCAAAGGCAGTGGATCTGGGACAGAGTACACTCTCACCATCAGCGACCTGGAGTGTGACGATGCTGCCACTTACTACTGT | |
| | CAACAGGCTTATACTATTGGTAATATTGATAATGCTTTCGGCGGAGGGACCGAGGTGGTGGTCAAA | |
| 74E4 | TATGATATGACCCAGACTCCAGCCTCCGTGGAGGCAGCTGTGGGAGGCACAGTCACCATCAAGTGCCAGGCCAGTGAGGACATTTAT | |
| | AGGTTATTGGCCTGGTATCAGCAGAAACCAGGGCAGCCTCCCAAGCTCCTGATCTATGGTGCATCCAATCTGGAATCTGGGGTCCCA | |
| | TCGCGGTTCAAAGGCAGTGGATCTGGGACAGAGTACACTCTCACCATCAGCGACCTGGAGTGTGACGATGCTGCCACTTACTACTGT | |
| | CAACAGGCTTATACTATTGGTAATATTGATAATGCTTTCGGCGGAGGGACCGAGGTGGTGGTCAAA | |
| 66A6 | TATGATATGACCCAGACTCCAGCCTCCGTGTCTGCAGCTGTGGGAGGCACAGTCACCATCAAGTGCCAGGCCAGTGAGGACATTTAT | |
| | AGCTTATTGGCCTGGTATCAGCAGAAACCAGGGCAGCCTCCCAAGCTCCTGATCTATGATGCATCCGATCTGGCATCTGGGGTCCCA | |
| | TCGCGGTTCAAAGGCAGTGGATCTGGGACAGAGTACACTCTCACCATCAGCGACCTGGAGTGTGACGATGCTGCCACTTACTACTGT | |
| | CAACAGGCTTATACTATTGGTAATATTGATAATACTTTCGGCGGAGGGACCGAGGTGGTGGTCAAA | |
| 10E12 | TATGATATGACCCAGACTCCAGCCTCCGTGTCTGCAGCTGTGGGAGGCACAGTCACCATCAAGTGCCAGGCCAGTGAGGACATTTAT | |
| | AGCTTATTGGCCTGGTATCAGCAGAAACCAGGGCAGCCTCCCAAGCTCCTGATCTATGATGCATCCGATCTGGCATCTGGGGTCCCA | |
| | TCGCGGTTCAAAGGCAGTGGATCTGGGACAGAGTACACTCTCACCATCAGCGACCTGGAGTGTGACGATGCTGCCACTTACTACTGT | |
| | CAACAGGCTTATACTATTGGTAATATTGATAATGCTTTCGGCGGAGGGACCGAGGTGGTGGTCAAA | |
| 8H2 | TATGATATGACCCAGACTCCAGCCTCCGTGTCTGCAGCTGTGGGAGGCACAGTCACCATCAAGTGCCAGGCCAGTGAGGACATTTAT | |
| | AGCTTATTGGCCTGGTATCAGCAGAAACCAGGGCAGCCTCCCAAGCTCCTGATCTATGATGCATCCGATCTGGCATCTGGGGTCCCA | |
| | TCGCGGTTCAAAGGCAGTGGATCTGGGACAGAGTACACTCTCACCATCAGCGCCCTGGAGTGTGACGATGCTGCCACTTACTACTGT | |
| | CAACAGGCTTATACTATTGGTAATATTGATAATACTTTCGGCGGAGGGACCGAGGTGGTGGTCAAA | |
| 74H11 | TATGATATGACCCAGACTCCAGCCTCCGTGTCTGCAGCTGTGGGAGGCACAGTCACCATCAAGTGCCAGGCCAGTGAGGACATTTAT | |
| | AGCTTATTGGCCTGGTATCAGCAGAAACCAGGGCAGCCTCCCAAGCTCCTGATCTATGATGCATCCGATCTGGCATCTGGGGTCCCA | |
| | TCGCGGTTCAAAGGCAGTGGATCTGGGACAGAGTACACTCTCACCATCAGCGACCTGGAGTGTGACGATGCTGCCACTTACTACTGT | |
| | CAACAGGCTTATACTATTGGTAATATTGATAATGCTTTCGGCGGAGGGACCGAGGTGGTGGTCAAA | |
| 74A1 | TATGATATGACCCAGACTCCAGCCTCCGTGTCTGAACCTGTGGGAGGCACAGTCACCATCAAGTGCCAGGCCAGTCAGAGCATTTAC | |
| | AATAATTTTGCCTGGTATCAGCAGAAACCAGGGCAGCGTCCCAAGCTCCTGATCTATGATGCATCCGATCTGGCATCTGGGGTCCCA | |
| | TCGCGGTTCAAAGGCAGTGGATCTGGGACAGAGTACACTCTCACCATCAGCGGCGTGCAGTGTGCCGATGCAGCCACTTACTACTGT | |
| | CAACAGGCTTATACTATTGGTAATATTGATAATCCTTTCGGCGGAGGGACCGAGGTGGTGGTCAAA | |
| 10F12 | TATGATATGACCCAGACTCCAGCCTCCGTGTCTGCAGCTGTGGGAGGCACAGTCACCATCAAGTGCCAGGCCAGTGAGGACATTTAT | |
| | AGCTTATTGGCCTGGTATCAGCAGAAACCAGGGCAGCCTCCCAAGCTCCTGATCTTTGATGCATCCGATCTGGCATCTGGGGTCCCA | |
| | TCGCGGTTCAAAGGCAGTGGATCTGGGACAGAGTACACTCTCACCATCAGCGACCTGGAGTGTGACGATGCTGCCACTTACTACTGT | |
| | CAACAGGCTTATACTATTGGTAATATTGATAATGCTTTCGGCGGAGGGACCGAGGTGGTGGTCAAA | |
| 65E11 | TATGATATGACCCAGACTCCAGCCTCCGTGTCTGCAGCTGTGGGAGGCACAATCACCATCAAGTGCCAGGCCAGTGAGGACATTTAT | |
| | AGCTTATTGGCCTGGTATCAGCAGAAACCAGGGCAGCCTCCCAAGCTCCTGATCTTTGATGCATCCACTCTGGCATCTGGGGTCCCA | |
| | TCGCGGTTCAAAGGCAGTGGATCTGGGACAGAGTACACTCTCACCATCAGCGACCTGGAGTGTGACGATGCTGCCACTTACTACTGT | |
| | CAACAGGCTTATACTATTGGTAATATTGATAATGCTTTCGGCGGAGGGACCGAGGTGGTGGTCGAA | |
| 69C2 | TATGATATGACCCAGACTCCAGCCTCCGTGTCTGAACCTGTGGGAGGCACAGTCACCATCAAGTGCCAGGCCAGTGAGGACATTTAT | |
| | AAGTTATTGGCCTGGTATCAGCAGAAACCAGGGCAGCCTCCCAAGCTCCTGATCTATGCTGCATCCGATCTGGAATCTGGGGTCCCA | |
| | TCGCGGTTCAAAGGCAGTGGATCTGGGACAGAGTACACTCTCACCATCAGCGACCTGGAGTGTGCCGATGCTGCCACTTACTACTGT | |
| | CAACAGGCTTATACTATTGGTAATATTGATAATACTTTCGGCGGAGGGACCGAGGTGGTGGTCAAA | |
| 61F12 | TATGATATGACCCAGACTCCAGCCTCCGTGTCTGCAGCTGTGGGAGGCACAGTCACCATCAAGTGCCAGGCCAGTGAGGACATTTAT | |
| | AGTTTATTGGCCTGGTATCAGCAGAAACCAGGGCAGCCTCCCAAGCTCCTGATCTATGATGCATCCGATCTGGCATCTGGGGTCCCA | |
| | TCGCGGTTCAAAGGCAGTGGATCTGGGACAGAGTACACTCTCACCATCAGCGACCTGGAGTGTGACGATGCTGCCACTTACTACTGT | |
| | CAACAGGCTTATACTATTGGTAATATTGATAATTCTTTCGGCGGAGGGACCGAGGTGGTGGTCAAA | |
| 10D3 | TATGATATGACCCAGACTCCAGCCTCCGTGTCTGAACCTGTGGGAGGCACAGTCACCATCAAGTGCCAGGCCAGTGAGGACATTTAT | |
| | AGGTTATTGGCCTGGTATCAGCAGAAACCAGGGCAGCCTCCCAAGCTCCTGATCTATGATGCATCCGATCTGGCATCTGGGGTCCCA | |
| | TCGCGGTTCAAAGGCAGTGGATCTGGGACAGAGTACACTCTCACCATCAGCGGCTTGCAGTGTGCCGATGCAGCCACTTACTACTGT | |
| | CAACAGGGTTATACTATTGGTAATATTGATAATTCTTTCGGCGGAGGGACCGAGGTGGTGGTCAAA | |
| 67H4 | TATGATATGACCCAGACTCCAGCCTCCGTGTCTGAACCTGTGGGAGGCACAGTCACCATCAAGTGCCAGGCCAGTGAGGACATTTAT | |
| | AACTTATTGGCCTGGTATCAGCAGAAACCAGGGCAGCCTCCCAAGCTCCTGATCTATGCTGCATCCGATCTGGAATCTGGGGTCCCA | |
| | TCGCGGTTCAAAGGCAGTGGATCTGGGACAGAGTACACTCTCACCATCAGCGACCTGGAGTGTGACGATGCTGCCACTTACTACTGT | |
| | CAACAGGGTTATACTATTGGTAATATTGATAATGCTTTCGGCGGAGGGACCGAGGTGGTGGTCAAA | |
| 64G9 | TATGATATGACCCAGACTCCAGCCTCCGTGTCTGAACCTGTGGGAGGCACAGTCACCATCAAGTGCCAGGCCAGTGAGGACATTTAT | |
| | AACTTATTGGCCTGGTATCAGCAGAAACCAGGGCAGCCTCCCAAGCTCCTGATCTATGATGCATCCACTCTGACATCTGGGGTCCCA | |
| | TCGCGGTTCAAAGGCAGTGGATCTGGGACAGAGTACACTCTCACCATCAGCGACCTGGAGTGTGACGATGCTGCCACTTACTACTGT | |
| | CAACAGGCTTATACTATTGGTAATATTGATAATACTTTCGGCGGAGGGACCGAGGTGGTGGTCAAA | |
| 72G12 | TATGATATGACCCAGACTCCAGCCTCCGTGTCTGAACCTGTGGGAGGCACAGTCACCATCAAGTGCCAGGCCAGTGAGGACATTTAT | |
| | AAATTATTGGCCTGGTATCAGCAGAAACCAGGGCAGCCTCCCAAGCTCCTGATCTATGCTGCATCTGATCTGGAATCTGGGGTCCCA | |
| | TCGCGGTTCAAAGGCAGTGGATCTGGGACAGAGTATACTCTCACCATCAGCGACCTGGAGTGTGCCGATGCTGCCACTTACTACTGT | |
| | CAACAGGCTTATACTATCGGTAATCTTGATAATGCTTTCGGCGGAGGGACCGAGGTGGTGGTCAAA | |
| 65G8 | TATGATATGACCCAGACTCCAGCCTCCGTGTCTGAACCTGTGGGAGGCACAGTCACCATCAAGTGCCAGGCCAGTGAGGACATTTAT | |
| | AAGTTATTGGCCTGGTATCAGCAGAAACCAGGGCAGCCTCCCAAGCTCCTGATCTATGCTGCATCCGATCTGGAATCTGGGGTCCCA | |
| | TCGCGGTTCAAAGGCAGTGGATCTGGGACAGAGTACACTCTCACCATCAGCGACCTGGAGTGTGCCGATGCTGCCACTTACTACTGT | |
| | CAACAGGCTTATACTATTGGTAATATTGATAATACTTTCGGCGGAGGGACCGAGGTGGTGGTCAAA | |
| 69D8 | TATGATATGACCCAGACTCCAGCCTCCGTGTCTGCAGCTGTGGGAGGCACAATCACCATCAAGTGCCAGGCCAGTGAGGACATTTAT | |
| | AGCTTATTGGCCTGGTATCAGCAGAAACCAGGGCAGCCTCCCAAGCTCCTGATCTTTGATGCATCCACTCTGGCATCTGGGGTCCCA | |
| | TCGCGGTTCAAAGGCAGTGGATCTGGGACAGAGTACACTCTCACCATCAGCGACCTGGAGTGTGACGATGCTGCCACTTACTACTGT | |
| | CAACAGGCTTATACTATTGGTAATATTGATAATGCTTTCGGCGGAGGGACCGAGGIGGTGGTCGAA | |
| 2H5 | TATGATATGACCCAGACTCCAGCCTCCGTGTCTGAACCTGTGGGAGGCACAGTCACCATCAAGTGCCAGGCCAGTGAGGACATTTAT | |
| | AATTTATTGGCCTGGTATCAGCAGAAACCAGGGCAGCCTCCCAAGCTCCTGATCTATGCTGCATCCGATCTGGAATCTGGGGTCCCA | |
| | TCGCGGTTCAAAGGCAGTGGATCTGGGACAGAGTACACTCTCACCATCAGCGACCTGGAGTGTGCCGATGCTGCCACTTACTACTGT | |
| | CAACAGGCTTATACTGTCGGTAATATTGATAATGCTTTCGGCGGCGGGACCGAGGIGGTGGTCAAA | |
| 64A6 | TATGATATGACCCAGACTCCAGCCTCCGTGTCTGAACCTGTGGGAGGCACAGTCACCATCAAGTGCCAGGCCAGTGAGGACATTTAT | |
| | AAGTTATTGGCCTGGTATCAGCAGAAACCAGGGCAGCCTCCCAAGCTCCTGATCTATGCTGCATCCGATCTGGAATCTGGGGTCCCA | |
| | TCGCGGTTCAAAGGCAGTGGATCTGGGACAGACTACACTCTCACCATCAGCGACCTGGAGTGTGCCGATGCTGCCACTTACTACTGT | |
| | CAACAGGCITATACTATCGGTAATATTGATAATTCTTTCGGCGGAGGGACCGAGGIGGTGGTCAAA | |
| 62B10 | TATGATATGACCCAGACTCCAGCCTCCGTGTCTGAACCTGTGGGAGGCACAGTCACCATCAAGTGCCAGGCCAGTGAGGACATTTAT | |
| | AACTTATTGGCCTGGTATCAGCAGAAACCAGGGCAGCCTCCCAAGCTCCTGATCTATGATGCGTCCACTCTGACATCTGGGGTCCCA | |
| | TCGCGGTTCAAAGGCGGTGGATCTGGGACAGAGTACACTCTCACCATCAACGACCTGGAGTGTGACGATGCTGCCACTTACTACTGT | |
| | CAACAGGGTTATACTATTGGTAATATTGATAATACTTTCGGCGGAGGGACCGAGGTGGTGGTCAAA | |
| 5D4 | TTTGAAATGACCCAGACCCCCTCCTCCGTGAGCGAACCCGTGGGCGGAACCGTGACCATCAAGTGTCAGGCCTCCGAGGACATCTCC | |
| | TCCAATCTGGGATGGTATCAGCAGAAACCCGGCCAGCCCCCCAAGCTGCTGATCTATGGCGCCAGCACCCTGGCCAGCGGCGTGCCT | |
| | TCTAGGTTTAAGGGCAGCGGCAGCGGCACCGAGTTCACCCTGACTATCAGCGACCTGGAATGCGCCGACGCCGCCACCTACTACTGC | |
| | CAGACCAGCTACTACATTGACGATGGCGTGAACGGATTTGGCGGAGGCACAGAGGIGGTGGTGAAG | |
| 5D4-1 | TTTGAAATGACCCAGACCCCCTCCTCCGTGAGCGAACCCGTGGGCGGAACCGTGACCATCAAGTGTCAGGCCTCCGAGGACATCTCC | |
| | TCCAATCTGGGATGGTATCAGCAGAAACCCGGCCAGCCCCCCAAGCTGCTGATCTATGGCGCCAGCACCCTGGCCAGCGGCGTGCCT | |
| | TCTAGGTTTAAGGGCAGCGGCAGCGGCACCGAGTTCACCCTGACTATCAGCGACCTGGAACCCGCCGACGCCGCCACTTACTACTGC | |
| | CAGACCAGCTACTACATTGACGATGGGGTGAACGGATTTGGAGGGGGAACCGAGGTGGTGGTGAAG | |
| 5D4-2 | TTTGAAATGACCCAGACCCCCTCCTCCGTGAGCGAACCCGTGGGCGGAACCGTGACCATCAAGTGTCAGGCCTCCGAGGACATCTCC | |
| | TCCAATCTGGGATGGTATCAGCAGAAACCCGGCCAGCCCCCCAAGCTGCTGATCTATGGCGCCAGCACCCTGGCCAGCGGCGTGCCT | |
| | TCTAGGTTTAAGGGCAGCGGCAGCGGCACCGAGTTCACCCTGACTATCAGCGACCTGGAAGCTGCCGACGCCGCCACTTACTACTGC | |
| | CAGACCAGCTACTACATCGACGATGGGGTGAATGGCTTCGGCGGAGGCACAGAGGTGGTGGTGAAG | |
| 70G2 | TTCGAGATGACCCAGACTCCAGCCTCCGTGTCTGAACCTGTGGGAGGCACAGTCACCATCAAGTGCCAGGCCAGTGAGGATATTAGT | |
| | AGTAATTTAGCCTGGTATCAGCAGAAACCAGGACAGCCTCCCAAGCTCCTGATCTATGGTGCATCCACTCTGGCATCTGGGGTCCCA | |
| | TCGCGGTTCAAAGGCAGTGGATCTGGGACAGAGTTCACTCTCACCATCAGCGACCTGGAGTGTGCCGATGCTGCCACTTACTACTGT | |
| | CAAAGTTCTTATTATATTGATGATGGTGTAAATGGTTTCGGCGGAGGGACCGAGGTGGTGGTCAAA | |
| 4F2 | TATGATATGACCCAGACCCCCGCCAGTGTGGAAGTGACCGTGGGCGGAACCGTGACCATCAATTGTCAGGCCTCCGAATCCCTGTCC | |
| | TCCTATCTGGCCTGGTACCAGCAGAAACCCGGCCAGCCCCCCAAGCCCCTGATCTACAGAGCCGCCACCCTGGCCAGCGGCGTGCCT | |
| | AGCAGATTTAAGGGCTCCGGCTCCGGCACCGACTACACCCTGACCATCAGCGACCTGGAATGCGCCGACGCCGCCACCTACTACTGC | |
| | CAGCAGGGATACGGCTACAGCACCGTGGGCAACGCCTTCGGCGGCGGAACAGAGGIGGTGGTGAAG | |
| 62A10 | TATGACATGACCCAGACTCCAGCCTCTGTGGAGGTAGCTGTGGGAGGCACAGTCACCATCAATTGCCAGGCCAGTGAGAGCATTAGT | |
| | CGCTACTTAGCCTGGTATCAGCAGAAACCAGGGCAGTCTCCCAAGCCCCTGATCTACAGGGCTTCCACTCTGGCATCTGGGGTCCCA | |
| | TCACGATTCCAAGGCAGTGGATCTGGGACAGAATACACTCTCACCATCAGCGACCTGGAGTGTGCCGATGCTGCCACTTACTACTGT | |
| | CAACAGGGTTATGGTTATAGTACTGTTGACAATGCTTTCGGCGGAGGGACCGAGGTGGTGGTCAAA | |
| 70E2 | TATGACATGACCCAGACTCCAGCCTCTGTGGAGGTACCTGTGGGAGGCACAGTCACCATCAATTGCCAGGCCAGTGAGAACATTAAT | |
| | AGATACTTAGCCTGGTATCAGCAGAAACCAGGGCAGCCTCCCAAGCCCCTGATCTACAGGGCAGCCACTCTGGCATCTGGGGTCCCA | |
| | TCGCGGTTCAAAGGCAGTGGATCTGGGACAGAGTACACTCTCAGCATCAGCGACCTGGAGTGTGCCGATGCTGCCACTTACTACTGT | |
| | CAACAGGGTTATGGTTATAGTACTGTTGGGAATGCTTTCGGCGGAGGGACCGAGGTGGTGGTCAAA | |
| 71F10 | TATGACATGACCCAGACTCCAGCCTCGGTGTCTGCAGCTGTGGGAGGCACAGTCACCATCAATTGCCAGGCCAGTGAGAGCATTAGT | |
| | AGGTACTTAGCCTGGTATCAGCAGAAACCAGGGCAGCCTCCCAAGCCCCTGATCTACAGGGCATCCACTCTGGCATCTGGGGTCCCA | |
| | TCGCGGTTCAAAGGCAGTGGAACTGGGACAGACTACACTCTCACCATCAGCGACCTGGAGTGTGCCGATGCTGCCACTTACTACTGT | |
| | CAACAGGGTTATGGTTATAGTACTGTTGGGAATGCTTTCGGCGGAGGGACCGAGGTGGTGGTCAAA | |
| 73C2 | TATGACATGACCCAGACTCCAGCCTCTGTGGAGGTAGCTGTGGGAGGCACAGTCACCATCAATTGCCAGGCCAGTGAGAGCATTAAT | |
| | AGATACTTAGCCTGGTATCAACAGAAACGAGGGCAGCCTCCCAAGCCCCTGATCTACAGGGCAGCCACTCTGGCATCTGGGGTCCCA | |
| | TCGCGGTTCAAAGGCAGTGGATCTGGGACAGAGTACACTCTCACCATCAGCGACCTGGAGTGTGCCGATGCTGCCACTTACTACTGT | |
| | CAACAGGGTTATGGTTATAGTACTGTTGGGAATGCTTTCGGCGGAGGGACCGAGGTGGTGGTCAAA | |

In certain embodiments, the antigen binding molecules of the present invention, e.g., an antibody or an antigen binding fragment thereof, are modified after translation. Examples of the posttranslational modification include cleavage of lysine at the C terminal of the heavy chain by a carboxypeptidase; modification of glutamine or glutamic acid at the N terminal of the heavy chain and the light chain to pyroglutamic acid by pyroglutamylation; glycosylation; oxidation; deamidation; and glycation, and it is known that such posttranslational modifications occur in various antibodies (See journal of Pharmaceutical Sciences, 2008, Vol. 97, p. 2426-2447, incorporated by reference in its entirety). Examples of an antigen binding molecule, e.g., an antibody or antigen binding fragment thereof which have undergone posttranslational modification include an antigen binding molecule, e.g., an antibody or antigen binding fragments thereof which have undergone pyroglutamylation at the N terminal of the heavy chain variable region and/or deletion of lysine at the C terminal of the heavy chain. The sequences of the heavy chains of exemplary antigen binding molecules that undergo pyroglutamylation at the N-terminus are listed in Table 18. As used herein, “pE” refers to pyroglutamic acid when used to represent an amino acid in a polypeptide.

### 2. Variants of Antigen Binding Molecules

In certain embodiments, the Trop-2 antigen binding molecules of the present invention, e.g., the anti-Trop-2 antibodies, can be a monoclonal antibody, a chimeric antibody, a humanized antibody, a Fab, a (Fab)₂, a scFv or a multi-specific antibody comprising additional binding specificities described herein.

Accordingly, in certain embodiments the anti-Trop-2 antibodies described herein may be linked to an Fc comprising one or more modifications, typically to alter one or more functional properties of the antibody, such as serum half-life, complement fixation, Fc receptor binding, and/or antigen-dependent cellular cytotoxicity. Furthermore, an antibody described herein may be chemically modified (e.g., one or more chemical moieties can be attached to the antibody) or it may be modified to alter its glycosylation, to alter one or more functional properties of the antibody. More specifically, in certain embodiments, the antibodies in the present invention may include modifications in the Fc region in order to generate an Fc variant with (a) increased or decreased antibody-dependent cell-mediated cytotoxicity (ADCC), (b) increased or decreased complement mediated cytotoxicity (CDC), (c) increased or decreased affinity for Clq and/or (d) increased or decreased affinity for a Fc receptor relative to the parent Fc. Such Fc region variants will generally comprise at least one amino acid modification in the Fc region. Combining amino acid modifications is thought to be particularly desirable. For example, the variant Fc region may include two, three, four, five, etc. substitutions therein, e.g., of the specific Fc region positions identified herein.

For uses where effector function is to be desirable, certain effector function enhancing version of the Fc may be used. In some embodiments, when the antibody is intended to induce ADCC, ADCC-enhancing version of IgG1 S239D, A330L, 1332E or S298A, E333A, K334A or F243L, R292P, Y300L, V305I, P396L may be used. In certain embodiments, to enhance ADCC in a “Y” shaped bivalent antibody, the substitutions L234Y, L235Q, G236W, S239M, H268D, D270E, S298A are introduced into one heavy chain, and the substitutions D270E, K326D, A330M, K334E are introduced into the other heavy chain.

In some embodiments, when the antibody is intended to induce ADCP, the ADCP enhancing version of G236A, S239D, 1332E may be used. In certain embodiment, when the antibody is intended to induce CDC, the CDC enhancing version of K326W, E33S, or S267E, H268F, S324T, or E345R, E430G, S440Y may be used.

For uses where effector function is to be avoided altogether, e.g., when antigen binding alone is sufficient to generate the desired therapeutic benefit, and effector function leads to (or increases the risk of) undesired side effects, IgG4 antibodies or ADCC-null version of IgG1 L234F, L235E, P331S or L234A, L235A, P239G may be used, or antibodies or fragments lacking the Fc region or a substantial portion thereof can be devised, or the Fc may be mutated to eliminate glycosylation altogether (e.g., N297A or N297Q or N297G). Alternatively, a hybrid construct of human IgG2 (CH1 domain and hinge region) and human IgG4 (C_{H}2 and C_{H}3 domains) may be generated that is devoid of effector function, lacking the ability to bind FcγRs (like IgG2) and activate complement (like IgG4). When using an IgG4 constant domain, it is usually preferable to include the substitution S228P which mimics the hinge sequence in IgG1 and R409K mutation which prevents Fab arm exchange and thereby stabilizes IgG4 molecules, reducing Fab-arm exchange between the therapeutic antibody and endogenous IgG4 in the patient being treated.

In certain embodiments, the anti-Trop-2 antibody or fragment(s) thereof may be modified to provide increased biological half-life. Various approaches may be employed, including e.g., those that increase the binding affinity of the Fc region for FcRn. In one embodiment, the antibody is altered within the CH1 or CL region to contain a salvage receptor binding epitope taken from two loops of a CH2 domain of an Fc region of an IgG, as described in U.S. Pat. Nos. 5,869,046 and 6,121,022. The numbering of residues in the Fc region is that of the EU index of Kabat. Sequence variants disclosed herein are provided with reference to the residue number followed by the amino acid that is substituted in place of the naturally occurring amino acid, optionally preceded by the naturally occurring residue at that position. Where multiple amino acids may be present at a given position, e.g., if sequences differ between naturally occurring isotypes, or if multiple mutations may be substituted at the position, they are separated by slashes (e.g., “X/Y/Z”).

Exemplary Fc variants that increase binding to FcRn and/or improve pharmacokinetic properties include substitutions at positions 259, 308, and 434, including for example 2591, 308F, 428L, 428M, 434S, 434H, 434F, 434Y, and 434M. Other variants that increase Fc binding to FcRn include: 250E, 250Q, 428L, 428F, 250Q/428L (Hinton et al., 2004, J. Biol. Chem. 279(8): 6213-6216, Hinton et al. 2006 Journal of Immunology 176:346-356), 256A, 272A, 305A, 307A, 311A, 312A, 378Q, 380A, 382A, 434A (Shields et al. (2001) J. Biol. Chem., 276(9):6591-6604), 252F, 252Y, 252W, 254T, 256Q, 256E, 256D, 433R, 434F, 434Y, 252Y/254T/256E, 433K/434F/436H (Dall'Acqua et al. (2002) J. Immunol., 169:5171-5180, Dall'Acqua et al. (2006) J. Biol. Chem., 281:23514-23524, and U.S. Pat. No. 8,367,805.

Modification of certain conserved residues in IgG Fc (I253, H310, Q311, H433, N434), such as the N434A variant (Yeung et al. (2009) J. Immunol. 182:7663), have been proposed as a way to increase FcRn affinity, thus increasing the half-life of the antibody in circulation (WO 98/023289). The combination Fc variant comprising M428L and N434S has been shown to increase FcRn binding and increase serum half-life up to five-fold (Zalevsky et al. (2010) Nat. Biotechnol. 28:157). The combination Fc variant comprising T307A, E380A and N434A modifications also extends the half-life of IgG1 antibodies (Petkova et al. (2006) Int. Immunol. 18:1759). In addition, combination Fc variants comprising M252Y-M428L, M428L-N434H, M428L-N434F, M428L-N434Y, M428L-N434A, M428L-N434M, and M428L-N434S variants have also been shown to extend half-life (U.S. 2006/173170). Further, a combination Fc variant comprising M252Y, S254T and T256E was reported to increase half-life-nearly 4-fold. Dall'Acqua et al. (2006) J. Biol. Chem. 281:23514.

In certain embodiments, the Trop-2 antigen binding molecule of the present invention is a bispecific antibody, comprising: a first targeting domain that binds specifically to Trop-2 and a second targeting domain that binds specifically another epitope in Trop-2 or another protein. In some embodiments, the first targeting domain includes an antigen binding fragment from any of the Trop-2 antibodies of the present invention. In certain embodiments, the first targeting domain of the bispecific antibody binds specifically to Trop-2 and the second targeting domain specifically binds to a protein expressed on a surface of an immune cell, such as a T cell, a NK cell, a NK T cell, or a macrophage. Without wishing to be bound by any theory, it is hypothesized that a bispecific antibody may bind to Trop-2 on the surface of a tumor cell and a protein expressed on a surface of an immune cell. The bispecific antibody thus facilitates the killing of the tumor cell by the immune cell.

In certain embodiments, the antigen binding molecules, e.g., anti-Trop-2 antibodies or antigen binding fragments thereof, of the present invention are chemically conjugated to one or more agents. In certain embodiments, the agent is a therapeutically active agent, e.g., therapeutic peptides and/or small molecule drugs to form a conjugate, e.g., an antibody-drug conjugate (ADC). In certain embodiment, the agent is a diagnostic agent. Methods for making covalent or non-covalent conjugates of the agent, e.g., the peptides or small molecule drugs with antibodies are known in the art and any such known method may be utilized. In a conjugate, a peptide is an oligopeptide which includes no more than twenty (20) amino acids. Without wishing to be bound by any theory, it is hypothesized that the ADC according to the present disclosure can bind to Trop-2 expressed on the surface of a tumor cell. The internalization of the antibody-antigen complex thus introduces the drug into a tumor cell, thereby the tumor cell may be killed by the drug conjugated to the Trop-2 of the present disclosure.

In some embodiments, the peptide or small molecule drug is attached to the hinge region of a reduced antibody component via disulfide bond formation. Alternatively, such agents can be attached using a heterobifunctional cross-linkers, such as N-succinyl 3-(2-pyridyldithio)propionate (SPDP). General techniques for such conjugation are well-known in the art. In some embodiments, the peptide or small molecule drug is conjugated via a carbohydrate moiety in the Fc region of the antibody. The carbohydrate group can be used to increase the loading of the same agent that is bound to a thiol group, or the carbohydrate moiety can be used to bind a different therapeutic or diagnostic agent. Methods for conjugating peptide inhibitors or small molecule drugs to antibodies via antibody carbohydrate moieties is well-known to those of skill in the art. For example, in one embodiment, the method involves reacting to an antibody component having an oxidized carbohydrate portion with a carrier polymer that has at least one free amine function. This reaction results in an initial Schiff base (imine) linkage, which can be stabilized by reduction to a secondary amine to form the final conjugate. Exemplary methods for conjugating small molecule drugs and peptides to antibodies are described in U.S. Patent Application Publication No. 2014/0356385.

In certain embodiments, the antigen binding molecule, e.g., an antibody or antigen binding fragment thereof, of the present invention, is operably linked to a peptide or a protein to form a fusion protein. As used herein, a peptide or a protein in a fusion protein includes more than twenty amino acids.

The term “operably linked,” in the context of fusion protein, is intended to mean that the two or more polypeptides are linked such that each polypeptide can perform its intended function. In certain embodiments, the polypeptides are covalently linked via a peptide bond. In certain embodiments, the polypeptides are covalently linked via a linker. The term “linker,” refers to a molecule or group of molecules (such as a monomer or polymer) that connects two molecules and often serves to place the two molecules in a preferred configuration. A number of strategies may be used to covalently link molecules together. These include, but are not limited to polypeptide linkages between N- and C-terminus of proteins or protein domains, linkage via disulfide bonds, and linkage via chemical cross-linking reagents. In one aspect of this embodiment, the linker is a peptide bond, generated by recombinant techniques or peptide synthesis. In another embodiment the linker is a cysteine linker. In yet another embodiment it is a multi-cysteine linker. Choosing a suitable linker for a specific case where two polypeptide chains are to be connected depends on various parameters, including but not limited to the nature of the two polypeptide chains (e.g., whether they naturally oligomerize), the distance between the N- and the C-termini to be connected if known, and/or the stability of the linker towards proteolysis and oxidation. Furthermore, the linker may contain amino acid residues that provide flexibility. Thus, the linker peptide may predominantly include the following amino acid residues: Gly, Ser, Ala, or Thr. The linker peptide should have a length that is adequate to link two molecules in such a way that they assume the correct conformation relative to one another so that they retain the desired activity. Suitable lengths for this purpose include at least one and not more than 30 amino acid residues. In one embodiment, the linker is from about 1 to 30 amino acids in length. In another embodiment, the linker is from about 1 to 15 amino acids in length.

In addition, the amino acid residues selected for inclusion in the linker peptide should exhibit properties that do not interfere significantly with the activity of the polypeptide. Thus, the linker peptide on the whole should not exhibit a charge that would be inconsistent with the activity of the polypeptide, or interfere with internal folding, or form bonds or other interactions with amino acid residues in one or more of the monomers that would seriously impede the binding of receptor monomer domains. Useful linkers include glycine-serine polymers, glycine-alanine polymers, alanine-serine polymers, and other flexible linkers such as the tether for the shaker potassium channel, and a large variety of other flexible linkers, as will be appreciated by those in the art. Suitable linkers may also be identified by screening databases of known three-dimensional structures for naturally occurring motifs that can bridge the gap between two polypeptide chains. In one embodiment, the linker is not immunogenic when administered in a human subject. Thus, linkers may be chosen such that they have low immunogenicity or are thought to have low immunogenicity. Another way of obtaining a suitable linker is by optimizing a simple linker, e.g., (Gly4 Ser)n, through random mutagenesis. Alternatively, once a suitable polypeptide linker is defined, additional linker polypeptides can be created to select amino acids that more optimally interact with the domains being linked. Other types of linkers that may be used in the compositions and methods provided herein include artificial polypeptide linkers and inteins. In another embodiment, disulfide bonds are designed to link the two molecules. In another embodiment, linkers are chemical cross-linking agents. For example, a variety of bifunctional protein coupling agents may be used, including but not limited to N-succinimidyl-3-(2-pyridyldithiol) propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). In another embodiment, chemical linkers may enable chelation of an isotope. For example, Carbon-14-labeled 1-isothiocyanatobenzyl-3-methyldiethylene triaminepentaacetic acid (MX-DTPA) is an exemplary chelating agent for conjugation of radionucleotide to the antibody. The linker may be cleavable, facilitating release of the cytotoxic drug in the cell. For example, an acid-labile linker, peptidase-sensitive linker, dimethyl linker or disulfide-containing linker (Chari et al., 1992, Cancer Research 52: 127-131) may be used. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers, that may find use to link the components of the conjugates of the compositions and methods provided herein.

In certain embodiments, the fusion protein includes an antigen binding molecule, e.g., an antibody or antigen binding fragment thereof, of the present invention and a different protein or peptide. In certain embodiments, the different protein or peptide is a cytokine or chemokine or a functional domain of thereof. The term, “functional domain,” as used herein, refers to a portion of a protein that has one or more functions or properties thereof. For example, a “functional domain” of IL-2 includes a portion of the IL-2 cytokine, which can have one or more function of IL-2, such as promoting the growth and development of T cells, increasing the cell killing activity of both NK and cytotoxic T cells. In some embodiments, the cytokine or the chemokine regulate the infiltration of different immune cell subsets into tumors and, as such, affect tumor immunity and influence therapeutic outcomes in patients. In certain embodiments, the cytokine is selected from the group consisting of IL-2, IL-5, IL-7, IL-12, IL-15, IL-21, and GM-CSF. In some embodiments, the chemokine is selected from the group consisting of CXCL8, CXCL9, CXCL10, and CXCL14.

In certain embodiments, the different protein or peptide is a trap protein or peptide. As used herein, a “trap” protein or peptide refers to a polypeptide that blocks the binding a ligand of the polypeptide to the polypeptide. The polypeptide may be a receptor that, upon binding to a ligand thereof, induces immunosuppression of an immune cell. In some embodiments, the trap protein or peptide is an extracellular domain of transforming growth factor beta receptor.

In certain embodiments, the different protein or peptide is operably linked to the antigen binding molecule, e.g., the antibody or antigen binding fragment thereof, of the present invention. In certain embodiments, the different protein or peptide is operably linked to a C-terminus of the antigen binding molecule, e.g., the antibody or antigen binding fragment thereof. In certain embodiments, the different protein or peptide is operably linked to an N-terminus of the antigen binding molecule, e.g., the antibody or antigen binding fragment thereof. In certain embodiments, more than one different protein or peptide is operably linked to the antigen binding molecule, e.g., the antibody or antigen binding fragment thereof.

### 3. Biological Characteristics of the Antibodies and Antigen Binding Molecules

The present invention includes antibodies and antigen binding fragments thereof that bind human and cynomolgus Trop-2.

The present invention includes Trop-2 antigen binding molecules, e.g., anti-Trop-2 antibodies, or antigen binding fragments thereof, which bind to human Trop-2 protein specifically. In certain embodiments, the binding of the antigen binding molecules of the present invention to a Trop-2 family member other than Trop-2, e.g., Trop-1, or Trop-2 from certain non-human mammal, e.g., murine Trop-2 is either undetectable or very weak, as determined using an assay as set forth in Example 3, or a substantially similar assay.

The present invention includes Trop-2 antigen binding molecules, e.g., Trop-2 antibodies or antigen binding fragments thereof, which specifically bind to non-human primate Trop-2, e.g., cynomolgus Trop-2 expressed on a cell surface. In certain embodiment, the Trop-2 antigen binding molecules, e.g., Trop-2 antibodies or antigen binding fragments therefore, bind to a non-human primate Trop-2 with similar affinity, as determined using an assay as set forth in Example 3, or a substantially similar assay. In certain embodiments, the Trop-2 antigen binding molecule, e.g., anti-Trop-2 antibodies or antigen binding fragments thereof, of the present invention, bind to recombinant human or cynomolgus Trop-2 protein with an EC50 value of about 0.01 nM, about 0.02 nM, about 0.03 nM, about 0.04 nM, or between about 0.01 nM and about 0.04 nM, or less, as determined using an assay as set forth in Example 3, or a substantially similar assay. In certain embodiments, the Trop-2 antigen binding molecule, e.g., anti-Trop-2 antibodies or antigen binding fragments thereof, of the present invention, bind to human or cynomolgus Trop-2 expressing cell with an EC50 value of about 0.01 nM, about 0.02 nM, about 0.03 nM, about 0.04 nM, about 0.05 nM, about 0.06 nM, about 0.07 nM, about 0.08 nM, about 0.09 nM, about 0.1 nM, about 0.11 nM, about 0.12 nM, about 0.13 nM, about 0.14 nM, about 0.15 nM, about 0.16 nM, about 0.17 nM, about 0.18 nM, about 0.19 nM, about 0.2 nM, about 0.3 nM, about 0.4 nM, about 0.5 nM, about 0.6 nM, about 0.7 nM, about 0.8 nM, about 0.9 nM, about 1.0 nM, about 1.2 nM, about 1.4 nM, about 1.6 nM, about 1.8 nM, about 2 nM, between about 0.01 nM and about 2 nM, or less, as determined using an assay as set forth in Example 4, or a substantially similar assay.

In certain embodiments, the present invention provides Trop-2 antigen binding molecules, e.g., Trop-2 antibodies or antigen binding fragments thereof, which specifically bind to human Trop-2 with a K_{D} of about 0.01 nM, about 0.02 nM, about 0.03 nM, about 0.04 nM, about 0.05 nM, about 0.06 nM, about 0.07 nM, about 0.08 nM, about 0.09 nM, about 0.1 nM, about 0.2 nM, about 0.3 nM, about 0.4 nM, about 0.5 nM, about 0.6 nM, about 0.7 nM, about 0.8 nM, about 0.9 nM, about 1.0 nM, about 1.1 nM, about 1.2 nM, about 1.5 nM, about 1.7 nM, about 2.0 nM, about 2.2 nM, about 2.5 nM, about 2.7 nM, about 3.0 nM, about 4 nM, about 5 nM, between about 0.01 nM to about 5 nM, or less, as determined using an assay as set forth in Example 3, or a substantially similar assay. In certain embodiments, the Trop-2 antigen binding molecules, e.g., Trop-2 antibodies or antigen binding fragments thereof, of the present invention, bind to human Trop-2 with a K_{D} that is below LLOD (low limit of detection, e.g., 2 μm) of an assay as described in Example 3, or a substantially similar assay.

The present invention includes Trop-2 antigen binding molecules, e.g., Trop-2 antibodies or the antigen binding fragments thereof, which are capable of specifically binding to human and cynomolgus Trop-2 expressed on a cell surface and induces certain modification of the Trop-2 expressing cells, e.g., the killing of the Trop-2 expressing cells via ADCC, CDC, ADCP or the internalization of the anti-Trop-2 antibody or a conjugate. According to certain embodiments, the antigen binding molecules, e.g., the anti-Trop-2 antibodies or the antigen binding fragments thereof, of the present disclosure, induce ADCC killing of the cancer cells. The capacity of a Trop-2 antigen binding protein, e.g., a Trop-2 antibody or an antigen binding fragment thereof, to induce ADCC, can be assessed by the assays described in Example 5, or a substantially similar assay. Without wishing to be bound by any theory, it is recognized that the capacity of an antibody to induce ADCC is associated with its capacity to cross-link the Fc receptor CD16A (FcγRIIIA). In the assay described in Example 5, Jurkat-Lucia™ NFAT-CD16 cells were engineered from the human T-lymphocyte Jurkat cell line to stably express the cell surface Fc receptor CD16A (FcγRIIIA; V158 allotype3) and the Lucia luciferase reporter gene can be activated upon cross-linking of the cell surface CD16A engaged with the cell surface bound Trop-2 antibody. Under such an assay, antibodies displaying lower EC50 have higher ADCC potency. Other methods known in the art, such as measuring the cross-linking between cell surface Trop-2-bound antibodies and the Fc receptor CD16A (FcγRIIIA) at the surface of immune effector cells, e.g., Natural Killer cells, as described in Yamashita M et al, Sci. Rep. 2016,6:19772, or substantially similar methods, can also be used. The present invention includes antigen binding molecules, e.g., antibodies or antigen binding fragments thereof, which induce ADCC, with an EC₅₀ value of about 0.03 nM, about 0.04 nM, about 0.05 nM, about 0.06 nM, about 0.07 nM, about 0.08 nM, about 0.09 nM, about 0.10 nM, about 0.15 nM, about 0.20 nM, about 0.25 nM, about 0.30 nM, about 0.35 nM, about 0.40 nM, about 0.45 nM, about 0.50 nM, about 0.55 nM, about 0.60 nM, between about 0.03 nM to about 0.6 nM, or less, as determined using an assay as set forth in Example 5, or a substantially similar assay.

The present invention includes Trop-2 antigen binding molecules, e.g., Trop-2 antibodies or the antigen binding fragments thereof, or a conjugate thereof, which are capable of inducing internalization upon its binding to Trop-2 expressed on a cell surface. The capacity of a Trop-2 antigen binding protein, e.g., a Trop-2 antibody, or an antigen binding fragment thereof, or a conjugate, to induce internalization upon its binding to a Trop-2 expressed on a cell surface, can be assessed by the assays described in Example 6, or a substantially similar assay. In certain embodiments, the antigen binding molecules, e.g., antibodies or antigen binding fragments thereof, which induce the internalization of about 10%, about 15%, about 20%, about 25%, about 30%, about 35%, about 40%, about 45%, about 50%, about 55%, about 60%, about 65%, about 70%, about 75%, between about 10% to about 75%, or more, cell surface bound antibody, or antigen binding fragments thereof or a conjugate thereof of the present invention within 1 hour, 2 hours, or 4 hours upon binding to the cell surface.

### 4. Species Selectivity and Species Cross-Reactivity

The present invention, according to certain embodiments, provides antigen binding molecules that bind to human Trop-2 but not to Trop-2 from other species. The present invention also includes antigen binding molecules that bind to human Trop-2 and to Trop-2 from one or more non-human species, e.g., non-human primates.

According to certain exemplary embodiments of the invention, antigen binding molecules are provided which bind to human Trop-2 and may bind or not bind, as the case may be, to one or more of mouse, rat, guinea pig, hamster, gerbil, pig, cat, dog, rabbit, goat, sheep, cow, horse, camel, cynomolgus, marmoset, rhesus or chimpanzee Trop-2. For example, in a particular exemplary embodiment of the present invention, antigen binding molecules are provided comprising an antigen binding domain that binds human Trop-2 and non-human primate, e.g., cynomolgus Trop-2, but does not bind to mouse or rat Trop-2.

### III. Therapeutic Use of the Anti-Trop-2 Antigen Binding Molecules

The anti-Trop-2 antigen binding molecules of the present invention, including antibodies, antigen binding fragment thereof, and multispecific antibodies thereof, have numerous in vitro, in vivo and ex vivo utilities associated with specifically targeting Trop-2 expressing cells, e.g., cancer cells. Without wishing to be bound by any theory, it is hypothesized that the antigen binding molecules of the present invention, e.g., anti-Trop-2 antibodies or antigen binding fragments thereof, binds to Trop-2 expressed on cell surface and induces changes in the cells, e.g., changes in the biochemistry, metabolism, physiology, or survival, of the cells. Accordingly, the antigen binding molecules of the invention (and therapeutic compositions comprising the same) are useful, inter alia, for treating any disease or disorder in which the binding of a Trop-2 antigen binding molecules, e.g., antibodies or antigen binding fragments thereof, would be beneficial. In view of the expression of Trop-2 of various cancer cells, the anti-Trop-2 antigen binding molecules, e.g., antibodies or the antigen binding fragments thereof of the present invention may be used individually or in combination with a variety of active agents for treating a broad scope of diseases or disorders, including a variety of cancers.

Accordingly, the present invention provides a method of binding Trop-2 expressed on a cell surface with antigen binding molecules, e.g., anti-Trop-2 antibodies or antigen binding fragments thereof, including contacting the cell with the antigen binding molecules of the present invention, e.g., anti-Trop-2 antibodies or antigen binding fragment thereof, with a cell, thereby binding the Trop-2 express on a cell surface with the antigen binding molecules, e.g., anti-Trop-2 antibodies or antigen binding fragments thereof. The binding of the antigen binding molecules of the present invention, e.g., anti-Trop-2 antibodies or antigen binding fragment thereof, to Trop-2 expressed on a cell surface can be measure by a method as described in Example 4, or a substantially similar method. Such bindings can be beneficial for treating various diseases.

In certain embodiments, the present invention provides a method of killing a cancer cell by ADCC, including contacting the cancer cell with the antigen binding molecules of the present invention, e.g., anti-Trop-2 antibodies or antigen binding fragment thereof, thereby killing the cancer cell. The killing of a tumor cell by ADCC can be measured by a method as described in Example 5, or a substantially similar method.

In certain embodiments, the present invention provides a method of inducing internalization of Trop-2 expressed on a cell surface, e.g., a cancer cell surface, including contacting the cell with the antigen binding molecules of the present invention, e.g., anti-Trop-2 antibodies or antigen binding fragment thereof, thereby induce the internalization of Trop-2 into the cell. The internalization of Trop-2 can be measured by a method as described in Example 6, or a substantially similar method. In certain embodiment, the antigen binding molecules of the present invention, e.g., anti-Trop-2 antibodies or antigen binding fragment thereof, induces the internalization of about 10%, about 20%, about 30%, about 40%, about 50%, about 60%, about 70%, about 80%, or about 90% Trop-2 expressed on a cell surface.

In some embodiments, the antigen binding molecules, e.g., anti-Trop-2 antibodies or antigen binding fragments thereof, of the present invention are administered to cells in culture (in vitro) or to human subjects, in vivo or ex vivo, to enhance immunity in a variety of diseases. Accordingly, in one embodiment, a method for stimulating an immune response in a subject in need thereof includes administering to the subject an anti-Trop-2 antibody, antigen binding fragments thereof (e.g., anti-Trop-2 HCVRs and LCVRs) or multispecific anti-Trop-2 antibodies described herein, such that an immune response is enhanced, stimulated, up-regulated in the subject, for example, to inhibit tumor growth, stimulate anti-tumor T-cell immunity and/or stimulate antimicrobial immunity.

In one embodiment, a method for inhibiting the growth of tumor cells in a subject, e.g., a human, comprises administering to the subject an anti-Trop-2 antibody described herein such that growth of the tumor is inhibited in the subject. The inhibition of tumor growth can be measured by various methods. The tumor growth can be measured using methods, e.g., as described in Talkington, A and Durrett, R, Estimating Tumor Growth Rates in vivo, Bull Math Biol., 2015 Oct.: 77 (10): 1934-54, available at https://www.ncbi.nlm.nih.gov/pmc/articles/PMC4764475/, the entire contents of which are incorporated herein by reference. The inhibition of tumor growth can also be measured by the reduction of tumor size. In certain embodiment, the methods of the invention inhibit the tumor growth by at least about 10%, about 20%, about 50%, about 60%, about 70%, about 80%, about 90%, about 95%, or more, as compared to a baseline level.

In certain preferred embodiments, the subject, e.g., human has a cell proliferative disease or cancer. Binding to Trop-2 expressed on a cancer cell surface by the antigen binding molecules of the present invention, e.g., anti-Trop-2 antibodies or antigen binding fragment thereof, can induce ADCC killing of the cancer cell. Therefore, the present invention provides methods for treating a subject having cancer, comprising administering to the subject an anti-Trop-2 antigen binding molecule, e.g., an antibody or the antigen binding fragment thereof, as described herein, such that the subject is treated, e.g., such that growth of a cancerous tumor is inhibited or reduced and/or that the tumor regresses. The anti-Trop-2 antibody can be used alone to inhibit the growth of cancerous tumors. Alternatively, the anti-Trop-2 antibody can be used in conjunction with targeting one or more other active agents, e.g., other anti-cancer targets, immunogenic agents, standard cancer treatments, or other antibodies, as described below. The antigen binding molecules of the present invention may be used to treat, e.g., primary and/or metastatic tumors. The present invention also includes methods for treating residual cancer in a subject. As used herein, the term “residual cancer” means the existence or persistence of one or more cancerous cells in a subject following treatment with an anti-cancer therapy.

Accordingly, in one aspect, a method of treating cancer includes the step of administering to a subject in need thereof, a therapeutically effective amount of an anti-Trop-2 antibody as described herein. Preferably, the antibody inhibits the activity of human anti-Trop-2 and includes one or more HCVRs and LCVRs described herein. Further, the anti-Trop-2 antigen binding molecules, e.g., antibodies for use in this method may include chimeric or humanized non-human anti-Trop-2 antibodies therefrom. The efficacy of treating a cancer can be measured by various methods. For example, the efficacy of treating a cancer can be measured by improvements in survival, or reduction in tumor size. In certain embodiments, the methods of the invention increase the efficacy of treating a cancer by at least about 10%, about 20%, about 50%, about 60%, about 70%, about 80%, about 90%, about 95%, about 1-fold, about 2 folds, about 4 folds, or more, as compared to a baseline level. The baseline level, as used in the context of cancer treatment, refers to the efficacy using a placebo if the Trop-2 antigen binding molecule of the invention is the sole therapeutic agent, or the efficacy using a placebo or an additional therapeutic agent if the Trop-2 antigen binding molecule of the invention is used in combination with the additional therapeutic agent.

Cancers whose growth may be inhibited using the antibodies of the invention include a broad variety of cancers, especially those that are unresponsive or that have a tendency to become unresponsive to monotherapies with other antibodies or chemotherapeutic agents. Non-limiting examples of cancers for treatment include squamous cell carcinoma, small-cell lung cancer, non-small cell lung cancer, squamous non-small cell lung cancer (NSCLC), non NSCLC, glioma, gastrointestinal cancer, renal cancer (e.g., clear cell carcinoma), ovarian cancer, liver cancer, colorectal cancer, endometrial cancer, kidney cancer (e.g., renal cell carcinoma (RCC)), prostate cancer (e.g., hormone refractory prostate adenocarcinoma), thyroid cancer, neuroblastoma, pancreatic cancer, glioblastoma (glioblastoma multiforme), cervical cancer, stomach cancer, bladder cancer, hepatoma, breast cancer, colon carcinoma, and head and neck cancer (or carcinoma), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally-induced cancers including those induced by asbestos, virus-related cancers (e.g., human papilloma virus (HPV)—related tumor), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CIVIL), undifferentiated AML (MO), myeloblastic leukemia (M1), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NEIL), B-cell lymphomas, T-cell lymphomas, lymphoplasmacytoid lymphoma, monocytoid B-cell lymphoma, mucosa-associated lymphoid tissue (MALT) lymphoma, anaplastic (e.g., Ki 1+) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukemia (T-Lbly/T-ALL), peripheral T-cell lymphoma, lymphoblastic lymphoma, post-transplantation lymphoproliferative disorder, true histiocytic lymphoma, primary central nervous system lymphoma, primary effusion lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of the central and peripheral nervous, including astrocytoma, schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) preferably of the T-cell type; a/d T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland; acute myeloid lymphoma, as well as any combinations of said cancers. The methods described herein may also be used for treatment of metastatic cancers, refractory cancers (e.g., cancers refractory to previous immunotherapy, e.g., with a blocking CTLA-4 or PD-1 antibody), and recurrent cancers.

In some embodiments, treatment of a cancer patient with an anti-Trop-2 antibody and/or other active agents according to the present invention may lead to a long-term durable response relative to the current standard of care, including long term survival of at least 1, 2, 3, 4, 5, 10 or more years and/or recurrence free survival of at least 1, 2, 3, 4, 5, or 10 or more years. In certain embodiments, treatment of a cancer patient with an anti-Trop-2 antibody and/or other active agents according to the present invention prevents recurrence of cancer or delays recurrence of cancer by, e.g., 1, 2, 3, 4, 5, or 10 or more years. The anti-Trop-2 treatment can be used as a primary or secondary line of treatment.

Suitable routes for administering the antigen binding molecules, e.g., anti-Trop-2 antibodies or antigen binding fragment thereof, of the present invention (e.g., humanized monoclonal antibodies, multi-specific antibodies, and antibody conjugates) described herein in vivo, ex vivo or in vitro are well known in the art and can be selected by those of ordinary skill. For example, the antibody compositions can be administered by parenteral injection (e.g., intravenous or subcutaneous). Suitable dosages will depend on the age and weight of the subject and the concentration and/or formulation of the antibody composition as further described below.

The term “cell proliferative disorder” refers to a disorder characterized by abnormal proliferation of cells. A proliferative disorder does not imply any limitation with respect to the rate of cell growth, but merely indicates loss of normal controls that affect growth and cell division. Thus, in some embodiments, cells of a proliferative disorder can have the same cell division rates as normal cells but do not respond to signals that limit such growth. Within the ambit of “cell proliferative disorder” is a neoplasm, cancer or tumor.

The term “cancer” refers to any one of a variety of malignant neoplasms characterized by the proliferation of cells that have the capability to invade surrounding tissue and/or metastasize to new colonization sites, and includes carcinomas, sarcomas, adenocarcinomas, melanomas, leukemias, lymphomas, germ cell tumors and blastomas, including both solid and lymphoid cancers. Exemplary cancers that may be treated in accordance with the compositions and methods of the present invention include cancers of the brain, bladder, breast, cervix, colon, head and neck, kidney, lung, non-small cell lung, mesothelioma, ovary, prostate, stomach and uterus, leukemia, and medulloblastoma.

The term “carcinoma” refers to the malignant growth of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, pancreatic ductal adenocarcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “sarcoma” refers to a tumor made up of a substance like the embryonic connective tissue and is generally composed of closely packed cells embedded in a fibrillar or homogeneous substance. Exemplary sarcomas include, for example, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphomas (e.g., Non-Hodgkin Lymphoma), immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” refers to a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma subungal melanoma, and superficial spreading melanoma.

The term “lymphoma” refers to a group of cancers affecting hematopoietic and lymphoid tissues, which begins in lymphocytes, the blood cells that are found primarily in lymph nodes, spleen, thymus, and bone marrow. Two main types of lymphoma are non-Hodgkin's lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas include, but are not limited to, cutaneous T-cell lymphoma, peripheral T-cell lymphoma, anaplastic large cell lymphoma, mycosis fungoides, and precursor T-lymphoblastic lymphoma.

The term “leukemia” refers to progressive, malignant diseases of the blood-forming organs and is generally characterized by a distorted proliferation and development of leukocytes and their precursors in the blood and bone marrow. Exemplary leukemias include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

Additional cancers include, for example, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, thyroid cancer, neuroblastoma, esophageal cancer, genitourinary tract cancer, malignant hypercalcemia, cervical cancer, endometrial cancer, and adrenal cortical cancer.

### IV. Combination Therapies

In another aspect, the present invention provides therapeutic compositions and combination therapies for enhancing antigen-specific T cell responses, reducing immunosuppression, and/or reducing tumor growth in a subject. The present invention includes compositions and therapeutic formulations comprising any of the exemplary antigen binding molecules, e.g., herein in combination with one or more additional therapeutical agents, and methods of treatment comprising administering such combinations to subjects in need thereof. The term “additional therapeutic agent,” as used herein, refers to any agents, which can be used to treat a disease or disorder, and any method of treatment for certain disease or disorder. For example, radiotherapy and surgery are deemed as “additional therapeutic agent” when they are used in combination with the antigen binding molecules, e.g., anti-Trop-2 antibodies or antigen binding fragment thereof, of the invention.

In certain embodiments, the additional therapeutic agent may be an agent that blocks the interaction between PD-1 and PD-L1. Exemplary blocking agents for PD1/PD-L1 interaction include, but are not limited to pembrolizumab, nivolumab, atezolizumab, avelumab, durvalumab, BMS-936559, sintilimab, toripalimab, tislelizumab, camrelizumab, envafolimab, sugemalimab, penpulimab, cadonilimab, sulfamonomethoxine, and sulfamethizole.

In some embodiments, the anti-Trop-2 antigen binding molecule, e.g., an anti-Trop-2 antibody or antigen binding fragment thereof, of the present invention is co-administered with one or more additional therapeutical agents in amount(s) effective in stimulating an immune response and/or apoptosis so as to further enhance, stimulate or upregulate an immune response and/or apoptosis in a subject. In addition, the one or more additional therapeutically active agents are administered prior to or subsequent to treatment with the anti-Trop-2 antibody.

In certain embodiments, the anti-Trop-2 antibodies described herein are administered in combination with or concurrently combined with one or other more other active agents, such as anti-cancer antibodies or polypeptides, chemotherapeutic agents, and radiotoxic agents. In other embodiments, the anti-Trop-2 antibodies described herein are administered in combination with or concurrently combined with a standard cancer treatment, such as surgery or radiation.

Co-administration of the anti-Trop-2 antibodies with these active agents or treatment modalities may address clinical deficiencies with regard to drug resistance, changes in the antigenicity of the tumor cells that render them unreactive with the antibody, and toxicities (by administering lower doses of one or more agents). Trop-2 blockade is particularly well suited for use when combined with otherwise refractory chemotherapeutic regimes. In these instances, it may be possible to achieve enhanced efficacy, but to reduce the dose of chemotherapeutic reagent administered (Mokyr et al. (1998) Cancer Research 58: 5301-5304). The rationale for Trop-2 blockade with radiation or chemotherapy is predicated on promoting cell death as a consequence of the cytotoxic action of radiation and most chemotherapeutic compounds, which can further result in increased levels of tumor antigen in the antigen presentation pathway. Other combination therapies that may act additively or synergistically with Trop-2 inhibition through cell death are surgery and hormone deprivation or inhibition. Each of these protocols further creates a source of tumor antigen in the host.

In some embodiments the anti-Trop-2 antibodies described herein are linked to another active agent in the form of an immuno-complex, immunoconjugate, or fusion protein. Alternatively, the anti-Trop-2 antibodies can be administered separate from the other active agent. In this case, the anti-Trop-2 antibodies and other antagonists can be administered before, after or concurrently with the other active agent or they may be co-administered with other known therapies, e.g., other anti-cancer agents, radiation etc. Accordingly, the present invention provides compositions and methods for providing two or more anti-cancer agents operating additively or synergistically via different mechanisms to beneficially provide both cytotoxic and immunoprotective effects in human cancer cells.

For example, in some embodiments, the anti-Trop-2 antibodies described herein may be combined with an anti-cancer agent, such an alkylating agent; an anthracycline antibiotic; an anti-metabolite; a detoxifying agent; an interferon; a polyclonal or monoclonal antibody; an EGFR inhibitor; a HER2 inhibitor; a histone deacetylase inhibitor; a hormone; a mitotic inhibitor; a phosphatidylinositol-3-kinase (PI3K) inhibitor; an Akt inhibitor; a mammalian target of rapamycin (mTOR) inhibitor; a proteasomal inhibitor; a poly(ADP-ribose) polymerase (PARP) inhibitor; a Ras/MAPK pathway inhibitor; a centrosome declustering agent; a multi-kinase inhibitor; a serine/threonine kinase inhibitor; a tyrosine kinase inhibitor; a VEGF/VEGFR inhibitor; a taxane or taxane derivative, an aromatase inhibitor, an anthracycline, a microtubule targeting drug, a topoisomerase poison drug, an inhibitor of a molecular target or enzyme (e.g., a kinase or a protein methyltransferase), a cytidine analogue or combination thereof.

Exemplary alkylating agents include, but are not limited to, cyclophosphamide (Cytoxan; Neosar); chlorambucil (Leukeran); melphalan (Alkeran); carmustine (BiCNU); busulfan (Busulfex); lomustine (CeeNU); dacarbazine (DTIC-Dome); oxaliplatin (Eloxatin); carmustine (Gliadel); ifosfamide (Ifex); mechlorethamine (Mustargen); busulfan (Myleran); carboplatin (Paraplatin); cisplatin (CDDP; Platinol); temozolomide (Temodar); thiotepa (Thioplex); bendamustine (Treanda); or streptozocin (Zanosar).

Exemplary anthracycline antibiotics include, but are not limited to, doxorubicin (Adriamycin); doxorubicin liposomal (Doxil); mitoxantrone (Novantrone); bleomycin (Blenoxane); daunorubicin (Cerubidine); daunorubicin liposomal (DaunoXome); dactinomycin (Cosmegen); epirubicin (Ellence); idarubicin (Idamycin); plicamycin (Mithracin); mitomycin (Mutamycin); pentostatin (Nipent); or valrubicin (Valstar).

Exemplary anti-metabolites include, but are not limited to, fluorouracil (Adrucil); capecitabine (Xeloda); hydroxyurea (Hydrea); mercaptopurine (Purinethol); pemetrexed (Alimta); fludarabine (Fludara); nelarabine (Arranon); cladribine (Cladribine Novaplus); clofarabine (Clolar); cytarabine (Cytosar-U); decitabine (Dacogen); cytarabine liposomal (DepoCyt); hydroxyurea (Droxia); pralatrexate (Folotyn); floxuridine (FUDR); gemcitabine (Gemzar); cladribine (Leustatin); fludarabine (Oforta); methotrexate (MTX; Rheumatrex); methotrexate (Trexall); thioguanine (Tabloid); TS-1 or cytarabine (Tarabine PFS).

Exemplary detoxifying agents include, but are not limited to, amifostine (Ethyol) or mesna (Mesnex).

Exemplary interferons include, but are not limited to, interferon alfa-2b (Intron A) or interferon alfa-2a (Roferon-A).

Exemplary polyclonal or monoclonal antibodies include, but are not limited to, trastuzumab (Herceptin); ofatumumab (Arzerra); bevacizumab (Avastin); rituximab (Rituxan); cetuximab (Erbitux); panitumumab (Vectibix); tositumomab/odine131 tositumomab (Bexxar); alemtuzumab (Campath); ibritumomab (Zevalin; In-111; Y-90 Zevalin); gemtuzumab (Mylotarg); eculizumab (Soliris) ordenosumab.

Exemplary EGFR inhibitors include, but are not limited to, gefitinib (Iressa); lapatinib (Tykerb); cetuximab (Erbitux); erlotinib (Tarceva); panitumumab (Vectibix); PKI-166; canertinib (CI-1033); matuzumab (Emd7200) or EKB-569.

Exemplary HER2 inhibitors include, but are not limited to, trastuzumab (Herceptin); lapatinib (Tykerb) or AC-480.

Exemplary histone deacetylase inhibitors include, but are not limited to, vorinostat (Zolinza), valproic acid, romidepsin, entinostat abexinostat, givinostat, and mocetinostat.

Exemplary hormones include, but are not limited to, tamoxifen (Soltamox; Nolvadex); raloxifene (Evista); megestrol (Megace); leuprolide (Lupron; Lupron Depot; Eligard; Viadur); fulvestrant (Faslodex); letrozole (Femara); triptorelin (Trelstar LA; Trelstar Depot); exemestane (Aromasin); goserelin (Zoladex); bicalutamide (Casodex); anastrozole (Arimidex); fluoxymesterone (Androxy; Halotestin); medroxyprogesterone (Provera; Depo-Provera); estramustine (Emcyt); flutamide (Eulexin); toremifene (Fareston); degarelix (Firmagon); nilutamide (Nilandron); abarelix (Plenaxis); or testolactone (Teslac).

Exemplary mitotic inhibitors include, but are not limited to, paclitaxel (Taxol; Onxol; Abraxane); docetaxel (Taxotere); vincristine (Oncovin; Vincasar PFS); vinblastine (Velban); etoposide (Toposar; Etopophos; VePesid); teniposide (Vumon); ixabepilone (Ixempra); nocodazole; epothilone; vinorelbine (Navelbine); camptothecin (CPT); irinotecan (Camptosar); topotecan (Hycamtin); amsacrine or lamellarin D (LAM-D).

Exemplary phosphatidyl-inositol-3 kinase (PI3K) inhibitors include wortmannin an irreversible inhibitor of PI3K, demethoxyviridin a derivative of wortmannin, LY294002, a reversible inhibitor of PI3K; BKM120 (Buparlisib); Idelalisib (a P13K Delta inhibitor); duvelisib (IPI-145, an inhibitor of PI3K delta and gamma); alpelisib (BYL719), an alpha-specific PI3K inhibitor; TGR 1202 (previously known as RP5264), an oral PI3K delta inhibitor; and copanlisib (BAY 80-6946), an inhibitor PI3Kα,δ isoforms predominantly.

Exemplary Akt inhibitors include, but are not limited to miltefosine, AZD5363, GDC-0068, MK2206, Perifosine, RX-0201, PBI-05204, GSK2141795, and SR13668.

Exemplary MTOR inhibitors include, but are not limited to, everolimus (Afinitor) or temsirolimus (Torisel); rapamune, ridaforolimus; deforolimus (AP23573), AZD8055 (AstraZeneca), OSI-027 (OSI), INK-128, BEZ235, PI-103, Torin1, PP242, PP30, Ku-0063794, WAY-600, WYE-687, WYE-354, and CC-223.

Exemplary proteasomal inhibitors include, but are not limited to, bortezomib (PS-341), ixazomib (MLN 2238), MLN 9708, delanzomib (CEP-18770), carfilzomib (PR-171), YU101, oprozomib (ONX-0912), marizomib (NPI-0052), and disufiram.

Exemplary PARP inhibitors include, but are not limited to, olaparib, iniparib, velaparib, BMN-673, BSI-201, AG014699, ABT-888, GP121016, MK4827, INO-1001, CEP-9722, PJ-34, Tiq-A, Phen, PF-01367338 and combinations thereof.

Exemplary Ras/MAPK pathway inhibitors include, but are not limited to, trametinib, selumetinib, cobimetinib, CI-1040, PD0325901, AS703026, R04987655, R05068760, AZD6244, GSK1120212, TAK-733, U0126, MEK162, and GDC-0973.

Exemplary centrosome declustering agents include, but are not limited to, griseofulvin; noscapine, noscapine derivatives, such as brominated noscapine (e.g., 9-bromonoscapine), reduced bromonoscapine (RBN), N-(3-brormobenzyl) noscapine, aminonoscapine and water-soluble derivatives thereof; CW069; the phenanthridene-derived poly(ADP-ribose) polymerase inhibitor, PJ-34; N2-(3-pyridylmethyl)-5-nitro-2-furamide, N2-(2-thienylmethyl)-5-nitro-2-furamide, and N2-benzyl-5-nitro-2-furamide.

Exemplary multi-kinase inhibitors include, but are not limited to, regorafenib; sorafenib (Nexavar); sunitinib (Sutent); BIBW 2992; E7080; Zd6474; PKC-412; motesanib; or AP24534.

Exemplary serine/threonine kinase inhibitors include, but are not limited to, ruboxistaurin; eril/easudil hydrochloride; flavopiridol; seliciclib (CYC202; Roscovitrine); SNS-032 (BMS-387032); Pkc412; bryostatin; KAI-9803; SF1126; VX-680; Azd1152; Arry-142886 (AZD-6244); SCIO-469; GW681323; CC-401; CEP-1347 or PD 332991.

Exemplary tyrosine kinase inhibitors include, but are not limited to, erlotinib (Tarceva); gefitinib (Iressa); imatinib (Gleevec); sorafenib (Nexavar); sunitinib (Sutent); trastuzumab (Herceptin); bevacizumab (Avastin); rituximab (Rituxan); lapatinib (Tykerb); cetuximab (Erbitux); panitumumab (Vectibix); everolimus (Afinitor); alemtuzumab (Campath); gemtuzumab (Mylotarg); temsirolimus (Torisel); pazopanib (Votrient); dasatinib (Sprycel); nilotinib (Tasigna); vatalanib (Ptk787; ZK222584); CEP-701; SU5614; MLN518; XL999; VX-322; Azd0530; BMS-354825; SKI-606 CP-690; AG-490; WHI-P154; WHI-P131; AC-220; or AMG888.

Exemplary VEGF/VEGFR inhibitors include, but are not limited to, bevacizumab (Avastin); sorafenib (Nexavar); sunitinib (Sutent); ranibizumab; pegaptanib; or vandetinib.

Exemplary microtubule targeting drugs include, but are not limited to, paclitaxel, docetaxel, vincristin, vinblastin, nocodazole, epothilones and navelbine.

Exemplary topoisomerase poison drugs include, but are not limited to, teniposide, etoposide, adriamycin, camptothecin, daunorubicin, dactinomycin, mitoxantrone, amsacrine, epirubicin and idarubicin.

Exemplary taxanes or taxane derivatives include, but are not limited to, paclitaxel and docetaxol.

Exemplary general chemotherapeutic, anti-neoplastic, anti-proliferative agents include, but are not limited to, altretamine (Hexalen); isotretinoin (Accutane; Amnesteem; Claravis; Sotret); tretinoin (Vesanoid); azacitidine (Vidaza); bortezomib (Velcade) asparaginase (Elspar); levamisole (Ergamisol); mitotane (Lysodren); procarbazine (Matulane); pegaspargase (Oncaspar); denileukin diftitox (Ontak); porfimer (Photofrin); aldesleukin (Proleukin); lenalidomide (Revlimid); bexarotene (Targretin); thalidomide (Thalomid); temsirolimus (Torisel); arsenic trioxide (Trisenox); verteporfin (Visudyne); mimosine (Leucenol); (1M tegafur-0.4 M 5-chloro-2,4-dihydroxypyrimidine-1 M potassium oxonate) or lovastatin.

In some embodiments, the anti-Trop-2 antibody described herein may also be used in combination with bispecific antibodies that target Fcα or Fcγ receptor-expressing effectors cells to tumor cells (see, e.g., U.S. Pat. Nos. 5,922,845 and 5,837,243). Such bispecific antibodies can be used to target two separate antigens. For example, anti-Fc receptor/anti-tumor antigen (e.g., Her-2/neu) bispecific antibodies have been used to target macrophages to sites of tumor. This targeting may more effectively activate tumor specific responses. The T cell arm of these responses would be augmented by the inhibition of Trop-2. Alternatively, antigen may be delivered directly to DCs by the use of bispecific antibodies that bind to tumor antigen and a dendritic cell specific cell surface marker.

In all of the above methods, Trop-2 inhibition can be combined with other forms of immunotherapy such as cytokine treatment (e.g., interferons, GM-CSF, G-CSF, IL-2), or bispecific antibody therapy using two different binding specificities to provide enhanced presentation of tumor antigens.

In some embodiments, the additional therapeutic agent for use in any of the foregoing methods of treatment, uses of an antigen binding molecule or uses of a pharmaceutical composition is an immunostimulatory agent selected from (a) an agent that blocks signaling of an inhibitory receptor of an immune cell or a ligand thereof (collectively immune checkpoint protein) or a nucleic acid encoding such agent; (b) an agonist to a stimulatory receptor of an immune cell or a nucleic acid encoding such agonist; (c) a cytokine or chemokine or a nucleic acid encoding a cytokine or chemokine; (d) an oncolytic virus or a nucleic acid encoding an oncolytic virus; (e) a T cell expressing a chimeric antigen receptor; (f) a bi- or multi-specific T cell directed antibody or a nucleic acid encoding such antibody; (g) an anti-TGF-β antibody or a nucleic acid encoding such antibody; (h) a TGF-β trap or a nucleic acid encoding such trap; (i) a vaccine to a cancer-associated antigen, including such antigen or a nucleic acid encoding such antigen, (j) a cell therapy, and (k) combinations thereof. In some embodiments, the additional therapeutic agent is an agent that blocks signaling of an inhibitory receptor of an immune cell or a ligand thereof or a nucleic acid encoding such agent, and the inhibitory receptor or ligand thereof is selected from A2aR, CTLA-4, PD-1, PD-L1, PD-L2, TIGIT, LAG-3, TIM-3, B7-H3, B7-H4, A2aR, CD73, PVRIG/PVRL2, neuritin, BTLA, CECAM-1, CECAM-5, CECAM6, IL-1R8, VISTA, LAIRI, LILRB1, LILRB2, LILRB3, LILRB4, LILRB5, CD47, SIRPa, CD200R, CD96, CD112R, 2B4, TGFβ-R, KIR, NKG2A, SEMA4D, Axl, MerTK, GAS6, TNFR2, GARP, CCR8, IDO, NOX2, SIGLEC7, SIGLEC15, and any combinations thereof. In some embodiments, the additional therapeutic agent is an agonist to a stimulatory receptor of an immune cell or a nucleic acid encoding such agonist, and the stimulatory receptor of an immune cell is selected from OX40, CD2, CD3, CD7, CD27, CD28, CD30, CD40, ICAM-1, LFA-1 (CD11a/CD18), ICOS (CD278), 4-1BB (CD 137), GITR, BAFFR, HVEM, LIGHT, KG2C, SLAMF7, NKG2C, NKG2D, NKp46, NKp80, CD160, and combinations thereof. In some embodiments, the additional therapeutic agent is a cytokine or a nucleic acid encoding a cytokine selected from IL-2, IL-5, IL-7, IL-12, IL-15, IL-21, and any combinations thereof. In some embodiments, the additional therapeutic agent is an oncolytic virus or a nucleic acid encoding an oncolytic virus selected from herpes simplex virus, vesicular stomatitis virus, adenovirus, Newcastle disease virus, vaccinia virus, a maraba virus, and combinations thereof. In some embodiments, the additional therapeutic agent is a cell therapy. A cell therapy may include a T cell, NK cell, or macrophage with a chimeric antigen receptor (CAR). In some embodiments, the cell therapy includes a bi- or multi-specific T cell directed antibody.

As used herein, the term “immune checkpoint protein” refers to a receptor expressed on an immune cell, e.g., T cell, and/or a ligand thereof. The engagement of the ligand to the receptor reduces or inhibits immune responses of the immune cell. An immune checkpoint inhibitor is an agent that reduces or inhibits the engagement of the ligand to the receptor.

As used herein, the term “stimulatory receptor of an immune cell” refers to a receptor on an immune cell, which, upon the binding of the ligand thereof, enhances the immune response of the immune cell.

In certain embodiments, the present invention provides a method of treating a disease or disorder, e.g., cancer, in a subject. The method includes administering antigen binding molecules, e.g., anti-Trop-2 antibodies or antigen binding fragment thereof, of the present invention alone or in combination with a second one or more additional therapeutical agents into the subject, wherein the subject has previously received a treatment with a first one or more additional therapeutical agents.

In certain embodiment, the immune checkpoint inhibitor is an antibody that interacts specifically with an immune checkpoint. In some embodiments, the additional therapeutic agent comprises an immunostimulatory agent. In some aspects, the immune checkpoint inhibitor is an and -CTLA-4 antibody (e.g., ipilimumab), and combinations thereof. In some aspects, the immune checkpoint inhibitor is pembrolizumab. In some aspects, the immune checkpoint inhibitor is nivolumab. In some aspects, the immune checkpoint inhibitor is atezolizumab.

In some embodiments, the anti-Trop-2 antibody is administered in combination with or concurrently with an immunogenic agent. Non-limiting examples of immunogenic agents include cancer cells, tumor vaccines, and purified tumor antigens (including recombinant proteins, peptides, and carbohydrate molecules); an oncolytic virus; cells transfected with genes encoding immune stimulating cytokines etc.

In certain embodiments, the anti-Trop-2 antibody is administered together with an antigen of interest or an antigen known to be present in the subject to be treated (e.g., a tumor-bearing or virus-bearing subject) to enhance antigen-specific immunity. When an anti-Trop-2 antibody is administered together with another agent, the two can be administered separately or simultaneously.

In certain embodiments, the anti-Trop-2 antibodies described herein may be used to enhance antigen-specific immune responses by co-administration of one or more of any of these antibodies with an antigen of interest (e.g., a vaccine). Accordingly, in one embodiment, a method for enhancing an immune response to an antigen in a subject, includes the steps of administering to the subject: (i) the antigen; and (ii) a Trop-2-based antibody such that an immune response to the antigen in the subject is enhanced. The antigen can be, for example, a tumor antigen, a viral antigen, a bacterial antigen or an antigen from a pathogen. Non-limiting examples of such antigens include those discussed in the sections above, such as the tumor antigens (or tumor vaccines) discussed above, or antigens from the viruses, bacteria or other pathogens described above.

In view of the benefits associated with synergistic active agent compositions, in certain embodiments, each of the anti-Trop-2 antibody and the other active agents are administered to a subject in need thereof at subtherapeutic doses relative to the doses used in monotherapies with the same.

In certain embodiments, Trop-2 inhibition is combined with standard cancer treatments (e.g., surgery, radiation, and chemotherapy). In these instances, it may be possible to reduce the dose of chemotherapeutic reagent administered. It is believed that the combined use of Trop-2 inhibition and chemotherapy can enhance apoptosis and increase tumor antigen presentation for cytotoxic immunity. Other synergistic combination therapies include Trop-2 inhibition in combination with radiation, surgery or hormone deprivation or inhibition. Each of these protocols creates a source of tumor antigen in the host.

The additional therapeutical agent may be administered prior to, concurrent with, or after the administration of an antigen binding molecule of the present invention; (for purposes of the present disclosure, such administration regimens are considered the administration of an antigen binding molecule “in combination with” an additional therapeutically active component).

The present invention includes pharmaceutical compositions in which an antigen binding molecule of the present invention is co-formulated with one or more of the additional therapeutical agents as described elsewhere herein.

### V. Nucleic Acids and Host Cells for Expressing Anti-Trop-2 Antibodies

In another aspect, the present invention provides nucleic acids encoding the antigen binding molecules, e.g., anti-Trop-2 antibodies or antigen binding fragments thereof, of the present invention, and expression vectors comprising such nucleic acids. In some embodiments, nucleic acids encode an HCVR and/or LCVR fragment of an antibody or fragment in accordance with the embodiments described herein, or any of the other antibodies and antibody fragments described herein.

DNA encoding an antigen binding site in a monoclonal antibody can be isolated and sequenced from the hybridoma cells using conventional procedures (e.g., by using oligonucleotide probes that are capable of binding specifically to genes encoding the heavy and light chains of the monoclonal antibodies). Alternatively, amino acid sequences from immunoglobulins of interest may be determined by direct protein sequencing, and suitable encoding nucleotide sequences can be designed according to a universal codon table. In other cases, nucleotide and amino acid sequences of antigen binding sites or other immunoglobulin sequences, including constant regions, hinge regions and the like may be obtained from published sources well known in the art.

Expression vectors may be used to synthesize the antibodies of the present disclosure in cultured cells in vitro or they may be directly administered to a patient to express the antibodies of the present disclosure in vivo or ex vivo. As used herein, an “expression vector” refers to a viral or non-viral vector comprising a polynucleotide encoding one or more antibodies of the present disclosure in a form suitable for expression from the polynucleotide(s) in a host cell for antibody preparation purposes or for direct administration as a therapeutic agent.

A nucleic acid sequence is “operably linked” to another nucleic acid sequence when the former is placed into a functional relationship with the latter. For example, a DNA for a presequence or signal peptide is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a signal peptide, contiguous and in reading phase. However, enhancers do not have to be contiguous. Linking is accomplished by ligation at convenient restriction sites. If such sites do not exist, synthetic oligonucleotide adaptors or linkers may be used in accordance with conventional practice.

Nucleic acid sequences for expressing the antibodies of the present disclosure typically include an N terminal signal peptide sequence, which is removed from the mature protein. Since the signal peptide sequences can affect the levels of expression, the polynucleotides may encode any one of a variety of different N-terminal signal peptide sequences. It will be appreciated by those skilled in the art that the design of the expression vector can depend on such factors as the choice of the host cell to be transformed, the level of expression of protein desired, and the like.

The above described “regulatory sequences” refer to DNA sequences necessary for the expression of an operably linked coding sequence in one or more host organisms. The term “regulatory sequences” is intended to include promoters, enhancers and other expression control elements (e.g., polyadenylation signals). Regulatory sequences include those which direct constitutive expression of a nucleotide sequence in many types of host cells or those which direct expression of the nucleotide sequence only in certain host cells (e.g., tissue-specific regulatory sequences). Expression vectors generally contain sequences for transcriptional termination, and may additionally contain one or more elements positively affecting mRNA stability.

The expression vector contains one or more transcriptional regulatory elements, including promoters and/or enhancers, for directing the expression of antibodies of the present disclosure. A promoter comprises a DNA sequence that functions to initiate transcription from a relatively fixed location in regard to the transcription start site. A promoter contains core elements required for basic interaction of RNA polymerase and transcription factors, and may operate in conjunction with other upstream elements and response elements.

As used herein, the term “promoter” is to be construed broadly so as to include e.g., transcriptional regulatory elements (TREs) from genomic genes or chimeric TREs therefrom, including the TATA box or initiator element for accurate transcription initiation, with or without additional TREs (i.e., upstream activating sequences, transcription factor binding sites, enhancers, and silencers) which regulate activation or repression of genes operably linked thereto in response to developmental and/or external stimuli, and trans-acting regulatory proteins or nucleic acids. A promoter may contain a genomic fragment or it may contain a chimera of one or more TREs combined together.

Preferred promoters are those capable of directing high-level expression in a target cell of interest. The promoters may include constitutive promoters (e.g., HCMV, SV40, elongation factor-la (EF-1α)) or those exhibiting preferential expression in a particular cell type of interest. Enhancers generally refer to DNA sequences that function away from the transcription start site and can be either 5′ or 3′ to the transcription unit. Furthermore, enhancers can be within an intron as well as within the coding sequence. They are usually between 10 and 300 bp in length, and they function in cis. Enhancers function to increase and/or regulate transcription from nearby promoters. Preferred enhancers are those directing high-level expression in the antibody producing cell. Cell or tissue-specific transcriptional regulatory elements (TREs) can be incorporated into expression vectors to restrict expression to desired cell types. An expression vector may be designed to facilitate expression of the antibodies of the present disclosure in one or more cell types.

To co-express the individual chains of the antibodies of the present disclosure, a suitable splice donor and splice acceptor sequences may be incorporated for expressing both products. Alternatively, an internal ribosome binding sequence (IRES) or a 2A peptide sequence, may be employed for expressing multiple products from one promoter. An IRES provides a structure to which the ribosome can bind that does not need to be at the 5′ end of the mRNA. It can therefore direct a ribosome to initiate translation at a second initiation codon within a mRNA, allowing more than one polypeptide to be produced from a single mRNA. A 2A peptide contains short sequences mediating co-translational self-cleavage of the peptides upstream and downstream from the 2A site, allowing production of two different proteins from a single transcript in equimolar amounts. CHYSEL is a non-limiting example of a 2A peptide, which causes a translating eukaryotic ribosome to release the growing polypeptide chain that it is synthesizing without dissociating from the mRNA. The ribosome continues translating, thereby producing a second polypeptide.

An expression vector may comprise a viral vector or a non-viral vector. A viral vector may be derived from an adeno-associated virus (AAV), adenovirus, herpesvirus, vaccinia virus, poliovirus, poxvirus, a retrovirus (including a lentivirus, such as HIV-1 and HIV-2), Sindbis and other RNA viruses, alphavirus, astrovirus, coronavirus, orthomyxovirus, papovavirus, paramyxovirus, parvovirus, picornavirus, togaviruses and the like. A non-viral vector is simply a “naked” expression vector that is not packaged with virally derived components (e.g., capsids and/or envelopes).

In certain cases, these vectors may be engineered to target certain diseases or cell populations by using the targeting characteristics inherent to the virus vector or engineered into the virus vector. Specific cells may be “targeted” for delivery of polynucleotides, as well as expression. Thus, the term “targeting”, in this case, may be based on the use of endogenous or heterologous binding agents in the form of capsids, envelope proteins, antibodies for delivery to specific cells, the use of tissue-specific regulatory elements for restricting expression to specific subset(s) of cells, or both.

In some embodiments, expression of the antibody chains is under the control of the regulatory element such as a tissue specific or ubiquitous promoter. In some embodiments, a ubiquitous promoter such as a CMV promoter, CMV-chicken beta-actin hybrid (CAG) promoter, a tissue specific or tumor-specific promoter to control the expression of a particular antibody heavy or light chain or single-chain derivative therefrom.

Non-viral expression vectors can be utilized for non-viral gene transfer, either by direct injection of naked DNA or by encapsulating the antibody-encoding polynucleotides in liposomes, microparticles, microcapsules, virus-like particles, or erythrocyte ghosts. Such compositions can be further linked by chemical conjugation to targeting domains to facilitate targeted delivery and/or entry of nucleic acids into desired cells of interest. In addition, plasmid vectors may be incubated with synthetic gene transfer molecules such as polymeric DNA-binding cations like polylysine, protamine, and albumin, and linked to cell targeting ligands such as asialoorosomucoid, insulin, galactose, lactose or transferrin.

Alternatively, naked DNA may be employed. Uptake efficiency of naked DNA may be improved by compaction or by using biodegradable latex beads. Such delivery may be improved further by treating the beads to increase hydrophobicity and thereby facilitate disruption of the endosome and release of the DNA into the cytoplasm.

### VI. Methods for Producing Anti-Trop-2 Antibodies

In another aspect, the present invention provides host cells transformed with the anti-Trop-2 HCVRs and/or LCVRs encoding nucleic acids or expression vectors. The host cells can be any bacterial or eukaryotic cell capable of expressing the anti-Trop-2 HCVRs and/or LCVRs encoding nucleic acids or expression vectors or any of the other co-administered antibodies or antagonists described herein.

In another aspect, a method of producing an antibody of the present disclosure comprises culturing a host cell transformed with one or anti-Trop-2 HCVRs and/or LCVRs encoding nucleic acids or expression vectors under conditions that allows production of the antibody or fragment, and purifying the antibody from the cell.

In a further aspect, the present invention provides a method for producing an antibody comprising culturing a cell transiently or stably expressing one or more constructs encoding one or more polypeptide chains in the antibody; and purifying the antibody from the cultured cells. Any cell capable of producing a functional antibody may be used. In preferred embodiments, the antibody-expressing cell is of eukaryotic or mammalian origin, preferably a human cell. Cells from various tissue cell types may be used to express the antibodies. In other embodiments, the cell is a yeast cell, an insect cell or a bacterial cell. Preferably, the antibody-producing cell is stably transformed with a vector expressing the antibody.

One or more expression vectors encoding the antibody heavy or light chains can be introduced into a cell by any conventional method, such as by naked DNA technique, cationic lipid-mediated transfection, polymer-mediated transfection, peptide-mediated transfection, virus-mediated infection, physical or chemical agents or treatments, electroporation, etc. In addition, cells may be transfected with one or more expression vectors for expressing the antibody along with a selectable marker facilitating selection of stably transformed clones expressing the antibody. The antibodies produced by such cells may be collected and/or purified according to techniques known in the art, such as by centrifugation, chromatography, etc.

Examples of suitable selectable markers for mammalian cells include dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are CHO DHFR cells and mouse LTV cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, mycophenolic acid, or hygromycin. The three examples employ bacterial genes under eukaryotic control to convey resistance to the appropriate drug G418 or neomycin (geneticin), xgpt (mycophenolic acid) or hygromycin, respectively. Others include the neomycin analog G418 and puromycin.

Exemplary antibody-expressing cells include human Jurkat, human embryonic kidney (HEK) 293, Chinese hamster ovary (CHO) cells, mouse WEHI fibrosarcoma cells, as well as unicellular protozoan species, such as Leishmania tarentolae. In addition, stably transformed, antibody producing cell lines may be produced using primary cells immortalized with c-myc or other immortalizing agents.

In one embodiment, the cell line comprises a stably transformed Leishmania cell line, such as Leishmania tarentolae. Leishmania are known to provide a robust, fast-growing unicellular host for high level expression of eukaryotic proteins exhibiting mammalian-type glycosylation patterns. A commercially available Leishmania eukaryotic expression kit is available (Jena Bioscience GmbH, Jena, Germany).

In some embodiments, the cell lines express at least 1 mg, at least 2 mg, at least 5 mg, at least 10 mg, at least 20 mg, at least 50 mg, or at least 100 mg of the antibody/liter of culture.

The antibodies in the present invention may be isolated from antibody expressing cells following culture and maintenance in any appropriate culture medium, such as RPMI, DMEM, and AIM V®. The antibodies can be purified using conventional protein purification methodologies (e.g., affinity purification, chromatography, etc.), including the use of Protein-A or Protein-G immunoaffinity purification. In some embodiments, antibodies are engineered for secretion into culture supernatants for isolation therefrom.

### VII. Pharmaceutical Compositions and Dosing Methodologies

In one aspect, a pharmaceutical composition of the present invention includes an antigen binding molecule, e.g., a Trop-2 antibody or antigen binding fragment(s) thereof as described herein in combination with a pharmaceutically acceptable carrier. In other embodiments, the Trop-2 antibody or antigen binding fragment(s) thereof are administered in combination with a pharmaceutically acceptable carrier. Anti-Trop-2 compositions may include one or more different antibodies, one or more multispecific antibodies, one or more fusion proteins, one or more immunoconjugates, or a combination thereof as described herein.

The present invention provides pharmaceutical compositions comprising the antigen binding molecules of the present invention. The pharmaceutical compositions of the invention are formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of antigen binding molecule administered to a patient may vary depending upon the age and the size of the patient, target disease, conditions, route of administration, and the like. The preferred dose is typically calculated according to body weight or body surface area. When a bispecific antigen binding molecule of the present invention is used for therapeutic purposes in an adult patient, it may be advantageous to intravenously administer the bispecific antigen binding molecule of the present invention normally at a single dose of about 0.01 to about 20 mg/kg body weight, more preferably about 0.02 to about 7, about 0.03 to about 5, or about 0.05 to about 3 mg/kg body weight. Depending on the severity of the condition, the frequency and the duration of the treatment can be adjusted. Effective dosages and schedules for administering a bispecific antigen binding molecule may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, it is preferred that the aforesaid antibody is contained in about 5 to about 100 mg and in about 10 to about 250 mg for the other dosage forms.

In another aspect, a method for treating a cell proliferative disorder, such as cancer, a chronic infection, or an immunologically compromised disease state includes administering to a subject in need thereof a pharmaceutical composition containing an anti-Trop-2 antibody or antigen binding fragment as described herein in combination with a pharmaceutically acceptable carrier. In some embodiments, the method restores, potentiates or enhances the activity of lymphocytes in a subject in need thereof. In certain preferred embodiments, the antibody or fragment is a human or humanized anti-Trop-2 antibody that reduces or abrogates signaling through the Trop-2.

Non-limiting cancers for treatment using the antigen binding molecules, e.g., anti-Trop-2 antibodies or antigen binding fragments thereof, of the present invention include, for example, liver cancer, bone cancer, pancreatic cancer, skin cancer, cancer of the head or neck, breast cancer, lung cancer, non-small cell lung cancer (NSCLC), castrate resistant prostate cancer (CRPC), melanoma, uterine cancer, colon cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally induced cancers including those induced by asbestos, hematologic malignancies including, for example, multiple myeloma, B-cell lymphoma, Hodgkin lymphoma/primary mediastinal B-cell lymphoma, non-Hodgkin's lymphomas, acute myeloid lymphoma, chronic myelogenous leukemia, chronic lymphoid leukemia, follicular lymphoma, diffuse large B-cell lymphoma, Burkitt's lymphoma, immunoblastic large cell lymphoma, precursor B-lymphoblastic lymphoma, mantle cell lymphoma, acute lymphoblastic leukemia, mycosis fungoides, anaplastic large cell lymphoma, T-cell lymphoma, and precursor T-lymphoblastic lymphoma, or any combination of these cancers. The present disclosure is also applicable to treatment of metastatic cancers. Patients can be tested or selected for one or more of the above-described clinical attributes prior to, during or after treatment.

In one embodiment, the anti-Trop-2 antibody is administered an amount effective to achieve and/or maintain in an individual (e.g., for 1, 2, 3, 4 weeks, and/or until the subsequent administration of antigen binding compound) a blood concentration of at least the EC₅₀, optionally the EC₇₀, optionally substantially the EC₁₀₀, for neutralization of the enzymatic activity of Trop-2. In one embodiment, the active amount of anti-Trop-2 antibody is an amount effective to achieve the EC₅₀, optionally the EC₇₀, optionally substantially the EC₁₀₀, for neutralization of the enzymatic activity of Trop-2 in an extravascular tissue of an individual. In one embodiment, the active amount of anti-Trop-2 antibody is an amount effective to achieve (or maintain) in an individual the EC₅₀, optionally the EC₇₀, optionally substantially the EC₁₀₀, for inhibition of neutralize the enzymatic activity of Trop-2.

Optionally, in one embodiment, in contrast to some antibodies that are directed to the depletion of Trop-2-expressing tumor cells by ADCC (which, e.g., can provide full efficacy at concentrations equal or substantially lower than that which provides receptor saturation), the anti-Trop-2 antibody is a mainly blocker (no substantial Fcγ receptor-mediated activity) and is administered in an amount effective to neutralize the enzymatic activity of Trop-2 for a desired period of time, e.g., 1 week, 2 weeks, a month, until the next successive administration of anti-Trop-2 antibody.

In one embodiment, provided is a method for treating or preventing cancer in an individual, the method comprising administering to an individual having disease an anti-Trop-2 antibody in an amount that achieves or maintains for a specified period of time a concentration in circulation, optionally in an extravascular tissue of interest (e.g., the tumor or tumor environment), that is higher than the concentration required for 50%, 70%, or full (e.g., 90%) receptor saturation Trop-2-expressing cells in circulation (for example as assessed in PBMC). Optionally the concentration achieved is at least 20%, 50% or 100% higher than the concentration required for the specified receptor saturation.

In one embodiment, provided is a method for treating or preventing cancer in an individual, the method comprising administering to the individual an anti-Trop-2 antibody in an amount that achieves or maintains for a specified period of time a concentration in circulation, optionally in an extravascular tissue of interest (e.g., the tumor or tumor environment), that is higher than the EC₅₀, optionally EC₇₀ or optionally EC₁₀₀, for binding to Trop-2-expressing cells. Optionally the concentration achieved is at least 20%, 50% or 100% higher than the EC₅₀, optionally EC₇₀ or optionally EC₁₀₀, for binding to Trop-2-expressing cells.

In any embodiment, the antibody can for example have an EC₅₀, optionally EC₇₀ or optionally EC₁₀₀, for binding to Trop-2-expressing cells in human PBMC of between 0.5-100 ng/ml, optionally 1-100 ng/ml, optionally 30-100 ng/ml, e.g., about 30-90 ng/ml. For example, the EC₅₀ may be about 30, 37, 39, 43, 57, 58, 61, 62, 90, 95, 143 ng/ml.

When tissues outside of the vasculature are targeted (the tumor environment, e.g., in the treatment of solid tumors), an approximately 10-fold higher dose is typically believed to be needed, compared to the dose that provides the corresponding concentration in circulation. An amount of anti-Trop-2 antibody administered so at to achieve (and/or maintain) a concentration in circulation (blood) of about 1 μg/ml, 2 μg/ml, 10 μg/ml, or 20 μg/ml is expected to achieve (and/or maintain) an extravascular tissue (e.g., tumor tissue) concentration of about 0.1 μg/ml, 0.2 μg/ml, 1 μg/ml, 2 μg/ml, respectively.

In one embodiment, an anti-Trop-2 antibody is for example administered in an amount so at to achieve and/or maintain a tissue (e.g., tumor environment) concentration of at least 0.1 μg/ml, optionally at least 0.2 μg/ml, optionally at least 1 μg/ml, or optionally at least 2 μg/ml. The antibody can for example be administered in an amount to achieve and/or maintained a blood concentration of at least about 1 μg/ml, 2 μg/ml, 10 μg/ml, or 20 μg/ml, e.g., between 1-100 μg/ml, 10-100 μg/ml, 1-50 μg/ml, 1-20 μg/ml, or 1-10 μg/ml. The amount administered can be adjusted to as to provide for maintenance of the desired concentration for the duration of a specified period of time following administration (e.g., 1, 2, 3, 4 weeks, etc.).

In some embodiments, an amount of anti-Trop-2 antibody is administered so as to obtain a concentration in blood (serum) or an extravascular tissue (e.g., tumor environment) that corresponds to at least the EC₇₀ or the EC₁₀₀ for binding of Trop-2. The antibody can for example be administered in an amount to achieve and/or maintained a blood concentration or an extravascular tissue (e.g., tumor environment) of at least about 1 μg/ml, 2 μg/ml, 10 μg/ml, or 20 μg/ml.

EC₅o, EC₇o and EC₁₀₀ values for a given Trop-2 antibody can be assessed for example in a cellular assay for binding to Trop-2 using a method described in Example 3 or a substantially similar method. “EC₅₀” with respect to binding to Trop-2, refers to the concentration of anti-Trop-2 antibody which produces 50% of its maximum binding to Trop-2. “EC₇₀” with respect to binding to Trop-2, refers to the concentration of anti-Trop-2 antibody which produces 70% of its maximum binding to Trop-2. “EC₁₀₀” with respect to binding to Trop-2, refers to the concentration of anti-Trop-2 antibody which produces maximum binding to Trop-2.

In some embodiments, particularly for the treatment of solid tumors, the concentration achieved is designed to lead to a concentration in tissues (outside of the vasculature, e.g., in the tumor or tumor environment) that corresponds to at least the EC₅₀ for binding to Trop-2, optionally at about, or at least about, the EC₁₀₀.

In one embodiment, the amount of anti-Trop-2 antibody is between 1 and 20 mg/kg body weight. In one embodiment, the amount is administered to an individual weekly, every two weeks, monthly or every two months.

In one embodiment, a method of treating a cancer in a subject in need thereof, includes administering to the individual an effective amount of an anti-Trop-2 antibody of the disclosure for at least one administration cycle (optionally at least 2, 3, 4 or more administration cycles), wherein the cycle is a period of eight weeks or less, wherein for each of the at least one cycles, one, two, three or four doses of the anti-Trop-2 antibody are administered at a dose of 1-20 mg/kg body weight. In one embodiment, the anti-Trop-2 antibody is administered by intravenous infusion.

Suitable treatment protocols for treating e.g., a human subject include, for example, administering to the patient an amount as disclosed herein of an anti-Trop-2 antibody, wherein the method includes at least one administration cycle in which at least one dose of the anti-Trop-2 antibody is administered. Optionally, at least 2, 3, 4, 5, 6, 7 or 8 doses of the anti-Trop-2 antibody are administered. In one embodiment, the administration cycle is between 2 weeks and 8 weeks.

In one embodiment, a method for treating or preventing a disease (e.g., a cancer, a solid tumor, a hematological tumor) in an individual, includes administering to the individual an anti-Trop-2 antibody that neutralizes the enzymatic activity of Trop-2 for at least one administration cycle, the administration cycle comprising at least a first and second (and optionally a 3rd, 4th, 5th 6th, 7th and/or 8th or further) administration of the anti-Trop-2 antibody, wherein the anti-Trop-2 antibody is administered in an amount effective to achieve, or to maintain between two successive administrations, a blood (serum) concentration of anti-Trop-2 antibody of at least 0.1 μg/ml, at least 0.2 μg/ml, at least 1 μg/ml, at least 2 μg/ml, at least 10 μg/ml, at least 20 μg/ml, between 1-100 μg/ml, between 1-50 μg/ml, between 1-20 μg/ml, between 1-10 μg/ml or a range between any of the aforementioned concentrations.

In one embodiment, a specified continuous blood concentration is maintained, wherein the blood concentration does not drop substantially below the specified blood concentration for the duration of the specified time period (e.g., between two administrations of antibody, number of weeks, 1 week, 2 weeks, 3 weeks, 4 weeks). In other words, although the blood concentration can vary during the specified time period, the specified blood concentration maintained represents a minimum or “trough” concentration.

In one embodiment, a therapeutically active amount of an anti-Trop-2 antibody is an amount of such antibody capable of providing (at least) the EC₅₀ concentration, optionally the EC₇₀ concentration optionally the EC₁₀₀ concentration, in blood and/or in a tissue for binding to Trop-2 for a period of at least about 1 week, about 2 weeks, or about one month, following administration of the antibody.

Prior to or during a course of treatment with an anti-Trop-2 antibody of the disclosure, expression levels of Trop-2; percentages of Trop-2-expressing, can be assessed within and/or adjacent to a patient's tumor to assess whether the patient is suitable for treatment and is likely to respond to treatment. Increased levels or expression of the foregoing may indicate an individual is suitable for treatment with (e.g., likely to benefit from) an anti-Trop-2 antibody of the present disclosure.

In some embodiments, assessing the expression levels of Trop-2 within and/or adjacent to a patient's tumor the tissue sample includes the step of obtaining from a subject a biological sample of a human tissue selected from the group consisting of tissue from a cancer patient, e.g., cancer tissue, tissue proximal to or at the periphery of a cancer, cancer adjacent tissue, adjacent non-tumorous tissue or normal adjacent tissue, and expression levels of Trop-2 within the tissue. The expression levels or nucleotide concentrations from the patient can be comparing the level to a reference level, e.g., corresponding to a healthy individual.

In view of the foregoing, in certain embodiments, the method includes the steps of: (a) determining the expression levels of Trop-2 in the tumor environment, optionally within the tumor and/or within adjacent tissue, and upon a determination that tumor environment exhibits levels of Trop-2 that is/are increased compared to their corresponding reference level(s), (b) administering to the individual an anti-Trop-2 antibody.

In certain embodiments, determining the levels of Trop-2, and/or Trop-2 within the tumor environment includes the step of obtaining from the subject a biological containing cancer tissue and/or tissue proximal to or at the periphery of a cancer (e.g., cancer adjacent tissue, adjacent non-tumorous tissue or normal adjacent tissue), and detecting levels and/or relative percentages of Trop-2. Trop-2—expressing cells may include, for example, cancer cells. Expression levels of Trop-2, may be determined by evaluating their mRNA expression (by e.g., RT-PCR) or polypeptide expression (by e.g., western blotting, immunofluorescent staining) compared to a reference level corresponding to a healthy subject or compared to a reference level before treatment using techniques well known to those of ordinary skill in the art.

A subject with cancer can be treated with the anti-Trop-2 antibody with or without assessing the Trop-2 levels in the tumor microenvironment (e.g., on cancer cells).

A determination that a biological sample includes cells overexpressing Trop-2 compared to a reference, indicates that the subject has a cancer that may benefit from treatment with an agent that binds to Trop-2. In some embodiments, the term “overexpressed” is used with reference to a Trop-2 that is expressed in a substantial number of cells taken from a given patient, for example, on at least 10%, at least 20%, at least 30%, at least 40%, at least 50%, at least 60%, at least 70%, at least 80%, at least 90%, at least 95% or more of the tumor cells or lymphocytes taken from a subject.

In one embodiment, a method for the treatment or prevention of a cancer in an subject in need thereof includes the steps of: (a) detecting the percentage of cells and/or extent of expression corresponding to Trop-2, and/or PD-1 within the tumor environment, optionally within the tumor and/or within adjacent tissue, and upon a determination that the tumor environment includes cells overexpressing Trop-2, and/or PD-1, optionally at level(s) that are increased compared to suitable reference levels, (b) administering to the subject an anti-Trop-2 antibody. In one embodiment, the cells are tumor cells. In another embodiment, the cells within the tumor environment, tumor and/or adjacent tissue are non-malignant immune cells, e.g., T cells.

In some embodiments, determining the extent of Trop-2, and/or PD-1 expression within the tumor environment includes the step of obtaining from the individual a biological sample that comprises cancer tissue and/or tissue proximal to or at the periphery of a cancer (e.g., cancer adjacent tissue, adjacent non-tumorous tissue or normal adjacent tissue), contacting the cells with an antibody that binds an Trop-2 polypeptide, and/or PD-1 polypeptide and detecting the percentage of cells and/or the extent of expression corresponding to the Trop-2, and/or PD-1. In certain embodiments, expression of Trop-2, and/or PD-1 is evaluated by their cell surface expression using an immunohistochemistry assay.

The antibody compositions may be used in as monotherapy or combined treatments with one or more other therapeutic agents, including agents normally utilized for the particular therapeutic purpose for which the antibody is being administered. See “Combination therapies” above. The additional therapeutic agent will normally be administered in amounts and treatment regimens typically used for that agent in a monotherapy for the particular disease or condition being treated.

Such therapeutic agents include, but are not limited to anti-cancer agents and chemotherapeutic agents.

As described above, methods for using the pharmaceutical compositions described herein include the step of administering to a subject in need thereof an effective amount of the pharmaceutical composition according to the present disclosure.

Any suitable route or mode of administration can be employed for providing the patient with a therapeutically or prophylactically effective dose of the antibody. Exemplary routes or modes of administration include parenteral (e.g., intravenous, intraarterial, intramuscular, subcutaneous, intratumoral), oral, topical (nasal, transdermal, intradermal or intraocular), mucosal (e.g., nasal, sublingual, buccal, rectal, vaginal), inhalation, intralymphatic, intraspinal, intracranial, intraperitoneal, intratracheal, intravesical, intrathecal, enteral, intrapulmonary, intralymphatic, intracavital, intraorbital, intracapsular and transurethral, as well as local delivery by catheter or stent.

A pharmaceutical composition comprising an anti-Trop-2 antibody in accordance with the present disclosure may be formulated in any pharmaceutically acceptable carrier(s) or excipient(s). As used herein, the term “pharmaceutically acceptable carrier” includes any and all solvents, dispersion media, coatings, antibacterial and antifungal agents, isotonic and absorption delaying agents, and the like that are physiologically compatible. Pharmaceutical compositions may comprise suitable solid or gel phase carriers or excipients. Exemplary carriers or excipients include but are not limited to, calcium carbonate, calcium phosphate, various sugars, starches, cellulose derivatives, gelatin, and polymers such as polyethylene glycols. Exemplary pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the therapeutic agents.

In certain preferred embodiments, the therapeutically active agents can be incorporated into a pharmaceutical composition suitable for parenteral administration. Pharmaceutical composition for parenteral administration may be formulated by injection e.g., by bolus injection or continuous infusion.

Suitable buffers include but are not limited to, sodium succinate, sodium citrate, sodium phosphate or potassium phosphate. Sodium chloride can be used to modify the toxicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 2-4%). Stabilizers can be used in both liquid and lyophilized dosage forms, principally 1-50 mM L-Methionine (optimally 5-10 mM). Other suitable bulking agents include glycine, arginine, can be included as 0-0.05% polysorbate-80 (optimally 0.005-0.01%). Additional surfactants include but are not limited to polysorbate 20 and BRIJ surfactants.

Therapeutic agent preparations can be lyophilized and stored as sterile powders, preferably under vacuum, and then reconstituted in bacteriostatic water (containing, for example, benzyl alcohol preservative) or in sterile water prior to injection. The therapeutic agents in the pharmaceutical compositions may be formulated in a “therapeutically effective amount” or a “prophylactically effective amount”. A “therapeutically effective amount” refers to an amount effective, at dosages and for periods of time necessary, to achieve the desired therapeutic result. A therapeutically effective amount of an antibody or active agent may vary depending on the condition to be treated, the severity and course of the condition, the mode of administration, whether the antibody or agent is administered for preventive or therapeutic purposes, the bioavailability of the particular agent(s), the ability of the antibody to elicit a desired response in the individual, previous therapy, the age, weight and sex of the patient, the patient's clinical history and response to the antibody, the type of the antibody used, discretion of the attending physician, etc. A therapeutically effective amount is also one in which any toxic or detrimental effects of the recombinant vector is outweighed by the therapeutically beneficial effects. A “prophylactically effective amount” refers to an amount effective, at dosages and for periods of time necessary, to achieve the desired prophylactic result.

Preferably, the polypeptide domains utilized in the antibodies or other active agents described herein are derived from the same host in which they are to be administered in order to reduce inflammatory responses against the administered therapeutic agents. As suggested above, the therapeutic agent(s) are suitably administered to the subject at one time or over a series of treatments and may be administered to the patient at any time from diagnosis onwards. The Trop-2 antibody may be administered as the sole treatment or in conjunction with other active agents or therapies useful in treating the condition in question.

As a general proposition, a therapeutically effective amount or prophylactically effective amount of the Trop-2 antibody (or other active agent) will be administered in a range from about 1 ng/kg body weight/day to about 100 mg/kg body weight/day whether by one or more administrations. In a particular embodiment, each Trop-2 antibody or active agent is administered in the range of from about 1 ng/kg body weight/day to about 10 mg/kg body weight/day, about 1 ng/kg body weight/day to about 1 mg/kg body weight/day, about 1 ng/kg body weight/day to about 100 μg/kg body weight/day, about 1 ng/kg body weight/day to about 10 μg/kg body weight/day, about 1 ng/kg body weight/day to about 1 μg/kg body weight/day, about 1 ng/kg body weight/day to about 100 ng/kg body weight/day, about 1 ng/kg body weight/day to about 10 ng/kg body weight/day, about 10 ng/kg body weight/day to about 100 mg/kg body weight/day, about 10 ng/kg body weight/day to about 10 mg/kg body weight/day, about 10 ng/kg body weight/day to about 1 mg/kg body weight/day, about 10 ng/kg body weight/day to about 100 μg/kg body weight/day, about 10 ng/kg body weight/day to about 10 μg/kg body weight/day, about 10 ng/kg body weight/day to about 1 μg/kg body weight/day, 10 ng/kg body weight/day to about 100 ng/kg body weight/day, about 100 ng/kg body weight/day to about 100 mg/kg body weight/day, about 100 ng/kg body weight/day to about 10 mg/kg body weight/day, about 100 ng/kg body weight/day to about 1 mg/kg body weight/day, about 100 ng/kg body weight/day to about 100 μg/kg body weight/day, about 100 ng/kg body weight/day to about 10 μg/kg body weight/day, about 100 ng/kg body weight/day to about 1 μg/kg body weight/day, about 1 μg/kg body weight/day to about 100 mg/kg body weight/day, about 1 μg/kg body weight/day to about 10 mg/kg body weight/day, about 1 μg/kg body weight/day to about 1 mg/kg body weight/day, about 1 μg/kg body weight/day to about 100 μg/kg body weight/day, about 1 μg/kg body weight/day to about 10 g/kg body weight/day, about 10 μg/kg body weight/day to about 100 mg/kg body weight/day, about 10 μg/kg body weight/day to about 10 mg/kg body weight/day, about 10 μg/kg body weight/day to about 1 mg/kg body weight/day, about 10 μg/kg body weight/day to about 100 μg/kg body weight/day, about 100 μg/kg body weight/day to about 100 mg/kg body weight/day, about 100 μg/kg body weight/day to about 10 mg/kg body weight/day, about 100 μg/kg body weight/day to about 1 mg/kg body weight/day, about 1 mg/kg body weight/day to about 100 mg/kg body weight/day, about 1 mg/kg body weight/day to about 10 mg/kg body weight/day, about 10 mg/kg body weight/day to about 100 mg/kg body weight/day.

In other embodiments, the Trop-2 antibody and/or active agent is administered at a dose of 500 μg to 20 g every three days, or 25 mg/kg body weight every three days.

In other embodiments, each Trop-2 antibody and/or active agent is administered in the range of about 10 ng to about 100 ng per individual administration, about 10 ng to about 1 μg per individual administration, about 10 ng to about 10 μg per individual administration, about 10 ng to about 100 μg per individual administration, about 10 ng to about 1 mg per individual administration, about 10 ng to about 10 mg per individual administration, about 10 ng to about 100 mg per individual administration, about 10 ng to about 1000 mg per injection, about 10 ng to about 10,000 mg per individual administration, about 100 ng to about 1 μg per individual administration, about 100 ng to about 10 μg per individual administration, about 100 ng to about 100 μg per individual administration, about 100 ng to about 1 mg per individual administration, about 100 ng to about 10 mg per individual administration, about 100 ng to about 100 mg per individual administration, about 100 ng to about 1000 mg per injection, about 100 ng to about 10,000 mg per individual administration, about 1 μg to about 10 μg per individual administration, about 1 μg to about 100 μg per individual administration, about 1 μg to about 1 mg per individual administration, about 1 μg to about 10 mg per individual administration, about 1 μg to about 100 mg per individual administration, about 1 μg to about 1000 mg per injection, about 1 μg to about 10,000 mg per individual administration, about 10 μg to about 100 μg per individual administration, about 10 μg to about 1 mg per individual administration, about g to about 10 mg per individual administration, about 10 μg to about 100 mg per individual administration, about 10 μg to about 1000 mg per injection, about 10 μg to about 10,000 mg per individual administration, about 100 μg to about 1 mg per individual administration, about 100 μg to about 10 mg per individual administration, about 100 μg to about 100 mg per individual administration, about 100 μg to about 1000 mg per injection, about 100 μg to about 10,000 mg per individual administration, about 1 mg to about 10 mg per individual administration, about 1 mg to about 100 mg per individual administration, about 1 mg to about 1000 mg per injection, about 1 mg to about 10,000 mg per individual administration, about 10 mg to about 100 mg per individual administration, about 10 mg to about 1000 mg per injection, about 10 mg to about 10,000 mg per individual administration, about 100 mg to about 1000 mg per injection, about 100 mg to about 10,000 mg per individual administration and about 1000 mg to about 10,000 mg per individual administration. The antibodies of the present disclosure may be administered daily, every 2, 3, 4, 5, 6 or 7 days, or every 1, 2, 3 or 4 weeks.

In other particular embodiments, the amount of each Trop-2 antibody or active agent may be administered at a dose of about 0.0006 mg/day, 0.001 mg/day, 0.003 mg/day, 0.006 mg/day, 0.01 mg/day, 0.03 mg/day, 0.06 mg/day, 0.1 mg/day, 0.3 mg/day, 0.6 mg/day, 1 mg/day, 3 mg/day, 6 mg/day, 10 mg/day, 30 mg/day, 60 mg/day, 100 mg/day, 300 mg/day, 600 mg/day, 1000 mg/day, 2000 mg/day, 5000 mg/day or 10,000 mg/day.

In certain embodiments, the coding sequences for the Trop-2 antibody and/or other active agent(s) are incorporated into a suitable expression vector (e.g., viral or non-viral vector) for expressing an effective amount of the Trop-2 antibody or other active agent in a subject in need of treatment in accordance with the above-described methods. In certain embodiments comprising administration of e.g., one or more recombinant AAV (rAAV) viruses, the pharmaceutical composition may comprise the rAAVs in an amount comprising at least 10¹⁰, at least 10¹¹, at least 10¹², at least 10¹³, or at least 10¹⁴ genome copies (GC) or recombinant viral particles per kg, or any range thereof. In certain embodiments, the pharmaceutical composition comprises an effective amount of the recombinant virus, such as rAAV, in an amount comprising at least 10¹⁰, at least 10¹¹, at least 10¹², at least 10¹³, at least 10¹⁴, at least 10¹⁵ genome copies or recombinant viral particles genome copies per subject, or any range thereof.

Dosages can be tested in one or several art-accepted animal models suitable for any particular cell proliferative disorder or immune-compromised disease state.

Delivery methodologies may also include the use of polycationic condensed DNA linked or unlinked to killed viruses, ligand linked DNA, liposomes, eukaryotic cell delivery vehicles cells, deposition of photopolymerized hydrogel materials, use of a handheld gene transfer particle gun, ionizing radiation, nucleic charge neutralization or fusion with cell membranes, particle mediated gene transfer and the like.

### VIII. Diagnostic Uses of the Antibodies

The antigen binding molecules, e.g., antibodies or the antigen binding fragment thereof, of the present invention may also be used to detect and/or measure human or cynomolgus Trop-2, or human or cynomolgus Trop-2 expressing cells in a sample, e.g., for diagnostic purposes. For example, an anti-Trop-2 antibody, or the antigen binding fragment thereof, may be used to diagnose a condition or disease characterized by aberrant expression (e.g., over-expression, under-expression, lack of expression, etc.) of Trop-2. Exemplary diagnostic assays for Trop-2, e.g., contacting a sample, obtained from a patient, with an antibody of the invention, wherein the antibody is labeled with a detectable label or reporter molecule. Alternatively, an unlabeled antibody can be used in diagnostic applications in combination with a secondary antibody which is itself detectably labeled. The detectable label or reporter molecule can be a radioisotope, such as ³H, ¹⁴C, ¹⁸F, ³²p, ³⁵S, or ¹²⁵I; a fluorescent or chemiluminescent moiety such as fluorescein isothiocyanate, or rhodamine; or an enzyme such as alkaline phosphatase, betagalactosidase, horseradish peroxidase, or luciferase.

Specific exemplary assays that can be used to detect or measure Trop-2 in a sample include enzyme-linked immunosorbent assay (ELISA), radioimmunoassay (RIA), and fluorescence-activated cell sorting (FACS). Samples that can be used in Trop-2 diagnostic assays according to the present invention include any tissue or fluid sample obtainable from a patient which contains detectable quantities of Trop-2 protein, or fragments thereof, under normal or pathological conditions. Generally, levels of Trop-2 in a particular sample obtained from a healthy patient (e.g., a patient not afflicted with a disease or condition associated with abnormal Trop-2 levels or activity) will be measured to initially establish a baseline, or standard, level of Trop-2. This baseline level of Trop-2 can then be compared against the levels of Trop-2 measured in samples obtained from individuals suspected of having a Trop-2 related disease or condition.

Moreover, the anti-Trop-2 antibodies described herein can be used to purify human Trop-2 via immunoaffinity purification.

### IX. Kits

Any of the compositions described herein, e.g., the anti-Trop-2 antigen binding molecules of the present invention, and/or the additional therapeutic agent, may be comprised in a kit. In a non-limiting example, the kit comprises an antigen binding molecule, e.g., an antibody or antigen binding fragment thereof. In certain embodiments, the kit further includes an additional therapeutic agent described herein.

The kit may further include reagents or instructions for treating a disease or disorder. It may also include one or more buffers.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also generally contains a second, third or other additional container into which the additional components may be separately placed. The kits may also comprise a second container means for containing a sterile, pharmaceutically acceptable buffer and/or other diluent. However, various combinations of components may be comprised in a vial. The kits of the present invention also typically include a means for containing the compositions of the invention, e.g., the anti-Trop-2 antigen binding molecules and/or the additional therapeutic agent, and any other reagent containers in close confinement for commercial sale.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

The present invention is further illustrated by the following examples which should not be construed as limiting. The contents of all references, patents and published patent applications cited throughout this application, as well as the Figures and Tables are incorporated herein by reference.

### EXAMPLES

### Example 1: Generation of rabbit anti human Trop-2 monoclonal antibodies

Immunization of rabbits

Two New Zealand White rabbits (4-6 weeks of age) were immunized by injecting human Trop-2 protein into the rabbits under an IACUC approved protocol. Briefly, the recombinant protein containing human Trop-2 extracellular domain (ECD) and rabbit Fc (custom order, BonOpus Bioscience) was used to immunize the rabbits. Serum samples were taken prior to the first immunization and 14 days after the 2^{nd} and 3^{rd} immunization. Table 22 below summarizes the immunization schedule.

TABLE 22
Immunization Schedule

| Day | Immunization | |
|---|---|---|
| 1 | 400 μg of antigen + adjuvant S.Q.* | |
| 21 | 200 μg of antigen + adjuvant S.Q. | |
| 42 | 200 μg of antigen + adjuvant S.Q. | |
| 59 | 400 μg of antigen I.V.** | |
| 63 | | Sacrifice rabbit #1 |
| 77 | 400 μg of antigen I.V. | |
| 81 | | Sacrifice rabbit #2 |

*S.Q. subcutaneous
**I.V. intravenous

Serum titration was performed using antigen specific enzyme-linked immunoassay (ELISA) assay. Briefly, 384-well microplates were precoated with 25 μl/well of 0.5 μg/ml human or cynomolgus Trop-2-His protein. Rabbit sera were diluted at 1:1000, followed by 7-point 3-fold serial dilution. The serially diluted sera were loaded in assay buffer. HRP-conjugated goat anti-rabbit IgG (Jackson Immuno Research) antibodies were used for detection.

Rabbits with high specific titers were euthanized to isolate spleen aseptically.

### Sorting of B-cells

Biotinylated human Trop-2 ECD-hFc-Avi (Sino Biological), human Trop-2 ECD-His protein (Kactus Biosystems), or cynomolgus Trop-2 ECD-His (Kactus Biosystems) were used for staining of rabbit B cells. If sorting antigens are not biotinylated, biotinylation will be performed. Briefly, 10 mM stock solution of Sulfo-NHS-LC-Biotin (ThermoFisher) were prepared by dissolve 1 mg biotin in 180 μL deionized water on ice. Based on protein amount to be biotinylated, 20 times of molar ratio of biotin were added to protein solutions and the mixture were incubated on ice for 2 hours. Free biotin was removed by dialyzing biotinylated protein against 1× PBS, with at least 2 buffer changes with 3 hours minimum interval in Slide-A-Lyzer™ mini dialysis cup (3.5K MWCO, 0.1 mL) (ThermoFisher). For each sorting, multiple vials of splenocytes from selected rabbit were thawed and cultured in B-cell culture media (RPMI-1640, 15% FBS, 1×HEPES, 1×2-ME (2-Mercaptoethanol), 1% Penicillin/Streptomycin) overnight before sorting.

Ninety-six well (96-well) B cell feeding plates were also prepared one day before sorting. Briefly, irradiated HuT78 feeder cells in B cell culture media containing IL-2, CD40L and phorbol myristate acetate were dispensed into 96-well culture plates (120 μL/well).

On the day of sorting, suspended and loosely attached splenocytes were collected by gently pipetting medium against the culturing surface of flask. The cells were then transferred to conical tube for centrifugation at 400×g for 3 minutes. The cell pellets were washed twice with ice cold FACS buffer (1×PBS+0.5% BSA) twice and stained with biotinylated Trop-2 protein at a final concentration of 5 μg/ml. The mixture was incubated at room temperature for 20 min. The staining mixture were then centrifuged at 400×g for 3 min and the cells were resuspended in FACS buffer. The suspended cells were transferred into 1.5 ml amber Eppendorf tube. NeutrAvidin-Dy594 (1:300 dilution, Invitrogen cat #22842) and FITC-conjugated anti-rabbit IgM antibody (1:500 dilution, Novus, cat #MB7173) were then added to the cell suspension and the mixture were incubated at 4° C. for 15-30 min. The staining mixture were centrifuged at 400×g for 3 min. The cells were washed twice with ice-cold FACS buffer. The washed cell pellets were resuspended at −10⁷ cells/ml in ice-cold 1×PBS+1% FBS. At least 10 minutes before sorting, 7-AAD (1 μg/ml, final concentration) were added for live/dead cell discrimination. Single 7-AAD-/IgM-/Dy594+ cell was sorted into each well of 96-well plate seeded with feeder cells. Sorted B cells in 96-well B cell culture plates were cultured at 37° C. with 5% CO₂ for 9-12 days.

### Screening of Single B Cell Culture

On day 9 to day 12 post sorting, 15 μL of B cell culture supernatants were collected for antigen specific ELISA assay similar to serum titer determination as described above. Briefly, 384-well microplates were precoated with 25 μl/well of 0.5 μg/ml human or cynomolgus Trop-2-His protein. The plate was blocked, washed, loaded with rabbit B cell culture supernatants and incubated at RT for 2h. HRP-conjugated goat anti-rabbit IgG (Jackson Immuno Research) antibodies were used for detection. On day 10 to day 13 post sorting, the B cell culture plates were centrifuged at 400×g for 3 min. Supernatants from positive clones (OD greater than cutoff 0.9 from antigen specific ELISA) were collected and the cell pellets were preserved in 100 μL DNA/RNA shield (Zymo Cat #R1100-250) and transferred to 250 μL PCR tubes.

Collected supernatants were subject to cell binding assay to screen for hTrop-2 cell binders. Briefly, MDA-MB-231 or MCF-7 cells (known to express human Trop-2) were first added to 96-well plate (1×10⁵ cells per well). Fifty microliters (50 μl) of B cell culture (undiluted) supernatant samples were incubated with cells for 30 minutes on ice. After being washed with FACS buffer (2% Fetal bovine serum in DPBS), one hundred microliters (100 μl) of 1 μg/ml of Alex Fluor 647-conjugated goat anti-rabbit IgG for B cell culture detection were added and incubated for 30 minutes on ice. Cells were resuspended with 100 μl of FACS buffer after two washes. One hundred microliters (100 μl) of 1 μg/ml of 7-AAD for live/dead staining was added to the cell suspension and incubated for 5 min on ice, cells are then ready for FACS analysis using BD Accuri C6 plus. Trodelvy (Sacituzumab govitecan, a commercially available anti-Trop-2 antibody and topoisomerase inhibitor drug conjugate) was used as positive control. Anti-HEL.hIgG1 or rabbit IgG format was used as isotype control.

### Cloning (or Sequencing) of VH and VL Encoding Gene

Total RNAs from selected clones were purified from cell pellets preserved in DNA/RNA shield using RNeasy Mini Kit (Zymo) following manufacturer's protocol. 36 μL of nuclease-free water (Ambion) were used to elute total RNA. Eleven microliters (11 IL) of total RNA from each clone were mixed with 1 μL oligo (dT)12-18 primer (Invitrogen) and 1 μL dNTPs (10 mM, ThermoScientific) and then were heated at 65° C. for 5 min. Reverse transcription reaction was then set up and carried out at 50° C. for 1 hour followed by incubation at 75° C. for 15 minutes using SuperScript III enzyme (Thermo Scientific). After cDNA synthesis, VH and VL genes were amplified separately via PCR with 5 μl of cDNA template, VH or VL variable region primer pairs using Go-Taq enzyme mix (ThermoFisher), Platinum™ Taq DNA Polymerase (ThermoFisher) or Kapa HIFI HotStart enzyme (Roche). The VH and VL PCR products were separated on 1% agarose gel electrophoresis system. The expected size of VH and VL amplicon is −500 bp. The corresponding bands of VH and VL PCR products for each clone were cut from gel and the VH and VL PCR products were extracted from gel with NucleoSpin® Gel and PCR Clean-Up Kit (Macherey-Nagel, Cat #740609.250) following manufacturer's protocol. Twelve to thirty microliters (12-30) l of elution buffer were used to eluted VH and VL PCR products depending on the amount of PCR products. The linear expression module cassette (LEM) PCR products were constructed by overlapping PCR using LongAmp Taq DNA Polymerase (NewEngland BioLabs, Cat #E5200S) with a C fragment containing CMV promotor, VH or VL, and an H fragment containing rabbit IgG heavy-chain CHI fragment, human IgG1 C_{H}2 and C_{H}3 fragment (for VH), or light-chain constant region (for VL), followed by SV40 transcription terminator and poly A signal sequences. Five microliters (5 l) of PCR product were used to verify the size of the products and evaluate the magnitude of amplification by 0.8% agarose gel electrophoresis. The remaining PCR products were purified with NucleoSpin® Gel and PCR Clean-Up Kit following manufacturer's protocol. Thirty-two microliters (3241 of elution buffer were used to eluted LEM PCR products. Sequencing was performed for LEM constructs by Quintara Bioscience (Cambridge, MA).

### Screening of LEM Transfection

Antigen specific ELISA (as described above) was performed on the supernatant samples obtained from LEM transfection. Briefly, 384-well microplates were precoated with 25 μl/well of 0.5 μg/ml human or cynomolgus Trop-2-His protein. The plate was blocked, washed, loaded with 1:3 fold serial diluted LEM transfection supernatants starting from 1:2 dilution and incubated at RT for 2h. HRP-conjugated goat anti-human IgG (Jackson Immuno Research) antibodies were used for detection. Sacituzuwmab govitecan was used as positive control. Anti-HEL.hIgG1 was used as isotype control.

Cell binding assay (as described above) was performed on the LEM transfection supernatant samples positive for antigen specific ELISA. Briefly, fifty microliters (50 μl) LEM transfection supernatant samples (undiluted or diluted at 1:5, or 1:25) was incubated with cells for 30 minutes on ice. After being washed with FACS buffer (2% Fetal bovine serum in DPBS), 100 μl of 1 μg/ml of Alex Fluor 647-conjugated anti-human IgG was added and incubated for 30 minutes on ice. Cells were resuspended with 100 μl of FACS buffer after two washes. One hundred microliters of 1 μg/ml of 7-AAD for live/dead staining was added to the cell suspension, cells were then ready for FACS analysis using BD Accuri C6 plus. Sacituzumab govitecan was used as positive control. Anti-HEL.hIgG1 was used as isotype control.

Binding affinity assay was performed on the LEM transfection supernatant samples positive for cell binding assay, according to the protocol recommended by Gator Bio Inc. Briefly, human Trop-2-His (Kactus, Cat #TRP-HM121) or cynmolgus Trop-His (Kactus, Cat #TRP-CM121) was diluted to 5 or 2.5 μg/ml. After three times of regeneration and neutralization of HFC probe (Gator, Cat #160003), LEM transfection supernatant samples to be tested in K buffer was diluted 1:1 in K buffer and immobilized onto HFC probe. Human or cynomolgus Trop-2-His protein was loaded at 5 g/ml, or 2.5 μg/ml, for 2-point affinity measurement. The association and dissociation of human or cynomolgus Trop-2-His antigen from antibody was measured on BLI machine Gator Prime. Affinity was calculated using Gator Bio analysis software. Blank control was used for reference subtraction. 1 μg/ml of Sacituzumab govitecan was used as positive control.

Table 23 below summarizes the binding affinity of exemplary anti-Trop-2 antibodies in LEM transfection supernatant samples to human and cynomolgus Trop-2 using Gator.

TABLE 23
Binding affinity of anti-Trop-2 LEM anitbodies in transfection
supernatant samples to human and cynomolgus Trop-2 protein

| | human Trop-2 | | | Cynomolgus Trop-2 | | |
|---|---|---|---|---|---|---|
| Clone | koff(1/s) | kon(1/Ms) | K_{D}(M) | koff(1/s) | kon(1/Ms) | K_{D}(M) |
| 9F7 | 5.8E−05 | 5.30E+05 | 1.10E−10 | 5.8E−05 | 7.74E+05 | 1.45E−10 |
| 2H3 | 1.3E−04 | 4.88E+05 | 2.69E−10 | 1.3E−04 | 7.27E+05 | 1.13E−10 |
| 69H10 | 4.6E−04 | 4.65E+05 | 9.89E−10 | 5.3E−04 | 5.92E+05 | 8.93E−10 |
| 65D5 | 4.6E−04 | 7.06E+05 | 6.56E−10 | 6.1E−04 | 9.72E+05 | 6.28E−10 |
| 3E9 | 3.8E−04 | 5.68E+05 | 6.75E−10 | 3.8E−04 | 8.88E+05 | 5.38E−10 |
| 9A5 | 4.1E−04 | 5.29E+05 | 7.67E−10 | 4.1E−04 | 9.19E+05 | 6.98E−10 |
| 12G6 | 4.7E−04 | 3.86E+05 | 1.22E−09 | 4.7E−04 | 6.03E+05 | 9.11E−10 |
| 1D10 | 2.7E−04 | 5.18E+05 | 5.30E−10 | 2.7E−04 | 6.88E+05 | 3.61E−10 |
| 62E3 | 3.8E−04 | 6.07E+05 | 6.23E−10 | 4.5E−04 | 6.51E+05 | 6.85E−10 |
| 3A4 | 6.9E−04 | 5.00E+05 | 1.38E−09 | 6.9E−04 | 7.29E+05 | 1.17E−09 |
| 63H3 | 1.6E−04 | 7.04E+05 | 2.20E−10 | 1.6E−04 | 7.40E+05 | 2.18E−10 |
| 66A1 | 2.8E−04 | 6.56E+05 | 4.26E−10 | 3.2E−04 | 8.69E+05 | 3.67E−10 |
| 2E12 | 6.7E−04 | 5.42E+05 | 1.23E−09 | 6.7E−04 | 8.02E+05 | 9.08E−10 |
| 72F12 | 3.8E−04 | 6.66E+05 | 5.74E−10 | 4.7E−04 | 8.98E+05 | 5.26E−10 |
| 74F11/15F5 | 1.7E−04 | 5.26E+05 | 3.23E−10 | 1.7E−04 | 7.58E+05 | 2.64E−10 |
| 9D7 | 3.9E−05 | 3.42E+05 | 1.13E−10 | 3.9E−05 | 5.28E+05 | 1.17E−10 |
| 10A9 | 5.3E−05 | 3.72E+05 | 1.42E−10 | 5.3E−05 | 5.69E+05 | 1.08E−10 |
| 10E12 | 6.8E−05 | 3.91E+05 | 1.73E−10 | 6.8E−05 | 6.05E+05 | 1.68E−10 |
| 8H2 | 9.6E−05 | 4.23E+05 | 2.27E−10 | 9.6E−05 | 6.77E+05 | 2.21E−10 |
| 10F12 | 6.8E−05 | 3.91E+05 | 1.73E−10 | 1.0E−05 | 4.26E+05 | 1.32E−10 |
| 10D3 | 1.9E−04 | 3.39E+05 | 5.62E−10 | 1.9E−04 | 4.54E+05 | 4.55E−10 |
| 2H5 | 2.7E−04 | 3.31E+05 | 8.22E−10 | 2.7E−04 | 4.60E+05 | 5.82E−10 |
| 5D4 | 1.0E−04 | 3.58E+05 | 2.80E−10 | 1.0E−04 | 5.77E+05 | 2.56E−10 |
| 4F2 | 6.7E−04 | 4.18E+05 | 1.60E−09 | 6.7E−04 | 9.18E+05 | 1.17E−09 |
| 62A10 | 5.1E−04 | 5.32E+05 | 9.59E−10 | 9.2E−04 | 7.26E+05 | 1.27E−09 |
| 70E2 | 3.8E−04 | 4.30E+05 | 8.71E−10 | 5.9E−04 | 6.57E+05 | 8.92E−10 |
| 71F10 | 3.2E−04 | 4.59E+05 | 6.96E−10 | 5.4E−04 | 7.21E+05 | 7.55E−10 |
| 73C2 | 1.1E−03 | 2.84E+05 | 3.85E−09 | 1.7E−03 | 3.64E+05 | 4.63E−09 |
| Sacituzumab | 2.2E−04 | 2.85E+05 | 7.55E−10 | 2.2E−04 | 4.05E+05 | 6.14E−10 |
| govitecan | 1.4E−04 | 2.81E+05 | 4.97E−10 | 2.5E−04 | 3.26E+05 | 7.72E−10 |

### Example 2: Expression of genes encoding antibody variable regions of anti-TROP-2 antibodies

Exemplary anti-Trop-2 antibodies of the present disclosure were produced in small scale using the LEM PCR products directly. Briefly, about 5 million (˜5×10⁶) HEK293T cells were seeded to a well in a 12-well plate one day before transfection. For each clone, about 500 ng of Heavy chain LEM PCR product and about 500 ng of Light chain LEM PCR product were diluted with 45 μL Opti-MEM medium (Gibco). Three microliters (3 μL) of Lipofectamine 2000 Reagent (Invitrogen) were diluted in 47 μL Opti-MEM medium. The diluted LEM PCR products and diluted Lipofectamine were mixed and incubate it at RT for 10 minutes. The HEK293T medium in each well was replaced with 1 mL of Opti-MEM medium. The mixture of LEM PCR product and Lipofectamine was added into each well dropwise. The plate was gently swirled to mix transfecting reagent and medium well. Transfected cells were incubated at 37° C., 5% CO₂ for 48-72 hours. The supernatants of the cell cultures were collected for further screening.

Alternatively, the genes encoding VH and V_{L} of various clones were sent to CRO (Biolntron or BonOpus Bioscience) for gene synthesis, gene cloning and small-scale antibody production in CHO or Expi293 cells. Antibodies in culture supernatants were affinity purified with Protein A agarose.

### Example 3: Specific binding of anti-Trop-2 antibodies to Trop-2 protein

### 1. Binding Specificity

Purified antibodies were tested by ELISA to confirm their binding and affinity against human and cynomolgus Trop-2-His protein. Briefly, 96-well Maxisorp plates (Nunc) were coated with 100 μl/well of 0.5 μg/ml human (Kactus Cat #TRP-HM121, or Sino Biological, Cat #10428-HO8H) or cynomolgus Trop-2 protein (Kactus Cat #TRP-CM121, or Sino Biological, Cat #90893-CO8H) diluted in 1×PBS (GIBCO) overnight at 4° C. Plates were washed four times with 300 μl/well washing buffer (1×PBST, diluted from 20×PBST (Thermo Scientific) with distilled water)) and then blocked with 300 μl/well of blocking buffer (1×PBST/3% BSA (BSA, Sigma)) for 2 hours at RT on plate shaker at 100 rpm. Plates were washed and added with 100 μl/well of testing antibodies, which were serial diluted with assay buffer (PBST/1% BSA). The antibodies were incubated at room temperature for 2 hours on plate shaker at 100 rpm. Plates were washed. One hundred microliters (100 μl) of 1:10,000 diluted horseradish peroxidase (HRP) conjugated anti-hIgG antibody (Jackson Immuno Research) was added into each well and incubated at room temperature for 1 hour on plate shaker at 100 rpm. Plates were washed and added with 100 μl/well TMB substrate for color development in dark at room temperature for up to 10 minutes. After addition of 50 μl/well of stop solution (1N H₂SO₄), plates were read at 450 nm on a plate reader (SpectraMax M5). EC₅₀ values were calculated using Graphpad Prism. Sacituzumab govitecan, was used as positive control. Anti-HEL.hIgG1 was used as isotype control. Table 24 below shows that the exemplary antibodies of the invention specifically bind to both human and cynomolgus Trop-2.

TABLE 24
Binding of anti-Trop-2 antibodies
to human and cynomolgus Trop-2

| | Human Trop-2 | Cynomolgus Trop-2 |
|---|---|---|
| Clone ID | EC50 (nM) | EC50 (nM) |
| 62E3 | 0.018 | 0.016 |
| 63H3 | 0.029 | 0.033 |
| 65D5 | 0.019 | 0.015 |
| 66A1 | 0.013 | 0.014 |
| 72F12 | 0.013 | 0.013 |
| 74F11/15F5 | 0.013 | 0.014 |
| 9F7 | 0.033 | 0.030 |
| 1D10 | 0.016 | 0.017 |
| 9D7 | 0.017 | 0.016 |
| 9D7-2 | 0.020 | 0.014 |
| 10A9 | 0.017 | 0.015 |
| 10A9-2 | 0.014 | 0.016 |
| 5D4 | 0.016 | 0.014 |
| 5D4-1 | 0.018 | 0.023 |
| 5D4-2 | 0.015 | 0.018 |
| Sacituzumab govitecan | 0.014 | 0.017 |
| anti-HEL hIgG1 | n.a. | n.a. |

Similar assays were performed to test whether the exemplary antibodies of the present disclosure bind to human Trop-1 (EpCAM) or mouse Trop-2 (Kactus, Cat #TRP-MM121) or rat Trop-2 (kactus, Cat #TRP_RM121). No binding of the exemplary antibodies of the present disclosure to human Trop-1 (EpCAM) or mouse or rat Trop-2 has been detected.

### 2. Binding Affinity

The affinity of the binding of the exemplary anti-Trop-2 antibodies of the present disclosure were determined. Assay was performed using Gator Plus according to the protocol recommended by Gator Bio Inc.. Briefly, human Trop-2-His (Kactus) was 1:4 fold serially diluted starting at 1 μg/ml. After three times of regeneration and neutralization of HFC probe, 1 μg/ml of antibody to be tested in Q buffer was immobilized onto HFC probe. Human Trop-2-His was loaded at 1 μg/ml, or 0.33 μg/ml, for 2-point affinity measurement (association time 100 sec, dissociation time 300 sec). The association and dissociation of human Trop-2-His antigen from antibody was measured on BLI machine Gator Plus. Affinity was calculated using Gator Bio analysis software. Blank control was used for reference subtraction. Sacituzumab govitecan was used as positive control.

Table 25 below summarizes the binding affinity to human Trop-2 using Gator.

TABLE 25
Binding affinity of anti-Trop-2 antibodies to human Trop-2

| | Kon | Koff | |
|---|---|---|---|
| | (× 10⁵, | (× 10⁻⁴, | KD |
| | 1/Ms) | 1/s) | (nM) |
| 62E3 | 7.26 | 4.29 | 0.59 |
| 63H3 | 7.66 | 0.25 | 0.03 |
| 65D5 | 8.19 | 8.98 | 1.10 |
| 66A1 | 7.88 | 1.93 | 0.25 |
| 72F12 | 8.65 | 5.41 | 0.63 |
| 74F11/15F5 | 7.07 | 0.09 | 0.01 |
| 9F7 | 6.34 | 0.28 | 0.04 |
| 1D10 | 6.04 | 1.31 | 0.22 |
| 9D7 | 4.32 | <0.01 | <0.002 |
| 9D7-2 | 4.38 | 12.60 | 2.86 |
| 10A9 | 4.48 | 0.82 | 0.18 |
| 10A9-2 | 3.35 | 8.90 | 2.66 |
| 5D4 | 5.22 | 0.13 | 0.03 |
| 5D4-1 | 4.89 | 0.51 | 0.10 |
| 5D4-2 | 4.87 | 0.60 | 0.12 |
| Sacituzumab | 2.39 | 6.35 | 0.27 |
| govitecan | | | |

### Example 4: Specific binding of anti-Trop-2 antibodies to Trop-2 expressing cells

The assay describe in this example is to determine whether the anti-Trop-2 antibodies of the present disclosure specifically binds to Trop-2 expressing cells.

In this assay, MDA-MB-231-GFP cells (known to express human Trop-2) were collected 5 from fresh culture. Cells were then resuspended in FACS buffer (1×DPBS/1% FBS) at 10⁶/ml concentration. One hundred microliter (100 μl) of cells were aliquoted into a single well in 96 well plates. The antibodies to be tested were serially diluted at 1:3 fold starting at 15 μg/mL and were then added into each well. The antibodies were mixed with cells and the mixtures were incubated on ice for 1 hour. After washing with FACS buffer, one hundred microliter (100 μl) of 1:400 diluted PE-conjugated anti-human Fc secondary antibody (Jackson Immuno Research) was added into each well and incubated for 30 minutes on ice. After washing with FACS buffer, stained cells were resuspended at diluted 7-ADD (Biolegend, used at 1:100 dilution) and analyzed on Guava 11HT or 5HT (Luminex). Data was analyzed using Flowjo software. EC₅₀ values were calculated using GraphPad Prism. Sacitnzurnab govitecan was used as positive control. Anti-HEL.hIgG1 was used as isotype negative control. Similar assay was performed using MCF-7 cells (known to express human Trop-2). Table 26 below shows that the exemplary antibodies of the present disclosure specifically bind to Trop-2 expressed on MDA-MD-231-GFP and MCF-7 cell surface.

TABLE 26
Binding of anti-Trop-2 antibodies to cell surface Trop-2

| | MDA-MB-231-GFP | MCF-7 |
|---|---|---|
| Clone ID | EC50 (nM) | EC50 (nM) |
| 62E3 | 0.01 | 0.7 |
| 63H3 | 0.02 | 0.6 |
| 65D5 | 0.01 | 0.8 |
| 66A1 | 0.01 | 0.6 |
| 72F12 | 0.01 | 0.7 |
| 74F11/15F5 | 0.02 | 0.9 |
| 9F7 | 0.03 | 0.3 |
| 1D10 | 0.01 | 0.9 |
| 9D7 | 0.03 | 1.2 |
| 9D7-2 | 0.1 | 1.0 |
| 10A9 | 0.04 | 1.4 |
| 10A9-2 | 0.04 | 1.8 |
| 5D4 | 0.01 | 1.2 |
| 5D4-1 | 0.01 | 1.0 |
| 5D4-2 | 0.04 | 0.8 |
| Sacituzumab govitecan | 0.09 | 2.2 |
| Anti-HEL hIgG1 | n.a. | n.a. |

### Example 5: ADCC of the anti-Trop-2 antibodies

Antibody-dependent cellular cytotoxicity (ADCC) is an immune mechanism through which Fc receptor-bearing effector cells can recognize and kill antibody binded target cells expressing antigens on their surface. ADCC is triggered by the cross-linking between antigen-bound antibodies and the Fc receptor CD16A (FcγRIIIA) at the surface of immune effector cells, such as Natural Killer cells. These interactions induce the increase of intracellular calcium concentrations and the translocation of the NFAT transcription factor to the nucleus, where it can bind to the promoter regions of ADCC relevant genes.

Antibody-dependent cellular cytotoxicity (ADCC) of the exemplary anti-Trop-2 antibodies of the present disclosure was measured in this experiment.

NFAT-CD16 Lucia Luciferase Reporter T Lymphocytes was purchased from InvivoGen. Twenty microliters (20 μl) of antibodies serially dilute at 1:3 fold starting at 3 μg/mL were added into a well in a flat-bottom 96-well plate. Subsequently, ninety microliters (90 μl) of MDA-MB-231 cell suspension (˜100,000 cells, known to express human Trop-2) were added to each well. After incubation of the plate at 37° C. in a CO2 incubator for 1 hour, ninety microliters (90 μl) of Jurkat-Lucia™ NFAT CD16 cell suspension (˜200,000 cells) was added into each well, and the plate was incubated at 37° C. in a CO2 incubator for 6 hours. Fifty microliters (50 μl) of co-incubated MDA-MB-231 and Jurkat-Lucia™ NFAT CD16 cell supernatant from each well was transferred into a single well in a 96-well white (opaque, Coming), followed by addition of 50 μl of QUANTI-Luc™ substrate (InvivoGen, freshly prepared following vendor's instruction)) into each well. Plates were proceeded immediately for measurement of luminescence signal on a plate reader (SpectraMax M5). Sacituzumab govitecan was used as a positive control. Anti-HEL.hIgG1 was used as isotype negative control. Similar assay was performed using MCF-7 cells (known to express human Trop-2).

Table 27 below summarizes the EC₅o of the ADCC of the exemplary anti-Trop-2 antibodies of the present disclosure.

TABLE 27
EC₅₀ Value of the ADCC of anti-Trop-2 antibodies

| | MDA-MB-231 | MCF-7 |
|---|---|---|
| Clone ID | EC50 (nM) | EC50 (nM) |
| 62E3 | 0.04 | 0.40 |
| 63H3 | 0.03 | 0.17 |
| 65D5 | 0.05 | 0.32 |
| 66A1 | 0.06 | 0.22 |
| 72F12 | 0.07 | 0.16 |
| 74F11/15F5 | 0.09 | 0.27 |
| 9F7 | 0.03 | 0.18 |
| 1D10 | 0.04 | 0.28 |
| 9D7 | 0.09 | 0.28 |
| 9D7-2 | 0.11 | 0.23 |
| 10A9 | 0.06 | 0.32 |
| 10A9-2 | 0.07 | 0.28 |
| 5D4 | 0.07 | 0.57 |
| 5D4-1 | 0.11 | 0.61 |
| 5D4-2 | 0.08 | 0.52 |
| Sacituzumab govitecan | 0.55* | 2.72* |
| anti-HEL hlgG1 | n.a. | n.a. |

*Average from 3 plates

### Example 6: Internalization of anti-Trop-2 antibodies

This example is to measure the internalization of the exemplary anti-Trop-2 antibodies of the present disclosure upon binding to Trop-2 expressed on cell surface.

About one hundred thousand (˜105) MDA-MB-231 cells were incubated with antibodies to be tested at indicated concentration (2, 1, 0.5 and 0.25 μg/ml) on ice. Trodelvy (sacituzumab govitecan) was used as positive controls. Anti-HEL.hIgG1 was used as isotype negative control. One hour after incubation, two aliquots were taken out and transferred into 37° C. incubator for 2 or 4 hours of incubation, respectively. Samples collected at different time points were washed with ice cold FACS buffer for two times and then stained with PE-conjugated anti-human Fc secondary antibody (Jackson Immuno Research) for 1 hour on ice. After two washes with ice cold FACS buffer, cells were stained with 7-AAD and then fixed with 2% of paraformaldehyde. MDA-MB-231 cells were known to express Trop-2. Sacituzumab was known to be an anti-Trop2 antibody and induce internalization upon binding to Trop2 on cell surface.

Samples were then analyzed on Guava 11HT or 5HT (Luminex) and data analysis was done with Flowjo software. Internalization rate for each antibody at each concentration was calculated as:

$$\% of internalization = \{ [ ( MFI of sample on ice ) - ( MFI of sample of indicated incubation time at 37 ° C . ) ] / ( MFI of sample on ice ) \} \times 100$$

Similar assay was preformed using MCF-7 cells. Tables 28 and 29 below summarizes the internationalization of different anti-Trop-2 antibodies. The results show that the exemplary anti-Trop-2 antibodies induces internalization upon binding to cell surface Trop-2.

TABLE 28
Internalization induced by anti-Trop-2 antibodies
(Internalization %) (MDA-MB-231 cells)

| | 2 μg/ml | | 1 μg/ml | | 0.5 μg/ml | | 0.25 μg/ml | |
|---|---|---|---|---|---|---|---|---|
| Clone ID | 2 h | 4 h | 2 h | 4 h | 2 h | 4 h | 2 h | 4 h |
| 62E3 | 43 | 48 | 43 | 49 | 42 | 45 | 41 | 46 |
| 63H3 | 37 | 39 | 38 | 49 | 39 | 45 | 40 | 48 |
| 65D5 | 31 | 41 | 37 | 48 | 43 | 51 | 34 | 51 |
| 66A1 | 36 | 43 | 39 | 49 | 46 | 50 | 45 | 51 |
| 72F12 | 37 | 45 | 35 | 45 | 42 | 48 | 38 | 48 |
| 74F11/15F5 | 39 | 46 | 42 | 48 | 37 | 45 | 42 | 52 |
| 9F7 | 28 | 31 | 33 | 41 | 34 | 46 | 34 | 38 |
| 1D10 | 39 | 44 | 41 | 51 | 48 | 52 | 46 | 53 |
| 9D7 | 42 | 49 | 43 | 54 | 47 | 55 | 42 | 52 |
| 9D7-2 | 48 | 59 | 51 | 62 | 49 | 58 | 52 | 63 |
| 10A9 | 38 | 49 | 40 | 55 | 47 | 53 | 54 | 57 |
| 10A9-2 | 45 | 58 | 43 | 57 | 44 | 55 | 46 | 55 |
| 5D4 | 60 | 66 | 65 | 51 | 49 | 66 | 56 | 65 |
| 5D4-1 | 57 | 63 | 59 | 64 | 56 | 61 | 58 | 66 |
| 5D4-2 | 57 | 62 | 52 | 58 | 48 | 57 | 55 | 61 |
| Sacituzumab | 43 | 48 | 43 | 49 | 42 | 45 | 41 | 46 |
| govitecan | | | | | | | | |
| Anti-HEL | n.a. | n.a. | n.a. | n.a. | n.a. | n.a. | n.a. | n.a. |
| hIgG1 | | | | | | | | |

TABLE 29
Internalization induced by anti-Trop-2 antibodies
(Internalization %) (MCF-7 Cells)

| | 0.25 μg/ml | | | 0.5 μg/ml | | |
|---|---|---|---|---|---|---|
| Clone ID | 1 h | 2 h | 4 h | 1 h | 2 h | 4 h |
| 62E3 | 23 | 39 | 45 | 21 | 41 | 40 |
| 63H3 | 15 | 26 | 36 | 23 | 37 | 44 |
| 65D5 | 18 | 36 | 41 | 23 | 41 | 42 |
| 66A1 | 20 | 36 | 45 | 33 | 45 | 53 |
| 72F12 | 21 | 37 | 47 | 24 | 39 | 48 |
| 74F11/15F5 | 22 | 35 | 64 | 22 | 54 | 50 |
| 9F7 | 2 | 31 | 36 | 29 | 44 | 49 |
| 1D10 | 30 | 56 | 49 | 44 | 54 | 65 |
| 9D7 | 42 | 75 | 69 | 46 | 60 | 65 |
| 9D7-2 | 29 | 42 | 57 | 28 | 42 | 52 |
| 10A9 | 15 | 33 | 45 | 26 | 39 | 49 |
| 10A9-2 | 28 | 37 | 48 | 31 | 47 | 64 |
| 5D4 | 44 | 52 | 64 | 62 | 59 | 74 |
| 5D4-1 | 59 | 57 | 62 | 42 | 56 | 73 |
| 5D4-2 | 51 | 67 | 58 | 23 | 43 | 61 |
| Sacituzumab | 30 | 43 | 44 | −4 | 41 | 55 |
| govitecan | | | | | | |
| Anti-HEL hIgG1 | n.a. | n.a. | n.a. | n.a. | n.a. | n.a. |

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the claimed components and steps in any sequence which is effective to meet the objectives there intended, unless the context specifically indicates the contrary.

1. An isolated antibody, or antigen-binding fragment thereof, that binds to human trophoblast cell surface antigen 2 (Trop-2), comprising
a heavy chain variable (V_{H}) domain comprising from N-terminus to C-terminus, three heavy chain complementarity-determining regions (CDRs), HCDR1, HCDR2, and HCDR3; and
a light chain variable (VL) domain comprising from N-terminus to C-terminus, three light chain complementarity-determining regions (CDRs), LCDR1, LCDR2, and LCDR3; wherein
(a) the HCDR1 comprises an amino acid sequence selected from the group consisting of Y-G-X1-X2, Y-G-X3-S, and Y-G-V-X4, wherein X1 is M or V, X2 is S or T, X3 is M or V, and X4 is S or T;
(b) the HCDR2 comprises an amino acid sequence selected from the group consisting of Y-I-Y-P-X44-X45-X46-N-X47-Y-Y-A-X48-W-V-N-G (SEQ ID NO: 316), Y-I-Y-P-A-X49-H-N-X50-Y-Y-A-X51-W-V-N-G or (SEQ ID NO: 317), YIYPTYHNTYYATWVNG (SEQ ID NO: 16), and YIYPAFPNTYYATWVNG (SEQ ID NO: 19), wherein X44 is A or T, X45 is F or Y, X46 is H or P, X47 is A, R, or T, and X48 is N, S or T, X49 is F or Y, X50 is A, R, or T, and X51 is N, S, or T;
(c) the HCDR3 comprises an amino acid sequence D-X99-G-X100-X101-D-Y-X102-X103-N-L (SEQ ID NO: 333) or D-A-G-X104-T-D-Y-X105-X106-N-L (SEQ ID NO: 334), wherein X99 is A or T, X100 is G, N, S, or T, X101 is T or V, X102 is A, K, N, or Y, X103 is F, L, or Y, X104 is G, N, S, or T, X105 is A, K, N, Y, X106 is F, L or Y;
(d) the LCDR1 comprises an amino acid sequence Q-A-S-X135-X136-I-X137-X138-X139-X140-X141 (SEQ ID NO: 345) or Q-A-S-X142-X143-I-X144-X145-Y-L-X146 (SEQ ID NO: 346), wherein X135 is E, K, or Q, X136 is D, N, or S, X137 is D, E, S, or Y, X138 is N, R, or S, X139 is N or Y, X140 is L or S, X141 is A or S, X142 is E or Q, X143 is D, N, or S, X144 is E, S, or Y, X145 is N, R, or S, X146 is A or S;
(e) the LCDR2 comprises an amino acid sequence selected from the group consisting of X170-A-X171-X172-L-X173-S(SEQ ID NO: 357), X174-A-S-X175-L-X176-S(SEQ ID NO: 358), and X177-A-S-X178-L-A-S(SEQ ID NO: 359), wherein X170 is A, D, E, or K, X171 is S or T, X172 is K or T, X173 is A, P, or T, X174 is A, D, E, or K, X175 is K or T, X176 is A, or P, X177 is A, D, E, or K, X178 is K or T; and
(f) the LCDR3 comprises an amino acid sequence X183-Q-X184-L-T-X185-G-X186-V-D-N-P (SEQ ID NO: 362) or Q-Q-X187-L-T-X188-G-X189-V-D-N-P (SEQ ID NO: 363), wherein X183 is H or Q, X184 is A, D, G, or V, X185 is I or V, X186 is D, N, or Y, X187 is A, D, G, or V, X188 is I or V, and X189 is D, N, or Y.

2-7. (canceled)

8. An isolated antibody, or antigen-binding fragment thereof, that binds to human Trop-2, comprising:
a heavy chain variable (V_{H}) domain comprising from N-terminus to C-terminus, three heavy chain complementarity-determining regions (CDRs), HCDR1, HCDR2, and HCDR3; and
a light chain variable (VL) domain comprising from N-terminus to C-terminus, three light chain complementarity-determining regions (CDRs), LCDR1, LCDR2, and LCDR3; wherein
(a) the HCDR1 comprises an amino acid sequence selected from the group consisting of Y-X5-X6-L, Y-A-X7-L, and Y-S-X8-L wherein X5 is A or S, X6 is I or M, X7 is I or M, and X8 is I or M;
(b) the HCDR2 comprises an amino acid sequence X52-X53-Y-X54-S-G-X55-X56-T-Y-X57-A-X58-W-A-X59-G (SEQ ID NO: 318), or X60-I-Y-I-S-G-G-X61-T-Y-X62-A-X63-W-A-X64-G (SEQ ID NO: 319), wherein X52 is C or S, X53 is I or L, X54 is F or I, X55 is A or G, X56 is S or T, X57 is F or Y, X58 is N or S, X59 is K or T, X60 is C or S, X61 is S or T, X62 is F or Y, X63 is N or S, and X64 is K or T;
(c) the HCDR3 comprises an amino acid sequence X107-D-G-X108-X109-X110-Y-Y-L-N-L (SEQ ID NO: 335), or D-D-G-X111-X112-S-Y-Y-L-N-L (SEQ ID NO: 336), wherein X107 is D or N, X108 is S or T, X109 is A, T or V, X110 is N or S, X111 is S or T, and X112 is A, T or V;
(d) the LCDR1 comprises an amino acid sequence Q-A-S-X147-X148-I-Y-X149-X150-X151-A (SEQ ID NO: 347), or Q-A-S-E-D-I-Y-X152-L-L-A (SEQ ID NO: 348), wherein X147 is E or Q, X148 is D or S, X149 is N, K, R, or S, X150 is L or N, X151 is F or L, and X152 is N, K, R, or S;
(e) the LCDR2 comprises an amino acid sequence X179-A-S-X180-L-X181-S(SEQ ID NO: 360); wherein X179 is A, D, or G, X180 is D, N, or T, X181 is A, E, or T; and
(f) the LCDR3 comprises an amino acid sequence Q-Q-X190-Y-T-X191-G-N-I-D-N-X192 (SEQ ID NO: 364), wherein X190 is A, or G, X191 is I or V, X192 is A, P, S or T.

9-14. (canceled)

15. An isolated antibody, or the antigen binding fragment thereof, that binds human Trop-2, comprising:
a heavy chain variable (V_{H}) domain comprising from N-terminus to C-terminus, three heavy chain complementarity-determining regions (CDRs), HCDR1, HCDR2, and HCDR3; and
a light chain variable (VL) domain comprising from N-terminus to C-terminus, three light chain complementarity-determining regions (CDRs), LCDR1, LCDR2, and LCDR3; wherein
(a) the HCDR1 comprises an amino acid sequence selected from the group consisting of T-Y-W-M-W (SEQ ID NO: 8) and T-Y-W-M-C(SEQ ID NO: 9);
(b) the HCDR2 comprises an amino acid sequence X65-I-Y-V-G-S-G-X66-S-T-Y-Y-A-S-W-A-K-G (SEQ ID NO: 320), wherein X65 is C, P, or S, and X66 is G or S;
(c) the HCDR3 comprises an amino acid sequence G-A-T-N-N-V-F-M-N-Y-F-N-L (SEQ ID NO: 57), or G-A-T-N-N-V-F-R-N-Y-F-N-L (SEQ ID NO: 58);
(d) the LCDR1 comprises an amino acid sequence Q-A-S-E-D-I-S-S-N-L-A (SEQ ID NO:76) or Q-A-S-E-D-I-S-S-N-L-G (SEQ ID NO: 75);
(e) the LCDR2 comprises an amino acid sequence G-A-S-T-L-A-S(SEQ ID NO: 91); and
(f) the LCDR3 comprises an amino acid sequence Q-S-S-Y-Y-I-D-D-G-V-N-G (SEQ ID NO: 111) or Q-T-S-Y-Y-I-D-D-G-V-N-G (SEQ ID NO: 110).

16-21. (canceled)

22. An isolated antibody, or antigen binding fragment thereof, that binds human Trop-2, comprising:
a heavy chain variable (V_{H}) domain comprising from N-terminus to C-terminus, three heavy chain complementarity-determining regions (CDRs), HCDR1, HCDR2, and HCDR3; and
a light chain variable (VL) domain comprising from N-terminus to C-terminus, three light chain complementarity-determining regions (CDRs), LCDR1, LCDR2, and LCDR3; wherein
(a) the HCDR1 comprises an amino acid sequence X10-A-X11-T, optionally wherein the HCDR1 comprises an amino acid sequence Y-A-X12-T or X13-A-M-T, wherein X10 is N or Y, X1I is M or V, X12 is M or V, and X13 is N or Y;
(b) the HCDR2 comprises an amino acid sequence selected from the group consisting of F-X67-X68-X69-X70-G-X71-X72-Y-Y-A-N-W-A-K-G (SEQ ID NO: 321), F-X73-G-I-X74-G-X75-X76-Y-Y-A-N-W-A-K-G (SEQ ID NO: 322), F-X77-X78-X79-R-G-X80-I-Y-Y-A-N-W-A-K-G (SEQ ID NO: 323), and F-I-G-I-R-G-X81-I-Y-Y-A-N-W-A-K-G (SEQ ID NO: 324) wherein X67 is I or V, X68 is A or G, X69 is I or L, X70 is R or Y, X71 is D, H, or N, X72 is F or I, X73 is I or V, X74 is R or Y, X75 is D, N, H, X76 is F or I, X77 is I or V, X78 is A or G, X79 is I or L, X80 is H or N, and X81 is H or N;
(c) the HCDR3 comprises an amino acid sequence G-G-L-X114-T-G-X115-S-Y-F-D-L (SEQ ID NO: 338), wherein X114 is W or Y, and X115 is N or Y;
(d) the LCDR1 comprises an amino acid sequence Q-A-S-E-X154-X155-X156-X157-Y-L-A (SEQ ID NO: 350), or Q-A-S-E-X158-I-X159-R-Y-L-A (SEQ ID NO: 351) or Q-A-S-E-S-L-S-S-Y-L-A (SEQ ID NO: 77), wherein X154 is N or S, X155 is I or L, X156 is N or S, X157 is R or S, X158 is N or S, and X159 is N or S;
(e) the LCDR2 comprises an amino acid sequence R-A-A-T-L-A-S(SEQ ID NO: 92) or R-A-S-T-L-A-S(SEQ ID NO: 93); and
(f) the LCDR3 comprises an amino acid sequence Q-Q-G-Y-G-Y-S-T-V-D-N-A (SEQ ID NO: 211) or Q-Q-G-Y-G-Y-S-T-V-G-N-A (SEQ ID NO: 112).

23-29. (canceled)

30. The isolated antibody, or the antigen binding fragment thereof, of claim 1, wherein the antibody, or the antigen binding fragment thereof, (i) competes for binding to human Trop-2 with a monoclonal antibody of selected from the group consisting of 9F7, 2H3, 69H10, 65D5, 3E9, 9A5, 12G6, 1D10, 62E3, 3A4, 63H3, 66A1, 2E12, 72F12, 74F11, and 15F5;
(ii) specifically binds to a human and/or a cynomolgus Trop-2;
(iii) specifically binds to a cell surface human Trop-2;
(iv) induces ADCC;
(v) induces ADCP;
(vi) induces CDC;
(vii) induces internalization of the antibody, or the antigen binding fragment thereof, or a conjugate thereof; or
(viii) any combination of (i) to (vii).

31. (canceled)

32. The antibody, or the antigen binding fragment thereof, of claim 1, wherein the antibody is a humanized antibody or a chimeric antibody, or antigen binding fragment thereof.

33. The antibody, or the antigen binding fragment thereof, of claim 1, wherein the antibody, or antigen binding fragment thereof, comprises a heavy chain constant region of a class selected from IgA, IgD, IgE, IgG, or IgM.

34. The antibody, or the antigen binding fragment thereof, of claim 33, wherein the antibody, or antigen binding fragment thereof, comprises a heavy chain constant region of the class IgG, and wherein the IgG is selected from the group consisting of IgG1, IgG2, IgG3, and IgG4.

35. An isolated polynucleotide encoding the antibody, or the antigen binding fragment thereof, of claim 1, an HCVR thereof, an LCVR thereof, a light chain thereof, a heavy chain thereof, or an antigen binding fragment thereof.

36-38. (canceled)

39. A pharmaceutical composition comprising the antibody, or the antigen binding fragment thereof, of claim 1, and a pharmaceutically acceptable carrier or diluent.

40-47. (canceled)

48. A method of treating cancer in a subject, comprising administering an isolated antibody, or the antigen binding fragment thereof, of claim 1 thereby treating the cancer.

49. The method of claim 48, further comprising administering an additional therapeutic agent.

50-60. (canceled)

61. The isolated antibody, or the antigen binding fragment thereof, of claim 8, wherein the antibody, or the antigen binding fragment thereof,
(i) competes for binding to human Trop-2 with a monoclonal antibody of selected from the group consisting of 9D7, 9D7-2, 10A9, 10A9-2, 62A2, 74E4, 66A6, 10E12, 8H2, 74H11, 74A1, 10F12, 65E11, 69C2, 61F12, 10D3, 67H4, 64G9, 72G12, 65G8, 69D8, 2H5, 64A6, and 62B10;
(ii) specifically binds to a human and/or a cynomolgus Trop-2;
(iii) specifically binds to a cell surface human Trop-2;
(iv) induces ADCC;
(v) induces ADCP;
(vi) induces CDC;
(vii) induces internalization of the antibody, or the antigen binding fragment thereof, or a conjugate thereof; or
(viii) any combination of (i) to (vii).

62. The antibody, or the antigen binding fragment thereof, of claim 8, wherein the antibody is a humanized antibody or a chimeric antibody, or antigen binding fragment thereof.

63. The antibody, or the antigen binding fragment thereof, of claim 8, wherein the antibody, or antigen binding fragment thereof, comprises a heavy chain constant region of a class selected from IgA, IgD, IgE, IgG, or IgM.

64. The antibody, or the antigen binding fragment thereof, of claim 63, wherein the antibody, or antigen binding fragment thereof, comprises a heavy chain constant region of the class IgG, and wherein the IgG is selected from the group consisting of IgG1, IgG2, IgG3, and IgG4.

65. An isolated polynucleotide encoding the antibody, or the antigen binding fragment thereof, of claim 8, an HCVR thereof, an LCVR thereof, a light chain thereof, a heavy chain thereof, or an antigen binding fragment thereof.

66. A pharmaceutical composition comprising the antibody, or the antigen binding fragment thereof, of claim 8, and a pharmaceutically acceptable carrier or diluent.

67. A method of treating cancer in a subject, comprising administering an isolated antibody, or the antigen binding fragment thereof, of claim 8, thereby treating the cancer.

68. The method of claim 67, further comprising administering an additional therapeutic agent.
